(19) United States
(12) Patent Application Publication
(10) Pub. No.: US 2024/0368078 A1
(43) Pub. Date: Nov. 7, 2024

(54) ARYL HETEROCYCLIC COMPOUNDS AS Kv1.3 POTASSIUM SHAKER CHANNEL BLOCKERS

(71) Applicant: D.E. SHAW RESEARCH, LLC, New York, IN (US)

(72) Inventors: Fabrizio GIORDANETTO, New York, NY (US); Morten Østergaard JENSEN, Hellerup (DK); Vishwanath JOGINI, Hyderabad (IN); Roger John SNOW, Danbury, CT (US)

(21) Appl. No.: 18/553,339

(22) PCT Filed: Mar. 29, 2022
(86) PCT No.: PCT/US2022/022230

§ 371 Date: Sep. 29, 2023

Related U.S. Application Data
(60) Provisional application No. 63/168,056, filed on Mar. 30, 2021.

(51) Int. Cl. C07D 207/09; A61K 31/40; A61K 31/4025; A61K 31/404; A61K 31/4155; A61K 31/4178; A61K 31/4196; A61K 31/422; A61K 31/4245; A61K 31/427; A61K 31/4439; A61K 31/444; A61K 31/454; A61K 31/4725; A61K 31/506; A61K 31/53; C07D 401/06; C07D 401/12; C07D 401/14; C07D 403/06; C07D 403/14; C07D 405/04; C07D 405/14; C07D 409/12; C07D 413/06

(52) U.S. Cl. CPC C07D 207/09; A61K 31/40; A61K 31/4025; A61K 31/404; A61K 31/4155; A61K 31/4178; A61K 31/4196; A61K 31/422; A61K 31/4245; A61K 31/427; A61K 31/4439; A61K 31/444; A61K 31/454; A61K 31/4725; A61K 31/506; A61K 31/53; C07D 401/06; C07D 401/12; C07D 401/14; C07D 403/06; C07D 403/14; C07D 405/04; C07D 405/14; C07D 409/12; C07D 413/06

(57) ABSTRACT

A compound of Formula I

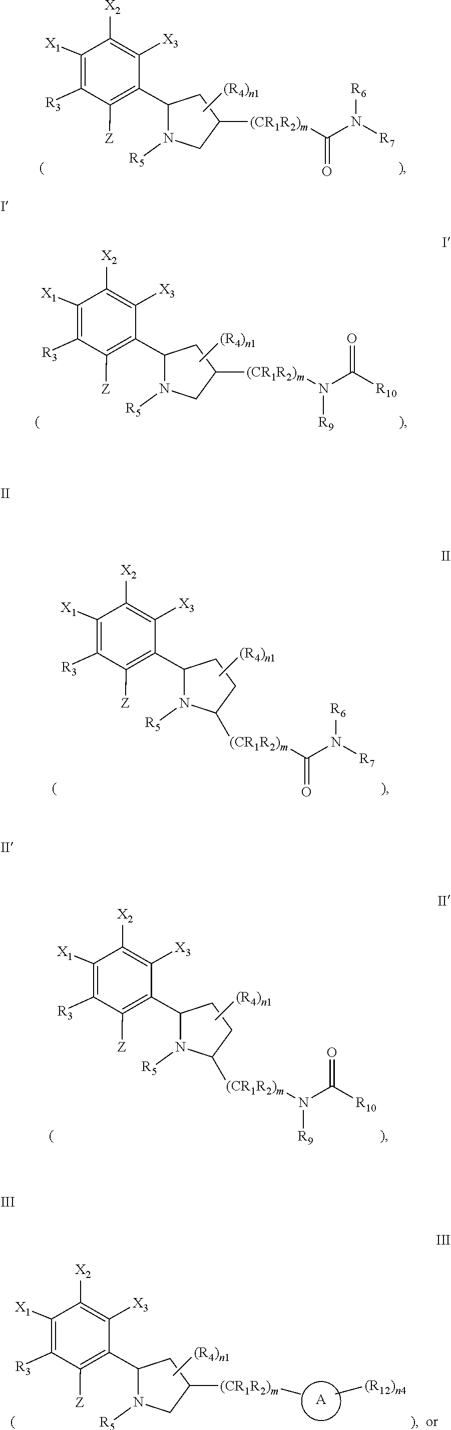

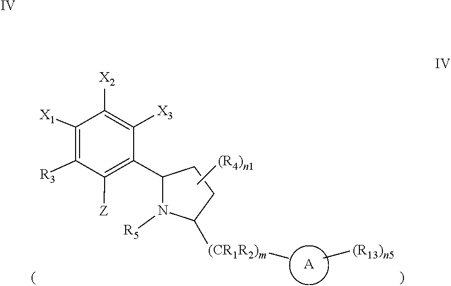

or a pharmaceutically-acceptable salt thereof, is described, wherein the substituents are as defined herein. Pharmaceutical compositions comprising the same and method of using the same are also described.

This application claims the benefit of and priority to U.S. Provisional Patent Application No. 63/168,056, filed on Mar. 30, 2021, the content of which is hereby incorporated by reference in its entirety.

This patent disclosure contains material that is subject to copyright protection. The copyright owner has no objection to the facsimile reproduction of the patent document or the patent disclosure as it appears in the U.S. Patent and Trademark Office patent file or records, but otherwise reserves any and all copyright rights.

### INCORPORATION BY REFERENCE

All documents cited herein are incorporated herein by reference in their entirety.

### FIELD OF THE INVENTION

The invention relates generally to the field of pharmaceutical science. More particularly, the invention relates to compounds and compositions useful as pharmaceuticals as potassium channel blockers.

### BACKGROUND

Voltage-gated Kv1.3 potassium (K⁺) channels are expressed in lymphocytes (T and B lymphocytes), the central nervous system, and other tissues, and regulate a large number of physiological processes such as neurotransmitter release, heart rate, insulin secretion, and neuronal excitability. Kv1.3 channels can regulate membrane potential and thereby indirectly influence calcium signaling in human effector memory T cells. Effector memory T cells are mediators of several conditions, including multiple sclerosis, type I diabetes mellitus, psoriasis, spondylitis, parodontitis, and rheumatoid arthritis. Upon activation, effector-memory T cells increase expression of the Kv1.3 channel. Amongst human B cells, naive and early memory B cells express small numbers of Kv1.3 channels when they are quiescent. In contrast, class-switched memory B cells express high numbers of Kv1.3 channels. Furthermore, the Kv1.3 channel promotes the calcium homeostasis required for T-cell receptor-mediated cell activation, gene transcription, and proliferation (Panyi, G., et al., 2004, Trends Immunol., 565-569). Blockade of Kv1.3 channels in effector memory T cells suppresses activities like calcium signaling, cytokine production (interferon-gamma, interleukin 2), and cell proliferation.

Autoimmune disease is a family of disorders resulting from tissue damage caused by attack from the body's own immune system. Such diseases may affect a single organ, as in multiple sclerosis and type I diabetes mellitus, or may involve multiple organs, as in the case of rheumatoid arthritis and systemic lupus erythematosus. Treatment is generally palliative, with anti-inflammatory and immunosuppressive drugs, which can have severe side effects. A need for more effective therapies has led to a search for drugs that can selectively inhibit the function of effector memory T cells, known to be involved in the etiology of autoimmune diseases. These inhibitors are thought to be able to ameliorate autoimmune diseases symptoms without compromising the protective immune response. Effector memory T cells (“TEMs”) express high numbers of the Kv1.3 channel and depend on these channels for their function. In vivo, Kv1.3 channel blockers paralyze TEMs at the sites of inflammation and prevent their reactivation in inflamed tissues. Kv1.3 channel blockers do not affect the motility within lymph nodes of naive and central memory T cells. Suppressing the function of these cells by selectively blocking the Kv1.3 channel offers the potential for effective therapy of autoimmune diseases with minimal side effects.

Multiple sclerosis (“MS”) is caused by autoimmune damage to the central nervous system (“CNS”). Symptoms include muscle weakness and paralysis, which severely affect quality of life for patients. MS progresses rapidly and unpredictably and eventually leads to death. The Kv1.3 channel is also highly expressed in auto-reactive TEMs from MS patients (Wulff H., et al., 2003, J. Clin. Invest., 1703-1713; Rus H., et al., 2005, PNAS, 11094-11099). Animal models of MS have been successfully treated using blockers of the Kv1.3 channel.

Compounds which are selective Kv1.3 channel blockers are thus potential therapeutic agents as immunosuppressants or immune system modulators. The Kv1.3 channel is also considered as a therapeutic target for the treatment of obesity and for enhancing peripheral insulin sensitivity in patients with type 2 diabetes mellitus. These compounds can also be utilized in the prevention of graft rejection and the treatment of immunological (e.g., autoimmune) and inflammatory disorders.

Tubulointerstitial fibrosis is a progressive connective tissue deposition on the kidney parenchyma, leading to renal function deterioration, is involved in the pathology of chronic kidney disease, chronic renal failure, nephritis, and inflammation in glomeruli, and is a common cause of end-stage renal failure. Overexpression of Kv1.3 channels in lymphocytes can promote their proliferation, leading to chronic inflammation and overstimulation of cellular immunity, which are involved in the underlying pathology of these renal diseases and are contributing factors in the progression of tubulointerstitial fibrosis. Inhibition of the lymphocyte Kv1.3 channel currents suppress proliferation of kidney lymphocytes and ameliorate the progression of renal fibrosis (Kazama I., et al., 2015, Mediators Inflamm., 1-12).

Kv1.3 channels also play a role in gastroenterological disorders including inflammatory bowel diseases (“IBDs”) such as ulcerative colitis (“UC”) and Crohn's disease. UC is a chronic IBD characterized by excessive T cell infiltration and cytokine production. UC can impair quality of life and can lead to life-threatening complications. High levels of Kv1.3 channels in CD4 and CD8 positive T cells in the inflamed mucosa of UC patients have been associated with production of pro-inflammatory compounds in active UC. Kv1.3 channels are thought to serve as a marker of disease activity and pharmacological blockade might constitute a novel immunosuppressive strategy in UC. Present treatment regimens for UC, including corticosteroids, salicylates, and anti-TNF-α reagents, are insufficient for many patients (Hansen L. K., et al., 2014, J. Crohns Colitis, 1378-1391). Crohn's disease is a type of IBD which may affect any part of the gastrointestinal tract. Crohn's disease is thought to be the result of intestinal inflammation due to a T cell-driven process initiated by normally safe bacteria. Thus, Kv1.3 channel inhibition can be utilized in treating the Crohn's disease.

In addition to T cells, Kv1.3 channels are also expressed in microglia, where the channel is involved in inflammatory cytokine and nitric oxide production and in microglia-mediated neuronal killing. In humans, strong Kv1.3 channel expression has been found in microglia in the frontal cortex of patients with Alzheimer's disease and on CD68⁺ cells in MS brain lesions. It has been suggested that Kv1.3 channel blockers might be able to preferentially target detrimental proinflammatory microglia functions. Kv1.3 channels are expressed on activated microglia in infarcted rodent and human brain. Higher Kv1.3 channel current densities are observed in acutely isolated microglia from the infarcted hemisphere than in microglia isolated from the contralateral hemisphere of a mouse model of stroke (Chen Y. J., et al., 2017, Ann. Clin. Transl. Neurol., 147-161).

Expression of Kv1.3 channels is elevated in microglia of human Alzheimer's disease brains, suggesting that Kv1.3 channel is a pathologically relevant microglial target in Alzheimer's disease (Rangaraju S., et al., 2015, J. Alzheimers Dis., 797-808). Soluble APO enhances microglial Kv1.3 channel activity. Kv1.3 channels are required for APO-induced microglial pro-inflammatory activation and neurotoxicity. Kv1.3 channel expression/activity is upregulated in transgenic Alzheimer's disease animals and human Alzheimer's disease brains. Pharmacological targeting of microglial Kv1.3 channels can affect hippocampal synaptic plasticity and reduce amyloid deposition in APP/PS1 mice. Thus, Kv1.3 channel may be a therapeutic target for Alzheimer's disease.

Kv1.3 channel blockers could be also useful for ameliorating pathology in cardiovascular disorders such as ischemic stroke, where activated microglia significantly contributes to the secondary expansion of the infarct.

Kv1.3 channel expression is associated with the control of proliferation in multiple cell types, apoptosis, and cell survival. These processes are crucial for cancer progression. In this context, Kv1.3 channels located in the inner mitochondrial membrane can interact with the apoptosis regulator Bax (Serrano-Albarras, A., et al., 2018, Expert Opin. Ther. Targets, 101-105). Thus, inhibitors of Kv1.3 channels may be used as anticancer agents.

A number of peptide toxins with multiple disulfide bonds from spiders, scorpions, and anemones are known to block Kv1.3 channels. A few selective, potent peptide inhibitors of the Kv1.3 channel have been developed. A synthetic derivative of stichodactyla toxin (“shk”) with an unnatural amino acid (shk-186) is the most advanced peptide toxin. Shk has demonstrated efficacy in preclinical models and is currently in a phase I clinical trial for treatment of psoriasis. Shk can suppress proliferation of TEMs, resulting in improved condition in animal models of multiple sclerosis. Unfortunately, Shk also binds to the closely-related Kvi channel subtype found in CNS and heart. There is a need for Kv1.3 channel-selective inhibitors to avoid potential cardio- and neuro-toxicity. Additionally, small peptides like shk-186 are rapidly cleared from the body after administration, resulting in short circulating half-lives and frequent administration events. Thus, there is a need for the development of long-acting, selective Kv1.3 channel inhibitors for the treatment of chronic inflammatory diseases.

Thus, there remains a need for development of novel Kv1.3 channel blockers as pharmaceutical agents.

### SUMMARY OF THE INVENTION

In one aspect, compounds useful as potassium channel blockers having a structure of

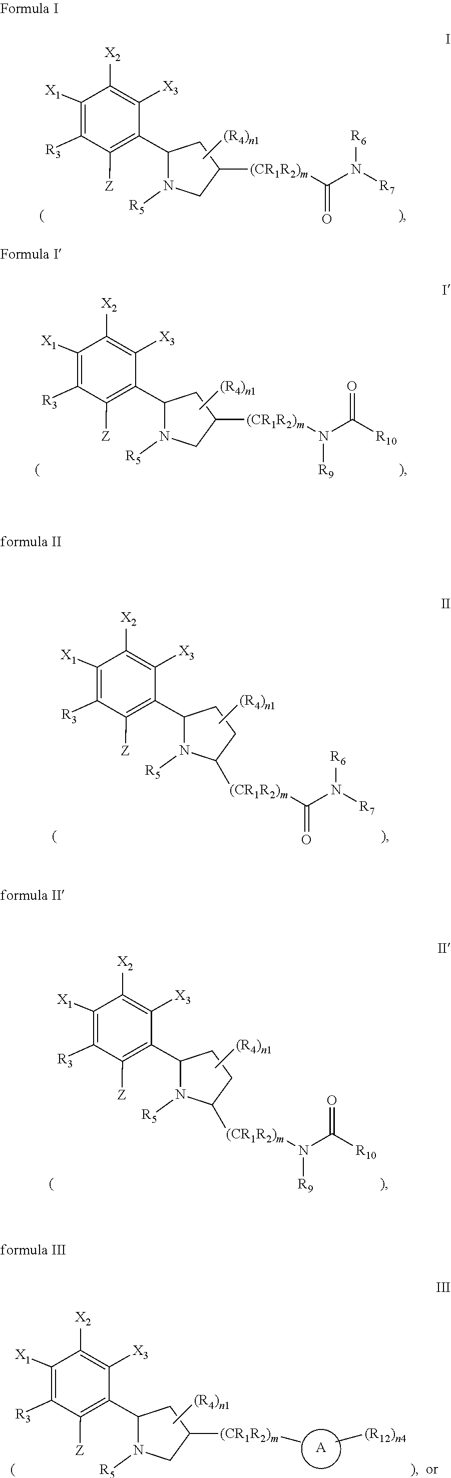

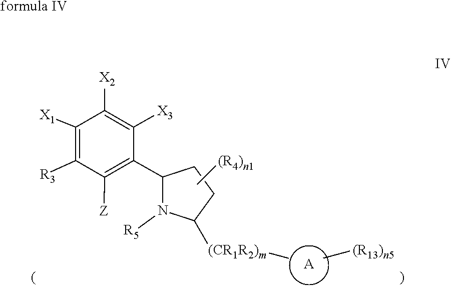

are described, where the various substituents are defined herein. The compounds of Formula I, I′, II, II′, III, or IV described herein can block Kv1.3 potassium (K⁺) channels and be used in the treatment of a variety of conditions. Methods for synthesizing these compounds are also described herein. Pharmaceutical compositions and methods of using these compositions described herein are useful for treating conditions in vitro and in vivo. Such compounds, pharmaceutical compositions, and methods of treatment have a number of clinical applications, including as pharmaceutically active agents and methods for treating cancer, an immunological disorder, a CNS disorder, an inflammatory disorder, a gastroenterological disorder, a metabolic disorder, a cardiovascular disorder, a kidney disease, or a combination thereof.

In one aspect, a compound of Formula I, I′, II, II′, III, or IV or a pharmaceutically-acceptable salt thereof is described,

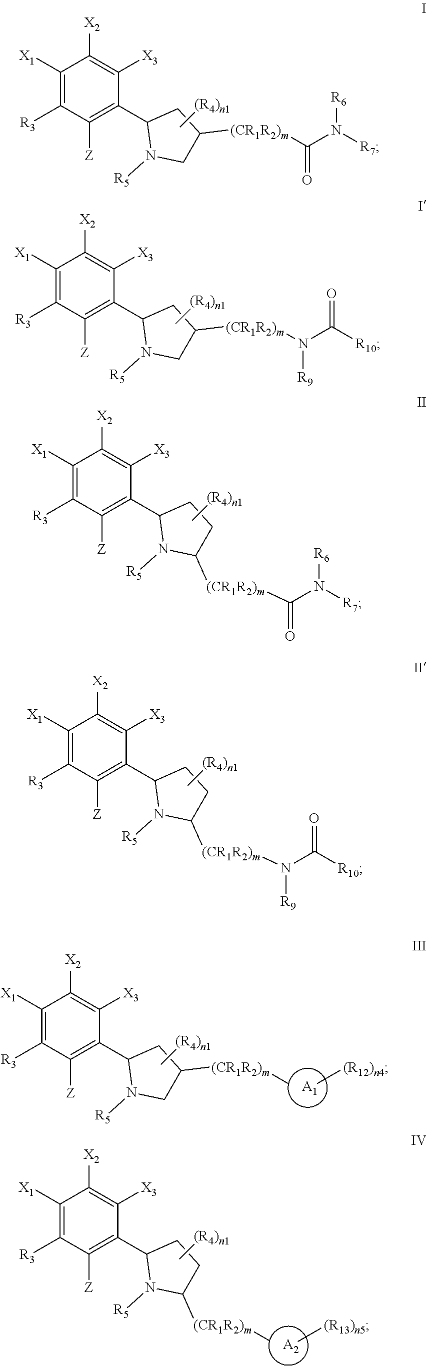

where
each occurrence of Z is independently ORₐ;
each occurrence of X₁ is independently H, halogen, CN, alkyl, cycloalkyl, halogenated cycloalkyl, or halogenated alkyl;
each occurrence of X₂ is independently H, halogen, CN, alkyl, cycloalkyl, halogenated cycloalkyl, or halogenated alkyl;
each occurrence of X₃ is independently H, halogen, CN, alkyl, cycloalkyl, halogenated cycloalkyl, or halogenated alkyl;
or alternatively X₁ and X₂ and the carbon atoms they are connected to taken together form an optionally substituted 5- or 6-membered aryl;
or alternatively X₂ and X₃ and the carbon atoms they are connected to taken together form an optionally substituted 5- or 6-membered aryl;
each occurrence of R₁ is independently H, alkyl, cycloalkyl, saturated heterocycle, aryl, heteroaryl, alkylaryl, alkylheteroaryl, CN, CF₃, OCF₃, ORₐ, SRₐ, halogen, NRₐR_{b}, or NR_{b}(C═O)Rₐ;
each occurrence of R₂ is independently H, alkyl, cycloalkyl, saturated heterocycle, aryl, heteroaryl, alkylaryl, alkylheteroaryl, CN, CF₃, OCF₃, ORₐ, SRₐ, halogen, NRₐR_{b}, or NR_{b}(C═O)Rₐ;
or alternatively R₁ and R₂ taken together with the carbon atom they are connected to form a cycloalkyl or saturated heterocycle;
each occurrence of R₃ is independently H, alkyl, cycloalkyl, saturated heterocycle, aryl, heteroaryl, CN, CF₃, OCF₃, ORₐ, SRₐ, halogen, NRₐR_{b}, or NR_{b}(C═O)Rₐ;
each occurrence of R₄ is independently H, alkyl, cycloalkyl, saturated heterocycle, (CRₐR_{b})ₙ₂ORₐ, or (CRₐR_{b})ₙ₂NRₐR_{b};
or alternatively two R₄ groups taken together with the carbon atom(s) that they are connected to form a 3-7 membered optionally substituted cycloalkyl or heterocycle;
each occurrence of R₅ is independently H, alkyl, cycloalkyl, saturated heterocycle, aryl, heteroaryl, alkylaryl, alkylheteroaryl, (C═O)Rₐ, (C═O)(CRₐR_{b})ₙ₂ORₐ, (C═O)(CRₐR_{b})ₙ₂NRₐR_{b}, or SO₂Rₐ;
each occurrence of R₆ is independently H, alkyl, cycloalkyl, heterocycle, aryl, heteroaryl, alkylaryl, or alkylheteroaryl;
each occurrence of R₇ is independently H, alkyl, cycloalkyl, heterocycle, aryl, heteroaryl, alkylaryl, or alkylheteroaryl;
or alternatively R₆ and R₇ taken together with the nitrogen atom they are connected to form a heterocycle comprising the nitrogen atom and 0-3 additional heteroatoms each selected from the group consisting of N, O, and S; wherein the heterocycle is optionally substituted by 1-4 substituents each independently selected from the group consisting of alkyl, cycloalkyl, halogenated cycloalkyl, halogenated alkyl, halogen, CN, OR₈, —(CH₂)₀₋₂OR₈, N(R₈)₂, (C═O)R₈, (C═O)N(R₈)₂, NR₈(C═O)R₈, and oxo where valence permits;
each occurrence of R₉ is independently H, alkyl, cycloalkyl, saturated heterocycle, aryl, heteroaryl, alkylaryl, alkylheteroaryl, (C═O)Rₐ, (C═O)(CRₐR_{b})ₙ₂ORₐ, (C═O)(CRₐR_{b})ₙ₂NRₐR_{b}, or SO₂Rₐ;
each occurrence of R₁₀ is independently H, alkyl, cycloalkyl, heterocycle, aryl, heteroaryl, alkylaryl, or alkylheteroaryl;
A₁ is aryl or heteroaryl;
A₂ is aryl or heteroaryl;
each occurrence of R₁₂ is independently H, alkyl, CN, CF₃, OCF₃, ORₐ, SRₐ, halogen, NRₐR_{b}, (CRₐR_{b})ₙ₂ORₐ, (C═O)NRₐR_{b}, (CRₐR_{b})ₙ₂NRₐR_{b}, or (CRₐR_{b})ₙ₂NR_{b}(C═O)Rₐ;
each occurrence of R₁₃ is independently H, alkyl, CN, CF₃, OCF₃, ORₐ, SRₐ, halogen, NRₐR_{b}, (CRₐR_{b})ₙ₂ORₐ, (C═O)NRₐR_{b}, (CRₐR_{b})ₙ₂NRₐR_{b}, or (CRₐR_{b})ₙ₂NR_{b}(C═O)Rₐ;
each occurrence of Rₐ and R_{b} are independently H, alkyl, alkenyl, cycloalkyl, saturated heterocycle comprising 1-3 heteroatoms each selected from the group consisting of N, O, and S, aryl, or heteroaryl; or alternatively Rₐ and R_{b} together with the carbon or nitrogen atom that they are connected to form a cycloalkyl or heterocycle comprising the nitrogen atom and 0-3 additional heteroatoms each selected from the group consisting of N, O, and S;
the alkyl, cycloalkyl, heterocycle, aryl, and heteroaryl in X₁, X₂, X₃, A₁, A₂, R₁, R₂, R₃, R₄, R₅, R₆, R₇, R₉, R₁₀, R₁₂, R₁₃, Rₐ, or R_{b}, where applicable, are optionally substituted by 1-4 substituents each independently selected from the group consisting of alkyl, cycloalkyl, halogenated cycloalkyl, halogenated alkyl, halogen, CN, OR₈, —(CH₂)₀₋₂OR₈, N(R₈)2, (C═O)R₈, (C═O)N(R₈)2, NR₈(C═O)R₈, and oxo where valence permits;
each occurrence of R₈ is independently H, alkyl, or optionally substituted heterocycle; or alternatively the two R₈ groups together with the nitrogen atom that they are connected to form an optionally substituted heterocycle comprising the nitrogen atom and 0-3 additional heteroatoms each selected from the group consisting of N, O, and S;
each occurrence of m is independently 1, 2, or 3;
each occurrence of n₁ is independently an integer from 0-3 wherein valence permits;
each occurrence of n₂ is independently an integer from 0-3; and
n₄ is an integer from 0-3;
n₅ is an integer from 0-3.

In any one of the embodiments described herein, each occurrence of R₄ is independently H, alkyl, cycloalkyl, saturated heterocycle, (CRₐR_{b})ₙ₂NRₐR_{b}, or (CRₐR_{b})n₂ORₐ; and each occurrence of R₅ is independently H, alkyl, cycloalkyl, or saturated heterocycle.

In any one of the embodiments described herein, each occurrence of m is independently 2 or 3.

In any one of the embodiments described herein, one or more occurrences of m are 1.

In any one of the embodiments described herein, the compound has the structure of Formula Ia, Ia′, IIa, IIa′, IIIa, or IVa:

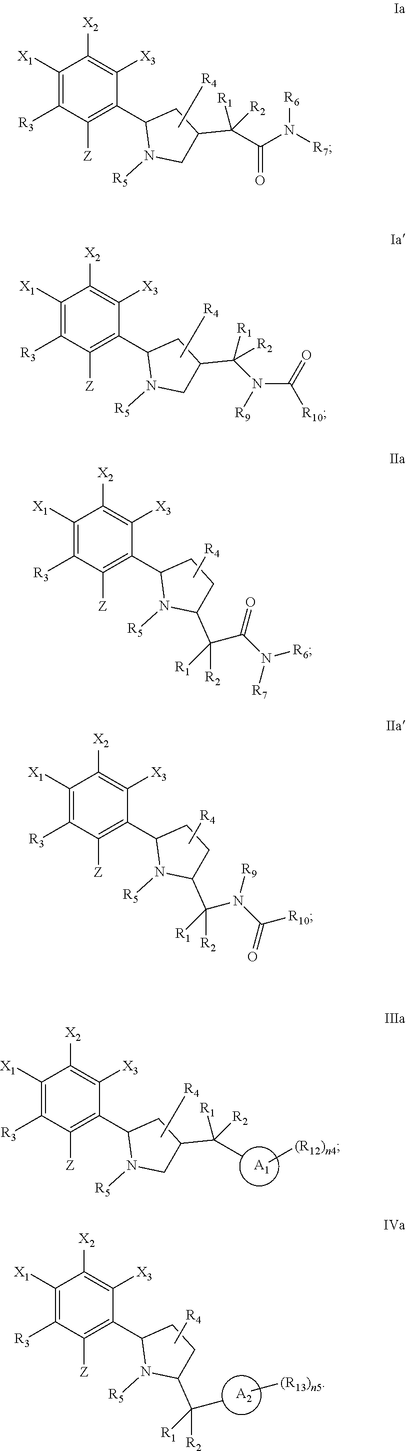

In any one of the embodiments described herein, the compound has the structure of Formula Ib, Ib′, IIb, IIb′, IIIb, or IVb.

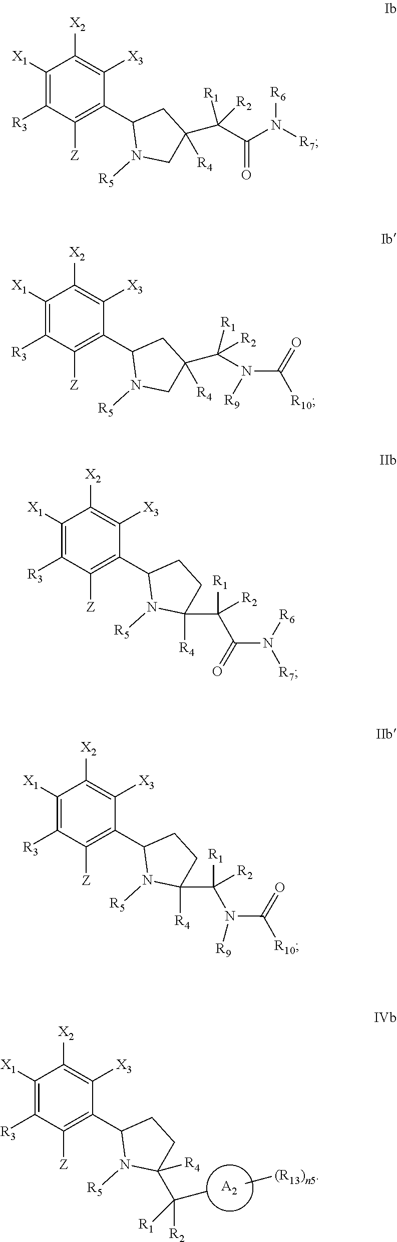

In any one of the embodiments described herein, one or more occurrences of R₄ are H, alkyl, cycloalkyl, or ORₐ.

In any one of the embodiments described herein, one or more occurrences of R₄ are H or alkyl.

In any one of the embodiments described herein, one or more occurrences of R₄ are H or CH₃.

In any one of the embodiments described herein, one or more occurrences of R₄ are saturated heterocycle, (CRₐR_{b})ₙ₂ORₐ, or (CRₐR_{b})ₙ₂NRₐR_{b}.

In any one of the embodiments described herein, one or more occurrences of n₁ are 1.

In any one of the embodiments described herein, one or more occurrences of n₁ are 0.

In any one of the embodiments described herein, each occurrence of R₅ is independently H, alkyl, cycloalkyl, or saturated heterocycle.

In any one of the embodiments described herein, each occurrence of R₅ is independently cycloalkyl or saturated heterocycle.

In any one of the embodiments described herein, each occurrence of R₅ is independently H or alkyl.

In any one of the embodiments described herein, each occurrence of R₅ is independently H or CH₃.

In any one of the embodiments described herein, each occurrence of R₁ and R₂ is independently cycloalkyl, saturated heterocycle, aryl, heteroaryl, alkylaryl, alkylheteroaryl, CN, CF₃, OCF₃, ORₐ, SRₐ, halogen, NRₐR_{b}, or NR_{b}(C═O)Rₐ.

In any one of the embodiments described herein, each occurrence of R₁ and R₂ is independently H, alkyl optionally substituted with OR₈, halogen, cycloalkyl, or fluorinated alkyl.

In any one of the embodiments described herein, each occurrence of R₁ and R₂ is

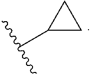 [SMILES: *C1CC1]

independently H, CH₃, CH₂CH₃, CH₂OH, CH₂CH₂OH, CH₂OCH₃, CH₂CH₂OCH₃, or.

In any one of the embodiments described herein, R₁ and R₂ are H and H, H and Me, Me and Me, H and Et, Me and Et, or Et and Et, H and CH₂OH, H and CH₂CH₂OH, H and

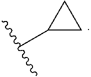 [SMILES: *C1CC1]

CH₂OCH₃, H and CH₂CH₂OCH₃, or H and.

In any one of the embodiments described herein, each occurrence of the structural moiety —(CR₁R₂)ₘ— is independently selected from the group consisting of —CH₂—, —CH(CH₃)—, —CH₂—C(CH₃)₂—,

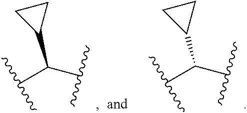

and

In any one of the embodiments described herein, each occurrence of R₆ and R₇ is—independently H, alkyl, cycloalkyl, or heterocycle; wherein the alkyl, cycloalkyl, heterocycle is optionally substituted by 1-2 substituents each independently selected from the group consisting of halogen, CN, OH, OMe, —(CH₂)₁₋₂OMe and —(CH₂)₁₋₂OH.

In any one of the embodiments described herein, each occurrence of R₆ and R₇ is independently H or alkyl; wherein the alkyl is optionally substituted by 1-2 substituents each independently selected from the group consisting of halogen, CN, and OH.

In any one of the embodiments described herein, each occurrence of R₆ and R₇ is independently H, —CH₃, —CH₂OH, —CH₂CH₂OH or —CH₂CH₂CH₂OH.

In any one of the embodiments described herein, R₆ and R₇ taken together with the nitrogen atom they are connected to form a heterocycle comprising the nitrogen atom and 0-3 additional heteroatoms each selected from the group consisting of N, O, and S; wherein the heterocycle is optionally substituted by 1-4 substituents each independently selected from the group consisting of alkyl, cycloalkyl, halogenated cycloalkyl, halogenated alkyl, halogen, CN, OR₈, —(CH₂)₀₋₂OR₈, N(R₈)₂, (C═O)N(R₈)₂, (C═O)R₈, NR₈(C═O)R₈, and oxo where valence permits.

In any one of the embodiments described herein, R₆ and R₇ taken together with the nitrogen atom they are connected to form a 4-, 5-, or 6-membered heterocycle; wherein the heterocycle is optionally substituted by 1-2 substituents each independently selected from the group consisting of alkyl, halogenated alkyl, halogen, CN, OH, and —(CH₂)₁₋₂OH.

In any one of the embodiments described herein, the 4-, 5-, or 6-membered heterocycle is azetidine, pyrrolidine, piperidine, or piperazine.

In any one of the embodiments described herein, the 4-, 5-, or 6-membered heterocycle is substituted by 1-2 substituents each independently selected from the group consisting of OH and —(CH₂)₁₋₂OH.

In any one of the embodiments described herein, R₆ and R₇ taken together with the nitrogen atom they are connected to form azetidine.

In any one of the embodiments described herein, R₆ and R₇ taken together with the nitrogen atom they are connected to form pyrrolidine.

In any one of the embodiments described herein, each occurrence of R₆ and R₇ is independently alkylaryl, or alkylheteroaryl.

In any one of the embodiments described herein, each occurrence of the structural moiety

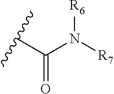

independently has the structure of

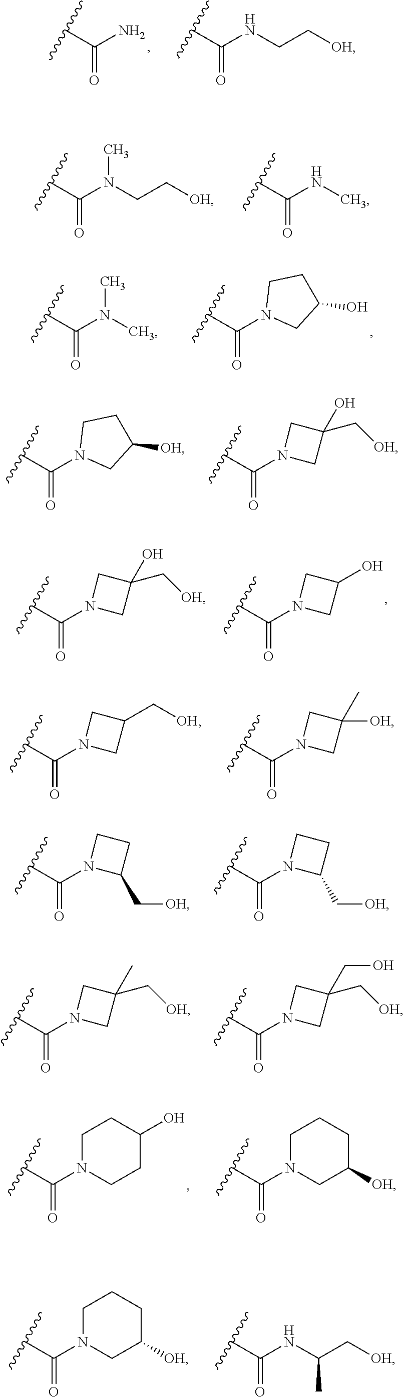

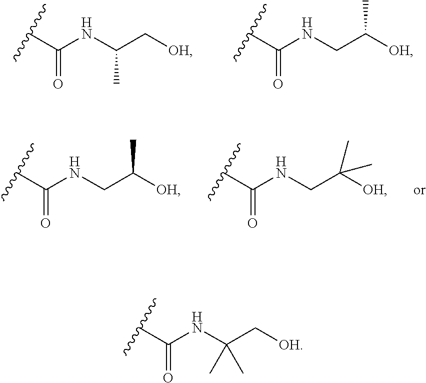

In any one of the embodiments described herein, each occurrence of R₉ is independently cycloalkyl, saturated heterocycle, aryl, heteroaryl, alkylaryl, or alkylheteroaryl.

In any one of the embodiments described herein, each occurrence of R₉ is independently (C═O)Rₐ, (C═O)(CRₐR_{b})ₙ₂ORₐ, (C═O)(CRₐR_{b})ₙ₂NRₐR_{b}, (C═O)NRₐR_{b}, or SO₂Rₐ.

In any one of the embodiments described herein, each occurrence of R₉ is independently H or alkyl.

In any one of the embodiments described herein, each occurrence of R₉ is independently H or CH₃.

In any one of the embodiments described herein, each occurrence of R₁₀ is independently H, alkyl, cycloalkyl, or heterocycle; wherein the alkyl, cycloalkyl, heterocycle is optionally substituted by 1-2 substituents each independently selected from the group consisting of halogen, CN, OH, OMe, —(CH₂)₁₋₂OMe and —(CH₂)₁₋₂OH.

In any one of the embodiments described herein, one or more occurrences of R₁₀ are alkyl; wherein the alkyl is optionally substituted by 1-2 substituents each independently selected from the group consisting of halogen, CN, and OH.

In any one of the embodiments described herein, each occurrence of R₁₀ is independently H, —CH₃, —CH₂OH, or —CH₂CH₂OH.

In any one of the embodiments described herein, A₁ is a 5- or 6-membered aryl or heteroaryl.

In any one of the embodiments described herein, A₁ is selected from the group consisting of

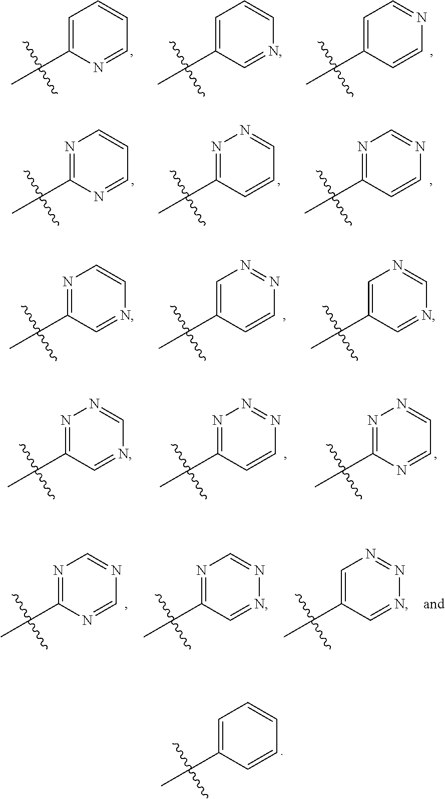

In any one of the embodiments described herein, A₁ is selected from the group consisting of

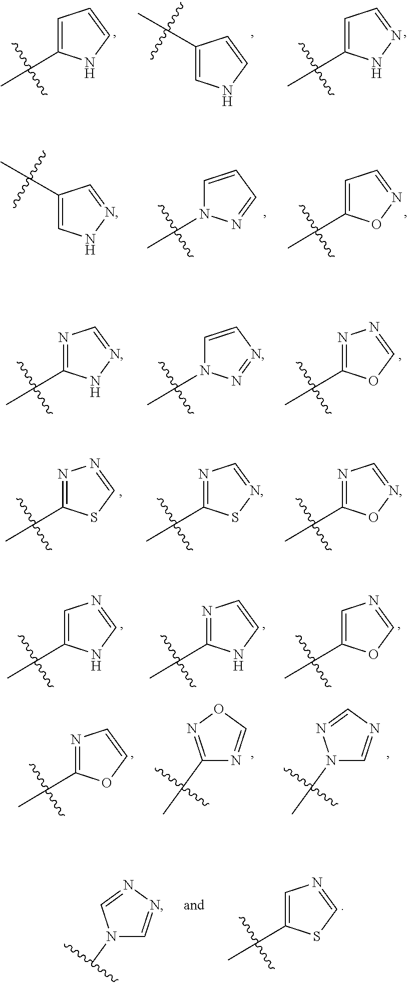

In any one of the embodiments described herein, A₁ is

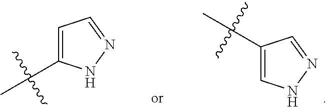 [SMILES: *c1ccn[nH]1 ; *c1cn[nH]c1]

In any one of the embodiments described herein, each occurrence of R₁₂ is independently H, halogen, fluorinated alkyl, or alkyl.

In any one of the embodiments described herein, one or more occurrences of R₁₂ are H.

In any one of the embodiments described herein, A₂ is a 5- or 6-membered aryl or heteroaryl.

In any one of the embodiments described herein, A₂ is selected from the group consisting of

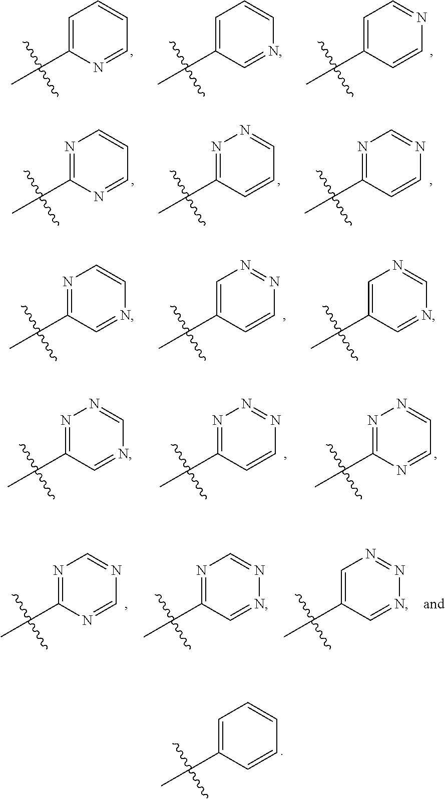

In any one of the embodiments described herein, A₂ is selected from the group consisting of

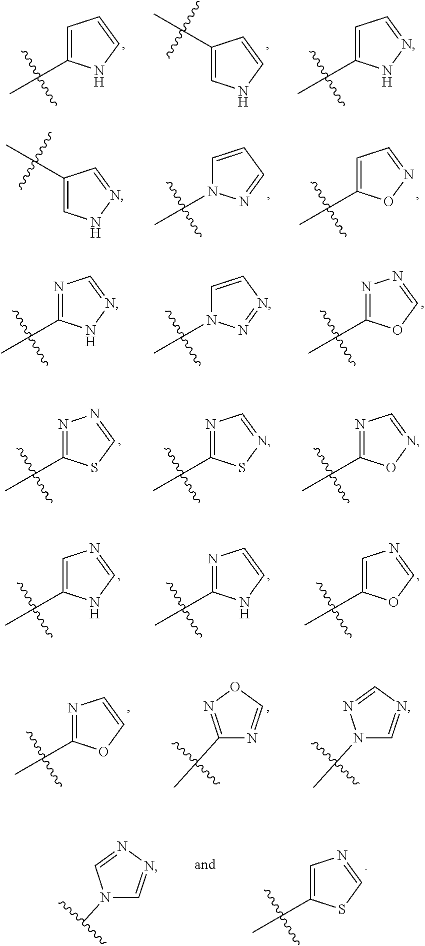

In any one of the embodiments described herein, A₂ is

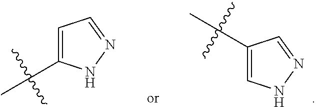 [SMILES: *c1ccn[nH]1 ; *c1cn[nH]c1]

In any one of the embodiments described herein, each occurrence of R₁₃ is independently H, halogen, fluorinated alkyl, or alkyl.

In any one of the embodiments described herein, one or more occurrences of R₁₃ are H.

In any one of the embodiments described herein, each occurrence of Z is independently OH or O(C₁-C₄ alkyl).

In any one of the embodiments described herein, each occurrence of Z is independently OMe, OEt, or OH.

In any one of the embodiments described herein, one or more occurrences of Z are OH.

In any one of the embodiments described herein, each occurrence of X₁ is independently H, halogen, fluorinated alkyl, or alkyl.

In any one of the embodiments described herein, each occurrence of X₁ is independently H, F, Cl, Br, Me, CF₂H, CF₂Cl, or CF₃.

In any one of the embodiments described herein, one or more occurrences of X₁ are H.

In any one of the embodiments described herein, each occurrence of X₂ is independently H, halogen, fluorinated alkyl, or alkyl.

In any one of the embodiments described herein, each occurrence of X₂ is independently H, F, Cl, Br, Me, CF₂H, CF₂Cl, or CF₃.

In any one of the embodiments described herein, one or more occurrences of X₂ are Cl.

In any one of the embodiments described herein, each occurrence of X₃ is independently H, halogen, fluorinated alkyl, or alkyl.

In any one of the embodiments described herein, each occurrence of X₃ is independently H, F, Cl, Br, Me, CF₂H, CF₂Cl, or CF₃.

In any one of the embodiments described herein, one or more occurrences of X₃ are Cl.

In any one of the embodiments described herein, each occurrence of R₃ is independently H, alkyl, CF₃, ORₐ, SRₐ, halogen, NRₐR_{b}, or NR_{b}(C═O)Rₐ.

In any one of the embodiments described herein, each occurrence of R₃ is independently H, halogen, fluorinated alkyl, or alkyl.

In any one of the embodiments described herein, one or more occurrences of R₃ are H.

In any one of the embodiments described herein, each occurrence of the structural moiety

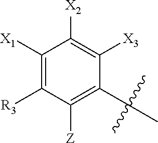

independently has the structure of

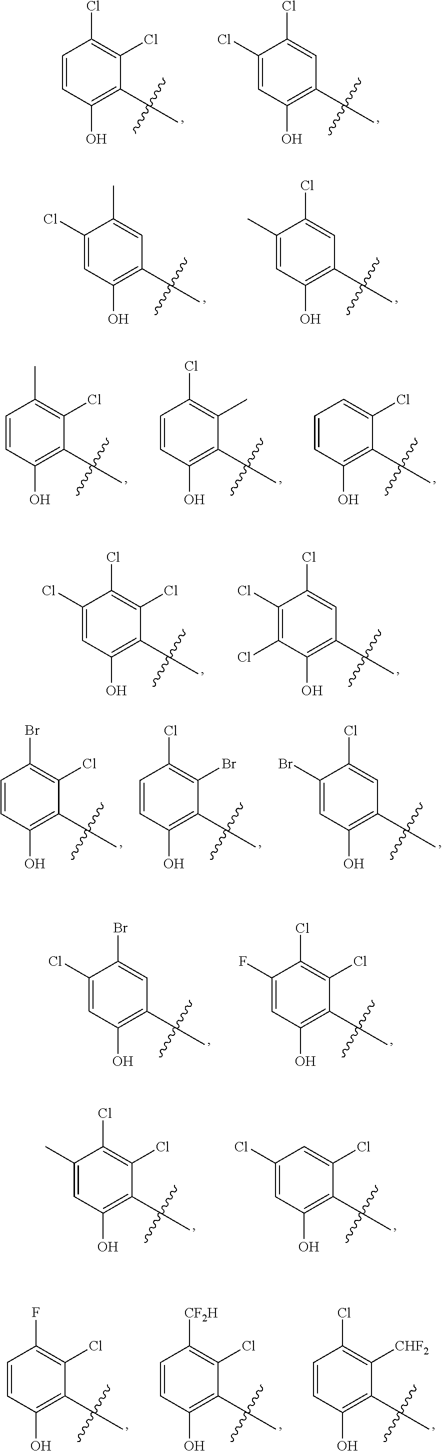

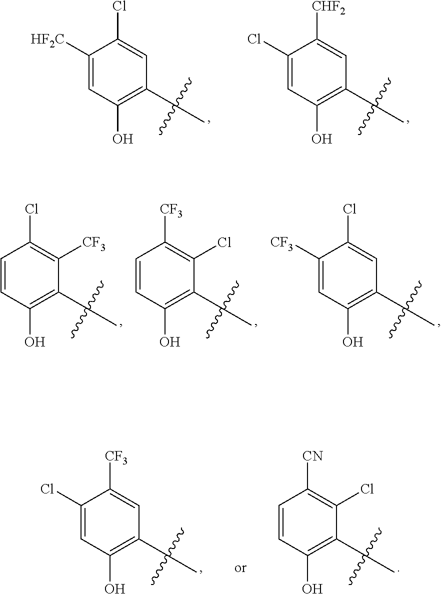

In any one of the embodiments described herein, at least one occurrence of the structural moiety

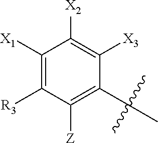

has the structure of

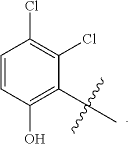 [SMILES: *c1c(O)ccc(Cl)c1Cl]

In any one of the embodiments described herein, the compound has a structure of Formula Ic, Ic′, Id, Id′, IIc, IIc′, IId, IId′, IIIc, IIId, IVc, or IVd:

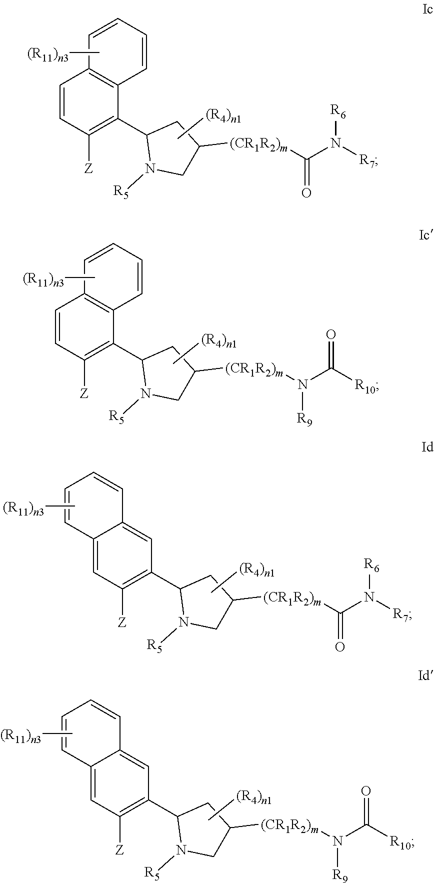

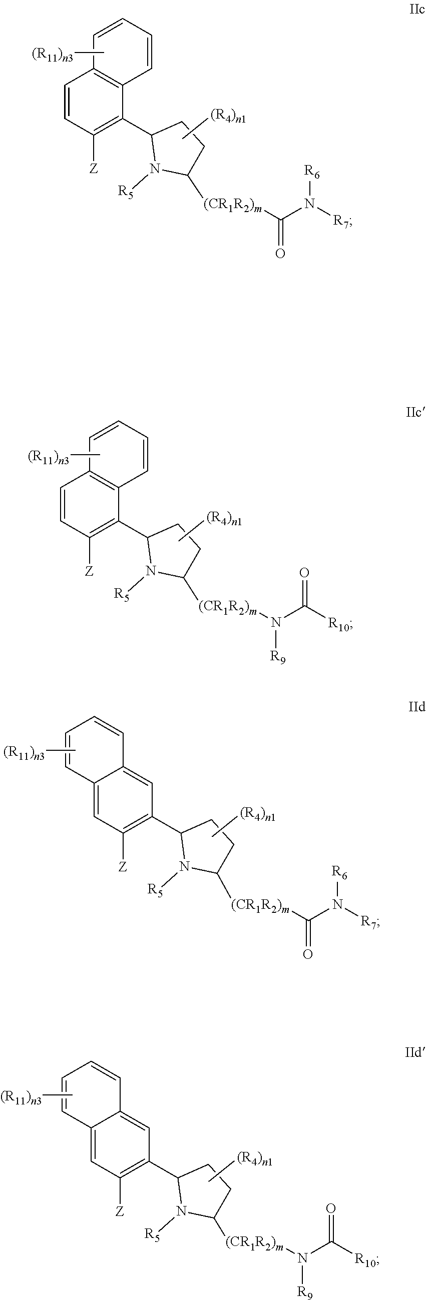

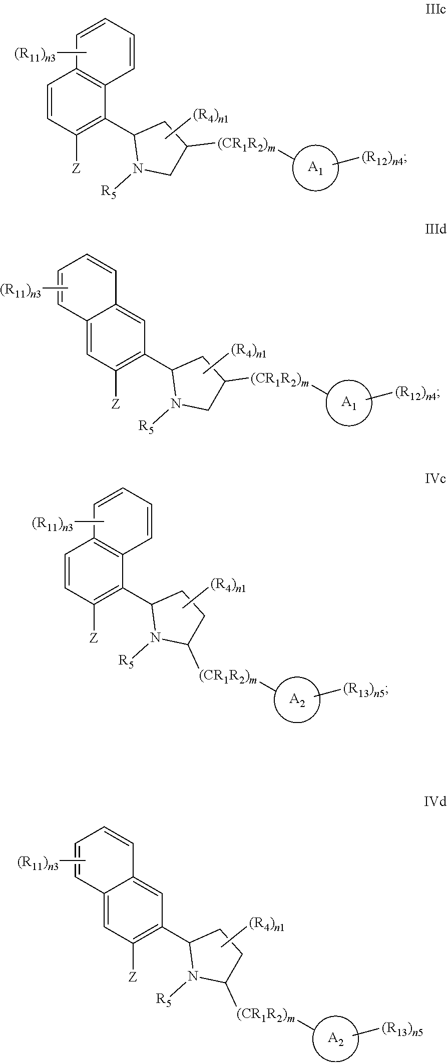

wherein each occurrence of R₁₁ is independently H, halogen, or alkyl; and each occurrence of n₃ is independently an integer from 0-3.

In any one of the embodiments described herein, each occurrence of n₃ is independently 0, 1, or 2.

In any one of the embodiments described herein, each occurrence of R₁₁ is independently H or alkyl.

In any one of the embodiments described herein, at least one occurrence of R₁₁ is halogen.

In any one of the embodiments described herein, at least one occurrence of Z is ORₐ.

In any one of the embodiments described herein, at least one occurrence of Z is OH, OMe, or OEt.

In any one of the embodiments described herein, at least one occurrence of Z is OH.

In any one of the embodiments described herein, at least one occurrence of Rₐ or R_{b} is independently H, alkyl, cycloalkyl, saturated heterocycle, aryl, or heteroaryl.

In any one of the embodiments described herein, at least one occurrence of Rₐ or R_{b} is independently H, Me, Et, Pr, or a heterocycle selected from the group consisting of

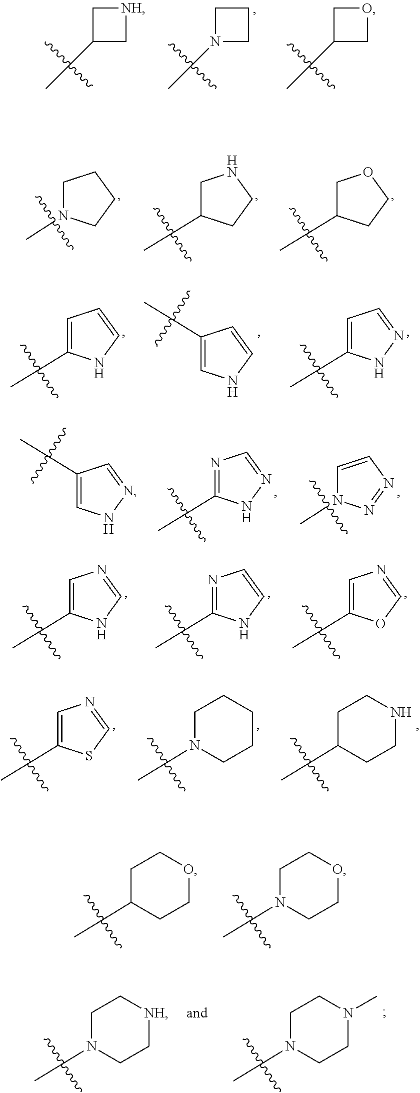

wherein the heterocycle is optionally substituted by alkyl, OH, oxo, or (C═O)C₁₋₄alkyl where valence permits.

In any one of the embodiments described herein, at least one occurrence of Rₐ or R_{b} is H, Me or

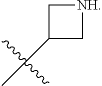 [SMILES: *C1CNC1]

In any one of the embodiments described herein, Rₐ and R_{b} together with the nitrogen atom that they are connected to form an optionally substituted heterocycle comprising the nitrogen atom and 0-3 additional heteroatoms each selected from the group consisting of N, O, and S.

In any one of the embodiments described herein, each occurrence of R₈ is independently H, alkyl, or heterocycle optionally substituted by alkyl, halogen, or OH.

In any one of the embodiments described herein, each occurrence of R₈ is independently H or alkyl.

In any one of the embodiments described herein, each occurrence of R₈ is independently H or Me.

In any one of the embodiments described herein, the compound is selected from the group consisting of compounds 31-79 as shown in Table 7.

In any one of the embodiments described herein, the compound is selected from the group consisting of compounds 1-15 as shown in Table 1, compounds 16-20 as shown in Table 2, compounds 1a-15a as shown in Table 3, compounds 16a-30a as shown in Table 4, compounds 1b-15b as shown in Table 5, and compounds 16b-30b as shown in Table 6.

In another aspect, a pharmaceutical composition is described, including at least one compound according to any one of the embodiments described herein or a pharmaceutically-acceptable salt thereof and a pharmaceutically-acceptable carrier or diluent.

In yet another aspect, a method of treating a condition in a mammalian species in need thereof is described, including administering to the mammalian species a therapeutically effective amount of at least one compound according to any one of the embodiments described herein, or a pharmaceutically-acceptable salt thereof, or a pharmaceutical composition thereof, where the condition is selected from the group consisting of cancer, an immunological disorder, a central nervous system disorder, an inflammatory disorder, a gastroenterological disorder, a metabolic disorder, a cardiovascular disorder, and a kidney disease.

In any one of the embodiments described herein, the immunological disorder is transplant rejection or an autoimmune disease.

In any one of the embodiments described herein, the autoimmune disease is rheumatoid arthritis, multiple sclerosis, systemic lupus erythematosus, or type I diabetes mellitus.

In any one of the embodiments described herein, the Central Nerve System (CNS) disorder is Alzheimer's disease.

In any one of the embodiments described herein, the inflammatory disorder is an inflammatory skin condition, arthritis, psoriasis, spondylitis, parodontitits, or an inflammatory neuropathy.

In any one of the embodiments described herein, the gastroenterological disorder is an inflammatory bowel disease.

In any one of the embodiments described herein, the metabolic disorder is obesity or type II diabetes mellitus.

In any one of the embodiments described herein, the cardiovascular disorder is an ischemic stroke.

In any one of the embodiments described herein, the kidney disease is chronic kidney disease, nephritis, or chronic renal failure.

In any one of the embodiments described herein, the condition is selected from the group consisting of cancer, transplant rejection, rheumatoid arthritis, multiple sclerosis, systemic lupus erythematosus, type I diabetes mellitus, Alzheimer's disease, inflammatory skin condition, inflammatory neuropathy, psoriasis, spondylitis, parodontitis, Crohn's disease, ulcerative colitis, obesity, type II diabetes mellitus, ischemic stroke, chronic kidney disease, nephritis, chronic renal failure, and a combination thereof.

In any one of the embodiments described herein, the mammalian species is human.

In yet another aspect, a method of blocking Kv1.3 potassium channel in a mammalian species in need thereof is described, including administering to the mammalian species a therapeutically effective amount of at least one compound according to any one of the embodiments described herein, or a pharmaceutically-acceptable salt thereof, or a pharmaceutical composition thereof.

In any one of the embodiments described herein, the mammalian species is human.

Any one of the embodiments disclosed herein may be properly combined with any other embodiment disclosed herein. The combination of any one of the embodiments disclosed herein with any other embodiments disclosed herein is expressly contemplated. Specifically, the selection of one or more embodiments for one substituent group can be properly combined with the selection of one or more particular embodiments for any other substituent group. Such combination can be made in any one or more embodiments of the application described herein or any formula described herein.

### DETAILED DESCRIPTION OF THE INVENTION

### Definitions

The following are definitions of terms used in the present specification. The initial definition provided for a group or term herein applies to that group or term throughout the present specification individually or as part of another group, unless otherwise indicated. Unless otherwise defined, all technical and scientific terms used herein have the same meaning as commonly understood by one of ordinary skill in the art.

The terms “alkyl” and “alk” refer to a straight or branched chain alkane (hydrocarbon) radical containing from 1 to 12 carbon atoms, preferably 1 to 6 carbon atoms. Exemplary “alkyl” groups include methyl, ethyl, propyl, isopropyl, n-butyl, t-butyl, isobutyl pentyl, hexyl, isohexyl, heptyl, 4,4-dimethylpentyl, octyl, 2,2,4-trimethylpentyl, nonyl, decyl, undecyl, dodecyl, and the like. The term “(C₁-C₄)alkyl” refers to a straight or branched chain alkane (hydrocarbon) radical containing from 1 to 4 carbon atoms, such as methyl, ethyl, propyl, isopropyl, n-butyl, t-butyl, and isobutyl. “Substituted alkyl” refers to an alkyl group substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR^{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle, and each occurrence of Rₑ is independently alkyl, cycloalkyl, alkenyl, cycloalkenyl, alkynyl, heterocycle, or aryl. In some embodiments, groups such as alkyl, cycloalkyl, alkenyl, alkynyl, cycloalkenyl, heterocycle, and aryl can themselves be optionally substituted.

The term “alkenyl” refers to a straight or branched chain hydrocarbon radical containing from 2 to 12 carbon atoms and at least one carbon-carbon double bond. Exemplary such groups include ethenyl or allyl. The term “C₂-C₆ alkenyl” refers to a straight or branched chain hydrocarbon radical containing from 2 to 6 carbon atoms and at least one carbon-carbon double bond, such as ethylenyl, propenyl, 2-propenyl, (E)-but-2-enyl, (Z)-but-2-enyl, 2-methy(E)-but-2-enyl, 2-methy(Z)-but-2-enyl, 2,3-dimethy-but-2-enyl, (Z)-pent-2-enyl, (E)-pent-1-enyl, (Z)-hex-1-enyl, (E)-pent-2-enyl, (Z)-hex-2-enyl, (E)-hex-2-enyl, (Z)-hex-1-enyl, (E)-hex-1-enyl, (Z)-hex-3-enyl, (E)-hex-3-enyl, and (E)-hex-1,3-dienyl. “Substituted alkenyl” refers to an alkenyl group substituted with one or more substituents, preferably 1 to 4 substituents, at any available point of attachment. Exemplary substituents include, but are not limited to, one or more of the following groups: hydrogen, halogen, alkyl, halogenated alkyl (i.e., an alkyl group bearing a single halogen substituent or multiple halogen substituents such as CF₃ or CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR^{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted.

The term “alkynyl” refers to a straight or branched chain hydrocarbon radical containing from 2 to 12 carbon atoms and at least one carbon to carbon triple bond. Exemplary groups include ethynyl. The term “C₂-C₆ alkynyl” refers to a straight or branched chain hydrocarbon radical containing from 2 to 6 carbon atoms and at least one carbon-carbon triple bond, such as ethynyl, prop-1-ynyl, prop-2-ynyl, but-1-ynyl, but-2-ynyl, pent-1-ynyl, pent-2-ynyl, hex-1-ynyl, hex-2-ynyl, or hex-3-ynyl. “Substituted alkynyl” refers to an alkynyl group substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₐ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, Rₑ and R_{d} is independently hydrogen, alkyl, cycloalkyl, heterocycle, aryl, or said R_{b} and Rₑ together with the N to which they are bonded optionally to form a heterocycle; and each occurrence of Rₑ is independently alkyl, cycloalkyl, alkenyl, cycloalkenyl, alkynyl, heterocycle, or aryl. The exemplary substituents can themselves be optionally substituted.

The term “cycloalkyl” refers to a fully saturated cyclic hydrocarbon group containing from 1 to 4 rings and 3 to 8 carbons per ring. “C₃-C₇ cycloalkyl” refers to cyclopropyl, cyclobutyl, cyclopentyl, cyclohexyl, or cycloheptyl. “Substituted cycloalkyl” refers to a cycloalkyl group substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₐ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, Rₑ and R_{d} is independently hydrogen, alkyl, cycloalkyl, heterocycle, aryl, or said R_{b} and R_{c} together with the N to which they are bonded optionally to form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “cycloalkenyl” refers to a partially unsaturated cyclic hydrocarbon group containing 1 to 4 rings and 3 to 8 carbons per ring. Exemplary such groups include cyclobutenyl, cyclopentenyl, cyclohexenyl, etc. “Substituted cycloalkenyl” refers to a cycloalkenyl group substituted with one more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₐ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c}, and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “aryl” refers to cyclic, aromatic hydrocarbon groups that have 1 to 5 aromatic rings, especially monocyclic or bicyclic groups such as phenyl, biphenyl or naphthyl. Where containing two or more aromatic rings (bicyclic, etc.), the aromatic rings of the aryl group may be joined at a single point (e.g., biphenyl), or fused (e.g., naphthyl, phenanthrenyl and the like). The term “fused aromatic ring” refers to a molecular structure having two or more aromatic rings wherein two adjacent aromatic rings have two carbon atoms in common. “Substituted aryl” refers to an aryl group substituted by one or more substituents, preferably 1 to 3 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}Rₑ, NR_{b}S(═O)₂Rₑ, NR_{b}P(═O)₂Rₑ, S(═O)₂NR_{b}Rₑ, P(═O)₂NR_{b}R_{c}, C(═O)OR_{d}, C(═O)Rₐ, C(═O)NR_{b}Rₑ, OC(═O)Rₐ, OC(═O)NR_{b}Rₑ, NR_{b}C(═O)ORₑ, NR_{d}C(═O)NR_{b}Rₑ, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, Rₑ and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include fused cyclic groups, especially fused cycloalkyl, fused cycloalkenyl, fused heterocycle, or fused aryl, where the aforementioned cycloalkyl, cycloalkenyl, heterocycle, and aryl substituents can themselves be optionally substituted.

The term “biaryl” refers to two aryl groups linked by a single bond. The term “biheteroaryl” refers to two heteroaryl groups linked by a single bond. Similarly, the term “heteroaryl-aryl” refers to a heteroaryl group and an aryl group linked by a single bond and the term “aryl-heteroaryl” refers to an aryl group and a heteroaryl group linked by a single bond. In certain embodiments, the numbers of the ring atoms in the heteroaryl and/or aryl rings are used to specify the sizes of the aryl or heteroaryl ring in the substituents. For example, 5,6-heteroaryl-aryl refers to a substituent in which a 5-membered heteroaryl is linked to a 6-membered aryl group. Other combinations and ring sizes can be similarly specified.

The term “carbocycle” or “carbon cycle” refers to a fully saturated or partially saturated cyclic hydrocarbon group containing from 1 to 4 rings and 3 to 8 carbons per ring, or cyclic, aromatic hydrocarbon groups that have 1 to 5 aromatic rings, especially monocyclic or bicyclic groups such as phenyl, biphenyl, or naphthyl. The term “carbocycle” encompasses cycloalkyl, cycloalkenyl, cycloalkynyl, and aryl as defined hereinabove. The term “substituted carbocycle” refers to carbocycle or carbocyclic groups substituted with one or more substituents, preferably 1 to 4 substituents, at any available point of attachment. Exemplary substituents include, but are not limited to, those described above for substituted cycloalkyl, substituted cycloalkenyl, substituted cycloalkynyl, and substituted aryl. Exemplary substituents also include spiro-attached or fused cyclic substituents at any available point or points of attachment, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle, and aryl substituents can themselves be optionally substituted.

The terms “heterocycle” and “heterocyclic” refer to fully saturated, or partially or fully unsaturated, including aromatic (i.e., “heteroaryl”) cyclic groups (for example, 3 to 7 membered monocyclic, 7 to 11 membered bicyclic, or 8 to 16 membered tricyclic ring systems) which have at least one heteroatom in at least one carbon atom-containing ring. Each ring of the heterocyclic group may independently be saturated, or partially or fully unsaturated. Each ring of the heterocyclic group containing a heteroatom may have 1, 2, 3, or 4 heteroatoms selected from the group consisting of nitrogen atoms, oxygen atoms and sulfur atoms, where the nitrogen and sulfur heteroatoms may optionally be oxidized and the nitrogen heteroatoms may optionally be quaternized. (The term “heteroarylium” refers to a heteroaryl group bearing a quaternary nitrogen atom and thus a positive charge.) The heterocyclic group may be attached to the remainder of the molecule at any heteroatom or carbon atom of the ring or ring system. Exemplary monocyclic heterocyclic groups include azetidinyl, pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, hexahydrodiazepinyl, 4-piperidonyl, pyridyl, pyrazinyl, pyrimidinyl, pyridazinyl, triazinyl, triazolyl, tetrazolyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, and the like. Exemplary bicyclic heterocyclic groups include indolyl, indolinyl, isoindolyl, benzothiazolyl, benzoxazolyl, benzoxadiazolyl, benzothienyl, benzo[d][1,3]dioxolyl, dihydro-2H-benzo[b][1,4]oxazine, 2,3-dihydrobenzo[b][1,4]dioxinyl, quinuclidinyl, quinolinyl, tetrahydroisoquinolinyl, isoquinolinyl, benzimidazolyl, benzopyranyl, indolizinyl, benzofuryl, benzofurazanyl, dihydrobenzo[d]oxazole, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl] or furo[2,3-b]pyridinyl), dihydroisoindolyl, dihydroquinazolinyl (such as 3,4-dihydro-4-oxo-quinazolinyl), triazinylazepinyl, tetrahydroquinolinyl, and the like. Exemplary tricyclic heterocyclic groups include carbazolyl, benzidolyl, phenanthrolinyl, acridinyl, phenanthridinyl, xanthenyl, and the like.

“Substituted heterocycle” and “substituted heterocyclic” (such as “substituted heteroaryl”) refer to heterocycle or heterocyclic groups substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₐ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, Rₑ and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents at any available point or points of attachment, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “oxo” refers to

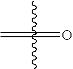 [SMILES: *=O]

substituent group, which may be attached to a carbon ring atom on a carboncycle or heterocycle. When an oxo substituent group is attached to a carbon ring atom on an aromatic group, e.g., aryl or heteroaryl, the bonds on the aromatic ring may be rearranged to satisfy the valence requirement. For instance, a pyridine with a 2-oxo substituent group may have the structure of

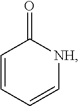

which also includes its tautomeric form of

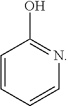

The term “alkylamino” refers to a group having the structure —NHR′, wherein R′ is hydrogen, alkyl or substituted alkyl, cycloalkyl or substituted cycloalkyl, as defined herein. Examples of alkylamino groups include, but are not limited to, methylamino, ethylamino, n-propylamino, iso-propylamino, cyclopropylamino, n-butylamino, tert-butylamino, neopentylamino, n-pentylamino, hexylamino, cyclohexylamino, and the like.

The term “dialkylamino” refers to a group having the structure —NRR′, wherein R and R′ are each independently alkyl or substituted alkyl, cycloalkyl or substituted cycloalkyl, cycloalkenyl or substituted cyclolalkenyl, aryl or substituted aryl, heterocycle or substituted heterocycle, as defined herein. R and R′ may be the same or different in a dialkyamino moiety. Examples of dialkylamino groups include, but are not limited to, dimethylamino, methyl ethylamino, diethylamino, methylpropylamino, di(n-propyl)amino, di(iso-propyl)amino, di(cyclopropyl)amino, di(n-butyl)amino, di(tert-butyl)amino, di(neopentyl)amino, di(n-pentyl)amino, di(hexyl)amino, di(cyclohexyl)amino, and the like. In certain embodiments, R and R′ are linked to form a cyclic structure. The resulting cyclic structure may be aromatic or non-aromatic. Examples of the resulting cyclic structure include, but are not limited to, aziridinyl, pyrrolidinyl, piperidinyl, morpholinyl, pyrrolyl, imidazolyl, 1,2,4-triazolyl, and tetrazolyl.

The terms “halogen” or “halo” refer to chlorine, bromine, fluorine, or iodine.

The term “substituted” refers to the embodiments in which a molecule, molecular moiety, or substituent group (e.g., alkyl, cycloalkyl, alkenyl, cycloalkenyl, alkynyl, heterocycle, or aryl group or any other group disclosed herein) is substituted with one or more substituents, where valence permits, preferably 1 to 6 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, alkyl, halogen-substituted alkyl, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, Rₑ and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. In the aforementioned exemplary substituents, groups such as alkyl, cycloalkyl, alkenyl, alkynyl, cycloalkenyl, heterocycle, and aryl can themselves be optionally substituted. The term “optionally substituted” refers to the embodiments in which a molecule, molecular moiety or substituent group (e.g., alkyl, cycloalkyl, alkenyl, cycloalkenyl, alkynyl, heterocycle, or aryl group or any other group disclosed herein) may or may not be substituted with aforementioned one or more substituents.

Unless otherwise indicated, any heteroatom with unsatisfied valences is assumed to have hydrogen atoms sufficient to satisfy the valences.

The compounds of the present invention may form salts which are also within the scope of this invention. Reference to a compound of the present invention is understood to include reference to salts thereof, unless otherwise indicated. The term “salt(s)”, as employed herein, denotes acidic and/or basic salts formed with inorganic and/or organic acids and bases. In addition, when a compound of the present invention contains both a basic moiety, such as but not limited to a pyridine or imidazole, and an acidic moiety such as but not limited to a phenol or carboxylic acid, zwitterions (“inner salts”) may be formed and are included within the term “salt(s)” as used herein. Pharmaceutically-acceptable (i.e., non-toxic, physiologically-acceptable) salts are preferred, although other salts are also useful, e.g., in isolation or purification steps which may be employed during preparation. Salts of the compounds of the present invention may be formed, for example, by reacting a compound described herein with an amount of acid or base, such as an equivalent amount, in a medium such as one in which the salt precipitates, or in an aqueous medium followed by lyophilization.

The compounds of the present invention which contain a basic moiety, such as but not limited to an amine or a pyridine or imidazole ring, may form salts with a variety of organic and inorganic acids. Exemplary acid addition salts include acetates (such as those formed with acetic acid or trihaloacetic acid; for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, hydroxyethanesulfonates (e.g., 2-hydroxyethanesulfonates), lactates, maleates, methanesulfonates, naphthalenesulfonates (e.g., 2-naphthalenesulfonates), nicotinates, nitrates, oxalates, pectinates, persulfates, phenylpropionates (e.g., 3-phenylpropionates), phosphates, picrates, pivalates, propionates, salicylates, succinates, sulfates (such as those formed with sulfuric acid), sulfonates, tartrates, thiocyanates, toluenesulfonates such as tosylates, undecanoates, and the like.

The compounds of the present invention which contain an acidic moiety, such as but not limited to a phenol or carboxylic acid, may form salts with a variety of organic and inorganic bases. Exemplary basic salts include ammonium salts, alkali metal salts such as sodium, lithium and potassium salts, alkaline earth metal salts such as calcium and magnesium salts, salts with organic bases (for example, organic amines) such as benzathines, dicyclohexylamines, hydrabamines (formed with N,N-bis(dehydroabietyl) ethylenediamine), N-methyl-D-glucamines, N-methyl-D-glycamides, t-butyl amines, and salts with amino acids such as arginine, lysine, and the like. Basic nitrogen-containing groups may be quaternized with agents such as lower alkyl halides (e.g., methyl, ethyl, propyl, and butyl chlorides, bromides, and iodides), dialkyl sulfates (e.g., dimethyl, diethyl, dibutyl, and diamyl sulfates), long chain halides (e.g., decyl, lauryl, myristyl and stearyl chlorides, bromides, and iodides), aralkyl halides (e.g., benzyl and phenethyl bromides), and others.

Prodrugs and solvates of the compounds of the invention are also contemplated herein. The term “prodrug” as employed herein denotes a compound that, upon administration to a subject, undergoes chemical conversion by metabolic or chemical processes to yield a compound of the present invention, or a salt and/or solvate thereof. Solvates of the compounds of the present invention include, for example, hydrates.

Compounds of the present invention, and salts or solvates thereof, may exist in their tautomeric form (for example, as an amide or imino ether). All such tautomeric forms are contemplated herein as part of the present invention. As used herein, any depicted structure of the compound includes the tautomeric forms thereof.

All stereoisomers of the present compounds (for example, those which may exist due to asymmetric carbons on various substituents), including enantiomeric forms and diastereomeric forms, are contemplated within the scope of this invention. Individual stereoisomers of the compounds of the invention may, for example, be substantially free of other isomers (e.g., as a pure or substantially pure optical isomer having a specified activity), or may be admixed, for example, as racemates or with all other, or other selected, stereoisomers. The chiral centers of the present invention may have the S or R configuration as defined by the International Union of Pure and Applied Chemistry (IUPAC) 1974 Recommendations. The racemic forms can be resolved by physical methods, such as, for example, fractional crystallization, separation or crystallization of diastereomeric derivatives, or separation by chiral column chromatography. The individual optical isomers can be obtained from the racemates by any suitable method, including without limitation, conventional methods, such as, for example, salt formation with an optically active acid followed by crystallization.

Compounds of the present invention are, subsequent to their preparation, preferably isolated and purified to obtain a composition containing an amount by weight equal to or greater than 90%, for example, equal to or greater than 95%, equal to or greater than 99% of the compounds (“substantially pure” compounds), which is then used or formulated as described herein. Such “substantially pure” compounds of the present invention are also contemplated herein as part of the present invention.

All configurational isomers of the compounds of the present invention are contemplated, either in admixture or in pure or substantially pure form. The definition of compounds of the present invention embraces both cis (Z) and trans (E) alkene isomers, as well as cis and trans isomers of cyclic hydrocarbon or heterocyclic rings.

Throughout the specification, groups and substituents thereof may be chosen to provide stable moieties and compounds.

Definitions of specific functional groups and chemical terms are described in more detail herein. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry”, Thomas Sorrell, University Science Books, Sausalito (1999), the entire contents of which are incorporated herein by reference.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomer mixtures.

The present invention also includes isotopically labeled compounds, which are identical to the compounds disclosed herein, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine, and chlorine, such as ²H, ³H, ¹³C, ¹¹C, ¹⁴C, ¹⁵N, ¹⁸O, ¹⁷O, ³P, ³²P, ³⁵S, ¹⁸F, and ³⁶Cl, respectively. Compounds of the present invention, or an enantiomer, diastereomer, tautomer, or pharmaceutically-acceptable salt or solvate thereof, which contain the aforementioned isotopes and/or other isotopes of other atoms are within the scope of this invention. Certain isotopically labeled compounds of the present invention, for example, those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example, increased in vivo half-life or reduced dosage requirements, and hence may be preferred in some circumstances. Isotopically-labeled compounds can generally be prepared by carrying out the procedures disclosed in the Schemes and/or in the Examples below, by substituting a readily-available isotopically-labeled reagent for a non-isotopically-labeled reagent.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this invention, heteroatoms such as nitrogen may have hydrogen substituents and/or any permissible substituents of organic compounds described herein which satisfy the valences of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example, of proliferative disorders. The term “stable,” as used herein, preferably refers to compounds which possess stability sufficient to allow manufacture and which maintain the integrity of the compound for a sufficient period of time to be detected and preferably for a sufficient period of time to be useful for the purposes detailed herein.

As used herein, the terms “cancer” and, equivalently, “tumor” refer to a condition in which abnormally replicating cells of host origin are present in a detectable amount in a subject. The cancer can be a malignant or non-malignant cancer. Cancers or tumors include, but are not limited to, biliary tract cancer; brain cancer; breast cancer; cervical cancer; choriocarcinoma; colon cancer; endometrial cancer; esophageal cancer; gastric (stomach) cancer; intraepithelial neoplasms; leukemias; lymphomas; liver cancer; lung cancer (e.g., small cell and non-small cell); melanoma; neuroblastomas; oral cancer; ovarian cancer; pancreatic cancer; prostate cancer; rectal cancer; renal (kidney) cancer; sarcomas; skin cancer; testicular cancer; thyroid cancer; as well as other carcinomas and sarcomas. Cancers can be primary or metastatic. Diseases other than cancers may be associated with mutational alternation of component of Ras signaling pathways and the compound disclosed herein may be used to treat these non-cancer diseases. Such non-cancer diseases may include: neurofibromatosis; Leopard syndrome; Noonan syndrome; Legius syndrome; Costello syndrome; cardio-facio-cutaneous syndrome; hereditary gingival fibromatosis type 1; autoimmune lymphoproliferative syndrome; and capillary malformation-arterovenous malformation.

As used herein, “effective amount” refers to any amount that is necessary or sufficient for achieving or promoting a desired outcome. In some instances, an effective amount is a therapeutically effective amount. A therapeutically effective amount is any amount that is necessary or sufficient for promoting or achieving a desired biological response in a subject. The effective amount for any particular application can vary depending on such factors as the disease or condition being treated, the particular agent being administered, the size of the subject, or the severity of the disease or condition. One of ordinary skill in the art can empirically determine the effective amount of a particular agent without necessitating undue experimentation.

As used herein, the term “subject” refers to a vertebrate animal. In one embodiment, the subject is a mammal or a mammalian species. In one embodiment, the subject is a human. In other embodiments, the subject is a non-human vertebrate animal, including, without limitation, non-human primates, laboratory animals, livestock, racehorses, domesticated animals, and non-domesticated animals.

Compounds Novel compounds as Kv1.3 potassium channel blockers are described. Applicants have surprisingly discovered that the compounds disclosed herein exhibit potent Kv1.3 potassium channel-inhibiting properties. Additionally, Applicants have surprisingly discovered that the compounds disclosed herein selectively block the Kv1.3 potassium channel and do not block the hERG channel and thus have desirable cardiovascular safety profiles.

In one aspect, a compound of Formula I, I′, II, II′, III, or IV, or a pharmaceutically-acceptable salt thereof is described,

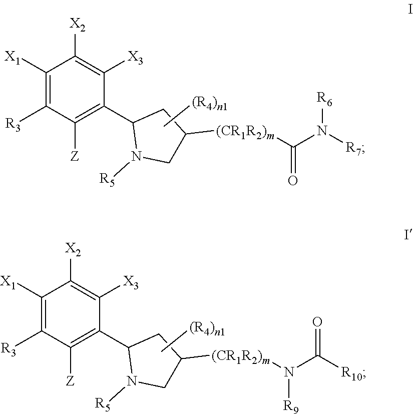

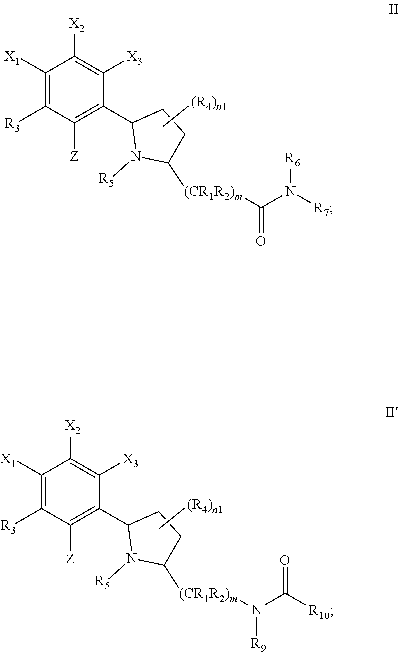

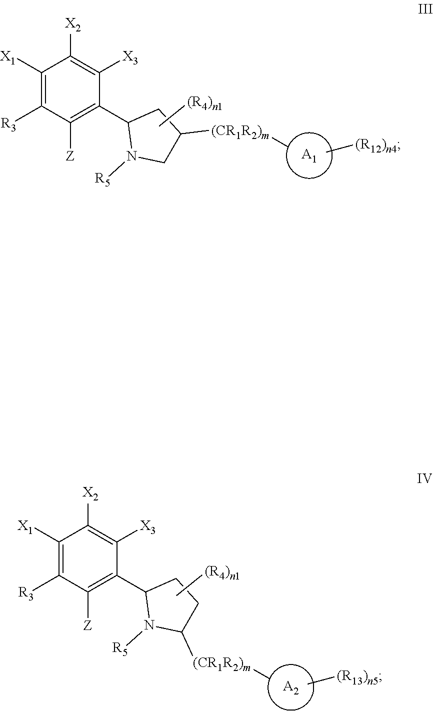

wherein
each occurrence of Z is independently ORₐ;
each occurrence of X₁ is independently H, halogen, CN, alkyl, cycloalkyl, halogenated cycloalkyl, or halogenated alkyl;
each occurrence of X₂ is independently H, halogen, CN, alkyl, cycloalkyl, halogenated cycloalkyl, or halogenated alkyl;
each occurrence of X₃ is independently H, halogen, CN, alkyl, cycloalkyl, halogenated cycloalkyl, or halogenated alkyl;
or alternatively X₁ and X₂ and the carbon atoms they are connected to taken together form an optionally substituted 5- or 6-membered aryl;
or alternatively X₂ and X₃ and the carbon atoms they are connected to taken together form an optionally substituted 5- or 6-membered aryl;
each occurrence of R₁ is independently H, alkyl, cycloalkyl, saturated heterocycle, aryl, heteroaryl, alkylaryl, alkylheteroaryl, CN, CF₃, OCF₃, ORₐ, SRₐ, halogen, NRₐR_{b}, or NR_{b}(C═O)Rₐ;
each occurrence of R₂ is independently H, alkyl, cycloalkyl, saturated heterocycle, aryl, heteroaryl, alkylaryl, alkylheteroaryl, CN, CF₃, OCF₃, ORₐ, SRₐ, halogen, NRₐR_{b}, or NR_{b}(C═O)Rₐ;
or alternatively R₁ and R₂ taken together with the carbon atom they are connected to form a cycloalkyl or saturated heterocycle;
each occurrence of R₃ is independently H, alkyl, cycloalkyl, saturated heterocycle, aryl, heteroaryl, CN, CF₃, OCF₃, ORₐ, SRₐ, halogen, NRₐR_{b}, or NR_{b}(C═O)Rₐ;
each occurrence of R₄ is independently H, alkyl, cycloalkyl, saturated heterocycle, aryl, heteroaryl, alkylaryl, alkylheteroaryl, halogen, CN, CF₃, ORₐ, (CRₐR_{b})ₙ₂ORₐ, oxo, (C═O)Rₐ, O(C═O)Rₐ, (C═O)ORₐ, or (CRₐR_{b})ₙ₂NRₐR_{b};
or alternatively two R₄ groups taken together with the carbon atom(s) that they are connected to form a 3-7 membered optionally substituted cycloalkyl or heterocycle;
each occurrence of R₅ is independently H, alkyl, cycloalkyl, saturated heterocycle, aryl, heteroaryl, alkylaryl, alkylheteroaryl, (C═O)Rₐ, (C═O)(CRₐR_{b})ₙ₂ORₐ, (C═O)(CRₐR_{b})ₙ₂NRₐR_{b}, or SO₂Rₐ;
each occurrence of R₆ is independently H, alkyl, cycloalkyl, heterocycle, aryl, heteroaryl, alkylaryl, or alkylheteroaryl;
each occurrence of R₇ is independently H, alkyl, cycloalkyl, heterocycle, aryl, heteroaryl, alkylaryl, or alkylheteroaryl;
or alternatively R₆ and R₇ taken together with the nitrogen atom they are connected to form a heterocycle comprising the nitrogen atom and 0-3 additional heteroatoms each selected from the group consisting of N, O, and S; wherein the heterocycle is optionally substituted by 1-4 substituents each independently selected from the group consisting of alkyl, cycloalkyl, halogenated cycloalkyl, halogenated alkyl, halogen, CN, OR₈, —(CH₂)₀₋₂OR₈, N(R₈)₂, (C═O)R₈, (C═O)N(R₈)₂, NR₈(C═O)R₈, and oxo where valence permits;
each occurrence of R₉ is independently H, alkyl, cycloalkyl, saturated heterocycle, aryl, heteroaryl, alkylaryl, alkylheteroaryl, (C═O)Rₐ, (C═O)(CRₐR_{b})ₙ₂ORₐ, (C═O)(CRₐR_{b})ₙ₂NRₐR_{b}, or SO₂Rₐ;
each occurrence of R₁₀ is independently H, alkyl, cycloalkyl, heterocycle, aryl, heteroaryl, alkylaryl, or alkylheteroaryl;
A₁ is aryl or heteroaryl;
A₂ is aryl or heteroaryl;
each occurrence of R₁₂ is independently H, alkyl, CN, CF₃, OCF₃, ORₐ, SRₐ, halogen, (CRₐR_{b})ₙ₂ORₐ, (C═O)NRₐR_{b}, (CRₐR_{b})ₙ₂NRₐR_{b}, or (CRₐR_{b})ₙ₂NR_{b}(C═O)Rₐ;
each occurrence of R₁₃ is independently H, alkyl, CN, CF₃, OCF₃, ORₐ, SRₐ, halogen, (CRₐR_{b})ₙ₂ORₐ, (C═O)NRₐR_{b}, (CRₐR_{b})ₙ₂NRₐR_{b}, or (CRₐR_{b})ₙ₂NR_{b}(C═O)Rₐ;
each occurrence of Rₐ and R_{b} are independently H, alkyl, alkenyl, cycloalkyl, saturated heterocycle comprising 1-3 heteroatoms each selected from the group consisting of N, O, and S, aryl, or heteroaryl; or alternatively Rₐ and R_{b} together with the carbon or nitrogen atom that they are connected to form a cycloalkyl or heterocycle comprising the nitrogen atom and 0-3 additional heteroatoms each selected from the group consisting of N, O, and S;
the alkyl, cycloalkyl, heterocycle, aryl, and heteroaryl in X₁, X₂, X₃, A₁, A₂, R₁, R₂, R₃, R₄, R₅, R₆, R₇, R₉, R₁₀, R₁₂, R₁₃, Rₐ, or R_{b}, where applicable, are optionally substituted by 1-4 substituents each independently selected from the group consisting of alkyl, cycloalkyl, halogenated cycloalkyl, halogenated alkyl, halogen, CN, OR₈, —(CH₂)₀₋₂OR₈, N(R₈)₂, (C═O)R₈, (C═O)N(R₈)₂, NR₈(C═O)R₈, and oxo where valence permits;
each occurrence of R₈ is independently H, alkyl, or optionally substituted heterocycle; or alternatively the two R₈ groups together with the nitrogen atom that they are connected to form an optionally substituted heterocycle comprising the nitrogen atom and 0-3 additional heteroatoms each selected from the group consisting of N, O, and S;
each occurrence of m is independently 0, 1, 2, or 3;
each occurrence of n₁ is independently an integer from 0-3 wherein valence permits;
each occurrence of n₂ is independently an integer from 0-3;
n₄ is an integer from 0-3; and
n₅ is an integer from 0-3.

In another aspect, a compound of Formula I, I′, II, II′, III, or IV, or a pharmaceutically-acceptable salt thereof is described,

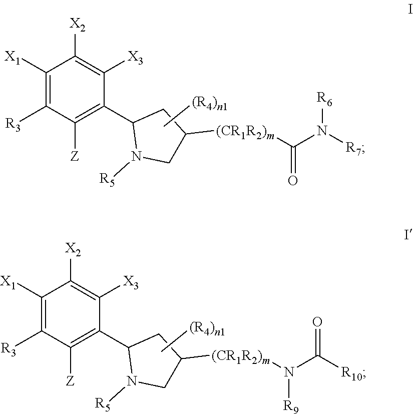

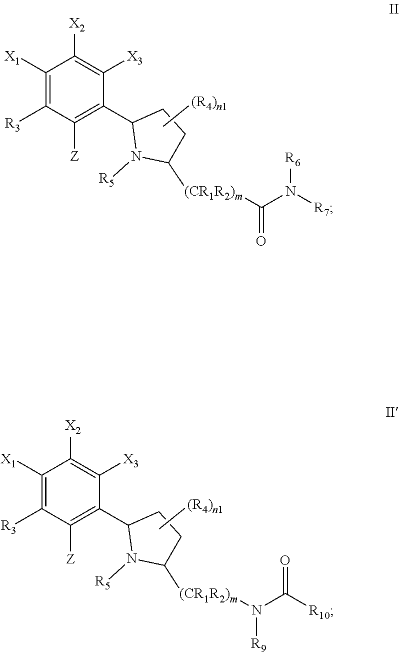

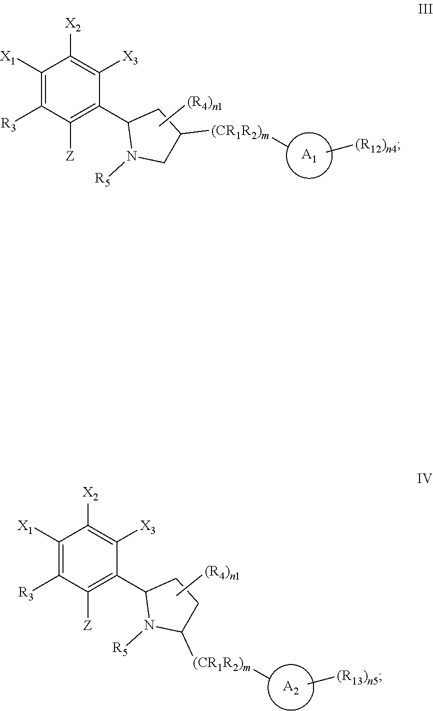

wherein
each occurrence of Z is independently ORₐ;
each occurrence of X₁ is independently H, halogen, CN, alkyl, cycloalkyl, halogenated cycloalkyl, or halogenated alkyl;
each occurrence of X₂ is independently H, halogen, CN, alkyl, cycloalkyl, halogenated cycloalkyl, or halogenated alkyl;
each occurrence of X₃ is independently H, halogen, CN, alkyl, cycloalkyl, halogenated cycloalkyl, or halogenated alkyl;
or alternatively X₁ and X₂ and the carbon atoms they are connected to taken together form an optionally substituted 5- or 6-membered aryl;
or alternatively X₂ and X₃ and the carbon atoms they are connected to taken together form an optionally substituted 5- or 6-membered aryl;
each occurrence of R₁ is independently H, alkyl, cycloalkyl, saturated heterocycle, aryl, heteroaryl, alkylaryl, alkylheteroaryl, CN, CF₃, OCF₃, ORₐ, SRₐ, halogen, NRₐR_{b}, or NR_{b}(C═O)Rₐ;
each occurrence of R₂ is independently H, alkyl, cycloalkyl, saturated heterocycle, aryl, heteroaryl, alkylaryl, alkylheteroaryl, CN, CF₃, OCF₃, ORₐ, SRₐ, halogen, NRₐR_{b}, or NR_{b}(C═O)Rₐ;
or alternatively R₁ and R₂ taken together with the carbon atom they are connected to form a cycloalkyl or saturated heterocycle;
each occurrence of R₃ is independently H, alkyl, cycloalkyl, saturated heterocycle, aryl, heteroaryl, CN, CF₃, OCF₃, ORₐ, SRₐ, halogen, NRₐR_{b}, or NR_{b}(C═O)Rₐ;
each occurrence of R₄ is independently H, alkyl, cycloalkyl, saturated heterocycle, (CRₐR_{b})ₙ₂ORₐ, or (CRₐR_{b})ₙ₂NRₐR_{b};
or alternatively two R₄ groups taken together with the carbon atom(s) that they are connected to form a 3-7 membered optionally substituted cycloalkyl or heterocycle;
each occurrence of R₅ is independently H, alkyl, cycloalkyl, saturated heterocycle, aryl, heteroaryl, alkylaryl, alkylheteroaryl, (C═O)Rₐ, (C═O)(CRₐR_{b})ₙ₂ORₐ, (C═O)(CRₐR_{b})ₙ₂NRₐR_{b}, or SO₂Rₐ;
each occurrence of R₆ is independently H, alkyl, cycloalkyl, heterocycle, aryl, heteroaryl, alkylaryl, or alkylheteroaryl;
each occurrence of R₇ is independently H, alkyl, cycloalkyl, heterocycle, aryl, heteroaryl, alkylaryl, or alkylheteroaryl;
or alternatively R₆ and R₇ taken together with the nitrogen atom they are connected to form a heterocycle comprising the nitrogen atom and 0-3 additional heteroatoms each selected from the group consisting of N, O, and S; wherein the heterocycle is optionally substituted by 1-4 substituents each independently selected from the group consisting of alkyl, cycloalkyl, halogenated cycloalkyl, halogenated alkyl, halogen, CN, OR₈, —(CH₂)₀₋₂OR₈, N(R₈)₂, (C═O)R₈, (C═O)N(R₈)₂, NR₈(C═O)R₈, and oxo where valence permits;
each occurrence of R₉ is independently H, alkyl, cycloalkyl, saturated heterocycle, aryl, heteroaryl, alkylaryl, alkylheteroaryl, (C═O)Rₐ, (C═O)(CRₐR_{b})ₙ₂ORₐ, (C═O)(CRₐR_{b})ₙ₂NRₐR_{b}, or SO₂Rₐ;
each occurrence of R₁₀ is independently H, alkyl, cycloalkyl, heterocycle, aryl, heteroaryl, alkylaryl, or alkylheteroaryl;
A₁ is aryl or heteroaryl;
A₂ is aryl or heteroaryl;
each occurrence of R₁₂ is independently H, alkyl, CN, CF₃, OCF₃, ORₐ, SRₐ, halogen, (CRₐR_{b})ₙ₂ORₐ, (C═O)NRₐR_{b}, (CRₐR_{b})ₙ₂NRₐR_{b}, or (CRₐR_{b})ₙ₂NR_{b}(C═O)Rₐ;
each occurrence of R₁₃ is independently H, alkyl, CN, CF₃, OCF₃, ORₐ, SRₐ, halogen, (CRₐR_{b})ₙ₂ORₐ, (C═O)NRₐR_{b}, (CRₐR_{b})ₙ₂NRₐR_{b}, or (CRₐR_{b})ₙ₂NR_{b}(C═O)Rₐ;
each occurrence of Rₐ and R_{b} are independently H, alkyl, alkenyl, cycloalkyl, saturated heterocycle comprising 1-3 heteroatoms each selected from the group consisting of N, O, and S, aryl, or heteroaryl; or alternatively Rₐ and R_{b} together with the carbon or nitrogen atom that they are connected to form a cycloalkyl or heterocycle comprising the nitrogen atom and 0-3 additional heteroatoms each selected from the group consisting of N, O, and S; the alkyl, cycloalkyl, heterocycle, aryl, and heteroaryl in X₁, X₂, X₃, A₁, A₂, R₁, R₂, R₃, R₄, R₅, R₆, R₇, R₉, R₁₀, R₁₂, R₁₃, Rₐ, or R_{b}, where applicable, are optionally substituted by 1-4 substituents each independently selected from the group consisting of alkyl, cycloalkyl, halogenated cycloalkyl, halogenated alkyl, halogen, CN, OR₈, —(CH₂)₀₋₂OR₈, N(R₈)₂, (C═O)R₈, (C═O)N(R₈)₂, NR₈(C═O)R₈, and oxo where valence permits;
each occurrence of R₈ is independently H, alkyl, or optionally substituted heterocycle; or alternatively the two R₈ groups together with the nitrogen atom that they are connected to form an optionally substituted heterocycle comprising the nitrogen atom and 0-3 additional heteroatoms each selected from the group consisting of N, O, and S;
each occurrence of m is independently 0, 1, 2, or 3;
each occurrence of n₁ is independently an integer from 0-3 wherein valence permits;
each occurrence of n₂ is independently an integer from 0-3;
n₄ is an integer from 0-3; and
n₅ is an integer from 0-3.

In some embodiments, each occurrence of R₄ is independently H, alkyl, cycloalkyl, saturated heterocycle, (CRₐR_{b})ₙ₂NRₐR_{b}, or (CRₐR_{b})ₙ₂ORₐ; and each occurrence of R₅ is independently H, alkyl, cycloalkyl, or saturated heterocycle.

In some embodiments, at least one occurrence of m is 0. In some embodiments, each occurrence of m is independently an integer from 1-3. In some embodiments, each occurrence of m is independently 2 or 3. In some embodiments, each occurrence of m is independently 1 or 2. In some embodiments, at least one occurrence of m is 1. In some embodiments, at least one occurrence of m is 2. In some embodiments, at least one occurrence of m is 3.

In some embodiments, the compound has the structure of Formula Ia, Ia′, IIa, IIa′, IIIa, or IVa:

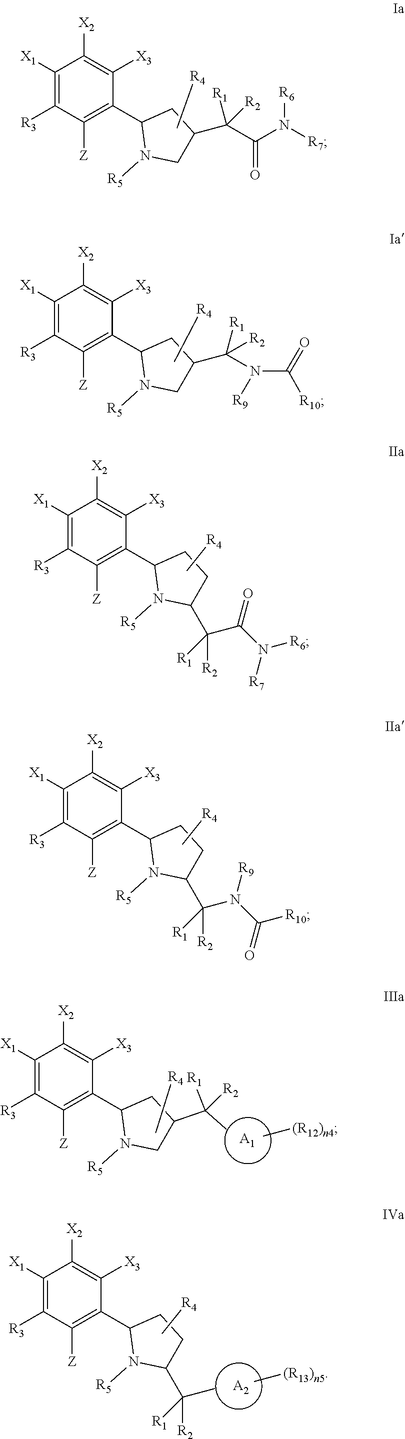

In some embodiments, the compound has the structure of Formula Ia:

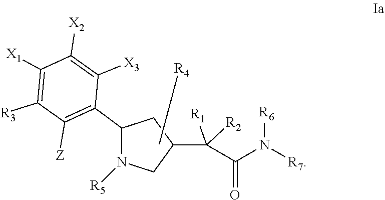

In some embodiments, the compound has the structure of Formula Ib, Ib′, IIb, IIb′, IIIb, or IVb:

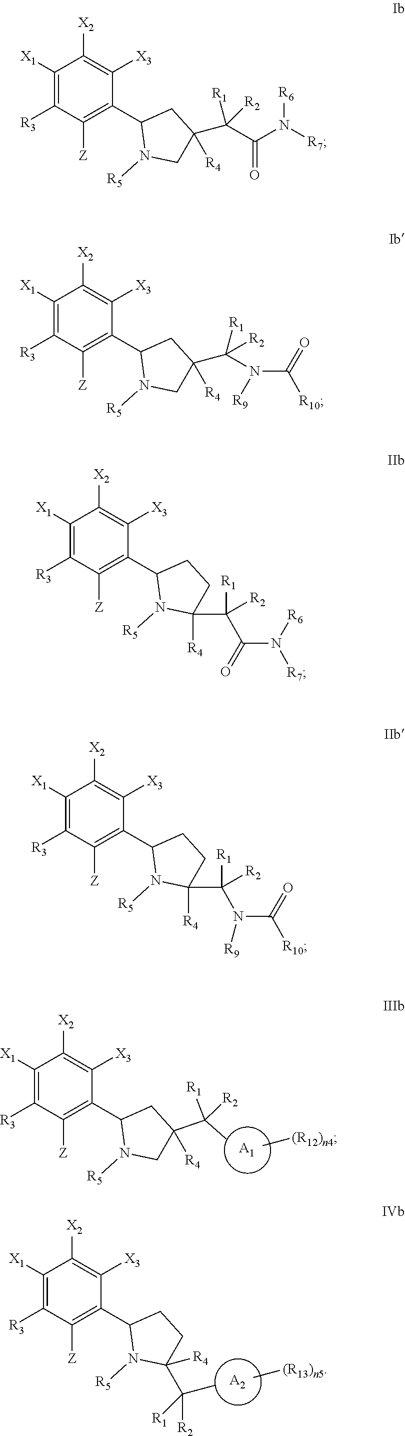

In some embodiments, the compound has the structure of Formula Ib.

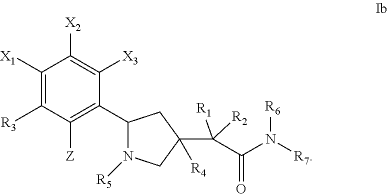

In some embodiments, at least one occurrence of R₄ is H, CN, alkyl, cycloalkyl, aryl, heteroaryl, CF₃, or ORₐ. In some embodiments, at least one occurrence of R₄ is halogen, saturated heterocycle, alkylaryl, alkylheteroaryl, (CRₐR_{b})ₙ₂ORₐ, oxo, (C═O)Rₐ, O(C═O)Rₐ, (C═O)ORₐ, or (CRₐR_{b})ₙ₂NRₐR_{b}. In some embodiments, at least one occurrence of R₄ is oxo, (C═O)Rₐ O(C═O)Rₐ, or (C═O)ORₐ. In some embodiments, at least one occurrence of R₄ is (CRₐR_{b})ₙ₂ORₐ. In some embodiments, at least one occurrence of R₄ is H or alkyl. Non-limiting examples of alkyl include methyl, ethyl, propyl, isopropyl, n-butyl, iso-butyl, sec-butyl, pentyl, hexyl, heptyl, and octyl. In some embodiments, at least one occurrence of R₄ is a cycloalkyl. Non-limiting examples of cycloalkyl include cyclopropyl, cyclobutyl, cyclopentyl, cyclohexyl, and cycloheptyl. In some embodiments, at least one occurrence of R₄ is halogen. Non-limiting examples of halogen include F, Cl, Br, and I.

In some embodiments, one or more occurrences of R₄ are (CRₐR_{b})ₙ₂ORₐ or (CRₐR_{b})ₙ₂NRₐR_{b}. In some embodiments, at least one occurrence of R₄ is (CRₐR_{b})ₙ₂NRₐR_{b}. In some embodiments, one or more occurrences of R₄ are ORₐ, NRₐR_{b}, —CH₂ORₐ, —CH₂NRₐR_{b}, —CH₂CH₂ORₐ, or —CH₂CH₂NRₐR_{b}.

In some specific embodiments, at least one occurrence of R₄ is NH₂, CH₂NH₂, or CH₂CH₂NH₂. In other specific embodiments, at least one occurrence of R₄ is OH, CH₂OH or CH₂NH₂.

In still other embodiments, at least one occurrence of R₄ is an optionally substituted 4-, 5-, 6- or 7-membered heterocycle containing 1-3 heteroatoms each selected from the group consisting of N, O, and S. In further embodiments, at least one occurrence of R₄ is a heterocycle selected from the group consisting of

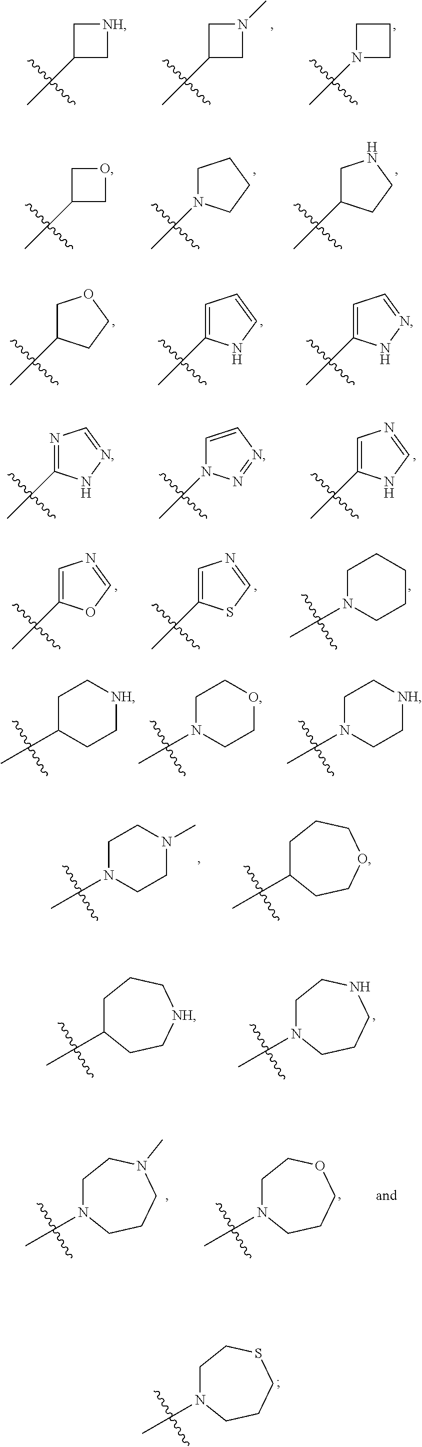

wherein the heterocycle is optionally substituted by alkyl, OH, oxo, or (C═O)C₁₋₄alkyl where valence permits. In some embodiments, at least one occurrence of R₄ is a N-containing heterocycle, wherein the heterocycle is optionally substituted by alkyl, OH, oxo, or (C═O)C₁₋₄alkyl where valence permits. Non-limiting examples of N-containing heterocycle include

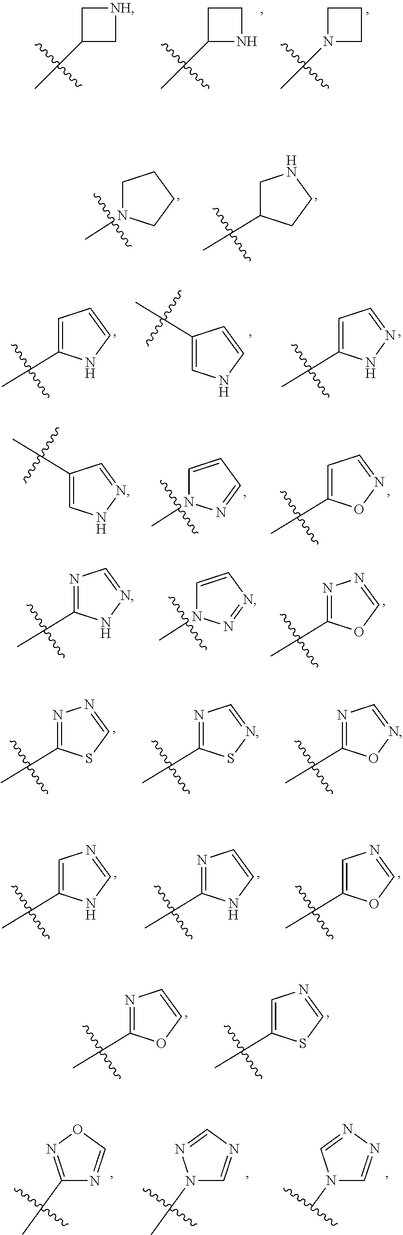

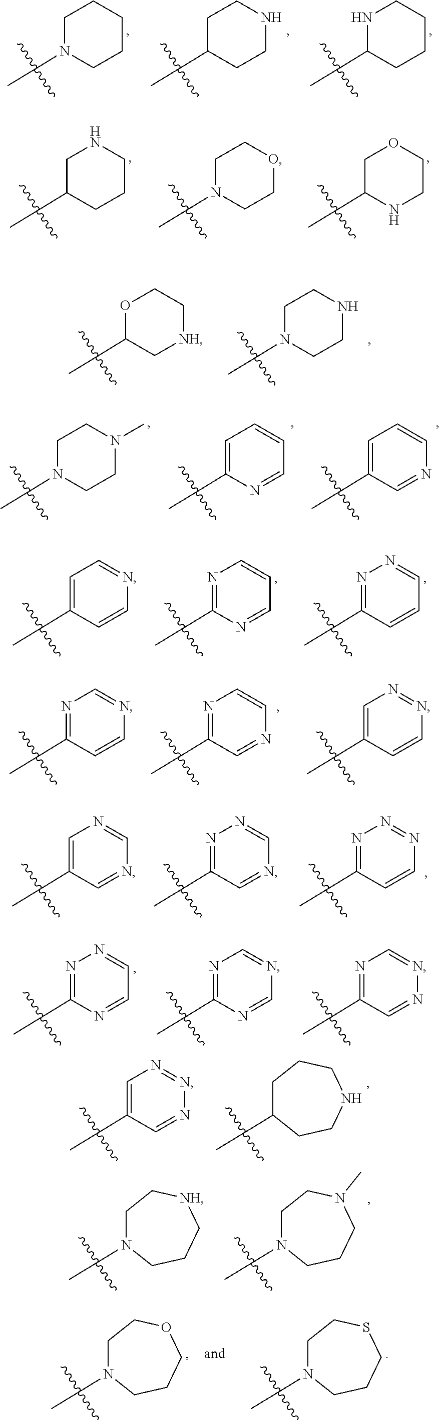

In some embodiments, each occurrence of R₄ is independently H, Me, Et, Pr, Bu, or a saturated heterocycle or heteroaryl selected from the group consisting of

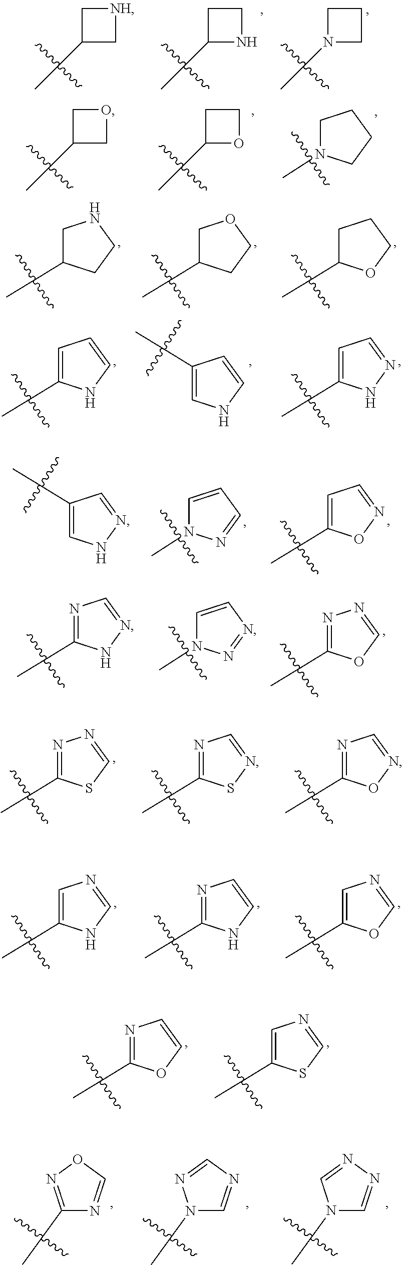

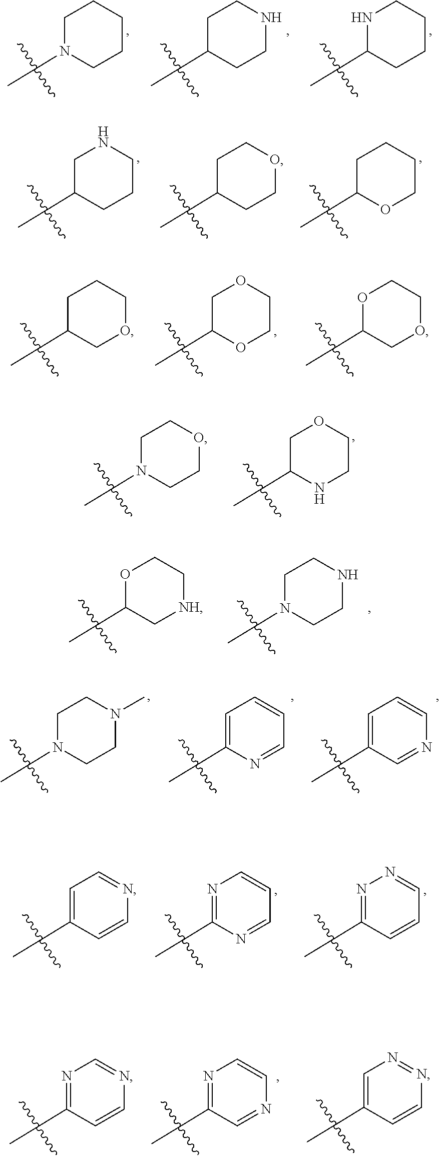

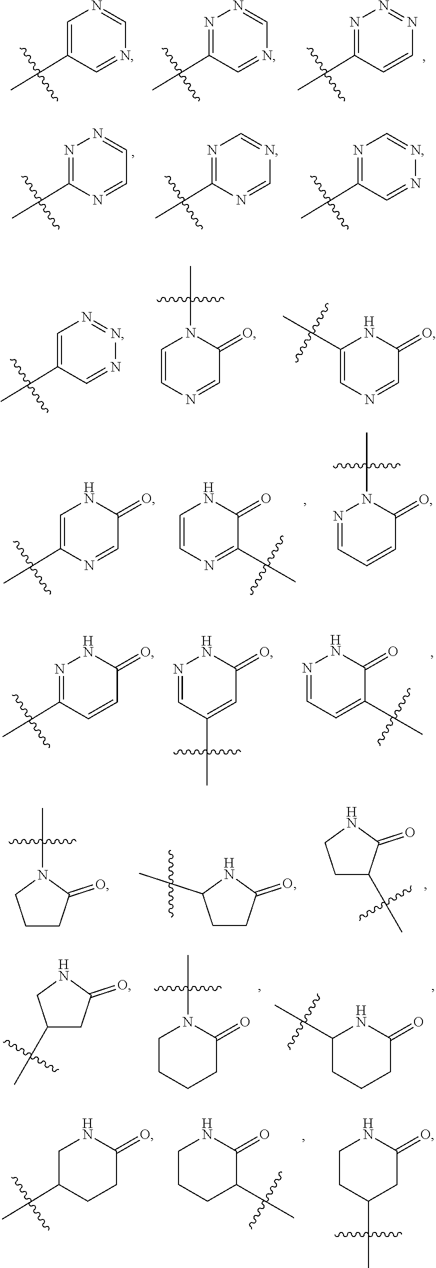

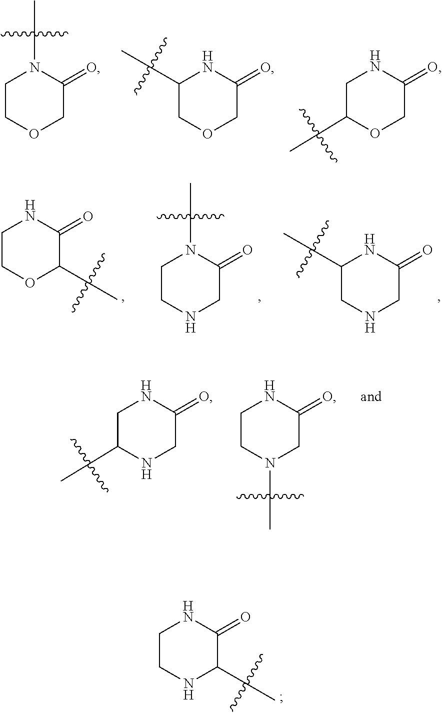

wherein the saturated heterocycle or heteroaryl is optionally substituted by cyano, cycloalkyl, fluorinated alkyl, fluorinated cycloalkyl, halogen, OH, NH₂, oxo, or (C═O)C₁₋₄ alkyl where valence permits.

In some specific embodiments, at least one occurrence of R₄ is H, halogen, alkyl, ORₐ, NRₐR_{b}, or oxo. In other specific embodiments, at least one occurrence of R₄ is H, F, Cl, Br, Me, Et, Pr, iso-Pr, Bu, iso-Bu, sec-Bu, or tert-Bu. In other specific embodiments, at least one occurrence of R₄ is OH, NH₂, NHMe, NMe₂, NHEt, NMeEt, NEt₂, or oxo. In still other specific embodiments, at least one occurrence of R₄ is H, halogen, alkyl, OH, NH₂, CN, CF₃, or OCF₃. In still other specific embodiments, at least one occurrence of R₄ is H, Me or Et.

In further embodiments, two R₄ groups taken together with the carbon atom(s) that they are connected to form a 3-7 membered optionally substituted cycloalkyl or heterocycle.

In some embodiments, at least one occurrence of n₁ is an integer from 0-2. In some embodiments, at least one occurrence of n₁ is 0 or 1. In some embodiments, at least one occurrence of n₁ is 0. In some embodiments, at least one occurrence of n₁ is 1.

In some specific embodiments, at least one occurrence of n₁ is 0 and at least one occurrence of R₅ is H or alkyl. In some specific embodiments, at least one occurrence of n₁ is 1 and at least one occurrence of R₅ is H or alkyl.

In some embodiments, each occurrence R₅ is independently H, alkyl, cycloalkyl, aryl, heteroaryl, (C═O)Rₐ, (C═O)(CRₐR_{b})ₙ₂ORₐ, (C═O)(CRₐR_{b})ₙ₂NRₐR_{b}, or SO₂Rₐ. In some embodiments, at least one occurrence of R₅ is H, alkyl, or cycloalkyl. In some embodiments, at least one occurrence of R₅ is aryl or heteroaryl. In some specific embodiments, at least one occurrence of R₅ is (C═O)Rₐ, (C═O)(CRₐR_{b})ₙ₂ORₐ, (C═O)(CRₐR_{b})ₙ₂NRₐR_{b}, or SO₂Rₐ. In some specific embodiments, at least one occurrence of R₅ is (C═O)Rₐ or (C═O)—(CRₐR_{b})₁₋₂—ORₐ. In some specific embodiments, at least one occurrence of R₅ is (C═O)—(CRₐR_{b})₁₋₂—NRₐR_{b} or (C═O)NRₐR_{b}. In some specific embodiments, at least one occurrence of R₅ is (C═O)NRₐR_{b}, (C═O)CH₂NRₐR_{b}, or (C═O)CH₂CH₂NRₐR_{b}. In some specific embodiments, at least one occurrence of R₅ is H. In other specific embodiments, at least one occurrence of R₅ is methyl. In other specific embodiments, at least one occurrence of R₅ is ethyl.

In some embodiments, each occurrence of R₁ and R₂ is independently H, alkyl optionally substituted with OR₈, halogen, cycloalkyl, or fluorinated alkyl. In some specific embodiments, each occurrence of R₁ and R₂ is independently H, CH₃, CH₂CH₃, CH₂OH, CH₂CH₂OH, CH₂OCH₃, CH₂CH₂OCH₃, or

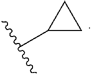 [SMILES: *C1CC1]

In other specific embodiments, each occurrence of R₁ and R₂ is independently H and H, H and Me, Me and Me, H and Et, Me and Et, Et and Et, H and CH₂OH, H and CH₂CH₂OH, H and CH₂OCH₃, H and CH₂CH₂OCH₃, or H and

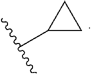 [SMILES: *C1CC1]

In still other embodiments, each occurrence of the structural moiety —(CR₁R₂)ₘ— is independently selected from the group consisting of —CH₂—, —CH(CH₃)—, —C(CH₃)₂—, —CH(CH₂CH₃)—, —CCH₃(CH₂CH₃)—, —CH(CH₂OH)—, —CH(CH₂OCH₃)—, —CH₂—CH₂—, —CH(CH₃)—CH₂—, —CH₂—C(CH₃)₂—,

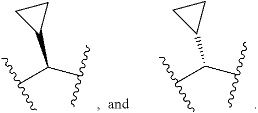

In some embodiments, at least one occurrence of R₁ or R₂ is independently cycloalkyl, saturated heterocycle, aryl, heteroaryl. In some embodiments, at least one occurrence of R₁ or R₂ is independently CN, CF₃, OCF₃, ORₐ, SRₐ, NRₐR_{b}, or NR_{b}(C═O)Rₐ.

In some embodiments, each occurrence of R₆ and R₇ is independently cycloalkyl or heterocycle; wherein the cycloalkyl or heterocycle is optionally substituted by 1-2 substituents each independently selected from the group consisting of halogen, CN, OH, OMe, —(CH₂)₁₋₂OMe and —(CH₂)₁₋₂OH. In some embodiments, each occurrence of R₆ and R₇ is independently H or alkyl; wherein the alkyl is optionally substituted by 1-2 substituents each independently selected from the group consisting of halogen, CN, OH, OMe, —(CH₂)₁₋₂OMe and —(CH₂)₁₋₂OH. In some specific embodiments, each occurrence of R₆ and R₇ is independently H, —CH₃, —CH₂OH, —CH₂CH₂OH or —CH₂CH₂CH₂OH. In some embodiments, each occurrence of R₆ and R₇ is independently alkylaryl or alkylheteroaryl, wherein the alkylaryl or alkylteteroaryl is optionally substituted by 1-2 substituents each independently selected from the group consisting of halogen, CN, OH, OMe, —(CH₂)₁₋₂OMe and —(CH₂)₁₋₂OH.

In some embodiments, R₆ and R₇ taken together with the nitrogen atom they are connected to form a heterocycle comprising the nitrogen atom and 0-3 additional heteroatoms each selected from the group consisting of N, O, and S; wherein the heterocycle is optionally substituted by 1-4 substituents each independently selected from the group consisting of alkyl, cycloalkyl, halogenated cycloalkyl, halogenated alkyl, halogen, CN, OR₈, —(CH₂)₀₋₂OR₈, N(R₈)₂, (C═O)R₈, (C═O)N(R₈)₂, NR₈(C═O)R₈, and oxo where valence permits.

In some embodiments, R₆ and R₇ taken together with the nitrogen atom they are connected to form a 4-, 5-, or 6-membered heterocycle; wherein the heterocycle is optionally substituted by 1-2 substituents each independently selected from the group consisting of alkyl, halogenated alkyl, halogen, CN, OH, and —(CH₂)₁₋₂OH. Non-limiting examples of 4-, 5-, or 6-membered heterocycle include azetidine, pyrrolidine, piperidine, and piperazine. In some specific embodiments, the 4-, 5-, or 6-membered heterocycle is substituted by 1-2 substituents each independently selected from the group consisting of OH and —(CH₂)₁₋₂OH. In some specific embodiments, the 4-, 5-, or 6-membered heterocycle is

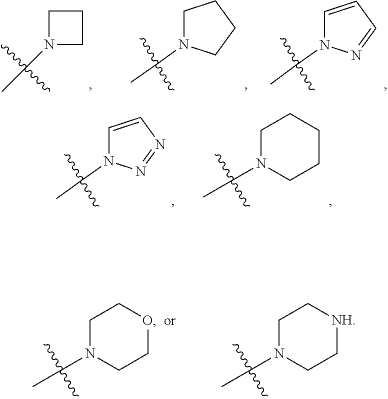

In some specific embodiments, R₆ and R₇ taken together with the nitrogen atom they are connected to form azetidine optionally substituted by alkyl or OH. In other specific embodiments, R₆ and R₇ taken together with the nitrogen atom they are connected to form pyrrolidine optionally substituted by alkyl or OH. In other specific embodiments, R₆ and R₇ taken together with the nitrogen atom they are connected to form piperidine optionally substituted by alkyl or OH.

In specific embodiments, at least one occurrence of the structural moiety

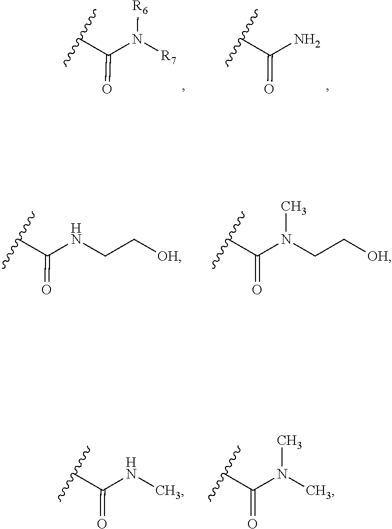

has the structure of

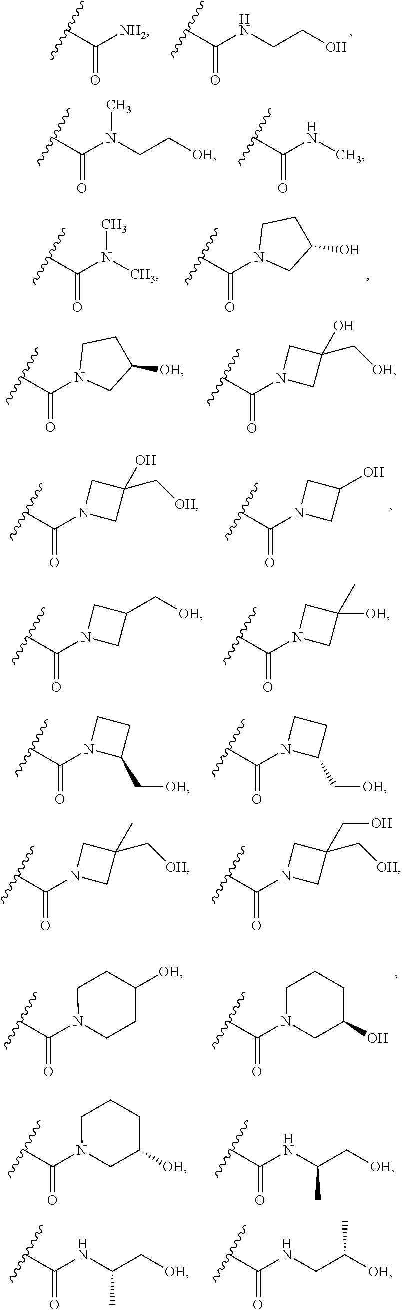

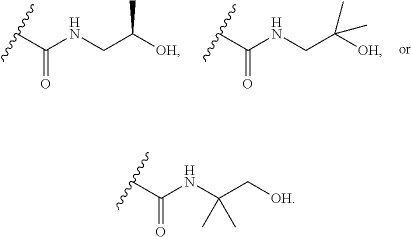

In some embodiments, each occurrence of R₉ is independently cycloalkyl, saturated heterocycle, aryl, heteroaryl, alkylaryl, or alkylheteroaryl. In some embodiments, at least one occurrence of R₉ is (C═O)Rₐ, (C═O)(CRₐR_{b})ₙ₂ORₐ, (C═O)(CRₐR_{b})ₙ₂NRₐR_{b}, (C═O)NRₐR_{b}, or SO₂Rₐ. In some specific embodiments, at least one occurrence of R₉ is H or alkyl. Non-limiting examples of alkyl include methyl, ethyl, propyl, isopropyl, n-butyl, iso-butyl, sec-butyl, pentyl, hexyl, heptyl, and octyl. In other specific embodiments, at least one occurrence of R₉ is H or CH₃.

In embodiments, at least one occurrence of R₁₀ is cycloalkyl, or heterocycle; wherein the cycloalkyl or heterocycle is optionally substituted by 1-2 substituents each independently selected from the group consisting of halogen, CN, OH, OMe, —(CH₂)₁₋₂OMe and —(CH₂)₁₋₂OH. In some embodiments, at least one occurrence of R₁₀ is H or alkyl; wherein the alkyl is optionally substituted by 1-2 substituents each independently selected from the group consisting of halogen, CN, OH, OMe, —(CH₂)₁₋₂OMe and —(CH₂)₁₋₂OH. In further embodiments, at least one occurrence of R₁₀ is alkyl that is optionally substituted by 1-2 substituents each independently selected from the group consisting of halogen, CN, and OH. In some specific embodiments, at least one occurrence of R₁₀ is H, —CH₃, —CH₂OH, or —CH₂CH₂OH.

In some embodiments, A₁ is a 5- or 6-membered heteroaryl containing 1-3 heteroatoms each selected from the group consisting of N, O, and S. In further embodiments, A₁ is a heteroaryl selected from the group consisting

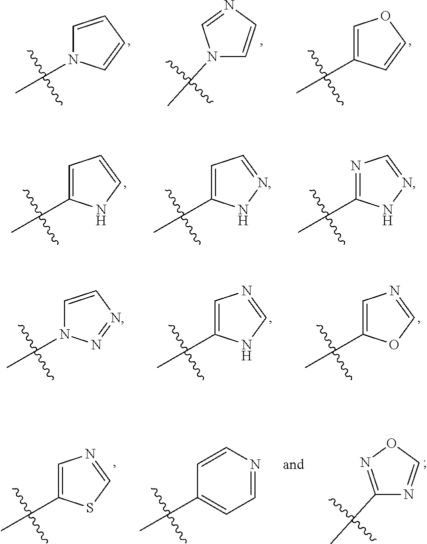

wherein the heterocycle is optionally substituted by alkyl, OH, oxo, or (C═O)C₁₋₄alkyl where valence permits.

In some embodiments, A₁ is a N-containing heteroaryl, wherein the heteroaryl is optionally substituted by alkyl, OH, oxo, or (C═O)C₁₋₄alkyl where valence permits. Non-limiting examples of N-containing heteroaryl include

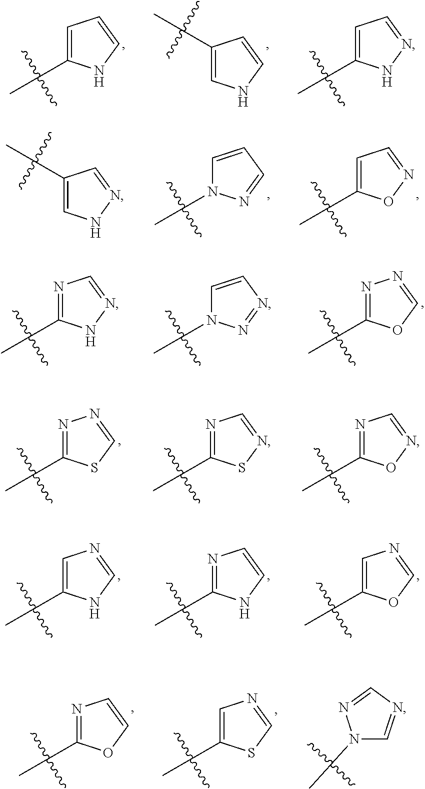

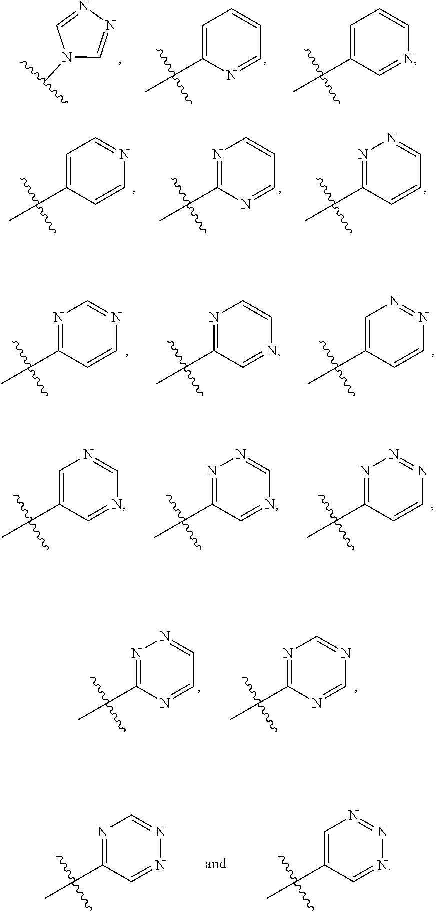

In some embodiments, A₁ is a 5- or 6-membered heteroaryl, or phenyl. In some embodiments, A₁ is a 5-membered heteroaryl. A₁ is selected from the group consisting of

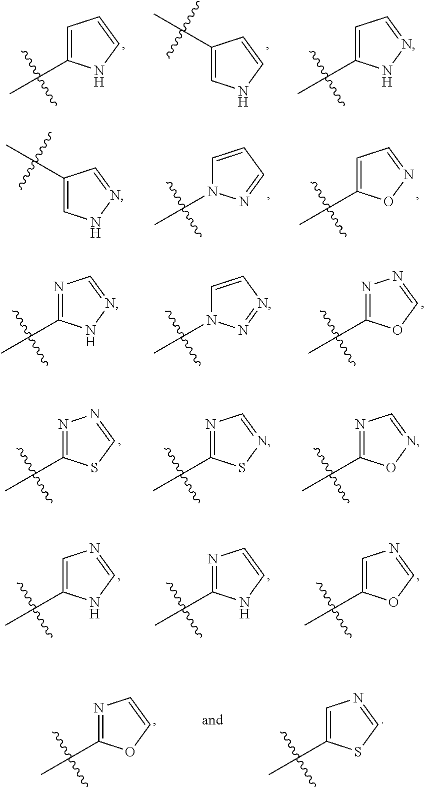

In some specific embodiments, A₁ is

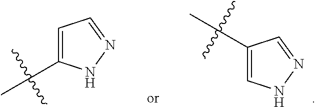 [SMILES: *c1ccn[nH]1 ; *c1cn[nH]c1]

In some embodiments, A₁ is a 7 to 11 membered bicyclic, or 8 to 16 membered tricyclic aryl or heteroaryl. Non-limiting examples of bicyclic or tricyclic rings include biphenyl, naphthyl, phenanthrenyl, indolyl, isoindolyl, benzothiazolyl, benzoxazolyl, benzoxadiazolyl, benzothienyl, quinolinyl, isoquinolinyl, benzimidazolyl, chromonyl, coumarinyl, cinnolinyl, quinoxalinyl, indazolyl, pyrrolopyridyl, furopyridinyl (such as furo[2,3-c]pyridinyl, furo[3,2-b]pyridinyl] or furo[2,3-b]pyridinyl), carbazolyl, phenanthrolinyl, acridinyl, and phenanthridinyl.

In some embodiments, A₁ is selected from the group consisting of

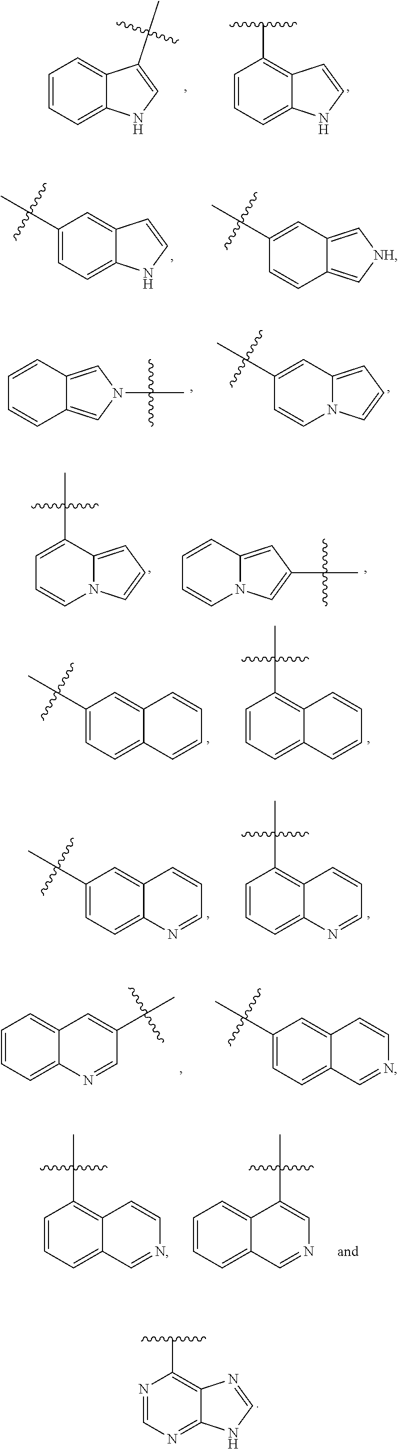

In some embodiments, A₂ is a 5- or 6-membered heteroaryl containing 1-3 heteroatoms each selected from the group consisting of N, O, and S. In further embodiments, A₂ is a heteroaryl selected from the group consisting of

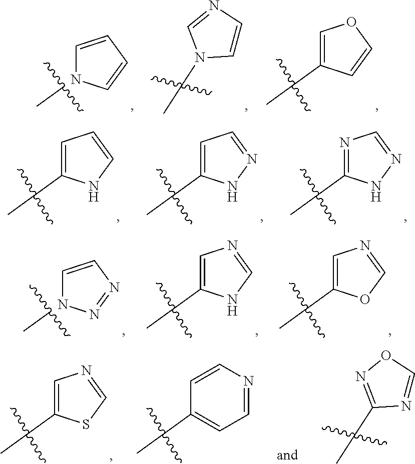

wherein the heterocycle is optionally substituted by alkyl, OH, oxo, or (C═O)C₁₋₄alkyl where valence permits.

In some embodiments, A₂ is a N-containing heteroaryl, wherein the heteroaryl is optionally substituted by alkyl, OH, oxo, or (C═O)C₁₋₄alkyl where valence permits. Non-limiting examples of N-containing heteroaryl include

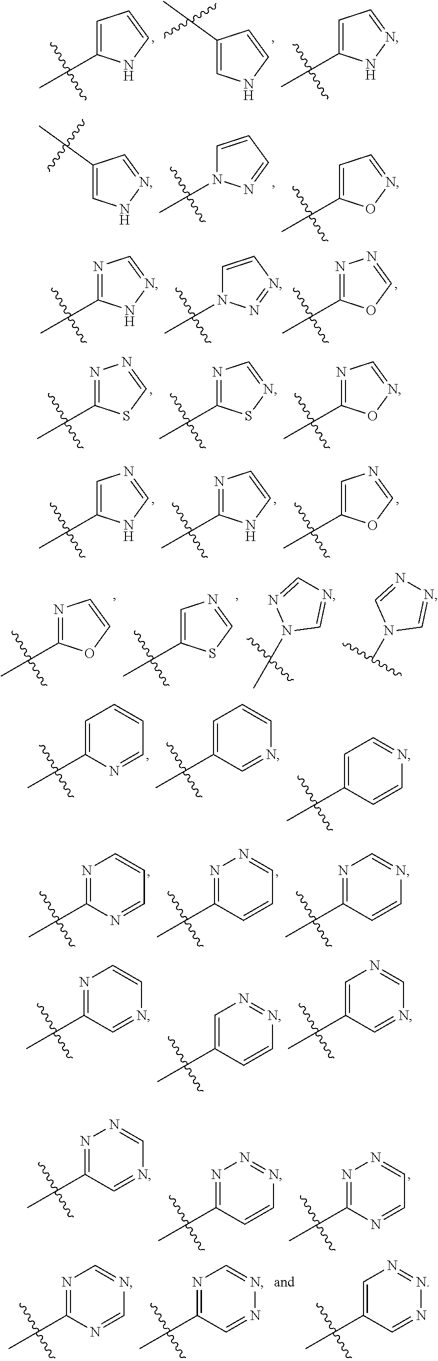

In some embodiments, A₂ is a 5- or 6-membered heterocycle, or phenyl. In some embodiments, A₂ is a 5-membered heteroaryl. In some embodiments, A₂ is selected from the group consisting of

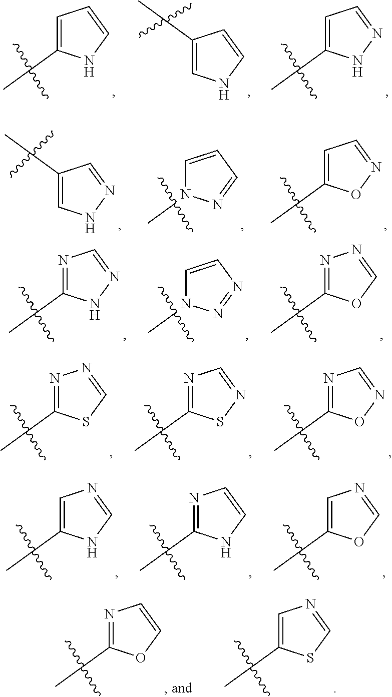

specific embodiments, A₂ is

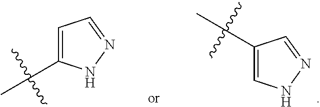 [SMILES: *c1ccn[nH]1 ; *c1cn[nH]c1]

In some embodiments, A₂ is a 7 to 11 membered bicyclic, or 8 to 16 membered tricyclic aryl or heteroaryl. Non-limiting examples of bicyclic or tricyclic rings include biphenyl, naphthyl, phenanthrenyl, indolyl, isoindolyl, benzothiazolyl, benzoxazolyl, benzoxadiazolyl, benzothienyl, quinolinyl, isoquinolinyl, benzimidazolyl, chromonyl, coumarinyl, cinnolinyl, quinoxalinyl, indazolyl, pyrrolopyridyl, furopyridinyl (such as furo[2,3-c]pyridinyl, furo[3,2-b]pyridinyl] or furo[2,3-b]pyridinyl), carbazolyl, phenanthrolinyl, acridinyl, and phenanthridinyl.

In some embodiments, A₂ is selected from the group consisting of

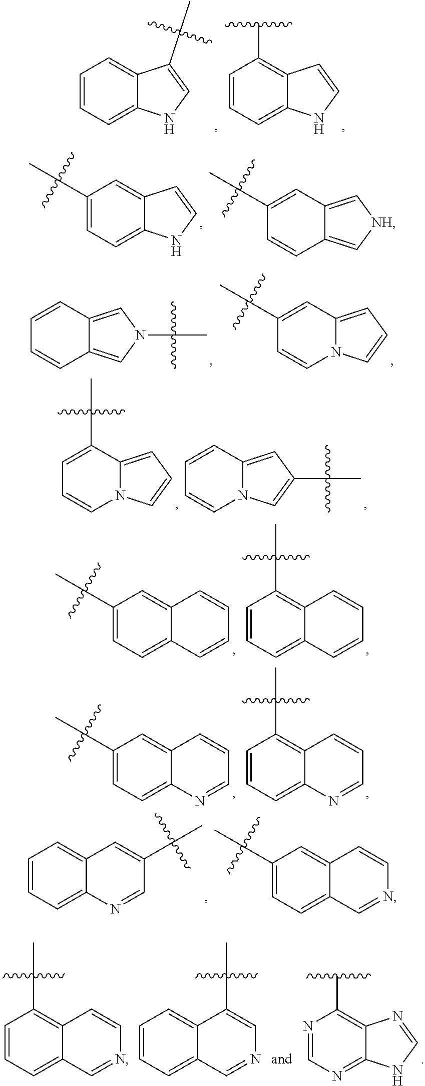

In some embodiments, each occurrence of R₁₂ is independently H, alkyl, CF₃, or halogen. In embodiments, at least one occurrence of R₁₂ is CN, CF₃, OCF₃, ORₐ, or SRₐ. In some embodiments, at least one occurrence of R₁₂ is halogen, NRₐR_{b}, or NR_{b}(C═O)Rₐ. In some embodiments, at least one occurrence of R₁₂ is ORₐ, SRₐ, or NRₐR_{b}. In some embodiments, at least one occurrence of R₁₂ is NR_{b}(C═O)Rₐ. In some embodiments, at least one occurrence of R₁₂ is (CRₐR_{b})ₙ₂ORₐ, (C═O)NRₐR_{b}, (CRₐR_{b})ₙ₂NRₐR_{b}, or (CRₐR_{b})ₙ₂NR_{b}(C═O)Rₐ. In some embodiments, at least one occurrence of R₁₂ is H, halogen, fluorinated alkyl, or alkyl. In some embodiments, at least one occurrence of R₁₂ is H, fluorinated alkyl, or alkyl. In some embodiments, at least one occurrence of R₁₂ is H, Me, Et, i-Pr, n-Bu, CF₂H, CF₂Cl, or CF₃. In some specific embodiments, at least one occurrence of R₁₂ is H.

In some embodiments, each occurrence of R₁₃ is independently H, alkyl, CF₃, or halogen. In some embodiments, at least one occurrence of R₁₃ is CN, CF₃, OCF₃, ORₐ, or SRₐ. In some embodiments, at least one occurrence of R₁₃ is halogen, NRₐR_{b}, or NR_{b}(C═O)Rₐ. In some embodiments, at least one occurrence of R₁₃ is ORₐ, SRₐ, or NRₐR_{b}. In some embodiments, at least one occurrence of R₁₃ is NR_{b}(C═O)Rₐ. In some embodiments, at least one occurrence of R₁₂ is (CRₐR_{b})ₙ₂ORₐ, (C═O)NRₐR_{b}, (CRₐR_{b})ₙ₂NRₐR_{b}, or (CRₐR_{b})ₙ₂NR_{b}(C═O)Rₐ. In some embodiments, at least one occurrence of R₁₃ is H, halogen, fluorinated alkyl, or alkyl. In some embodiments, at least one occurrence of R₁₃ is H, fluorinated alkyl, or alkyl. In some embodiments, at least one occurrence of R₁₃ is H, Me, Et, i-Pr, n-Bu, CF₂H, CF₂Cl, or CF₃. In some specific embodiments, at least one occurrence of R₁₃ is H.

In some embodiments, n₄ is an integer from 0-3. In some embodiments, n₄ is an integer from 1-3. In some embodiments, n₄ is 0. In some embodiments, n₄ is 1 or 2. In some embodiments, n₄ is 1.

In some embodiments, n₅ is an integer from 0-3. In some embodiments, n₅ is an integer from 1-3. In some embodiments, n₅ is 0. In some embodiments, n₅ is 1 or 2. In some embodiments, n₅ is 1.

In some embodiments, at least one occurrence of Z is ORₐ. In some embodiments, at least one occurrence of Z is OH or O—(C₁-C₄ alkyl). In some embodiments, at least one occurrence of Z is OH, OMe, OEt, OPr, Oi-Pr, OBu, Oi-Bu, Osec-Bu, or Ot-Bu. In some embodiments, at least one occurrence of Z is OH.

In some embodiments, each occurrence of X₁ is independently H, halogen, CN, alkyl, halogenated alkyl, cycloalkyl, or halogenated cycloalkyl. In some embodiments, at least one occurrence of X₁ is H, halogen, fluorinated alkyl, or alkyl. In some embodiments, at least one occurrence of X₁ is H or halogen. In other embodiments, at least one occurrence of X₁ is fluorinated alkyl or alkyl. In other embodiments, at least one occurrence of X₁ is cycloalkyl. In some embodiments, at least one occurrence of X₁ is H, F, Cl, Br, Me, CF₂H, CF₂Cl, or CF₃. In some embodiments, at least one occurrence of X₁ is H, F, or Cl. In some embodiments, at least one occurrence of X₁ is F or Cl. In some embodiments, at least one occurrence of X₁ is H or Cl. In some embodiments, at least one occurrence of X₁ is F. In some embodiments, at least one occurrence of X₁ is Cl. In some embodiments, at least one occurrence of X₁ is CF₃ or CF₂H. In some embodiments, at least one occurrence of X₁ is CF₂Cl. In some embodiments, at least one occurrence of X₁ is H.

In some embodiments, each occurrence of X₂ is independently H, halogen, CN, alkyl, halogenated alkyl, cycloalkyl, or halogenated cycloalkyl. In some embodiments, at least one occurrence of X₂ is H, halogen, fluorinated alkyl, or alkyl. In some embodiments, at least one occurrence of X₂ is H or halogen. In other embodiments, at least one occurrence of X₂ is fluorinated alkyl or alkyl. In other embodiments, at least one occurrence of X₂ is cycloalkyl. In some embodiments, at least one occurrence of X₂ is H, F, Cl, Br, Me, CF₂H, CF₂Cl, or CF₃. In some embodiments, at least one occurrence of X₂ is H, F, or Cl. In some embodiments, at least one occurrence of X₂ is F or Cl. In some embodiments, at least one occurrence of X₂ is H or Cl. In some embodiments, at least one occurrence of X₂ is F. In some embodiments, at least one occurrence of X₂ is Cl. In some embodiments, at least one occurrence of X₂ is CF₃ or CF₂H. In some embodiments, at least one occurrence of X₂ is CF₂Cl. In some embodiments, at least one occurrence of X₂ is H.

In some embodiments, each occurrence of X₃ is independently H, halogen, CN, alkyl, halogenated alkyl, cycloalkyl, or halogenated cycloalkyl. In some embodiments, at least one occurrence of X₃ is H, halogen, alkyl, or halogenated alkyl. In some embodiments, at least one occurrence of X₃ is H, halogen, fluorinated alkyl, or alkyl. In some embodiments, at least one occurrence of X₃ is H or halogen. In other embodiments, at least one occurrence of X₃ is fluorinated alkyl or alkyl. In some embodiments, at least one occurrence of X₃ is H, F, Cl, Br, Me, CF₂H, CF₂Cl, or CF₃. In some embodiments, at least one occurrence of X₃ is H, F, or Cl. In some embodiments, at least one occurrence of X₃ is F or Cl. In some embodiments, at least one occurrence of X₃ is H or Cl. In some embodiments, at least one occurrence of X₃ is F. In some embodiments, at least one occurrence of X₃ is Cl. In some embodiments, at least one occurrence of X₃ is CF₃ or CF₂H. In some embodiments, at least one occurrence of X₃ is CF₂Cl. In some embodiments, at least one occurrence of X₃ is H.

In embodiments, at least one occurrence of R₃ is H, alkyl, CF₃, or halogen. In embodiments, at least one occurrence of R₃ is cycloalkyl or saturated heterocycle. In embodiments, at least one occurrence of R₃ is aryl or heteroaryl. In embodiments, at least one occurrence of R₃ is CN, CF₃, OCF₃, ORₐ, or SRₐ. In embodiments, at least one occurrence of R₃ is halogen, NRₐR_{b}, or NR_{b}(C═O)Rₐ. In some embodiments, at least one occurrence of R₃ is ORₐ, SRₐ, or NRₐR_{b}. In some embodiments, at least one occurrence of R₃ is NR_{b}(C═O)Rₐ. In some embodiment, at least one occurrence of R₃ is H, halogen, fluorinated alkyl, or alkyl. In some embodiments, at least one occurrence of R₃ is H, fluorinated alkyl, or alkyl. In some embodiments, at least one occurrence of R₃ is H, Me, Et, i-Pr, n-Bu, CF₂H, CF₂Cl, or CF₃. In some specific embodiments, at least one occurrence of R₃ is H.

In some embodiments, at least one occurrence of the structural moiety

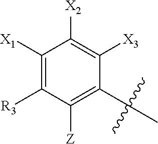

has the structure of

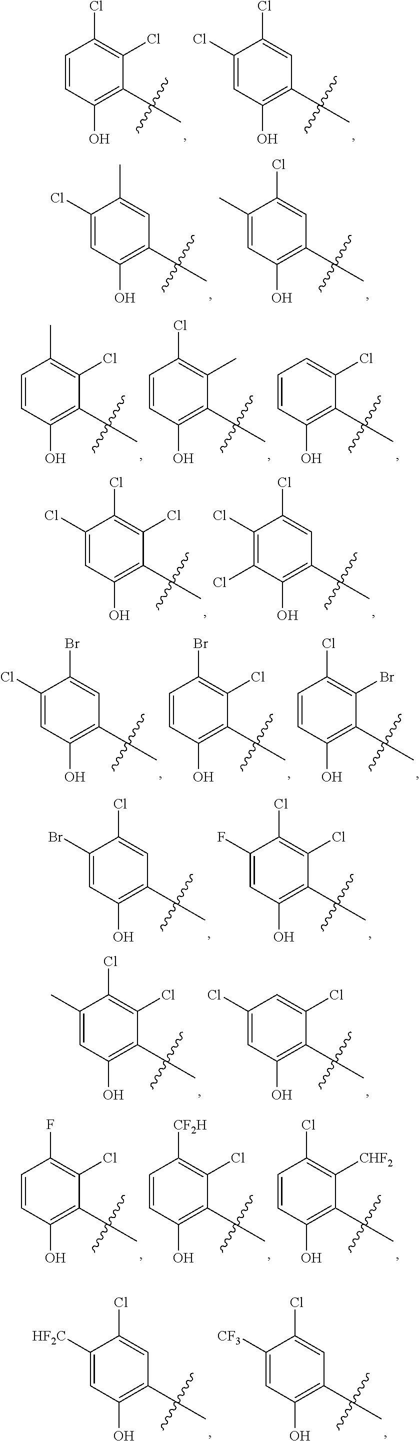

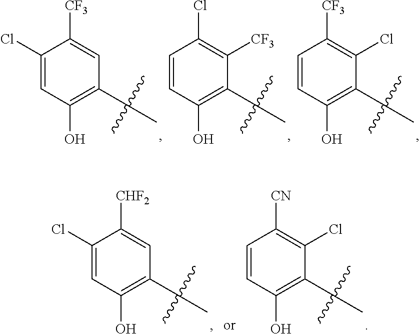

In some embodiments, at least one occurrence of the structural moiety

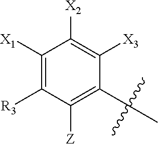

has the structure of OH

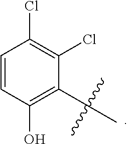 [SMILES: *c1c(O)ccc(Cl)c1Cl]

In some embodiments, the compound of Formula I, I′, II, II′, III, or IV has a structure of Formula Ic, Ic′, Id, Id′, IIc, IIc′, IId, IId′, IIIc, IIId, IVc, or IVd:

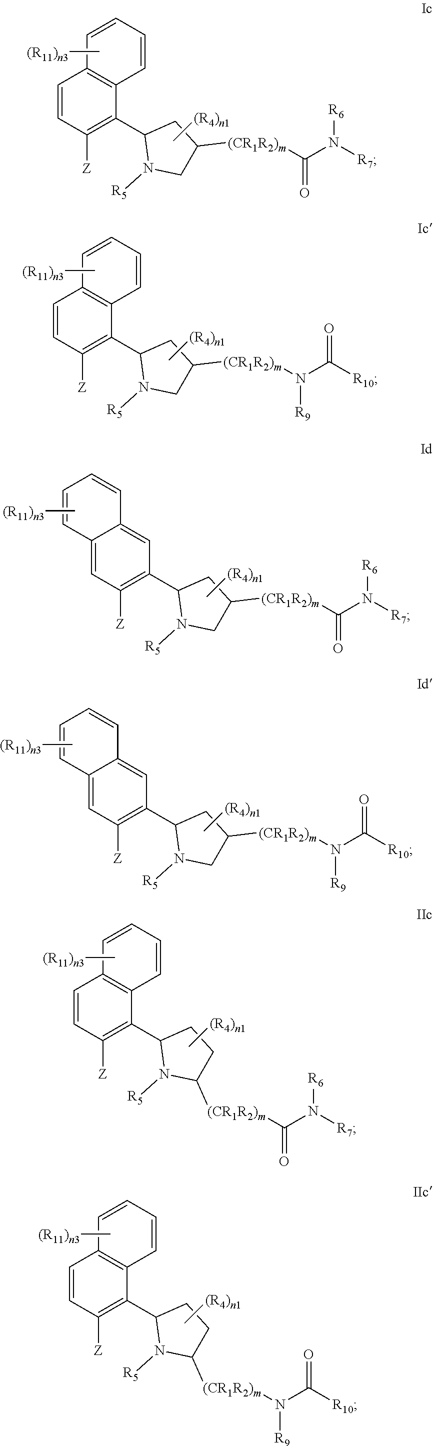

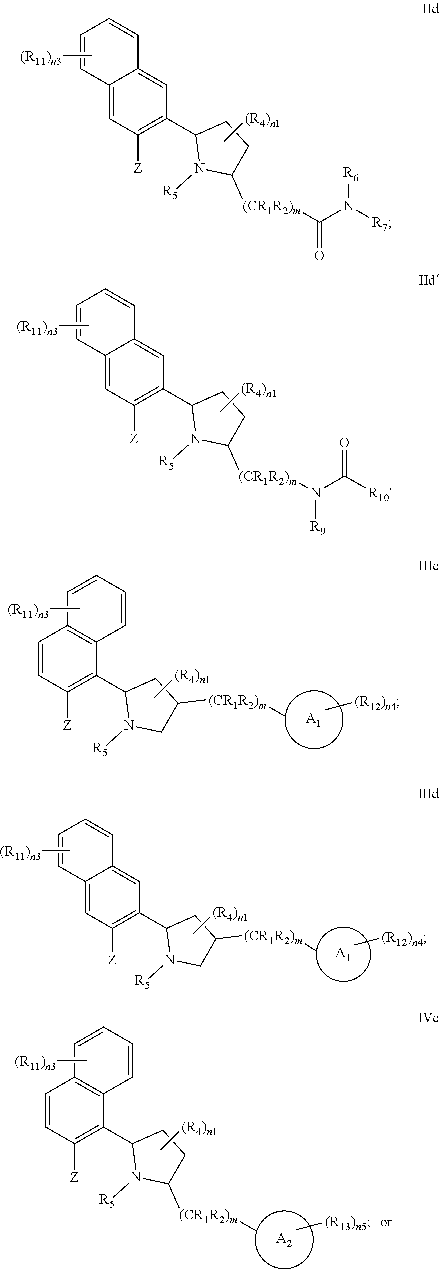

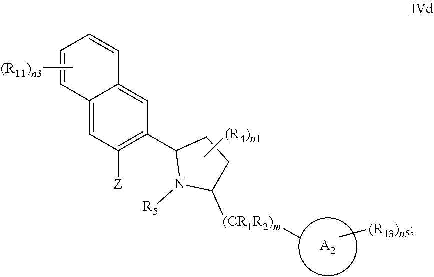

wherein each occurrence of R₁₁ is independently H, halogen, fluorinated alkyl, or alkyl; and each occurrence of n₃ is independently an integer from 0-3.

In some embodiments, each occurrence of n₃ is independently an integer from 0-3. In some embodiments, at least one occurrence of n₃ is an integer from 1-3. In some embodiments, at least one occurrence of n₃ is 0. In some embodiments, at least one occurrence of n₃ is 1 or 2. In some embodiments, at least one occurrence of n₃ is 1.

In some embodiments, each occurrence of R₁₁ is independently H, halogen, fluorinated alkyl, or alkyl. In some embodiments, at least one occurrence of R₁₁ is H or halogen. In some embodiments, at least one occurrence of R₁₁ is alkyl or fluorinated alkyl. In some embodiments, at least one occurrence of R₁₁ is H, Cl, Br, CF₃, CHF₂, or Me. In some embodiments, at least one occurrence of R₁₁ is H.

In some embodiments, at least one occurrence of Rₐ or R_{b} is independently H, alkyl, alkenyl, cycloalkyl, saturated heterocycle, aryl, or heteroaryl. In some embodiments, at least one occurrence of Rₐ or R_{b} is independently H, alkyl or alkenyl. In some embodiments, at least one occurrence of Rₐ or R_{b} is independently H, Me, Et, Pr, or Bu. In some embodiments, at least one occurrence of Rₐ or R_{b} is independently a heterocycle selected from the group consisting of

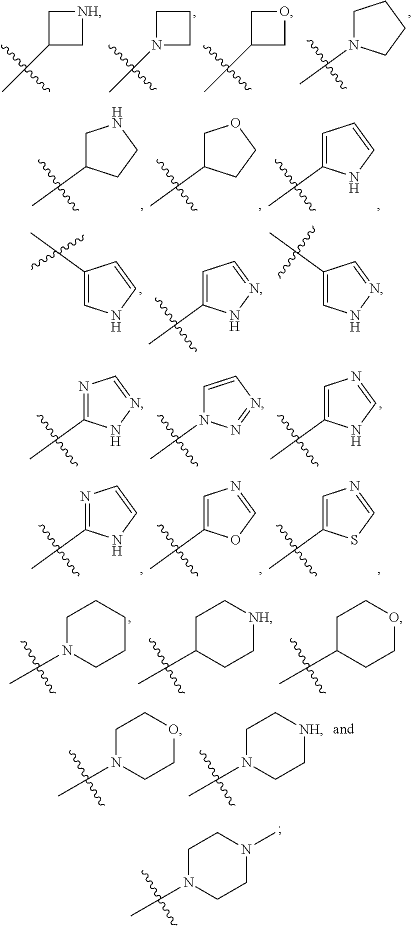

where the heterocycle is optionally substituted by alkyl, OH, oxo, or (C═O)C₁₋₄alkyl where valence permits. In some embodiments, at least one occurrence of Rₐ or R_{b} is independently H or

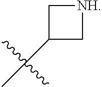 [SMILES: *C1CNC1]

In some embodiments, Rₐ and R_{b} together with the carbon atom that they are connected to form a cycloalkyl, optionally substituted by 1-4 substituents each independently selected from the group consisting of alkyl, cycloalkyl, halogenated cycloalkyl, halogenated alkyl, halogen, CN, OR₈, —(CH₂)₀₋₂OR₈, N(R₈)₂, (C═O)R₈, (C═O)N(R₈)₂, NR₈(C═O)R₈, and oxo where valence permits. Non-limiting examples of cycloalkyl include cyclopropyl, cyclobutyl, cyclopentyl, cyclohexyl, and cycloheptyl. In some embodiments, Rₐ and R_{b} together with the nitrogen atom that they are connected to form an optionally substituted heterocycle including the nitrogen atom and 0-3 additional heteroatoms each selected from the group consisting of N, O, and S, optionally substituted by 1-4 substituents each independently selected from the group consisting of alkyl, cycloalkyl, halogenated cycloalkyl, halogenated alkyl, halogen, CN, OR₈, —(CH₂)₀₋₂OR₈, N(R₈)₂, (C═O)R₈, (C═O)N(R₈)₂, NR₈(C═O)R₈, and oxo where valence permits. Non-limiting examples of heterocycle include

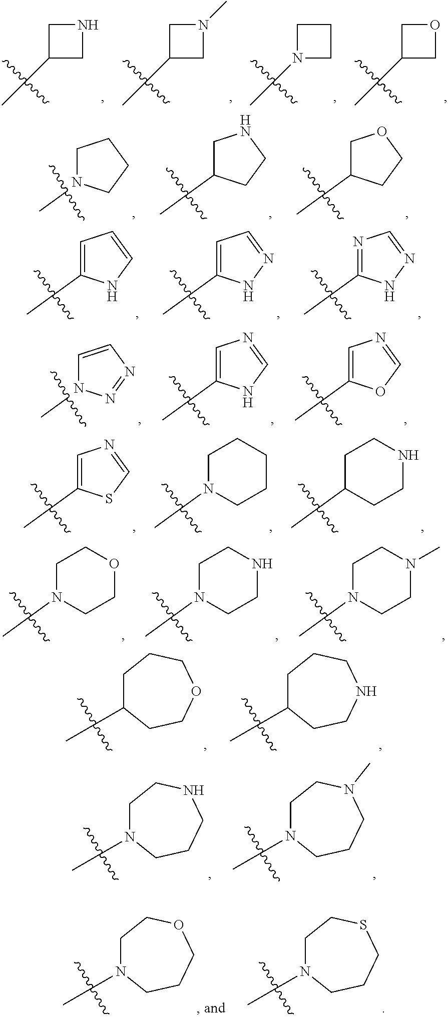

In some embodiments, the alkyl, cycloalkyl, heterocycle, aryl, and heteroaryl in X₁, X₂, and X₃ are optionally substituted by 1-4 substituents each independently selected from the group consisting of alkyl, cycloalkyl, halogenated cycloalkyl, halogenated alkyl, halogen, CN, OR₈, —(CH₂)₀₋₂OR₈, N(R₈)₂, (C═O)R₈, (C═O)N(R₈)₂, NR₈(C═O)R₈, and oxo where valence permits. In some embodiments, the aryl and heteroaryl in A₁, and A₂ are optionally substituted by 1-4 substituents each independently selected from the group consisting of alkyl, cycloalkyl, halogenated cycloalkyl, halogenated alkyl, halogen, CN, OR₈, —(CH₂)₀₋₂OR₈, N(R₈)₂, (C═O)R₈, (C═O)N(R₈)₂, NR₈(C═O)R₈, and oxo where valence permits. In some embodiments, the alkyl, cycloalkyl, heterocycle, aryl, and heteroaryl in R₁, and R₂ are optionally substituted by 1-4 substituents each independently selected from the group consisting of alkyl, cycloalkyl, halogenated cycloalkyl, halogenated alkyl, halogen, CN, OR₈, —(CH₂)₀₋₂OR₈, N(R₈)₂, (C═O)R₈, (C═O)N(R₈)₂, NR₈(C═O)R₈, and oxo where valence permits. In some embodiments, the alkyl, cycloalkyl, heterocycle, aryl, and heteroaryl in R₃ is optionally substituted by 1-4 substituents each independently selected from the group consisting of alkyl, cycloalkyl, halogenated cycloalkyl, halogenated alkyl, halogen, CN, OR₈, —(CH₂)₀₋₂OR₈, N(R₈)₂, (C═O)R₈, (C═O)N(R₈)₂, NR₈(C═O)R₈, and oxo where valence permits. In some embodiments, the alkyl, cycloalkyl, heterocycle, aryl, and heteroaryl in R₄ is optionally substituted by 1-4 substituents each independently selected from the group consisting of alkyl, cycloalkyl, halogenated cycloalkyl, halogenated alkyl, halogen, CN, OR₈, —(CH₂)₀₋₂OR₈, N(R₈)₂, (C═O)R₈, (C═O)N(R₈)₂, NR₈(C═O)R₈, and oxo where valence permits. In some embodiments, the alkyl, cycloalkyl, heterocycle, aryl, and heteroaryl in R₅ is optionally substituted by 1-4 substituents each independently selected from the group consisting of alkyl, cycloalkyl, halogenated cycloalkyl, halogenated alkyl, halogen, CN, OR₈, —(CH₂)₀₋₂OR₈, N(R₈)₂, (C═O)R₈, (C═O)N(R₈)₂, NR₈(C═O)R₈, and oxo where valence permits. In some embodiments, the alkyl, cycloalkyl, heterocycle, aryl, and heteroaryl in R₆ and R₇ are optionally substituted by 1-4 substituents each independently selected from the group consisting of alkyl, cycloalkyl, halogenated cycloalkyl, halogenated alkyl, halogen, CN, OR₈, —(CH₂)₀₋₂OR₈, N(R₈)₂, (C═O)R₈, (C═O)N(R₈)₂, NR₈(C═O)R₈, and oxo where valence permits. In some embodiments, the alkyl, cycloalkyl, heterocycle, aryl, and heteroaryl in R₉ is optionally substituted by 1-4 substituents each independently selected from the group consisting of alkyl, cycloalkyl, halogenated cycloalkyl, halogenated alkyl, halogen, CN, OR₈, —(CH₂)₀₋₂OR₈, N(R₈)₂, (C═O)R₈, (C═O)N(R₈)₂, NR₈(C═O)R₈, and oxo where valence permits. In some embodiments, the alkyl, cycloalkyl, heterocycle, aryl, and heteroaryl in R₁₀ is optionally substituted by 1-4 substituents each independently selected from the group consisting of alkyl, cycloalkyl, halogenated cycloalkyl, halogenated alkyl, halogen, CN, OR₈, —(CH₂)₀₋₂OR₈, N(R₈)₂, (C═O)R₈, (C═O)N(R₈)₂, NR₈(C═O)R₈, and oxo where valence permits. In some embodiments, the alkyl in R₁₂ and R₁₃ is optionally substituted by 1-4 substituents each independently selected from the group consisting of alkyl, cycloalkyl, halogenated cycloalkyl, halogenated alkyl, halogen, CN, OR₈, —(CH₂)₀₋₂OR₈, N(R₈)₂, (C═O)R₈, (C═O)N(R₈)₂, NR₈(C═O)R₈, and oxo where valence permits. In some embodiments, the alkyl, cycloalkyl, heterocycle, aryl, and heteroaryl in Rₐ and R_{b} are optionally substituted by 1-4 substituents each independently selected from the group consisting of alkyl, cycloalkyl, halogenated cycloalkyl, halogenated alkyl, halogen, CN, OR₈, —(CH₂)₀₋₂OR₈, N(R₈)₂, (C═O)R₈, (C═O)N(R₈)₂, NR₈(C═O)R₈, and oxo where valence permits.

In some embodiments, each occurrence of R₈ is independently H, alkyl, or heterocycle optionally substituted by alkyl, OH, or alkoxy. In some embodiments, each occurrence of R₈ is independently H or alkyl. In some embodiments, each occurrence of R₈ is substituted heterocycle. In some embodiments, the two R₈ groups together with the nitrogen atom that they are connected to form an optionally substituted heterocycle including the nitrogen atom and 0-3 additional heteroatoms each selected from the group consisting of N, O, and S. In some specific embodiments, each occurrence of R₈ is independently H or Me.

In some embodiments, the compound of Formula I is selected from the group consisting of compounds 1-15 as shown in Table 1 below. In some embodiments, the compound of Formula I′ is selected from the group consisting of compounds 16-30 as shown in Table 2 below. In some embodiments, the compound of Formula II is selected from the group consisting of compounds 1a-15a as shown in Table 3 below. In some embodiments, the compound of Formula II′ is selected from the group consisting of compounds 16a-30a as shown in Table 4 below. In some embodiments, the compound of Formula III is selected from the group consisting of compounds 1b-15b as shown in Table 5 below. In some embodiments, the compound of Formula IV is selected from the group consisting of compounds 16b-30b as shown in Table 6 below. The enumerated compounds in Tables 1-6 are representative and non-limiting compounds of the embodiments disclosed herein.

TABLE 1
Selected compounds of Formula I.

| No. | 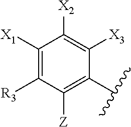 | 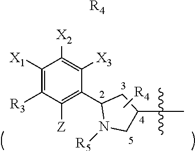 | R₅ | R₁ | R₂ | m | 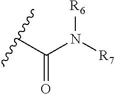 |
|---|---|---|---|---|---|---|---|
| 1 | 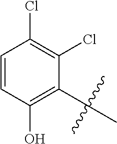 [SMILES: *c1c(O)ccc(Cl)c1Cl] | Me (4-position) | Me | OCH₂CH₃ | Et | 1 | 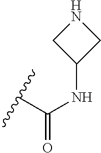 [SMILES: *C(=O)NC1CNC1] |
| 2 | 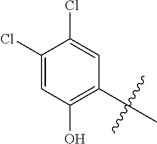 [SMILES: *c1cc(Cl)c(Cl)cc1O] | 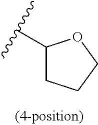 [SMILES: *C1CCCO1] | 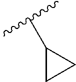 [SMILES: *C1CC1] | Et | Me | 2 | 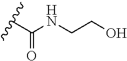 [SMILES: *C(=O)NCCO] |
| 3 | 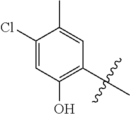 [SMILES: *c1cc(C)c(Cl)cc1O] | 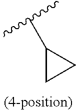 [SMILES: *C1CC1] | 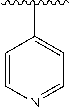 [SMILES: *c1ccncc1] | OCF₃ | Me | 3 | 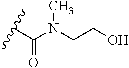 [SMILES: *C(=O)N(C)CCO] |
| 4 | 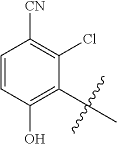 [SMILES: *c1c(O)ccc(C#N)c1Cl] | OH (4-position) | 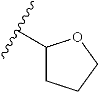 [SMILES: *C1CCCO1] | CN | H | 1 | 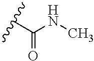 [SMILES: *C(=O)NC] |
| 5 | 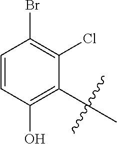 [SMILES: *c1c(O)ccc(Br)c1Cl] | CH₂CH₂OH (4-position) | 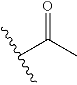 [SMILES: *C(C)=O] | SCH₂CH₃ | CF | 2 | 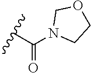 [SMILES: *C(=O)N1CCOC1] |
| 6 | 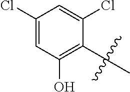 [SMILES: *c1c(O)cc(Cl)cc1Cl] | CH₂CH₂OCH₃ (4-position) | (C═O)CH₂OCH₃ | Cl | H | 3 | 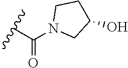 [SMILES: *C(=O)N1CC[C@H](O)C1] |
| 7 | 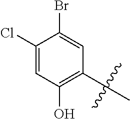 [SMILES: *c1cc(Br)c(Cl)cc1O] | 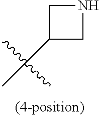 [SMILES: *C1CNC1] | (C═O)CH₂NHCH₃ | NH₂ | Me | 1 | 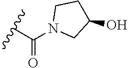 [SMILES: *C(=O)N1CC[C@@H](O)C1] |
| 8 | 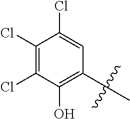 [SMILES: *c1cc(Cl)c(Cl)c(Cl)c1O] | 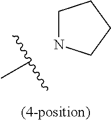 | SO₂CH₃ | NH(C═O)CH₃ | H | 2 | 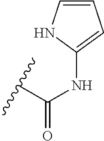 [SMILES: *C(=O)Nc1ccc[nH]1] |
| 9 | 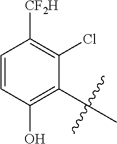 [SMILES: *c1c(O)ccc(C(F)F)c1Cl] | 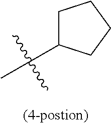 [SMILES: *C1CCCC1] | 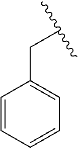 [SMILES: *Cc1ccccc1] | 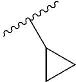 [SMILES: *C1CC1] | H | 3 | 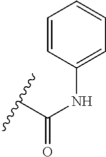 [SMILES: *C(=O)Nc1ccccc1] |
| 10 | 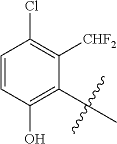 [SMILES: *c1c(O)ccc(Cl)c1C(F)F] | CH₂OCH₃ (4-position) | 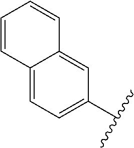 [SMILES: *c1ccc2ccccc2c1] | 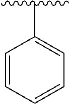 [SMILES: *c1ccccc1] | H | 1 | 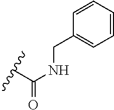 [SMILES: *C(=O)NCc1ccccc1] |
| 11 | 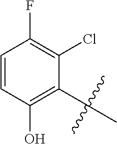 [SMILES: *c1c(O)ccc(F)c1Cl] | CH₂NHCH₃ (4-position) | 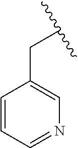 [SMILES: *Cc1cccnc1] | 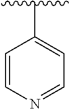 [SMILES: *c1ccncc1] | H | 2 | 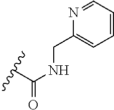 [SMILES: *C(=O)NCc1ccccn1] |
| 12 | 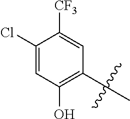 [SMILES: *c1cc(C(F)(F)F)c(Cl)cc1O] | Et (2-position) | CF₃ | 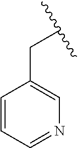 [SMILES: *Cc1cccnc1] | H | 3 | 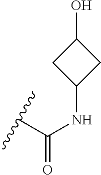 [SMILES: *C(=O)NC1CC(O)C1] |
| 13 | 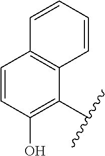 [SMILES: *c1c(O)ccc2ccccc12] | i-Pr (3-position) | CH₂CH₂OH | F | F | 1 | 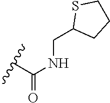 [SMILES: *C(=O)NCC1CCCS1] |
| 14 | 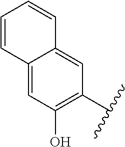 [SMILES: *c1cc2ccccc2cc1O] | Me (5-position) | (C═O)Et | 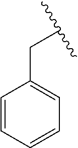 [SMILES: *Cc1ccccc1] | H | 2 | 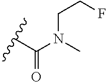 [SMILES: *C(=O)N(C)CCF] |
| 15 | 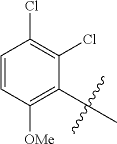 [SMILES: *c1c(OC)ccc(Cl)c1Cl] | Et (5-position) | Et | CH₂OH | H | 3 | 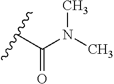 [SMILES: *C(=O)N(C)C] |

TABLE 2
Selected compounds of Formula I′.

| No. | 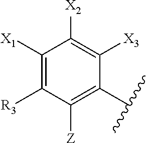 | 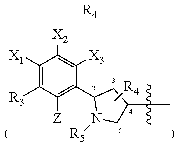 | R₅ | R₁ | R₂ | m | 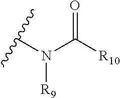 |
|---|---|---|---|---|---|---|---|
| 16 | 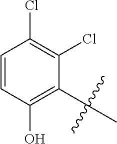 [SMILES: *c1c(O)ccc(Cl)c1Cl] | Me (4-position) | Me | OCH₂CH₃ | Et | 1 | 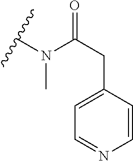 [SMILES: *N(C)C(=O)Cc1ccncc1] |
| 17 | 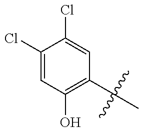 [SMILES: *c1cc(Cl)c(Cl)cc1O] | 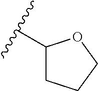 [SMILES: *C1CCCO1] (4-position) | 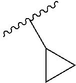 [SMILES: *C1CC1] | Et | Me | 2 | 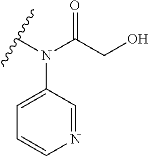 [SMILES: *N(C(=O)CO)c1cccnc1] |
| 18 | 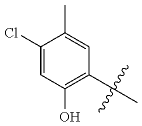 [SMILES: *c1cc(C)c(Cl)cc1O] | 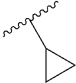 [SMILES: *C1CC1] (4-position) | 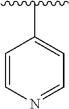 [SMILES: *c1ccncc1] | OCF₃ | Me | 3 | 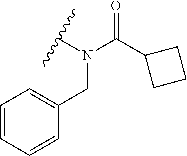 [SMILES: *N(Cc1ccccc1)C(=O)C1CCC1] |
| 19 | 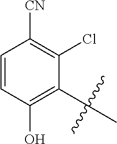 [SMILES: *c1c(O)ccc(C#N)c1Cl] | OH (4-position) | 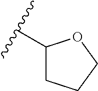 [SMILES: *C1CCCO1] | CN | H | 1 | 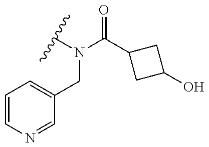 [SMILES: *N(Cc1cccnc1)C(=O)C1CC(O)C1] |
| 20 | 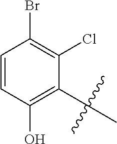 [SMILES: *c1c(O)ccc(Br)c1Cl] | CH₂CH₂OH (4-position) | 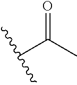 [SMILES: *C(C)=O] | SCH₂CH₃ | CF₃ | 2 | 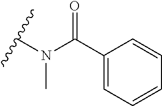 [SMILES: *N(C)C(=O)c1ccccc1] |
| 21 | 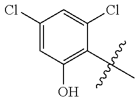 [SMILES: *c1c(O)cc(Cl)cc1Cl] | CH₂CH₂OCH₃ (4-position) | (C═O)CH₂OCH₃ | Cl | H | 3 | 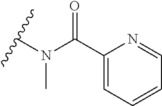 [SMILES: *N(C)C(=O)c1ccccn1] |
| 22 | 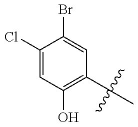 [SMILES: *c1cc(Br)c(Cl)cc1O] | 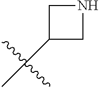 [SMILES: *C1CNC1] (4-position) | (C═O)CH₂NHCH₃ | NH₂ | Me | 1 | 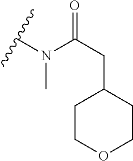 [SMILES: *N(C)C(=O)CC1CCOCC1] |
| 23 | 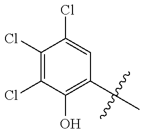 [SMILES: *c1cc(Cl)c(Cl)c(Cl)c1O] | 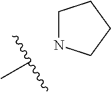 (4-position) | SO₂CH₃ | NH(C═O)CH₃ | H | 2 | 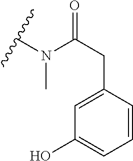 [SMILES: *N(C)C(=O)Cc1cccc(O)c1] |
| 24 | 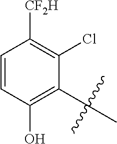 [SMILES: *c1c(O)ccc(C(F)F)c1Cl] | 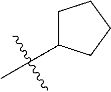 [SMILES: *C1CCCC1] (4-position) | 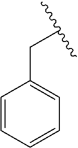 [SMILES: *Cc1ccccc1] | 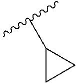 [SMILES: *C1CC1] | H | 3 | 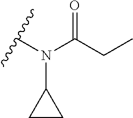 [SMILES: *N(C(=O)CC)C1CC1] |
| 25 | 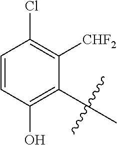 [SMILES: *c1c(O)ccc(Cl)c1C(F)F] | CH₂OCH₃ (4-position) | 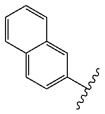 [SMILES: *c1ccc2ccccc2c1] | 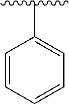 [SMILES: *c1ccccc1] | H | 1 | 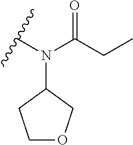 [SMILES: *N(C(=O)CC)C1CCOC1] |
| 26 | 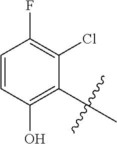 [SMILES: *c1c(O)ccc(F)c1Cl] | CH₂NHCH₃ (4-position) | 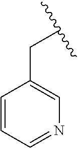 [SMILES: *Cc1cccnc1] | 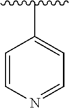 [SMILES: *c1ccncc1] | H | 2 | 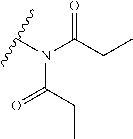 [SMILES: *N(C(=O)CC)C(=O)CC] |
| 27 | 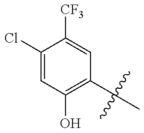 [SMILES: *c1cc(C(F)(F)F)c(Cl)cc1O] | Et (2-position) | CF₃ | 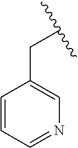 [SMILES: *Cc1cccnc1] | H | 3 | 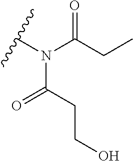 [SMILES: *N(C(=O)CC)C(=O)CCO] |
| 28 | 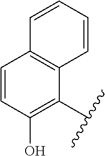 [SMILES: *c1c(O)ccc2ccccc12] | i-Pr (3-position) | CH₂CH₂OH | F | F | 1 | 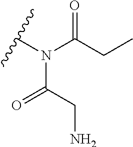 [SMILES: *N(C(=O)CC)C(=O)CN] |
| 29 | 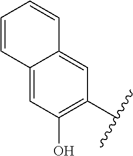 [SMILES: *c1cc2ccccc2cc1O] | Me (5-position) | (C═O)Et | 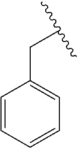 [SMILES: *Cc1ccccc1] | H | 2 | 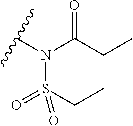 [SMILES: *N(C(=O)CC)S(=O)(=O)CC] |
| 30 | 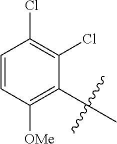 [SMILES: *c1c(OC)ccc(Cl)c1Cl] | Et (5-position) | Et | CH₂OH | H | 3 | 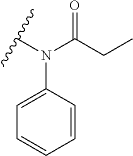 [SMILES: *N(C(=O)CC)c1ccccc1] |

TABLE 3
Selected compounds of Formula II.

| No. | 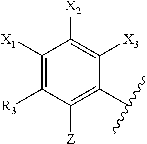 | 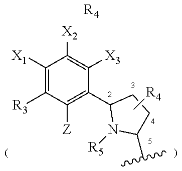 | R₅ | R₁ | R₂ | m | 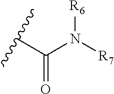 |
|---|---|---|---|---|---|---|---|
| 1a | 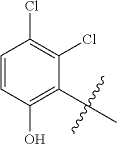 [SMILES: *c1c(O)ccc(Cl)c1Cl] | Me (5-position) | Me | OCH₂CH₃ | Et | 1 | 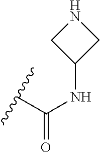 [SMILES: *C(=O)NC1CNC1] |
| 2a | 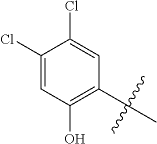 [SMILES: *c1cc(Cl)c(Cl)cc1O] | 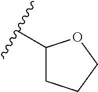 [SMILES: *C1CCCO1] (5-position) | 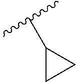 [SMILES: *C1CC1] | Et | Me | 2 | 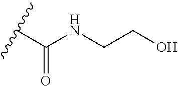 [SMILES: *C(=O)NCCO] |
| 3a | 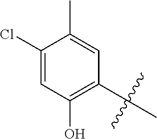 [SMILES: *c1cc(C)c(Cl)cc1O] | 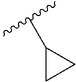 [SMILES: *C1CC1] (5-position) | 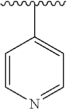 [SMILES: *c1ccncc1] | OCF₃ | Me | 3 | 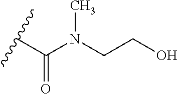 [SMILES: *C(=O)N(C)CCO] |
| 4a | 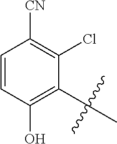 [SMILES: *c1c(O)ccc(C#N)c1Cl] | OH (5-position) | 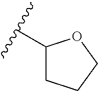 [SMILES: *C1CCCO1] | CN | H | 1 | 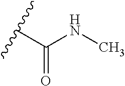 [SMILES: *C(=O)NC] |
| 5a | 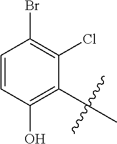 [SMILES: *c1c(O)ccc(Br)c1Cl] | CH₂CH₂OH (5-position) | 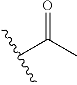 [SMILES: *C(C)=O] | SCH₂CH₃ | CF₃ | 2 | 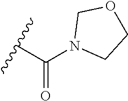 [SMILES: *C(=O)N1CCOC1] |
| 6a | 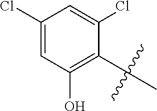 [SMILES: *c1c(O)cc(Cl)cc1Cl] | CH₂CH₂OCH₃ (5-position) | (C═O)CH₂OCH₃ | Cl | H | 3 | 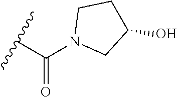 [SMILES: *C(=O)N1CC[C@H](O)C1] |
| 7a | 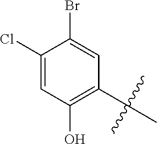 [SMILES: *c1cc(Br)c(Cl)cc1O] | 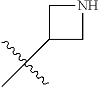 [SMILES: *C1CNC1] (5-position) | (C═O)CH₂NHCH₃ | NH₂ | Me | 1 | 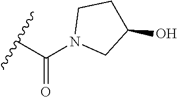 [SMILES: *C(=O)N1CC[C@@H](O)C1] |
| 8a | 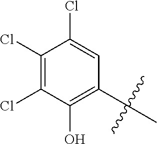 [SMILES: *c1cc(Cl)c(Cl)c(Cl)c1O] | 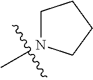 (5-position) | SO₂CH₃ | NH(C═O)CH₃ | H | 2 | 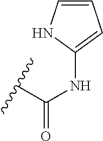 [SMILES: *C(=O)Nc1ccc[nH]1] |
| 9a | 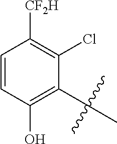 [SMILES: *c1c(O)ccc(C(F)F)c1Cl] | 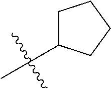 [SMILES: *C1CCCC1] (5-position) | 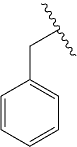 [SMILES: *Cc1ccccc1] | 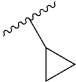 [SMILES: *C1CC1] | H | 3 | 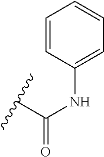 [SMILES: *C(=O)Nc1ccccc1] |
| 10a | 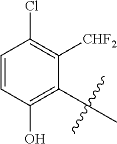 [SMILES: *c1c(O)ccc(Cl)c1C(F)F] | CH₂OCH₃ (5-position) | 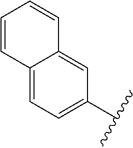 [SMILES: *c1ccc2ccccc2c1] | 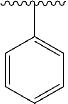 [SMILES: *c1ccccc1] | H | 1 | 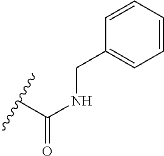 [SMILES: *C(=O)NCc1ccccc1] |
| 11a | 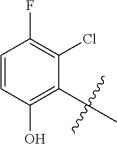 [SMILES: *c1c(O)ccc(F)c1Cl] | CH₂NHCH₃ (5-position) | 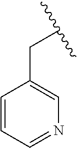 [SMILES: *Cc1cccnc1] | 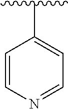 [SMILES: *c1ccncc1] | H | 2 | 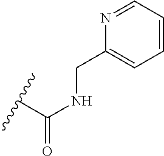 [SMILES: *C(=O)NCc1ccccn1] |
| 12a | 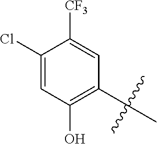 [SMILES: *c1cc(C(F)(F)F)c(Cl)cc1O] | Et (2-position) | CF₃ | 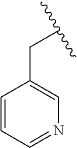 [SMILES: *Cc1cccnc1] | H | 3 | 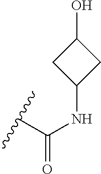 [SMILES: *C(=O)NC1CC(O)C1] |
| 13a | 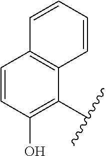 [SMILES: *c1c(O)ccc2ccccc12] | i-Pr (3-position) | CH₂CH₂OH | F | F | 1 | 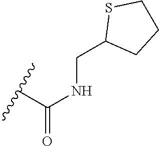 [SMILES: *C(=O)NCC1CCCS1] |
| 14a | 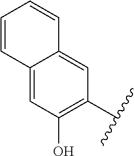 [SMILES: *c1cc2ccccc2cc1O] | Me (4-position) | (C═O)Et | 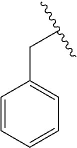 [SMILES: *Cc1ccccc1] | H | 2 | 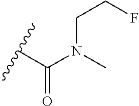 [SMILES: *C(=O)N(C)CCF] |
| 15a | 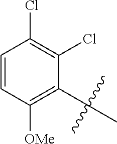 [SMILES: *c1c(OC)ccc(Cl)c1Cl] | Et (4-position) | Et | CH₂OH | H | 3 | 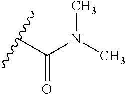 [SMILES: *C(=O)N(C)C] |

TABLE 4
Selected compounds of Formula II′.

| No. | 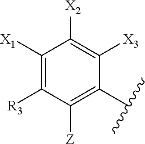 | 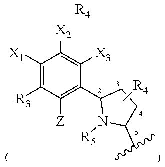 | R₅ | R₁ | R₂ | m | 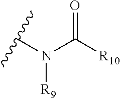 |
|---|---|---|---|---|---|---|---|
| 16a | 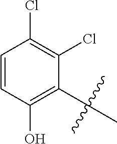 [SMILES: *c1c(O)ccc(Cl)c1Cl] | Me (5-position) | Me | OCH₂CH₃ | Et | 1 | 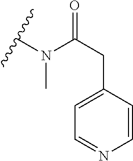 [SMILES: *N(C)C(=O)Cc1ccncc1] |
| 17a | 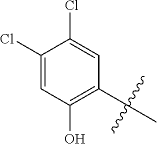 [SMILES: *c1cc(Cl)c(Cl)cc1O] | 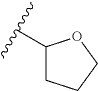 [SMILES: *C1CCCO1] (5-position) | 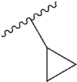 [SMILES: *C1CC1] | Et | Me | 2 | 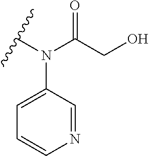 [SMILES: *N(C(=O)CO)c1cccnc1] |
| 18a | 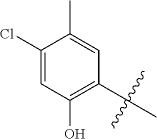 [SMILES: *c1cc(C)c(Cl)cc1O] | 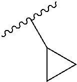 [SMILES: *C1CC1] (5-position) | 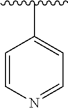 [SMILES: *c1ccncc1] | OCF₃ | Me | 3 | 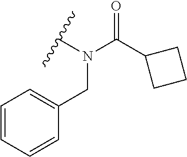 [SMILES: *N(Cc1ccccc1)C(=O)C1CCC1] |
| 19a | 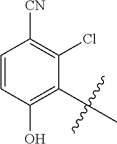 [SMILES: *c1c(O)ccc(C#N)c1Cl] | OH (5-position) | 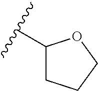 [SMILES: *C1CCCO1] | CN | H | 1 | 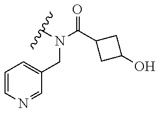 [SMILES: *N(Cc1cccnc1)C(=O)C1CC(O)C1] |
| 20a | 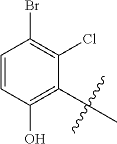 [SMILES: *c1c(O)ccc(Br)c1Cl] | CH₂CH₂OH (5-position) | 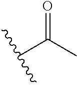 [SMILES: *C(C)=O] | SCH₂CH₃ | CF₃ | 2 | 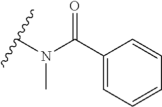 [SMILES: *N(C)C(=O)c1ccccc1] |
| 21a | 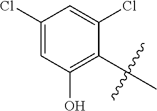 [SMILES: *c1c(O)cc(Cl)cc1Cl] | CH₂CH₂OCH₃ (5-position) | (C═O)CH₂OCH₃ | Cl | H | 3 | 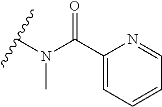 [SMILES: *N(C)C(=O)c1ccccn1] |
| 22a | 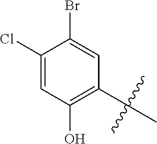 [SMILES: *c1cc(Br)c(Cl)cc1O] | 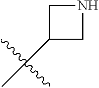 [SMILES: *C1CNC1] (5-position) | (C═O)CH₂NHCH₃ | NH₂ | Me | 1 | 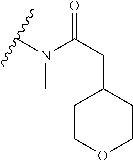 [SMILES: *N(C)C(=O)CC1CCOCC1] |
| 23a | 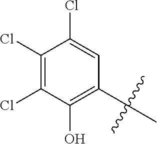 [SMILES: *c1cc(Cl)c(Cl)c(Cl)c1O] | 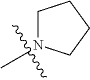 (5-position) | SO₂CH₃ | NH(C═O)CH₃ | H | 2 | 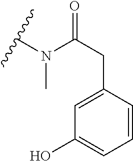 [SMILES: *N(C)C(=O)Cc1cccc(O)c1] |
| 24a | 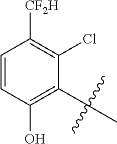 [SMILES: *c1c(O)ccc(C(F)F)c1Cl] | 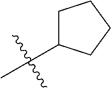 [SMILES: *C1CCCC1] (5-position) | 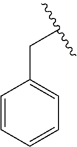 [SMILES: *Cc1ccccc1] | 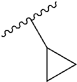 [SMILES: *C1CC1] | H | 3 | 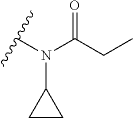 [SMILES: *N(C(=O)CC)C1CC1] |
| 25a | 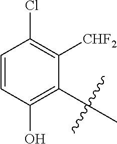 [SMILES: *c1c(O)ccc(Cl)c1C(F)F] | CH₂OCH₃ (5-position) | 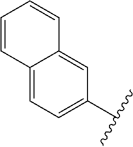 [SMILES: *c1ccc2ccccc2c1] | 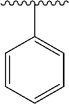 [SMILES: *c1ccccc1] | H | 1 | 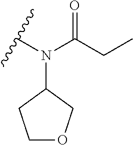 [SMILES: *N(C(=O)CC)C1CCOC1] |
| 26a | 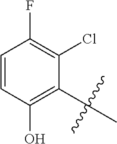 [SMILES: *c1c(O)ccc(F)c1Cl] | CH₂NHCH₃ (5-position) | 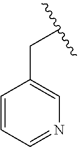 [SMILES: *Cc1cccnc1] | 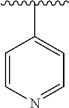 [SMILES: *c1ccncc1] | H | 2 | 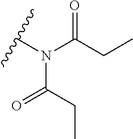 [SMILES: *N(C(=O)CC)C(=O)CC] |
| 27a | 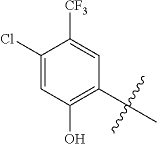 [SMILES: *c1cc(C(F)(F)F)c(Cl)cc1O] | Et (2-position) | CF₃ | 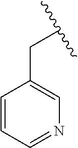 [SMILES: *Cc1cccnc1] | H | 3 | 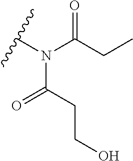 [SMILES: *N(C(=O)CC)C(=O)CCO] |
| 28a | 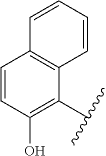 [SMILES: *c1c(O)ccc2ccccc12] | i-Pr (3-position) | CH₂CH₂OH | F | F | 1 | 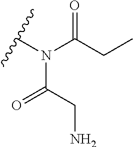 [SMILES: *N(C(=O)CC)C(=O)CN] |
| 29a | 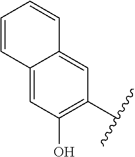 [SMILES: *c1cc2ccccc2cc1O] | Me (4-position) | (C═O)Et | 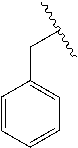 [SMILES: *Cc1ccccc1] | H | 2 | 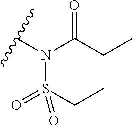 [SMILES: *N(C(=O)CC)S(=O)(=O)CC] |
| 30a | 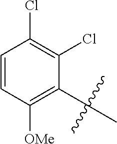 [SMILES: *c1c(OC)ccc(Cl)c1Cl] | Et (4-position) | Et | CH₂OH | H | 3 | 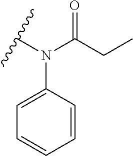 [SMILES: *N(C(=O)CC)c1ccccc1] |

TABLE 5
Selected compounds of Formula III.

| No. | 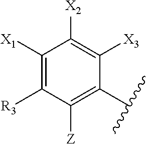 | 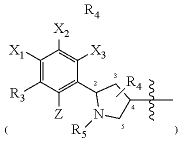 | R₅ | R₁ | R₂ | m | 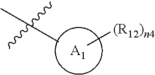 |
|---|---|---|---|---|---|---|---|
| 1b | 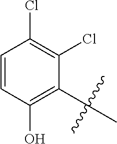 [SMILES: *c1c(O)ccc(Cl)c1Cl] | Me (4-position) | Me | OCH₂CH₃ | Et | 1 | 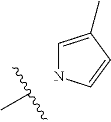 |
| 2b | 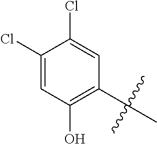 [SMILES: *c1cc(Cl)c(Cl)cc1O] | 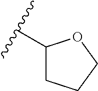 [SMILES: *C1CCCO1] (4-position) | 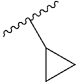 [SMILES: *C1CC1] | Et | Me | 2 | 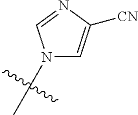 [SMILES: *n1cnc(C#N)c1] |
| 3b | 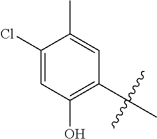 [SMILES: *c1cc(C)c(Cl)cc1O] | 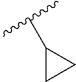 [SMILES: *C1CC1] (4-position) | 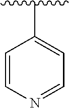 [SMILES: *c1ccncc1] | OCF₃ | Me | 3 | 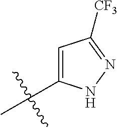 [SMILES: *c1cc(C(F)(F)F)n[nH]1] |
| 4b | 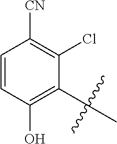 [SMILES: *c1c(O)ccc(C#N)c1Cl] | OH (4-position) | 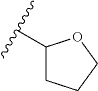 [SMILES: *C1CCCO1] | CN | H | 1 | 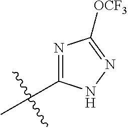 [SMILES: *c1nc(OC(F)(F)F)n[nH]1] |
| 5b | 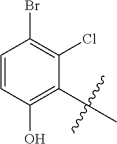 [SMILES: *c1c(O)ccc(Br)c1Cl] | CH₂CH₂OH (4-position) | 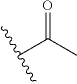 [SMILES: *C(C)=O] | SCH₂CH₃ | CF₃ | 2 | 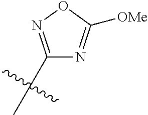 [SMILES: *c1noc(OC)n1] |
| 6b | 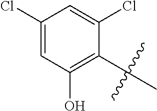 [SMILES: *c1c(O)cc(Cl)cc1Cl] | CH₂CH₂OCH₃ (4-position) | (C═O)CH₂OCH₃ | Cl | H | 3 | 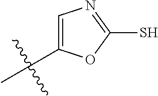 [SMILES: *c1cnc(S)o1] |
| 7b | 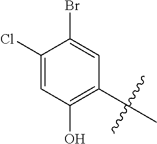 [SMILES: *c1cc(Br)c(Cl)cc1O] | 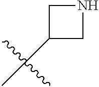 [SMILES: *C1CNC1] (4-position) | (C═O)CH₂NHCH₃ | NH₂ | Me | 1 | 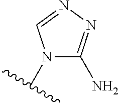 [SMILES: *n1cnnc1N] |
| 8b | 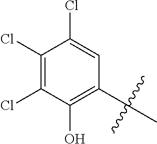 [SMILES: *c1cc(Cl)c(Cl)c(Cl)c1O] | 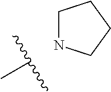 (4-position) | SO₂CH₃ | NH(C═O)CH₃ | H | 2 | 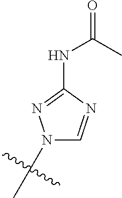 [SMILES: *n1cnc(NC(C)=O)n1] |
| 9b | 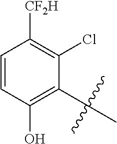 [SMILES: *c1c(O)ccc(C(F)F)c1Cl] | 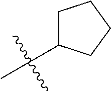 [SMILES: *C1CCCC1] (4-position) | 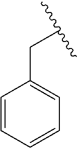 [SMILES: *Cc1ccccc1] | 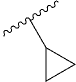 [SMILES: *C1CC1] | H | 3 | 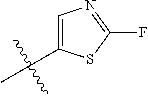 [SMILES: *c1cnc(F)s1] |
| 10b | 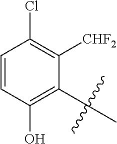 [SMILES: *c1c(O)ccc(Cl)c1C(F)F] | CH₂OCH₃ (4-position) | 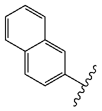 [SMILES: *c1ccc2ccccc2c1] | 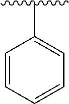 [SMILES: *c1ccccc1] | H | 1 | 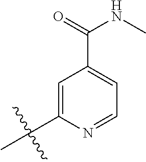 [SMILES: *c1cc(C(=O)NC)ccn1] |
| 11b | 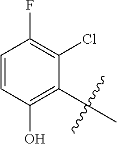 [SMILES: *c1c(O)ccc(F)c1Cl] | CH₂NHCH₃ (4-position) | 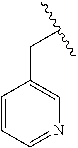 [SMILES: *Cc1cccnc1] | 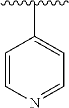 [SMILES: *c1ccncc1] | H | 2 | 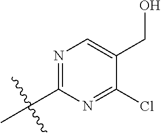 [SMILES: *c1ncc(CO)c(Cl)n1] |
| 12b | 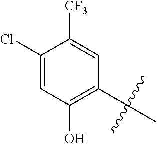 [SMILES: *c1cc(C(F)(F)F)c(Cl)cc1O] | Et (2-position) | CF₃ | 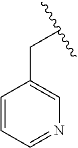 [SMILES: *Cc1cccnc1] | H | 3 | 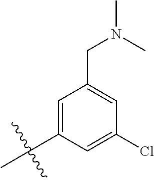 [SMILES: *c1cc(Cl)cc(CN(C)C)c1] |
| 13b | 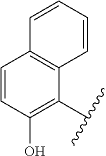 [SMILES: *c1c(O)ccc2ccccc12] | i-Pr (3-position) | CH₂CH₂OH | F | F | 1 | 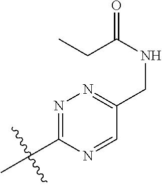 [SMILES: *c1ncc(CNC(=O)CC)nn1] |
| 14b | 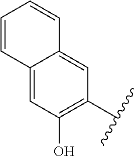 [SMILES: *c1cc2ccccc2cc1O] | Me (5-position) | (C═O)Et | 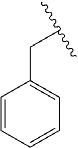 [SMILES: *Cc1ccccc1] | H | 2 | 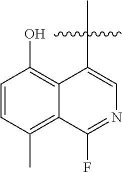 [SMILES: *c1cnc(F)c2c(C)ccc(O)c12] |
| 15b | 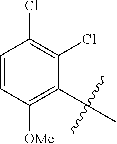 [SMILES: *c1c(OC)ccc(Cl)c1Cl] | Et (5-position) | Et | CH₂OH | H | 3 | 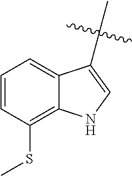 [SMILES: *c1c[nH]c2c(SC)cccc12] |

TABLE 6
Selected compounds of Formula IV.

| No. | 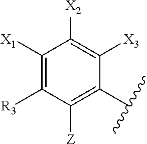 | 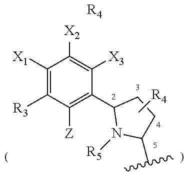 | R₅ | R₁ | R₂ | m | 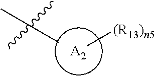 |
|---|---|---|---|---|---|---|---|
| 16b | 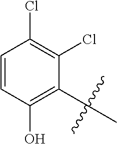 [SMILES: *c1c(O)ccc(Cl)c1Cl] | Me (5-position) | Me | OCH₂CH₃ | Et | 1 | 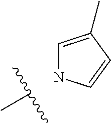 |
| 17b | 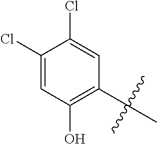 [SMILES: *c1cc(Cl)c(Cl)cc1O] | 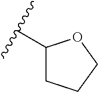 [SMILES: *C1CCCO1] (5-position) | 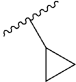 [SMILES: *C1CC1] | Et | Me | 2 | 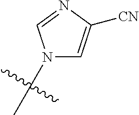 [SMILES: *n1cnc(C#N)c1] |
| 18b | 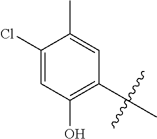 [SMILES: *c1cc(C)c(Cl)cc1O] | 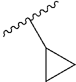 [SMILES: *C1CC1] (5-position) | 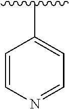 [SMILES: *c1ccncc1] | OCF₃ | Me | 3 | 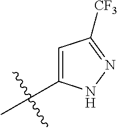 [SMILES: *c1cc(C(F)(F)F)n[nH]1] |
| 19b | 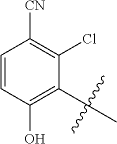 [SMILES: *c1c(O)ccc(C#N)c1Cl] | OH (5-position) | 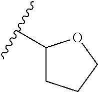 [SMILES: *C1CCCO1] | CN | H | 1 | 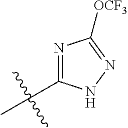 [SMILES: *c1nc(OC(F)(F)F)n[nH]1] |
| 20b | 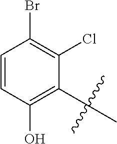 [SMILES: *c1c(O)ccc(Br)c1Cl] | CH₂CH₂OH (5-position) | 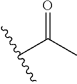 [SMILES: *C(C)=O] | SCH₂CH₃ | CF₃ | 2 | 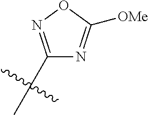 [SMILES: *c1noc(OC)n1] |
| 21b | 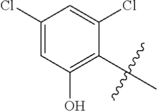 [SMILES: *c1c(O)cc(Cl)cc1Cl] | CH₂CH₂OCH₃ (5-position) | (C═O)CH₂OCH₃ | Cl | H | 3 | 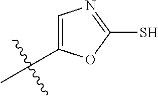 [SMILES: *c1cnc(S)o1] |
| 22b | 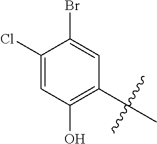 [SMILES: *c1cc(Br)c(Cl)cc1O] | 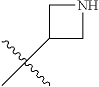 [SMILES: *C1CNC1] (5-position) | (C═O)CH₂NHCH₃ | NH₂ | Me | 1 | 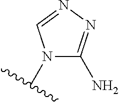 [SMILES: *n1cnnc1N] |
| 23b | 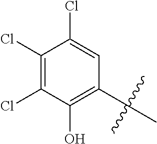 [SMILES: *c1cc(Cl)c(Cl)c(Cl)c1O] | 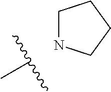 (5-position) | SO₂CH₃ | NH(C═O)CH₃ | H | 2 | 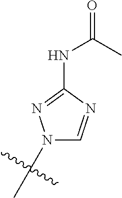 [SMILES: *n1cnc(NC(C)=O)n1] |
| 24b | 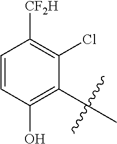 [SMILES: *c1c(O)ccc(C(F)F)c1Cl] | 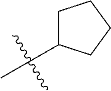 [SMILES: *C1CCCC1] (5-position) | 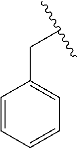 [SMILES: *Cc1ccccc1] | 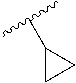 [SMILES: *C1CC1] | H | 3 | 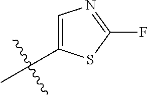 [SMILES: *c1cnc(F)s1] |
| 25b | 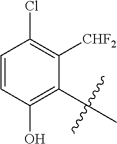 [SMILES: *c1c(O)ccc(Cl)c1C(F)F] | CH₂OCH₃ (5-position) | 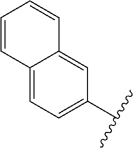 [SMILES: *c1ccc2ccccc2c1] | 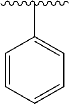 [SMILES: *c1ccccc1] | H | 1 | 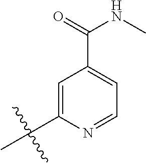 [SMILES: *c1cc(C(=O)NC)ccn1] |
| 26b | 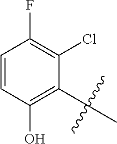 [SMILES: *c1c(O)ccc(F)c1Cl] | CH₂NHCH₃ (5-position) | 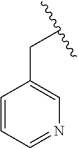 [SMILES: *Cc1cccnc1] | 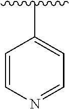 [SMILES: *c1ccncc1] | H | 2 | 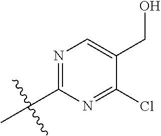 [SMILES: *c1ncc(CO)c(Cl)n1] |
| 27b | 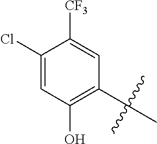 [SMILES: *c1cc(C(F)(F)F)c(Cl)cc1O] | Et (2-position) | CF₃ | 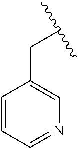 [SMILES: *Cc1cccnc1] | H | 3 | 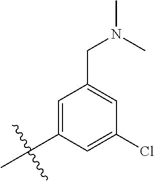 [SMILES: *c1cc(Cl)cc(CN(C)C)c1] |
| 28b | 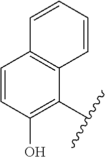 [SMILES: *c1c(O)ccc2ccccc12] | i-Pr (3-position) | CH₂CH₂OH | F | F | 1 | 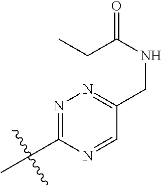 [SMILES: *c1ncc(CNC(=O)CC)nn1] |
| 29b | 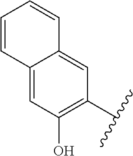 [SMILES: *c1cc2ccccc2cc1O] | Me (4-position) | (C═O)Et | 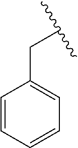 [SMILES: *Cc1ccccc1] | H | 2 | 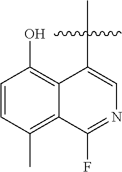 [SMILES: *c1cnc(F)c2c(C)ccc(O)c12] |
| 30b | 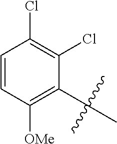 [SMILES: *c1c(OC)ccc(Cl)c1Cl] | Et (4-position) | Et | CH₂OH | H | 3 | 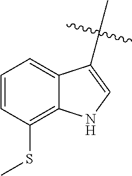 [SMILES: *c1c[nH]c2c(SC)cccc12] |

### Abbreviations

ACN Acetonitrile
floe or boc Tert-butyloxycarbonyl
DCM Dichloromethane
DMAP 4-Dimethyl aminopyridine
DME Dimethoxyethane
DMF Dimethyl formamide
DMSO Dimethyl sulfoxide
EA Ethyl acetate
HATU N-[(dimethylamino)(3H-1,2,3-triazolo(4,4-b)pyridin-3-yloxy)methylene]-N-methylmethaneaminium hexafluorophosphate
MeOH Methanol
MOM Methoxymethyl
PE Petroleum ether
SEM Trimethylsilylethoxymethyl
SEMCl 2-(Trimethylsilyl)ethoxymethyl chloride
TEA Triethylamine
TFA Trifluoroacetic acid
THF Tetrahydrofuran
TsCl Toluenesulfonyl chloride

### Methods of Preparation

Following are general synthetic schemes for manufacturing compounds of the present invention. These schemes are illustrative and are not meant to limit the possible techniques one skilled in the art may use to manufacture the compounds disclosed herein. Different methods will be evident to those skilled in the art. Additionally, the various steps in the synthesis may be performed in an alternate sequence or order to give the desired compound(s). All documents cited herein are incorporated herein by reference in their entirety. For example, the following reactions are illustrations, but not limitations of the preparation of some of the starting materials and compounds disclosed herein.

Schemes 1-3 below describe synthetic routes which may be used for the synthesis of compounds of the present invention, e.g., compounds having a structure of Formula I, I′, II, II′, III, or IV or a precursor thereof. Various modifications to these methods may be envisioned by those skilled in the art to achieve similar results to that of the inventions given below. In the embodiments below, the synthetic route is described using compounds having the structure of Formula I, I′, II, II′, III, or IV or a precursor thereof as examples. The general synthetic routes described in Schemes 1-3 and examples described in the Example section below illustrate methods used for the preparation of the compounds described herein.

Compound I-1 as shown in Scheme 1 can be prepared by any method known in the art and/or is commercially available. As shown in Scheme 1, PG refers to a protecting group. Non-limiting examples of the protecting groups include Me, methoxymethyl (MOM), trimethylsilylethoxymethyl (SEM), allyl, Ac, Boc, other alkoxycarbonyl group, dialkylaminocarbonyl, and another protecting group known in the art suitable for use as protecting groups for OH or an amine group. Other substituents are defined herein. Benzaldehyde I-1 can be reacted with (S)-t-butyl sulfinamide in the presence of a Lewis acid such as titanium tetraisopropoxide to provide the sulfinyl imine I-2S. Reformatsky reaction of I-2S with methyl 2-bromomethyl acrylate and zinc gives I-3 with the R configuration at the benzylic amine position. The sulfinamide is then removed by treatment with dilute acid to give amine I-4 that is reprotected as a toluene sulfonamide I-5 under standard conditions. Reaction of I-5 with a base such as sodium hydride in a polar aprotic solvent such as DMF and heating, for example at 100° C. yields the pyrrolidine ester I-6 as a mixture of epimers at the ester position. The tosyl group is removed by treatment with magnesium metal in methanol to give I-7. The amine I-7 is reprotected with for example a Boc group and the ester hydrolyzed to form I-8. I-8 is converted to amides by reaction with suitable amines R₆R₇NH and a coupling reagent such as HATU, and all the protecting groups are removed under standard conditions to provide pyrrolidine amides I-9.

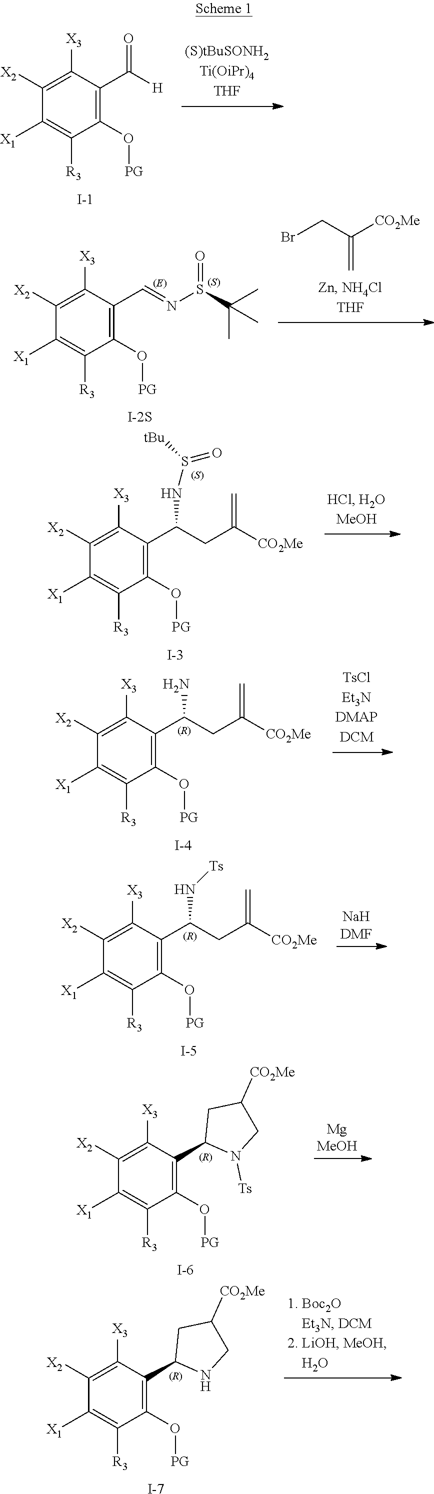

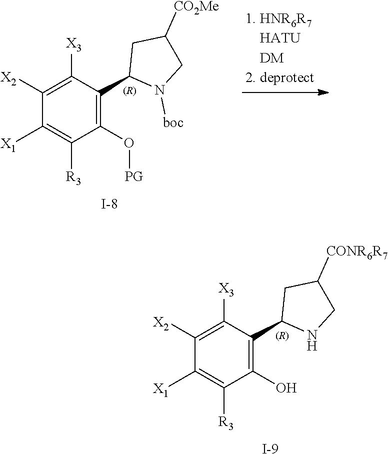

Compound I-10 as shown in Scheme 2 can be prepared by any method known in the art and/or is commercially available. As shown in Scheme 2, PG refers to a protecting group. Non-limiting examples of the protecting groups include Me, methoxymethyl (MOM), trimethylsilylethoxymethyl (SEM), allyl, Ac, Boc, other alkoxycarbonyl group, dialkylaminocarbonyl, and another protecting group known in the art suitable for use as protecting groups for OH. Other substituents shown in Scheme 2 are defined herein. As shown in Scheme 2, compound I-10 is deprotonated using a base such as n-butyl lithium, and reacted with 1-t-butyl 2-ethyl (2S)-5-oxopyrrolidine-1,2-dicarboxylate to form ketone I-11. Alternatively, a phenol with a halogen such as iodine or bromine next to the phenol may be used, and metallated by metal-halogen exchange using an agent such as isopropyl magnesium chloride/lithium chloride (Turbo Grignard) or an organolithium reagent such as n-butyl lithium. Removal of the Boc group with TFA leads to cyclization to the imine I-12. Reduction of the imine to the pyrrolidine can be carried out by a variety of methods including catalytic hydrogenation over a catalyst such as platinum oxide in a solvent such as ethyl acetate, or with sodium borohydride to give pyrrolidine I-13 as the cis isomer. The pyrrolidine nitrogen is then protected with a protecting group such as Boc to yield I-14.

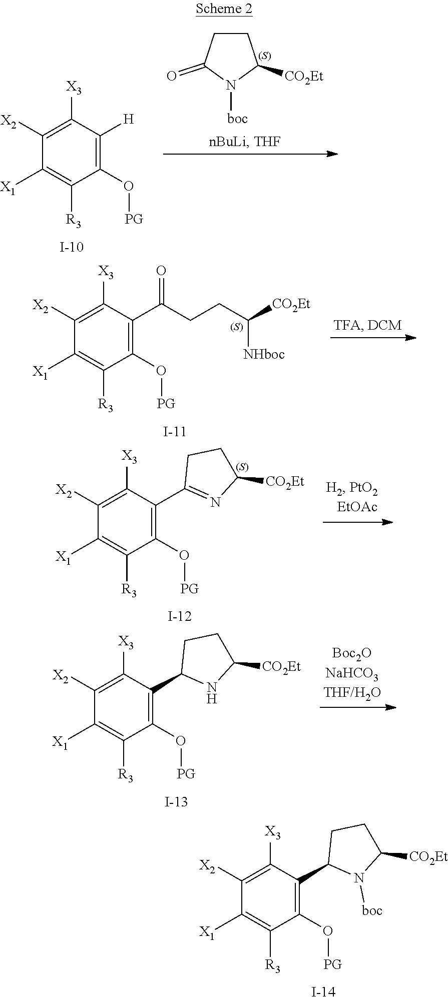

Compound I-2R as shown in Scheme 3 can be prepared by any method known in the art and/or is commercially available. As shown in Scheme 3, PG refers to a protecting group. Non-limiting examples of the protecting groups include Me, methoxymethyl (MOM), trimethylsilylethoxymethyl (SEM), allyl, Ac, Boc, other alkoxycarbonyl group, dialkylaminocarbonyl, and another protecting group known in the art suitable for use as protecting groups for OH or an amine group. Other substituents shown in Scheme 3 are defined herein. For compounds of Formula I disclosed herein where R₂ is H and m is 1, the pyrrolidine ring with an extended chain at C4 (see compound I-15 with the 4-position labelled) can be obtained by the synthesis described in Scheme 3. As shown in Scheme 3, benzene sulfinyl imine I-2R can undergo cycloaddition with the trimethylenemethane precursor 2-((trimethylsilyl)methyl)-prop-2-enyl acetate in the presence of a palladium catalyst such as tetrakis triphenylphosphine palladium in a solvent such as THE to produce 2R-phenyl pyrrolidine I-15. One way to introduce the side chain at C4 is by cross-metathesis with methyl acylate and Grubbs' 2^{nd} generation catalyst to form the unsaturated ester I-16. Hydrogenation of I-16 over a catalyst such as platinum oxide in a solvent such as methanol gives I-17 as a mixture of epimers. Ester I-17 can be converted to amides I-18 by hydrolysis with an agent such as lithium hydroxide and reaction of the resulting carboxylic acid with an amine R₆R₇NH and a coupling reagent such as HATU. Removal of the sulfinamide and phenol protecting groups using standard methods yields amide I-19 as a mixture of isomers that can be separated by chromatography. In an alternative route to I-19, 2R-phenyl pyrrolidine I-15 is first converted to the Boc-protected pyrrolidine I-20 by hydrolysis of the sulfinamide with acid and reprotection with Boc anhydride. Reaction of I-20 with an oxidizing agent such as ruthenium chloride forms the diol that is cleaved in situ with sodium periodate to give ketone I-22. Wittig reaction of I-22 with a suitably substituted triphenylphosphanylidene acetate or similar reagent provides unsaturated ester I-22 analogous to I-16 wherein R₁ can be H, alkyl, etc. Hydrogenation of I-22 over a catalyst such as platinum oxide provides ester I-23 as a mixture of isomers. Hydrolysis of I-23 gives the acid I-24 that is converted to amide I-19 by the same sequence of amide coupling and deprotection as I-16.

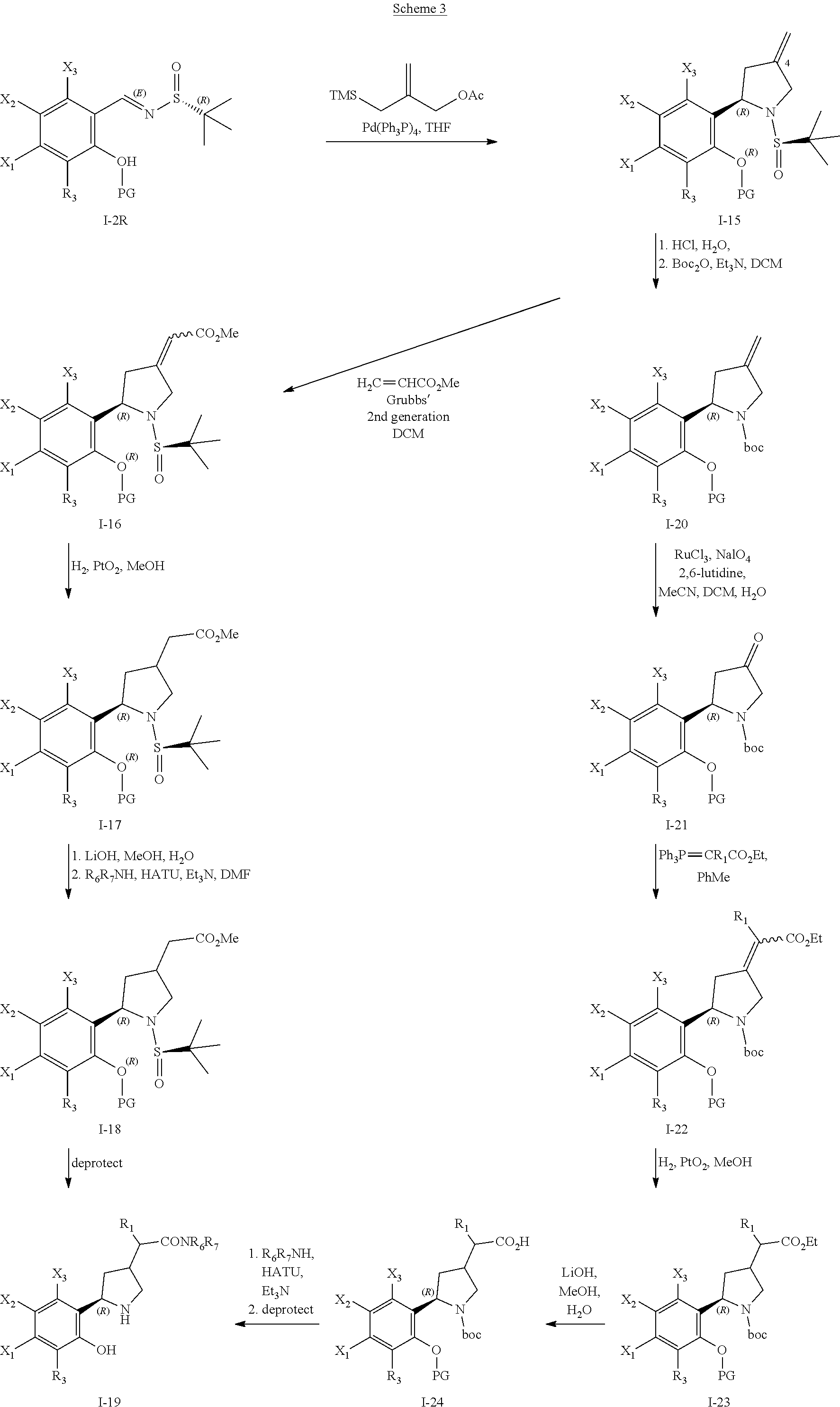

For compounds of Formula I disclosed herein where either R₁ or R₂, or both, are not H and m is 1, substitution on the extended chain can be obtained by the synthesis described in Scheme 4. Compound I-23a as shown in Scheme 4 can be obtained either from ketone I-21 by Wittig reaction with unsubstituted triphenylphosphanylidene acetate or from I-17 by exchanging the sulfinamide for Boc. As shown in Scheme 4, PG refers to a protecting group. Non-limiting examples of the protecting groups include Me, methoxymethyl (MOM), trimethylsilylethoxymethyl (SEM), allyl, Ac, Boc, other alkoxycarbonyl group, dialkylaminocarbonyl, and another protecting group known in the art suitable for use as protecting groups for OH or an amine group. Other substituents shown in Scheme 4 are defined herein. Alkylation of I-23a is carried out by forming the enolate with a strong base such as LDA and reacting with a halide R₁X to provide I-23. The alkylation can be repeated with a second R₂X that may be the same or different to give a geminally disubstituted ester I-23b. Hydrolysis, amide coupling and deprotection then provides the substituted amide I-19a.

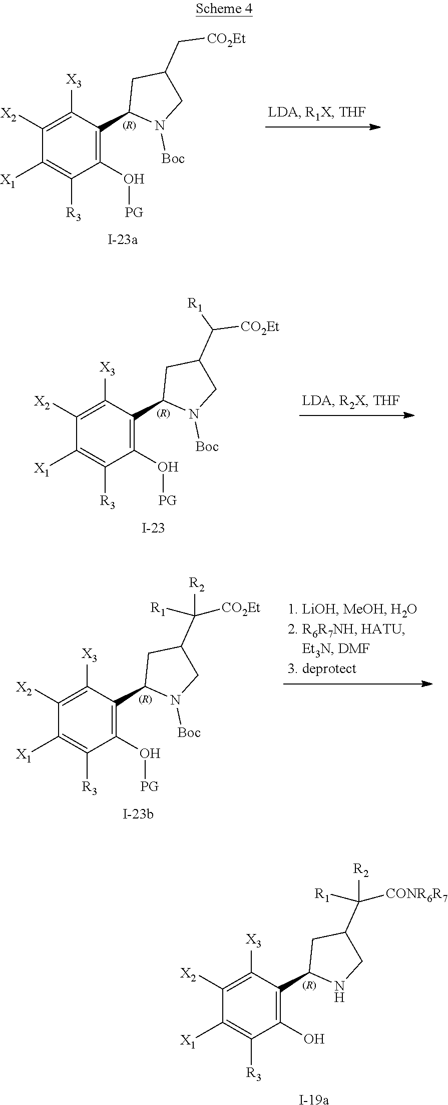

Compound I-24 as shown in Scheme 5 can be prepared by any method known in the art and/or is commercially available. As shown in Scheme 5, PG refers to a protecting group. Non-limiting examples of the protecting groups include Me, methoxymethyl (MOM), trimethylsilylethoxymethyl (SEM), allyl, Ac, Boc, other alkoxycarbonyl group, dialkylaminocarbonyl, and another protecting group known in the art suitable for use as protecting groups for OH or an amine group. Other substituents shown in Scheme 5 are defined herein. For compounds of Formula III disclosed herein where R₂ is H and m is 1, the aryl or heteroaryl ring can be obtained by a decarboxylative photoredox reaction as shown in Scheme 5. Reaction of an optionally protected aryl halide A₁X, where X is bromine or iodine, with carboxylic acid I-24 in the presence of an iridium catalyst [4,4′-bis(t-butyl)-2,2′-bipyridine-κN¹, κN¹]bis[3,5-difluoro-2-[5-(trifluoromethyl)-2-pyridinyl-κN¹]phenyl κC-iridium hexafluorophosphate, nickel chloride DME complex, di(t-butyl)-4,4′-bipyridine, phthalimide and t-butyl-tetramethylguanidine in DMSO under irradiation with blue light replaces the carboxylic acid with an aryl ring A₁ to yield I-25 after deprotection.

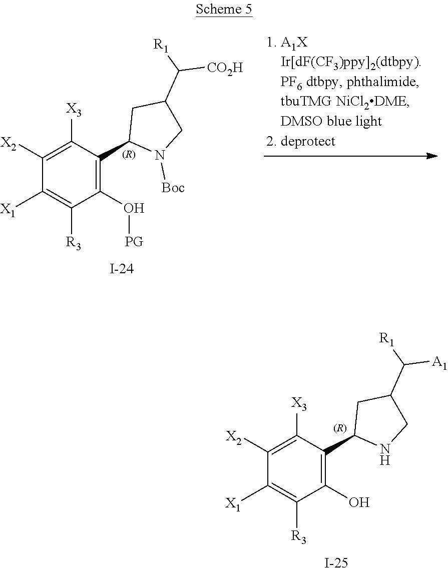

Compound I-8 as shown in Scheme 6 can be prepared by any method known in the art and/or is commercially available. As shown in Scheme 6, PG refers to a protecting group. Non-limiting examples of the protecting groups include Me, methoxymethyl (MOM), trimethylsilylethoxymethyl (SEM), allyl, Ac, Boc, other alkoxycarbonyl group, dialkylaminocarbonyl, and another protecting group known in the art suitable for use as protecting groups for OH or an amine group. Other substituents shown in Scheme 6 are defined herein. For compounds of Formula I′ disclosed herein where R₂ and R₉ are H and m is 1, the amide sidechain with the alternative orientation can be obtained by the synthesis described in Scheme 6. As shown in Scheme 6, carboxylic acid I-8 is converted to the primary amide I-26 by reacting with ammonium chloride, a coupling agent such as HATU and a base such as triethylamine in a solvent such as DMF. Reduction of I-26 to the primary amine I-27 is achieved by heating I-26 with a borane reducing agent such as borane-methyl sulfide complex in an ether solvent such as THE to provide I-27. Amine I-27 is then acylated with a carboxylic acid R₁₀CO₂H using a coupling agent such as HATU and a base such as triethylamine in a solvent such as DMF to yield amide I-28. Deprotection under standard conditions gives amide I-29 with R₁═H and R₂═H. To obtain compounds where R₁ is a substituent such as an alkyl group, acid I-8 is first converted to the Weinreb amide I-30 with N,O-dimethylhydroxylamine under amide coupling conditions. I-30 is then treated with a Grignard reagent R₁MgBr to give ketone I-31. Formation of the oxime of I-31 and reduction, for example by hydrogenation over Raney nickel, provides amine I-32. I-32 is acylated in the same way as I-27 and deprotected to yield I-29.

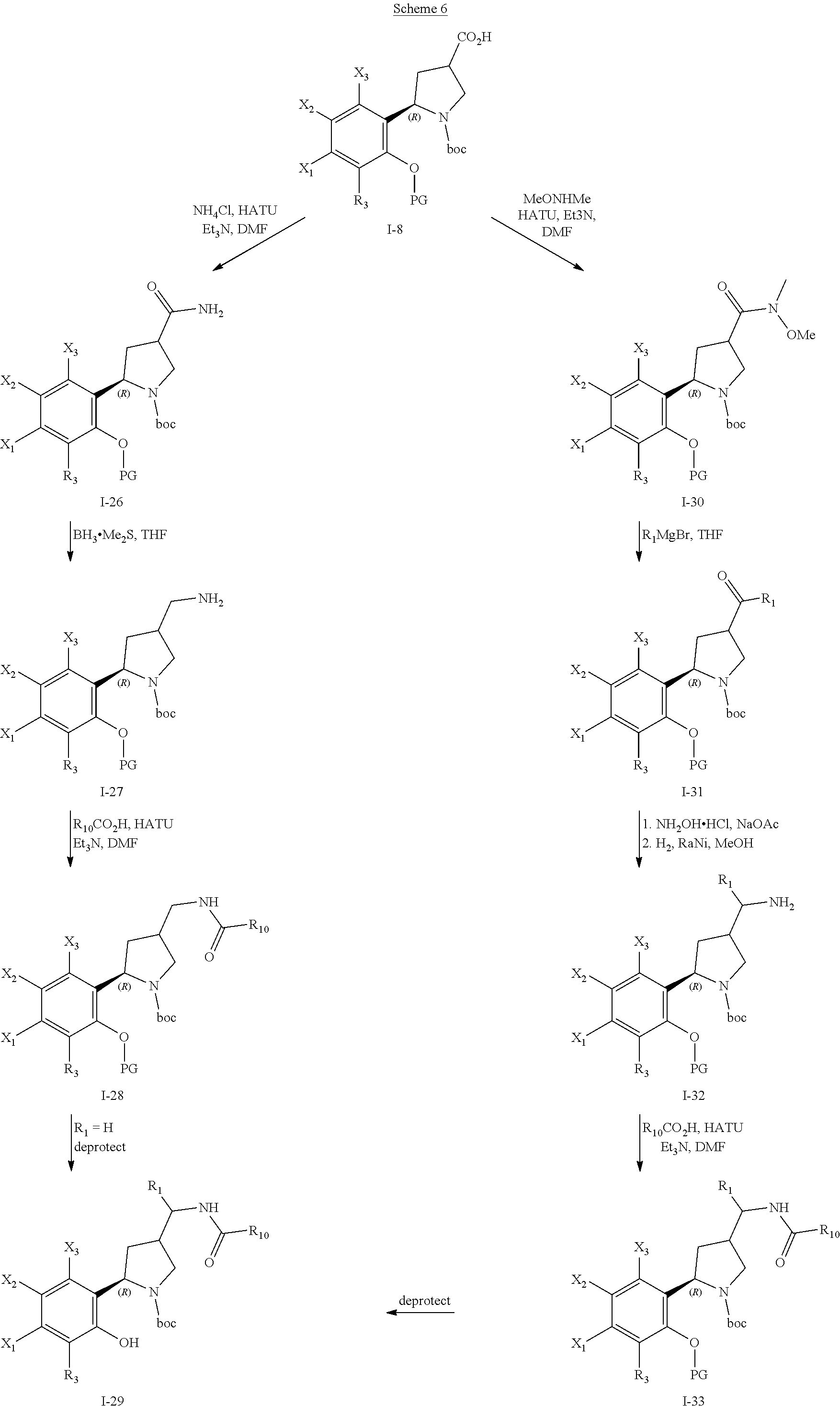

Compound I-14 as shown in Scheme 7 can be prepared by any method known in the art and/or is commercially available. As shown in Scheme 7, PG refers to a protecting group. Non-limiting examples of the protecting groups include Me, methoxymethyl (MOM), trimethylsilylethoxymethyl (SEM), allyl, Ac, Boc, other alkoxycarbonyl group, dialkylaminocarbonyl, and another protecting group known in the art suitable for use as protecting groups for OH or an amine group. Other substituents shown in Scheme 7 are defined herein. For compounds of Formula II disclosed herein where R₁ and R₂ are H and m is 1, the pyrrolidine ring with an extended chain at C5 (see compound I-14 with the 5-position labelled) can be obtained by the synthesis described in Scheme 7. As shown in Scheme 7. Ester I-14 is reduced to the alcohol I-34, for example with sodium borohydride. I-34 is then treated with an agent such as tosyl chloride and a base such as triethylamine, and the resulting tosylate is displaced by heating with tetrabutylammonium cyanide in a solvent such as DMF to form nitrile I-35. Hydrolysis of I-35 with sodium hydroxide and hydrogen peroxide yields primary amide I-36 that is then deprotected to give I-37. Substituted amides may be obtained by further hydrolysis of I-36 to the carboxylic acid I-38 followed by reaction with an amine R₆R₇NH and a coupling reagent such as HATU. The protecting groups are removed under standard conditions to provide pyrrolidine amide I-37a.

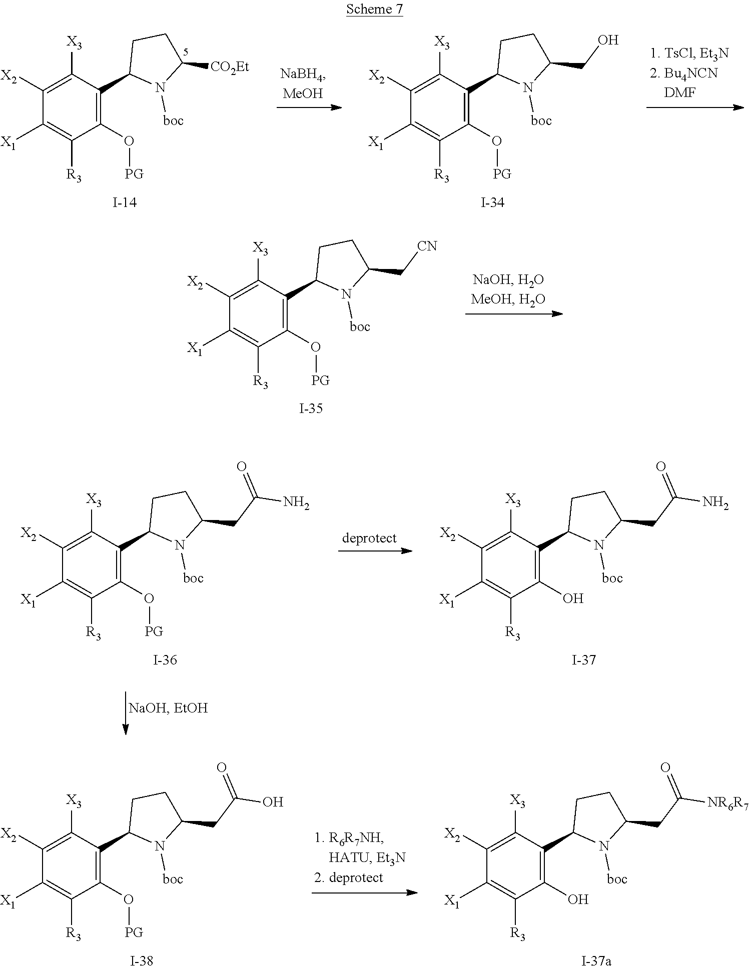

### Pharmaceutical Compositions

This invention also provides a pharmaceutical composition comprising at least one of the compounds as described herein or a pharmaceutically-acceptable salt or solvate thereof, and a pharmaceutically-acceptable carrier or diluent.

In yet another aspect, the present invention provides a pharmaceutical composition comprising at least one compound selected from the group consisting of compounds of Formula I, I′, II, II′, III, or IV as described herein and a pharmaceutically-acceptable carrier or diluent.

In certain embodiments, the composition is in the form of a hydrate, solvate or pharmaceutically-acceptable salt. The composition can be administered to the subject by any suitable route of administration, including, without limitation, oral and parenteral.

The phrase “pharmaceutically-acceptable carrier” as used herein means a pharmaceutically-acceptable material, composition or vehicle, such as a liquid or solid filler, diluent, excipient, solvent, or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose, and sucrose; starches, such as corn starch and potato starch; cellulose and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as butylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. The term “carrier” denotes an organic or inorganic ingredient, natural or synthetic, with which the active ingredient is combined to facilitate the application. The components of the pharmaceutical compositions also are capable of being comingled with the compounds of the present invention, and with each other, in a manner such that there is no interaction which would substantially impair the desired pharmaceutical efficiency.

As set out above, certain embodiments of the present pharmaceutical agents may be provided in the form of pharmaceutically-acceptable salts. The term “pharmaceutically-acceptable salt,” in this respect, refers to the relatively non-toxic, inorganic and organic acid salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts, and the like. See, e.g., Berge et al., (1977) “Pharmaceutical Salts”, J. Pharm. Sci. 66:1-19 (incorporated herein by reference in its entirety).

The pharmaceutically-acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, butionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like. See, e.g., Berge et al. (supra).

Wetting agents, emulsifiers, and lubricants, such as sodium lauryl sulfate, magnesium stearate, and polyethylene oxide-polybutylene oxide copolymer, as well as coloring agents, release agents, coating agents, sweetening, flavoring and perfuming agents, preservatives, and antioxidants can also be present in the compositions.

Formulations of the present invention include those suitable for oral, nasal, topical (including buccal and sublingual), rectal, vaginal, and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated and the particular mode of administration. The amount of active ingredient, which can be combined with a carrier material to produce a single dosage form will generally be that amount of the compound which produces a therapeutic effect. Generally, out of 100%, this amount will range from about 1% to about 99% of active ingredient, preferably from about 5% to about 70%, most preferably from about 10% to about 30%.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), and/or as mouthwashes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, and the like), the active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose, and/or acacia; humectants, such as glycerol; disintegrating agents, such as agar-agar, calcium carbonate, potato or tapioca starch, alginic acid, certain silicates, sodium carbonate, and sodium starch glycolate; solution retarding agents, such as paraffin; absorption accelerators, such as quaternary ammonium compounds; wetting agents, such as, for example, cetyl alcohol, glycerol monostearate, and polyethylene oxide-polybutylene oxide copolymer; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxybutylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills, and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxybutylmethyl cellulose in varying proportions, to provide the desired release profile, other polymer matrices, liposomes, and/or microspheres. They may be sterilized by, for example, filtration through a bacteria-retaining filter, or by incorporating sterilizing agents in the form of sterile solid compositions, which can be dissolved in sterile water or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions, which can be used include polymeric substances and waxes. The active ingredient can also be in micro-encapsulated form, if appropriate, with one or more of the above-described excipients.

Liquid dosage forms for oral administration of the compounds of the invention include pharmaceutically-acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents and emulsifiers, such as ethyl alcohol, isobutyl alcohol, ethyl carbonate, ethyl acetate, benzyl alcohol, benzyl benzoate, butylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Additionally, cyclodextrins, e.g., hydroxybutyl-β-cyclodextrin, may be used to solubilize compounds.

Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar, and tragacanth, and mixtures thereof.

Dosage forms for the topical or transdermal administration of a compound of this invention include powders, sprays, ointments, pastes, creams, lotions, gels, solutions, patches, and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and butane.

Transdermal patches have the added advantage of providing controlled delivery of a compound of the present invention to the body. Such dosage forms can be made by dissolving or dispersing the pharmaceutical agents in the proper medium. Absorption enhancers can also be used to increase the flux of the pharmaceutical agents of the invention across the skin. The rate of such flux can be controlled, by either providing a rate-controlling membrane or dispersing the compound in a polymer matrix or gel.

Ophthalmic formulations, eye ointments, powders, solutions, and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions, or emulsions; or sterile powders which may be reconstituted into sterile injectable solutions or dispersions just prior to use, which may contain antioxidants, buffers, bacteriostats, or solutes which render the formulation isotonic with the blood of the intended recipient or suspending or thickening agents.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally-administered drug form is accomplished by dissolving or suspending the drug in an oil vehicle. One strategy for depot injections includes the use of polyethylene oxide-polypropylene oxide copolymers wherein the vehicle is fluid at room temperature and solidifies at body temperature.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot-injectable formulations are also prepared by entrapping the drug in liposomes or microemulsions, which are compatible with body tissue.

When the compounds of the present invention are administered as pharmaceuticals, to humans and animals, they can be given per se or as a pharmaceutical composition containing, for example, 0.1% to 99.5% (more preferably, 0.5% to 90%) of active ingredient in combination with a pharmaceutically-acceptable carrier.

The compounds and pharmaceutical compositions of the present invention can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, the compound of the present invention may be administered concurrently with another anticancer agents).

The compounds of the invention may be administered intravenously, intramuscularly, intraperitoneally, subcutaneously, topically, orally, or by other acceptable means. The compounds may be used to treat arthritic conditions in mammals (e.g., humans, livestock, and domestic animals), racehorses, birds, lizards, and any other organism which can tolerate the compounds.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use, or sale of pharmaceuticals or biological products, which notice reflects approval by the agency of manufacture, use, or sale for human administration.

### Administration to a Subject

In yet another aspect, the present invention provides a method for treating a condition in a mammalian species in need thereof, the method comprising administering to the mammalian species a therapeutically effective amount of at least one compound selected from the group consisting of compounds of Formula I, I′, II, II′, III, or IV, or a pharmaceutically-acceptable salt thereof or a pharmaceutical composition thereof, wherein the condition is selected from the group consisting of cancer, an immunological disorder, a CNS disorder, an inflammatory disorder, a gastroenterological disorder, a metabolic disorder, a cardiovascular disorder, and a kidney disease.

In some embodiments, the cancer is selected from the group consisting of biliary tract cancer, brain cancer, breast cancer, cervical cancer, choriocarcinoma, colon cancer, endometrial cancer, esophageal cancer, gastric (stomach) cancer, intraepithelial neoplasms, leukemias, lymphomas, liver cancer, lung cancer, melanoma, neuroblastomas, oral cancer, ovarian cancer, pancreatic cancer, prostate cancer, rectal cancer, renal (kidney) cancer, sarcomas, skin cancer, testicular cancer, and thyroid cancer.

In some embodiments, the inflammatory disorder is an inflammatory skin condition, arthritis, psoriasis, spondylitis, parodontitits, or an inflammatory neuropathy. In some embodiments, the gastroenterological disorder is an inflammatory bowel disease such as Crohn's disease or ulcerative colitis.

In some embodiments, the immunological disorder is transplant rejection or an autoimmune disease (e.g., rheumatoid arthritis, MS, systemic lupus erythematosus, or type I diabetes mellitus). In some embodiments, the CNS disorder is Alzheimer's disease.

In some embodiments, the metabolic disorder is obesity or type II diabetes mellitus. In some embodiments, the cardiovascular disorder is an ischemic stroke. In some embodiments, the kidney disease is chronic kidney disease, nephritis, or chronic renal failure.

In some embodiments, the mammalian species is human.

In some embodiments, the condition is selected from the group consisting of cancer, transplant rejection, rheumatoid arthritis, multiple sclerosis, systemic lupus erythematosus, type I diabetes mellitus, Alzheimer's disease, inflammatory skin condition, inflammatory neuropathy, psoriasis, spondylitis, parodontitis, inflammatory bowel disease, obesity, type II diabetes mellitus, ischemic stroke, chronic kidney disease, nephritis, chronic renal failure, and a combination thereof.

In yet another aspect, a method of blocking Kv1.3 potassium channel in a mammalian species in need thereof is described, including administering to the mammalian species a therapeutically effective amount of at least one compound of Formula I, I′, II, II′, III, or IV, or a pharmaceutically-acceptable salt or pharmaceutical composition thereof.

In some embodiments, the compounds described herein is selective in blocking the Kv1.3 potassium channels with minimal or no off-target inhibition activities against other potassium channels, or against calcium or sodium channels. In some embodiments, the compounds described herein do not block the hERG channels and therefore have desirable cardiovascular safety profiles.

Some aspects of the invention involve administering an effective amount of a composition to a subject to achieve a specific outcome. The small molecule compositions useful according to the methods of the present invention thus can be formulated in any manner suitable for pharmaceutical use.

The formulations of the invention are administered in pharmaceutically-acceptable solutions, which may routinely contain pharmaceutically-acceptable concentrations of salt, buffering agents, preservatives, compatible carriers, adjuvants, and optionally other therapeutic ingredients.

For use in therapy, an effective amount of the compound can be administered to a subject by any mode allowing the compound to be taken up by the appropriate target cells. “Administering” the pharmaceutical composition of the present invention can be accomplished by any means known to the skilled artisan. Specific routes of administration include, but are not limited to, oral, transdermal (e.g., via a patch), parenteral injection (subcutaneous, intradermal, intramuscular, intravenous, intraperitoneal, intrathecal, etc.), or mucosal (intranasal, intratracheal, inhalation, intrarectal, intravaginal, etc.). An injection can be in a bolus or a continuous infusion.

For example the pharmaceutical compositions according to the invention are often administered by intravenous, intramuscular, or other parenteral means. They can also be administered by intranasal application, inhalation, topically, orally, or as implants; even rectal or vaginal use is possible. Suitable liquid or solid pharmaceutical preparation forms are, for example, aqueous or saline solutions for injection or inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops, or preparations with protracted release of active compounds in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of present methods for drug delivery, see Langer R (1990) Science 249:1527-33, which is incorporated herein by reference in its entirety.

The concentration of compounds included in compositions used in the methods of the invention can range from about 1 nM to about 100 μM. Effective doses are believed to range from about 10 picomole/kg to about 100 micromole/kg.

The pharmaceutical compositions are preferably prepared and administered in dose units. Liquid dose units are vials or ampoules for injection or other parenteral administration. Solid dose units are tablets, capsules, powders, and suppositories. For treatment of a patient, different doses may be necessary depending on activity of the compound, manner of administration, purpose of the administration (i.e., prophylactic or therapeutic), nature and severity of the disorder, age and body weight of the patient. The administration of a given dose can be carried out both by single administration in the form of an individual dose unit or else several smaller dose units. Repeated and multiple administration of doses at specific intervals of days, weeks, or months apart are also contemplated by the invention.

The compositions can be administered per se (neat) or in the form of a pharmaceutically-acceptable salt. When used in medicine the salts should be pharmaceutically acceptable, but non-pharmaceutically-acceptable salts can conveniently be used to prepare pharmaceutically-acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v); and thimerosal (0.004-0.02% w/v).

Compositions suitable for parenteral administration conveniently include sterile aqueous preparations, which can be isotonic with the blood of the recipient. Among the acceptable vehicles and solvents are water, Ringer's solution, phosphate buffered saline, and isotonic sodium chloride solution. In addition, sterile, fixed oils are conventionally employed as a solvent or suspending medium. For this purpose, any bland fixed mineral or non-mineral oil may be employed including synthetic mono- or diglycerides. In addition, fatty acids such as oleic acid find use in the preparation of injectables. Carrier formulations suitable for subcutaneous, intramuscular, intraperitoneal, intravenous, etc. administrations can be found in Remington's Pharmaceutical Sciences, Mack Publishing Company, Easton, PA; incorporated herein by reference in its entirety.

The compounds useful in the invention can be delivered in mixtures of more than two such compounds. A mixture can further include one or more adjuvants in addition to the combination of compounds.

A variety of administration routes is available. The particular mode selected will depend, of course, upon the particular compound selected, the age and general health status of the subject, the particular condition being treated, and the dosage required for therapeutic efficacy. The methods of this invention, generally speaking, can be practiced using any mode of administration that is medically acceptable, meaning any mode that produces effective levels of response without causing clinically unacceptable adverse effects. Preferred modes of administration are discussed above.

The compositions can conveniently be presented in unit dosage form and can be prepared by any of the methods well known in the art of pharmacy. All methods include the step of bringing the compounds into association with a carrier which constitutes one or more accessory ingredients. In general, the compositions are prepared by uniformly and intimately bringing the compounds into association with a liquid carrier, a finely divided solid carrier, or both, and then, if necessary, shaping the product.

Other delivery systems can include time-release, delayed release, or sustained-release delivery systems. Such systems can avoid repeated administrations of the compounds, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids, or neutral fats such as mono-di- and tri-glycerides; hydrogel release systems; silastic systems; peptide-based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974, and 5,407,686. In addition, pump-based hardware delivery systems can be used, some of which are adapted for implantation.

### Assays for Effectiveness of Kv1.3 Potassium Channel Blockers

In some embodiments, the compounds as described herein are tested for their activities against Kv1.3 potassium channel. In some embodiments, the compounds as described herein are tested for their Kv1.3 potassium channel electrophysiology. In some embodiments, the compounds as described herein are tested for their hERG electrophysiology.

### EQUIVALENTS

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art. The following examples contain important additional information, exemplification, and guidance which can be adapted to the practice of this invention in its various embodiments and equivalents thereof.

### EXAMPLES

Examples 1-11 describe various intermediates used in the syntheses of representative compounds of Formula I, I′, II, II′, III, or IV disclosed herein.

### Example 1. Intermediate 1a ((R)—N-[[2,3-dichloro-6-(methoxymethoxy)phenyl]methylidene]-2-methylpropane-2-sulfinamide) and Intermediate 1b ((S)—N-[[2,3-dichloro-6-(methoxymethoxy)phenyl]methylidene]-2-methylpropane-2-sulfinamide)

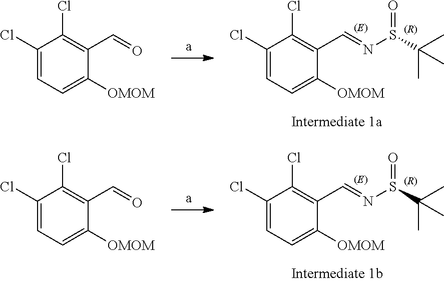

### Step a

To a stirred solution of 2,3-dichloro-6-(methoxymethoxy)benzaldehyde (2.00 g, 8.51 mmol) and (R)-2-methylpropane-2-sulfinamide (1.55 g, 12.8 mmol) in THE (20 mL) was added Ti(OEt)₄ (5.82 g, 25.52 mmol) at room temperature under nitrogen atmosphere. The resulting solution was stirred for 16 h, quenched with saturated aq. NaHCO₃ (50 mL), and extracted with EA (3×50 mL). The combined organic layers were washed with brine (3×50 mL) and dried over anhydrous Na₂SO₄. After filtration, the filtrate was concentrated under reduced pressure. The residue was purified by silica gel column chromatography, eluting with PE/EA (3/1) to afford ((R)—N-[[2,3-dichloro-6-(methoxymethoxy)phenyl]methylidene]-2-methylpropane-2-sulfinamide) as a light-yellow oil (2.60 g, 81%): LCMS (ESI) calc'd for C₁₃H₁₇Cl₂NO₃S [M+H]⁺: 338, 340 (3:2) found 338, 340 (3:2); ¹H NMR (300 MHz, CDCl₃) δ 8.91 (s, 1H), 7.49 (d, J=9.0 Hz, 1H), 7.13 (d, J=9.0 Hz, 1H), 5.23 (s, 2H), 3.48 (s, 3H), 1.31 (s, 9H). The (S) enantiomer Intermediate 1b was prepared in the same way using (R)-2-methylpropane-2-sulfinamide.

### Example 2. Intermediate 2 (tert-butyl (2R)-2-[2,3-dichloro-6-(methoxymethoxy)phenyl]-4-oxopyrrolidine-1-carboxylate)

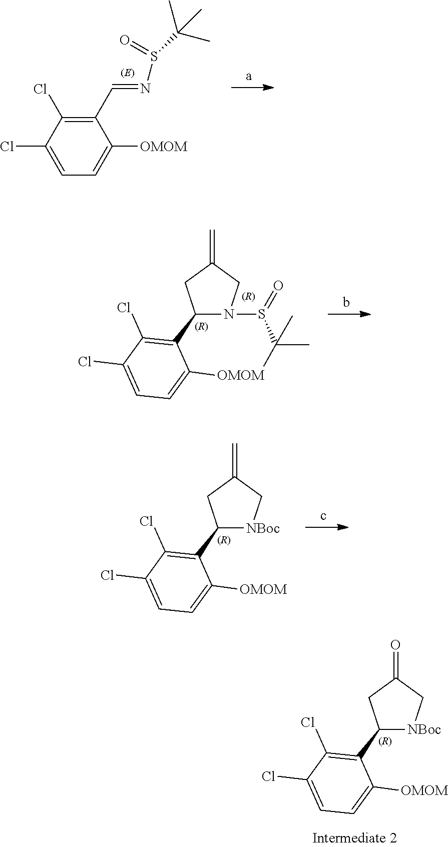

### Step a

To a stirred solution of (R)—N-[[2,3-dichloro-6-(methoxymethoxy)phenyl]methylidene]-2-methylpropane-2-sulfinamide (10.0 g, 29.6 mmol) and 2-[(trimethylsilyl)methyl]prop-2-en-1-yl acetate (8.26 g, 44.4 mmol) in THE (120 mL) was added Pd(PPh₃)₄ (3.42 g, 2.96 mmol) at room temperature under nitrogen atmosphere. The resulting reaction mixture was stirred at room temperature for 20 h, quenched with water (100 mL) and extracted with EA (3×150 mL). The combined organic layers were washed with brine (3×100 mL) and dried over anhydrous Na₂SO₄. After filtration, the filtrate was concentrated under reduced pressure. The residue was purified by silica gel column chromatography, eluting with PE/EA (3/1) to afford (2R)-2-[2,3-dichloro-6-(methoxymethoxy)phenyl]-4-methylidene-1-[(R)-2-methylpropane-2-sulfinyl]pyrrolidine as a light yellow oil (7.30 g, 60%): LCMS (ESI) calc'd for C₁₇H₂₃Cl₂NO₃S [M+H]⁺: 392, 394 (3:2) found 392, 394 (3:2); ¹H NMR (400 MHz, CDCl₃) δ 7.34 (d, J=8.9 Hz, 1H), 7.11 (d, J=9.1 Hz, 1H), 5.70-5.57 (m, 1H), 5.20-5.09 (m, 2H), 4.99-4.91 (m, 2H), 4.37 (d, J=13.9 Hz, 1H), 3.89 (d, J=13.9 Hz, 1H), 3.49 (s, 3H), 3.07-2.94 (m, 1H), 2.80-2.70 (m, 1H), 1.09 (s, 9H).

### Step b

To a stirred solution of (2R)-2-[2,3-dichloro-6-(methoxymethoxy)phenyl]-4-methylidene-1-[(R)-2-methylpropane-2-sulfinyl]pyrrolidine (7.30 g, 18.6 mmol) in MeOH (60 mL) was added aq. HCl (4 N, 15 mL) dropwise at room temperature. The resulting reaction solution was stirred at room temperature for 1 h, basified to pH 8 with saturated aq. NaHCO₃ and extracted with EA (3×100 mL). The combined organic layers were washed with brine (2×100 mL) and dried over anhydrous Na₂SO₄. After filtration, the filtrate was concentrated under reduced pressure. To the crude product Boc₂O (6.25 g, 28.6 mmol) in DCM (60 mL) and TEA (5.31 mL, 38.2 mmol) were added at room temperature. The reaction mixture was stirred at room temperature for 1 h and concentrated under reduced pressure. The residue was purified by silica gel column chromatography, eluting with PE/EA (5/1) to afford tert-butyl (2R)-2-[2,3-dichloro-6-(methoxymethoxy)phenyl]-4-methylidenepyrrolidine-1-carboxylate as a light yellow oil (7.30 g, 89%): LCMS (ESI) calc'd for C₁₈H₂₃Cl₂NO₄ [M+H]⁺: 388, 390 (3:2) found 388, 390 (3:2); ¹H NMR (400 MHz, CDCl₃) δ 7.32 (d, J=8.9 Hz, 1H), 7.02 (d, J=9.0 Hz, 1H), 5.73-5.55 (m, 1H), 5.21 (d, J=7.1 Hz, 1H), 5.11 (d, J=7.0 Hz, 1H), 5.05-4.93 (m, 2H), 4.23-4.13 (m, 2H), 3.45 (s, 3H), 3.11-3.00 (m, 1H), 2.82-2.73 (m, 1H), 1.17 (s, 9H).

### Step c

To a stirred mixture of tert-butyl (2R)-2-[2,3-dichloro-6-(methoxymethoxy)phenyl]-4-methylidenepyrrolidine-1-carboxylate (7.30 g, 18.8 mmol) in DCM (40 mL) and ACN (40 mL) were added NaIO₄ (12.1 g, 56.4 mmol), H₂O (60 mL), 2,6-lutidine (4.03 g, 37.6 mmol) and RuCl₃·H₂O (0.420 g, 1.88 mmol) at room temperature. The resulting reaction mixture was stirred for 1 h, quenched with saturated aq. NH₄HCO₃ (200 mL) and extracted with EA (3×200 mL). The combined organic layers were washed with brine (2×200 mL) and dried over anhydrous Na₂SO₄. After filtration, the filtrate was concentrated under reduced pressure. The residue was purified by silica gel column chromatography, eluting with PE/EA (4/1) to afford tert-butyl (2R)-2-[2,3-dichloro-6-(methoxymethoxy)phenyl]-4-oxopyrrolidine-1-carboxylate as a light yellow oil (5.60 g, 69%): LCMS (ESI) calc'd for C₁₇H₂₁C₁₂NO₅ [M+H−56]⁺: 334, 336 (3:2) found 334, 336 (3:2); ¹H NMR (300 MHz, CDCl₃) δ 7.55-7.31 (m, 1H), 7.02 (dd, J=20.2, 8.4 Hz, 1H), 6.12-5.89 (m, 1H), 5.20-5.08 (m, 2H), 4.00-3.88 (m, 2H), 3.45-3.37 (m, 3H), 3.15 (dd, J=18.8, 11.1 Hz, 1H), 2.61-2.48 (m, 1H), 1.28 (s, 9H).

### Example 3. Intermediate 3 (tert-butyl (2R,4Z)-2-[2,3-dichloro-6-(methoxymethoxy)phenyl]-4-(2-ethoxy-2-oxoethylidene)pyrrolidine-1-carboxylate)

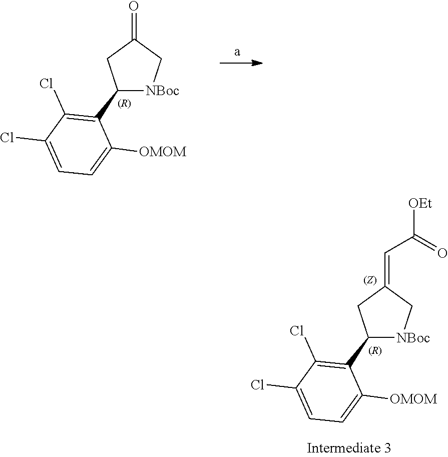

### Step a

A mixture of tert-butyl (2R)-2-[2,3-dichloro-6-(methoxymethoxy)phenyl]-4-oxopyrrolidine-1-carboxylate (0.800 g, 2.05 mmol) and ethyl 2-(triphenylphosphanylidene)acetate (1.07 g, 3.07 mmol) in toluene (8 mL) was stirred at 110° C. for 16 h. After cooling to room temperature, the resulting mixture was concentrated under reduced pressure. The residue was purified by silica gel column chromatography, eluting with PE/EA (3/1) to afford tert-butyl (2R,4Z)-2-[2,3-dichloro-6-(methoxymethoxy)phenyl]-4-(2-ethoxy-2-oxoethylidene)pyrrolidine-1-carboxylate as a colorless oil (0.840 g, 80%): LCMS (ESI) calc'd for C₂₁H₂₇Cl₂NO₆ [M+H]⁺: 460, 462 (3:2) found 460, 462 (3:2); ¹H NMR (400 MHz, CDCl₃) δ 7.36-7.29 (m, 1H), 7.04-6.98 (m, 1H), 5.78-5.74 (m, 1H), 5.19-5.14 (m, 1H), 5.12-5.02 (m, 1H), 4.77-4.58 (m, 2H), 4.46-4.32 (m, 1H), 4.27-4.19 (m, 2H), 3.40 (d, J=5.7 Hz, 3H), 3.36-3.19 (m, 1H), 2.92-2.73 (m, 1H), 1.35-1.29 (m, 3H), 1.23-1.18 (m, 9H).

### Example 4. Intermediate 4a (tert-butyl (2R)-2-[2,3-dichloro-6-(methoxymethoxy)phenyl]-4-(2-ethoxy-2-oxoethyl)pyrrolidine-1-carboxylate)

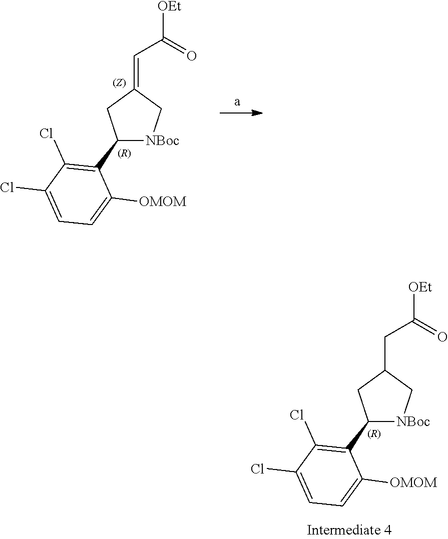

### Step a

To a stirred mixture of tert-butyl (2R,4Z)-2-[2,3-dichloro-6-(methoxymethoxy)phenyl]-4-(2-ethoxy-2-oxoethylidene)pyrrolidine-1-carboxylate (0.620 g, 1.35 mmol) in MeOH (6 mL) was added PtO₂ (0.130 g, 0.550 mmol) at room temperature. The reaction mixture was degassed under reduced pressure, purged with hydrogen three times and stirred under hydrogen atmosphere (1.5 atm) at room temperature for 2 h. The resulting mixture was filtered and the filter cake was washed with MeOH (3×5 mL). The filtrate was concentrated under reduced pressure to afford tert-butyl (2R)-2-[2,3-dichloro-6-(methoxymethoxy)phenyl]-4-(2-ethoxy-2-oxoethyl)pyrrolidine-1-carboxylate as a light yellow oil (0.600 g, 87%): LCMS (ESI) calc'd for C₂₁H₂₉Cl₂NO₆ [M+H]⁺: 462, 464 (3:2) found 462, 464 (3:2); ¹H NMR (300 MHz, CDCl₃) δ 7.34-7.29 (m, 1H), 7.06-6.97 (m, 1H), 5.57-5.39 (m, 1H), 5.31-5.07 (m, 2H), 4.23-4.11 (m, 2H), 4.01-3.68 (m, 1H), 3.57-3.08 (m, 4H), 2.91-2.58 (m, 1H), 2.55-2.20 (m, 3H), 2.161.76 (m, 1H), 1.331.24 (m, 3H), 1.15 (d, J=2.4 Hz, 9H).

### Example 5. Intermediate 4a (tert-butyl (2R,4S)-2-[2,3-dichloro-6-(methoxymethoxy)phenyl]-4-(2-ethoxy-2-oxoethyl)pyrrolidine-1-carboxylate) and Intermediate 4b (tert-butyl (2R,4R)-2-[2,3-dichloro-6-(methoxymethoxy)phenyl]-4-(2-ethoxy-2-oxoethyl)pyrrolidine-1-carboxylate)

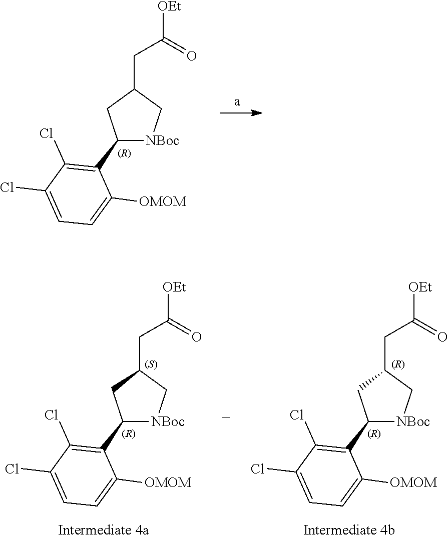

### Step a

tert-butyl (2R)-2-(2,3-dichloro-6-(methoxymethoxy)phenyl)-4-(2-ethoxy-2-oxoethyl)pyrrolidine-1-carboxylate (13.0 g) was separated by Prep-SFC with the following conditions: Column: OptiChiral-C9-5, 3×25 cm, 5 m; Mobile Phase A: CO₂, Mobile Phase B: MeOH (plus 0.1% 2M NH₃-MeOH); Flow rate: 250 mL/min; Gradient: isocratic 14% B; Column Temperature: 35° C.; Back Pressure: 100 bar; Wave Length: 220 nm; Retention time 1: 2.80 min; Retention time 2: 3.40 min; Sample Solvent: MeCN/MeOH=4/1; Injection Volume: 2 mL; Number Of Runs: 75. The faster-eluting enantiomer at 2.80 min was obtained tert-butyl (2R,4S)-2-[2,3-dichloro-6-(methoxymethoxy)phenyl]-4-(2-ethoxy-2-oxoethyl)pyrrolidine-1-carboxylate (Intermediate 4a) as a light yellow oil (9.44 g, 73% yield): LCMS (ESI) calc'd for C₂₁H₂₉Cl₂NO₆ [M+H]⁺: 462, 464 (3:2) found 462, 464 (3:2); ¹H NMR (300 MHz, CDCl₃) δ 7.31 (d, J=9.0 Hz, 1H), 7.02 (d, J=8.9 Hz, 1H), 5.45 (t, J=8.8 Hz, 1H), 5.27-5.07 (m, 2H), 4.16 (q, J=7.1 Hz, 2H), 3.95 (dd, J=10.3, 7.2 Hz, 1H), 3.53-3.43 (m, 3H), 3.14 (t, J=10.5 Hz, 1H), 2.71-2.56 (m, 1H), 2.55-2.38 (m, 3H), 1.86 (q, J=11.6 Hz, 1H), 1.28 (t, J=7.1 Hz, 3H), 1.14 (s, 9H). The slower-eluting enantiomer at 3.40 min was obtained tert-butyl (2R,4R)-2-[2,3-dichloro-6-(methoxymethoxy)phenyl]-4-(2-ethoxy-2-oxoethyl)pyrrolidine-1-carboxylate (Intermediate 4b) as a light yellow oil (1.33 g, 10% yield): LCMS (ESI) calc'd for C₂₁H₂₉Cl₂NO₆ [M+H]⁺: 462, 464 (3:2) found 462, 464 (3:2); ¹H NMR (300 MHz, CDCl₃) δ 7.31 (d, J=8.7 Hz, 1H), 7.02 (d, J=8.9 Hz, 1H), 5.45 (t, J=8.9 Hz, 1H), 5.30-5.07 (m, 2H), 4.16 (q, J=7.1 Hz, 2H), 3.95 (dd, J=10.3, 7.3 Hz, 1H), 3.52-3.45 (m, 3H), 3.14 (t, J=10.5 Hz, 1H), 2.72-2.58 (m, 1H), 2.44 (dt, J=13.8, 6.5 Hz, 3H), 1.86 (q, J=11.7 Hz, 1H), 1.28 (t, J=7.1 Hz, 3H), 1.13 (s, 9H).

### Example 6. Intermediate 5 ([(5R)-1-(tert-butoxycarbonyl)-5-[2,3-dichloro-6-(methoxymethoxy)phenyl]pyrrolidin-3-yl]acetic acid)

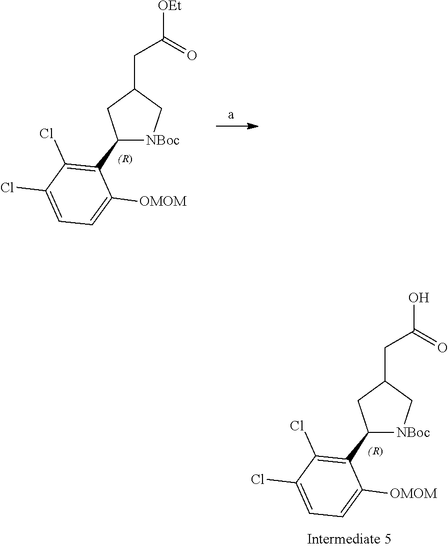

### Step a

To a stirred solution of tert-butyl (2R)-2-[2,3-dichloro-6-(methoxymethoxy)phenyl]-4-(2-ethoxy-2-oxoethyl)pyrrolidine-1-carboxylate (1.50 g, 3.24 mmol) in MeOH (15 mL) and H₂O (3 mL) was added LiOH (0.230 g, 9.73 mmol) at room temperature. The reaction mixture was stirred for 2 h, acidified to pH 2 with saturated aq. citric acid, diluted with water (50 mL) and extracted with EA (3×50 mL). The combined organic layers were washed with brine (3×50 mL) and dried over anhydrous Na₂SO₄. After filtration, the filtrate was concentrated under reduced pressure to afford [(5R)-1-(tert-butoxycarbonyl)-5-[2,3-dichloro-6-(methoxymethoxy)phenyl]pyrrolidin-3-yl]acetic acid as an off-white solid (1.20 g, 85%): LCMS (ESI) calc'd for C₁₉H₂₅C₁₂NO₆ [M+H]⁺: 434, 436 (3:2) found 434, 436 (3:2); ¹H NMR (300 MHz, CDCl₃) δ 7.32 (d, J=8.9 Hz, 1H), 7.03 (d, J=8.9 Hz, 1H), 5.60-5.36 (m, 1H), 5.36-5.06 (m, 2H), 4.01 (dd, J=10.3, 7.3 Hz, 1H), 3.56-3.43 (m, 4H), 3.18 (t, J=10.5 Hz, 1H), 2.78-2.34 (m, 3H), 1.88 (q, J=11.5 Hz, 1H), 1.15 (s, 9H).

### Example 7. Intermediate 5a ([(3S,5R)-1-(tert-butoxycarbonyl)-5-[2,3-dichloro-6-(methoxymethoxy)phenyl]pyrrolidin-3-yl]acetic acid)

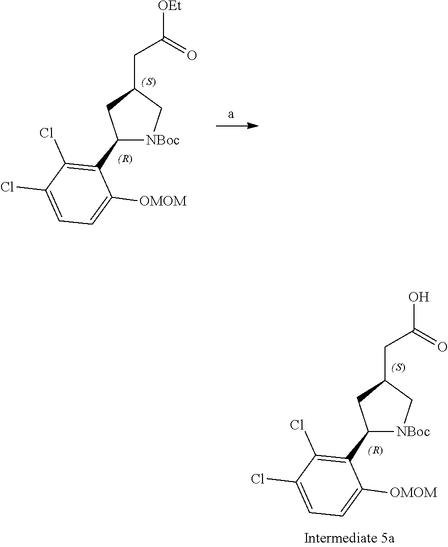

### Step a

To a stirred solution of tert-butyl (2R,4S)-2-[2,3-dichloro-6-(methoxymethoxy)phenyl]-4-(2-ethoxy-2-oxoethyl)pyrrolidine-1-carboxylate (0.550 g, 1.19 mmol) in H₂O (0.4 mL) and MeOH (2 mL) was added LiOH (85.0 mg, 3.54 mmol) at room temperature. The reaction mixture was stirred at room temperature for 2 h. The resulting mixture was acidified to pH 6 with citric acid followed by extracted with EA (3×30 mL). The combined organic layers were washed with brine (3×30 mL) and dried over anhydrous Na₂SO₄. After filtration, the filtrate was concentrated under reduced pressure to afford [(3S,5R)-1-(tert-butoxycarbonyl)-5-[2,3-dichloro-6-(methoxymethoxy)phenyl]pyrrolidin-3-yl]acetic acid as an white solid (0.450 g, 87%): LCMS (ESI) calc'd for C₁₉H₂₅Cl₂NO₆ [M+H]⁺: 434, 436 (3:2) found 434, 436 (3:2); ¹H NMR (400 MHz, CDCl₃) δ 7.32 (d, J=8.9 Hz, 1H), 7.03 (d, J=8.9 Hz, 1H), 5.47 (t, J=9.0 Hz, 1H), 5.32-5.08 (m, 2H), 4.01 (dd, J=10.3, 7.3 Hz, 1H), 3.48 (s, 3H), 3.18 (t, J=10.5 Hz, 1H), 2.67 (d, J=7.2 Hz, 1H), 2.62-2.38 (m, 3H), 1.88 (q, J=11.6 Hz, 1H), 1.22 (d, J=53.9 Hz, 9H).

### Example 8. Intermediate 6a ((ethyl (3S,5R)-5-[2,3-dichloro-6-(methoxymethoxy)phenyl]-1-(4-methylbenzenesulfonyl)pyrrolidine-3-carboxylate) and Intermediate 6b ((ethyl (3R,5R)-5-[2,3-dichloro-6-(methoxymethoxy)phenyl]-1-(4-methylbenzenesulfonyl)pyrrolidine-3-carboxylate)

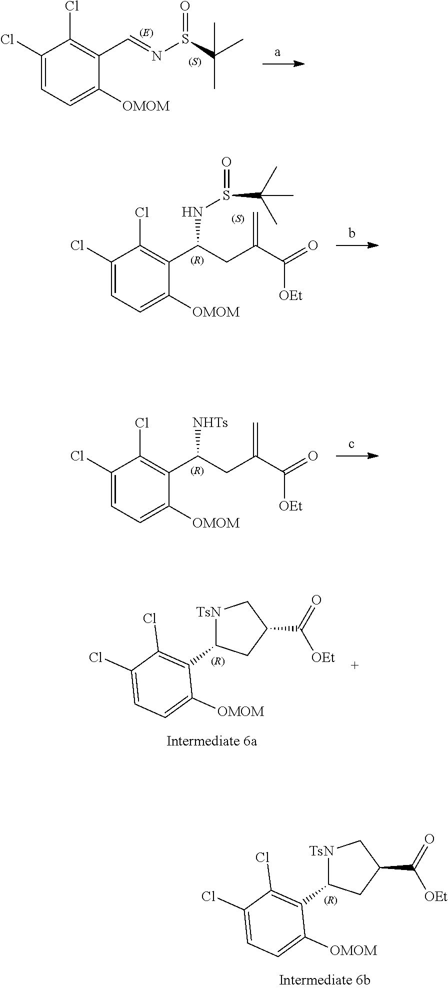

### Step a

To a stirred mixture of (S)—N-[[2,3-dichloro-6-(methoxymethoxy)phenyl]methylidene]-2-methylpropane-2-sulfinamide (1.00 g, 2.96 mmol) and ethyl 2-(bromomethyl)prop-2-enoate (1.71 g, 8.87 mmol) in NH₄Cl (8 mL) and THE (2 mL) was added Zn (0.580 g, 8.87 mmol) in portions at room temperature. The reaction mixture was stirred for 5 minutes, diluted with water (20 mL), and extracted with EA (3×30 mL). The combined organic layers were washed with brine (2×30 mL) and dried over anhydrous Na₂SO₄. After filtration, the filtrate was concentrated under reduced pressure. The residue was purified by reverse phase chromatography, eluting with 45% ACN in water (plus 10 mM NH₄HCO₃), to afford ethyl (4R)-4-[2,3-dichloro-6-(methoxymethoxy)phenyl]-2-methylidene-4-[[(S)-2-methylpropane-2-sulfinyl]amino]butanoate as a light-yellow oil (1.40 g, 94%): LCMS (ESI) calc'd for C₁₉H₂₇Cl₂NO₅S [M+H]⁺: 452, 454 (3:2) found 452, 454 (3:2); ¹H NMR (300 MHz, CD₃OD) δ 7.41-7.36 (m, 1H), 7.19-7.13 (m, 1H), 6.08 (d, J=1.4 Hz, 1H), 5.47 (d, J=9.9 Hz, 1H), 5.38-5.31 (m, 2H), 5.29-5.11 (m, 1H), 4.22-4.09 (m, 2H), 3.56 (s, 3H), 3.20-3.01 (m, 2H), 1.29 (q, J=6.8 Hz, 3H), 1.12 (s, 9H).

### Step b

To a stirred solution of ethyl (4R)-4-[2,3-dichloro-6-(methoxymethoxy)phenyl]-2-methylidene-4-[[(S)-2-methylpropane-2-sulfinyl]amino]butanoate (1.56 g, 3.45 mmol) in MeOH (10.50 mL) was added aq. HCl (2 M, 3.50 mL) at room temperature. The reaction mixture was stirred for 1 h, basified with saturated aq. NaHCO₃ to pH 8, and extracted with EA (3×20 mL). The combined organic layers were washed with brine (2×20 mL) and dried over anhydrous Na₂SO₄. After filtration, the filtrate was concentrated under reduced pressure. To a solution of the residue in DCM (10 mL) were added TsCl (0.660 g, 3.45 mmol), DMAP (0.110 g, 0.86 mmol), and TEA (1.00 mL, 7.18 mmol) at room temperature. The resulting solution was stirred for 2 h, diluted with water (20 mL), and extracted with EA (3×20 mL). The combined organic layers were washed with brine (2×20 mL) and dried over anhydrous Na₂SO₄. After filtration, the filtrate was concentrated under reduced pressure. The residue was purified by silica gel column chromatography, eluting with PE/EA (4/1) to afford ethyl (4R)-4-[2,3-dichloro-6-(methoxymethoxy)phenyl]-4-(4-methylbenzenesulfonamido)-2-methylidenebutanoate as a light-yellow solid (1.10 g, 76%): LCMS (ESI) calc'd for C₂₂H₂₅C₁₂NO₆S [M+Na]⁺: 524, 526 (3:2) found 524, 526 (3:2); ¹H NMR (400 MHz, CDCl₃) δ 7.57-7.51 (m, 2H), 7.14 (d, J=9.0 Hz, 1H), 7.07-7.02 (m, 2H), 6.82 (d, J=9.1 Hz, 1H), 6.22 (d, J=1.2 Hz, 1H), 5.94 (d, J=10.9 Hz, 1H), 5.60 (q, J=1.1 Hz, 1H), 5.30-5.25 (m, 1H), 5.25-5.18 (m, 2H), 4.18 (q, J=7.1 Hz, 2H), 3.57 (s, 3H), 3.00-2.90 (m, 1H), 2.73-2.64 (m, 1H), 2.31 (s, 3H), 1.30 (t, J=7.1 Hz, 3H).

### Step c

To a stirred solution of ethyl (4R)-4-[2,3-dichloro-6-(methoxymethoxy)phenyl]-4-(4-methylbenzenesulfonamido)-2-methylidenebutanoate (0.600 g, 1.19 mmol) in DMF (6 mL) was added NaH (53.0 mg, 0.12 mmol, 60% in oil) at room temperature. The reaction mixture was stirred at 110° C. for 16 h. The resulting mixture was quenched with water (20 mL) at room temperature and extracted with EA (3×20 mL). The combined organic layers were washed with brine (2×20 mL) and dried over anhydrous Na₂SO₄. After filtration, the filtrate was concentrated under reduced pressure. The residue was purified by Prep-TLC (PE/EA 3/1) to afford (ethyl (5R)-5-[2,3-dichloro-6-(methoxymethoxy)phenyl]-1-(4-methylbenzenesulfonyl)pyrrolidine-3-carboxylate isomer 1) as a light-yellow solid (0.150 g, 24%): LCMS (ESI) calc'd for C₂₂H₂₅C₁₂NO₆S [M+H]⁺: 502, 504 (3:2) found 502, 504 (3:2); ¹H NMR (300 MHz, CDCl₃) δ 7.73-7.61 (m, 2H), 7.37-7.28 (m, 3H), 7.04-6.91 (m, 1H), 5.52-5.38 (m, 1H), 5.22-5.02 (m, 2H), 4.16 (q, J=7.1 Hz, 2H), 4.14-4.01 (m, 1H), 3.78 (t, J=11.2 Hz, 1H), 3.59-3.46 (m, 4H), 2.79-2.60 (m, 1H), 2.50-2.38 (m, 4H), 1.26 (t, J=7.1 Hz, 3H) and (ethyl (5R)-5-[2,3-dichloro-6-(methoxymethoxy)phenyl]-1-(4-methylbenzenesulfonyl)pyrrolidine-3-carboxylate isomer 2) as a light-yellow solid (0.29 g, 46%): LCMS (ESI) calc'd for C₂₂H₂₅C12NO₆S [M+H]⁺: 502, 504 (3:2) found 502, 504 (3:2); ¹H NMR (300 MHz, CDCl₃) δ 7.65 (d, J=7.8 Hz, 2H), 7.32 (d, J=8.9 Hz, 1H), 7.25 (d, J=7.8H, 2H), 7.02 (d, J=9.0 Hz, 1H), 5.60-5.47 (m, 1H), 5.27-5.05 (m, 2H), 4.00-3.85 (m, 4H), 3.55 (s, 3H), 3.26-3.15 (m, 1H), 2.63-2.47 (m, 1H), 2.43 (s, 3H), 2.39-2.24 (m, 1H), 1.22 (t, J=7.1 Hz, 3H).

### Example 9. Intermediate 7 (methyl (5R)-5-[2,3-dichloro-6-(methoxymethoxy)phenyl]-1-(4-methylbenzenesulfonyl)pyrrolidine-3-carboxylate)

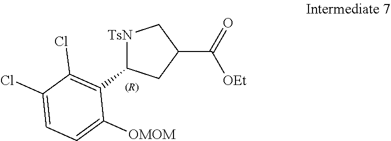

### Intermediate 7

The methyl ester was prepared by the procedure described in Example 8 substituting methyl 2-(bromomethyl)prop-2-enoate. The product was used in the next step without separating the isomers.

### Example 10. Intermediate 8a (1-(tert-butyl) 3-methyl (3S,5R)-5-(2,3-dichloro-6-(methoxymethoxy)phenyl)pyrrolidine-1,3-dicarboxylate) and Intermediate 8b (1-(tert-butyl) 3-methyl (3R,5R)-5-(2,3-dichloro-6-(methoxymethoxy)phenyl)pyrrolidine-1,3-dicarboxylate)

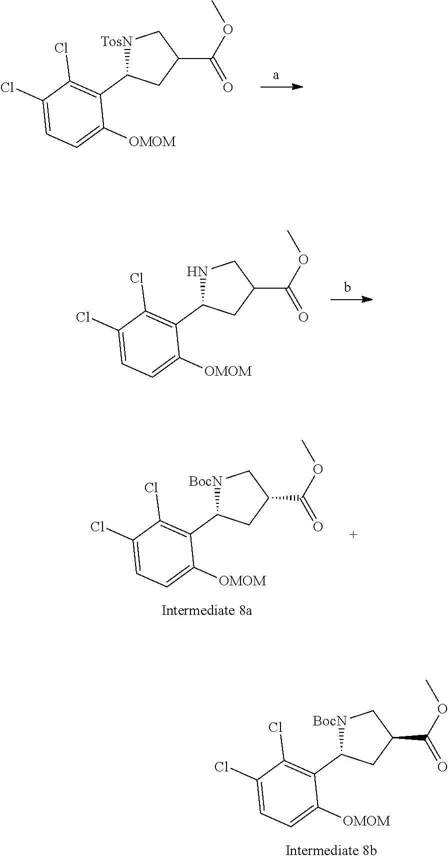

### Step a

To a stirred solution of methyl (5R)-5-[2,3-dichloro-6-(methoxymethoxy)phenyl]-1-(4-methylbenzenesulfonyl)pyrrolidine-3-carboxylate (9.00 g, 18.4 mmol) in MeOH (300 mL) was added Mg (6.72 g, 276 mmol) in portions at room temperature. The reaction mixture was stirred for 2 h, acidified to pH 5 with HCl (1 N, 50 mL), stirred for 10 min, neutralized to pH 7 with saturated aq. NaHCO₃ (50 mL) and extracted with DCM (3×100 mL). The combined organic layers were washed with brine (3×100 mL) and dried over anhydrous MgSO₄. After filtration, the filtrate was concentrated under reduced pressure to afford methyl (5R)-5-[2,3-dichloro-6-(methoxymethoxy)phenyl]pyrrolidine-3-carboxylate as a light yellow oil (6.00 g, 78%): LCMS (ESI) calc'd C₁₄H₁₇C₁₂NO₄ for [M+H]⁺: 334, 336 (3:2) found 334, 336 (3:2).

### Step b

To a stirred solution of methyl (5R)-5-[2,3-dichloro-6-(methoxymethoxy)phenyl]pyrrolidine-3-carboxylate (4.00 g, 11.9 mmol) and TEA (2.42 g, 23.9 mmol) in DCM (50.0 mL) was added Boc₂O (5.22 g, 23.9 mmol) dropwise at 0° C. under nitrogen atmosphere. The reaction mixture was stirred at room temperature for 2 h, diluted with water (100 mL) and extracted with EA (3×80 mL). The combined organic layers were washed with brine (4×80 mL) and dried over anhydrous Na₂SO₄. After filtration, the filtrate was concentrated under reduced pressure. The residue was purified by silica gel column chromatography, eluting with 20% EA in PE to afford the first-eluting component 1-(tert-butyl) 3-methyl (3R,5R)-5-(2,3-dichloro-6-(methoxymethoxy)phenyl)pyrrolidine-1,3-dicarboxylate (Intermediate 8b) as a light yellow oil (1.20 g, 23%): LCMS (ESI) calc'd for C₁₉H₂₅Cl₂NO₆ [M +H]⁺: 434, 436 (3:2) found 434, 436 (3:2); ¹H NMR (400 MHz, CDCl₃) δ 7.33 (d, J=9.0 Hz, 1H), 7.05 (d, J=9.0 Hz, 1H), 5.49 (t, J=9.1 Hz, 1H), 5.30-5.14 (m, 2H), 4.02 (dd, J=10.4, 8.0 Hz, 1H), 3.75 (s, 3H), 3.72-3.64 (m, 1H), 3.48 (s, 3H), 3.23-3.11 (m, 1H), 2.60-2.37 (m, 2H), 1.15 (s, 9H). And the second eluting component 1-(tert-butyl) 3-methyl (3S,5R)-5-(2,3-dichloro-6-(methoxymethoxy)phenyl)pyrrolidine-1,3-dicarboxylate (Intermediate 8a) as a light yellow solid (1.8 g, 35%): LCMS (ESI) calc'd for C₁₉H₂₅C₁₂NO₆ [M+H]⁺: 434, 436 (3:2) found 434, 436 (3:2). The trans isomer 1-(tert-butyl) 3-methyl (3S,5R)-5-(2,3-dichloro-6-(methoxymethoxy)phenyl)pyrrolidine-1,3-dicarboxylate (1.80 g, 4.11 mmol) was re-purified by Prep SFC with the following conditions: Column: CHIRALPAK IF, 3×25 cm, 5 m; Mobile Phase A: CO₂, Mobile Phase B: MeOH (0.1% 2M NH₃-MeOH); Flow rate: 50 mL/min; Gradient: isocratic 15% B; Column Temperature: 35° C.; Back Pressure: 100 bar; Wave Length: 220 nm; Retention Time: 9.98 min; Sample Solvent: MeOH-Preparative; Injection Volume: 0.5 mL. The fraction containing the desired product was collected and concentrated under reduced pressure to afford 1-(tert-butyl) 3-methyl (3S,5R)-5-(2,3-dichloro-6-(methoxymethoxy)phenyl)pyrrolidine-1,3-dicarboxylate as a light yellow oil (1.20 g, 66%): LCMS (ESI) calc'd for C₁₉H₂₅C₁₂NO₆ [M+H]⁺: 434, 436 (3:2) found 434, 436 (3:2). ¹H NMR (400 MHz, CDCl₃) δ 7.32 (d, J=8.9 Hz, 1H), 7.03 (d, J=9.2 Hz, 1H), 5.59 (t, J=8.1 Hz, 1H), 5.28-5.08 (m, 2H), 4.00 (d, J=11.0 Hz, 1H), 3.79-3.75 (s, 4H), 3.49 (s, 3H), 3.26-3.18 (m, 1H), 2.64 (t, J=9.7 Hz, 1H), 2.33-2.20 (m, 1H), 1.15 (s, 9H).

### Example 11. Intermediate 9a (tert-butyl (2R,43)-4-carbamoyl-2-[2,3-dichloro-6-(methoxymethoxy)phenyl]pyrrolidine-1-carboxylate) and Intermediate 9b (tert-butyl (2R,4R)-4-carbamoyl-2-[2,3-dichloro-6-(methoxymethoxy)phenyl]pyrrolidine-1-carboxylate)

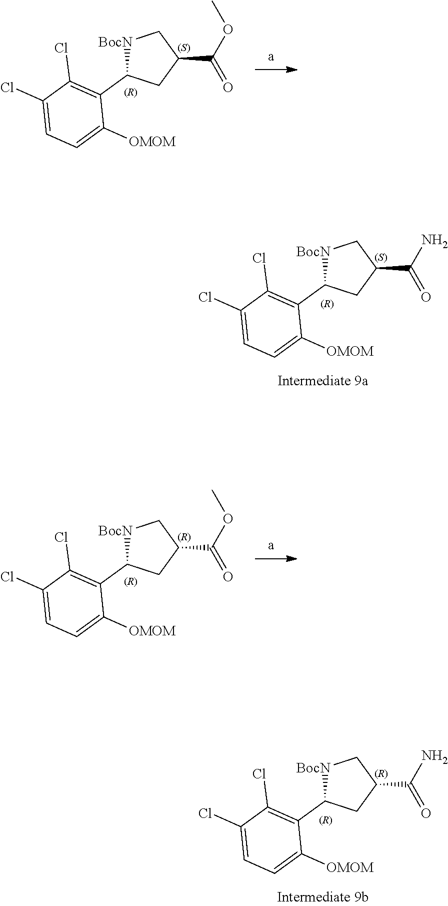

### Step a

To a stirred mixture of 1-tert-butyl 3-methyl (3S,5R)-5-[2,3-dichloro-6-(methoxymethoxy)phenyl]pyrrolidine-1,3-dicarboxylate (0.200 g, 0.460 mmol) in MeOH (2 mL) and H₂O (0.5 mL) was added LiOH H₂O (39.0 mg, 0.920 mmol) at room temperature. The reaction mixture was stirred at room temperature for 1 h and concentrated under reduced pressure. To a stirred mixture of the crude product, NH₄Cl (49.0 mg, 0.920 mmol) and HATU (0.350 g, 0.920 mmol) in DMF (2 mL) was added TEA (93.0 mg, 0.920 mmol) at room temperature. The reaction mixture was stirred at room temperature for 2 h, dissolved with MeOH (0.5 mL) and purified by reverse phase chromatography, eluting with 45% ACN in water (plus 0.05% TFA) to afford tert-butyl (2R,4S)-4-carbamoyl-2-[2,3-dichloro-6-(methoxymethoxy)phenyl]pyrrolidine-1-carboxylate (Intermediate 9a) as a light yellow oil (0.150 g, 77%): LCMS (ESI) calc'd for C₁₈H₂₄Cl₂N₂O₅ [M+H]⁺: 419, 421 (3:2) found 419, 421 (3:2); ¹H NMR (400 MHz, CDCL₃) δ 7.33 (d, J=8.9 Hz, 1H), 7.03 (d, J=9.0 Hz, 1H), 5.83-5.53 (m, 3H), 5.27-5.11 (m, 2H), 4.01-3.72 (m, 2H), 3.49 (s, 3H), 3.17-3.08 (m, 1H), 2.71-2.60 (m, 1H), 2.36-2.25 (m, 1H), 1.16 (s, 9H). The (R,R) diastereomer Intermediate 9b was prepared in the same way using 1-tert-butyl 3-methyl (3R,5R)-5-[2,3-dichloro-6-(methoxymethoxy)phenyl]pyrrolidine-1,3-dicarboxylate (0.200 g, 0.460 mmol). LCMS (ESI) calc'd for C₁₈H₂₄Cl₂N₂O₅ [M+H]⁺: 419, 421 (3:2); ¹H NMR (400 MHz, CDCl₃) δ 7.33 (d, J=8.9 Hz, 1H), 7.07-7.02 (m, 1H), 5.61-5.39 (m, 3H), 5.31-5.18 (m, 2H), 4.01 (t, J=9.2 Hz, 1H), 3.77-3.65 (m, 1H), 3.49 (s, 3H), 3.07-2.94 (m, 1H), 2.58-2.41 (m, 2H), 1.16 (s, 9H).

Examples 12-20 describe the syntheses of representative compounds of Formula I, I′, II, II′, III, or IV disclosed herein.

### Example 12. Compound 31 (2-[(3S,5R)-5-(2,3-dichloro-6-hydroxyphenyl)pyrrolidin-3-yl]acetamide) and Compound 32 (2-[(3S,5R)-5-(2,3-dichloro-6-hydroxyphenyl)pyrrolidin-3-yl]acetamide)

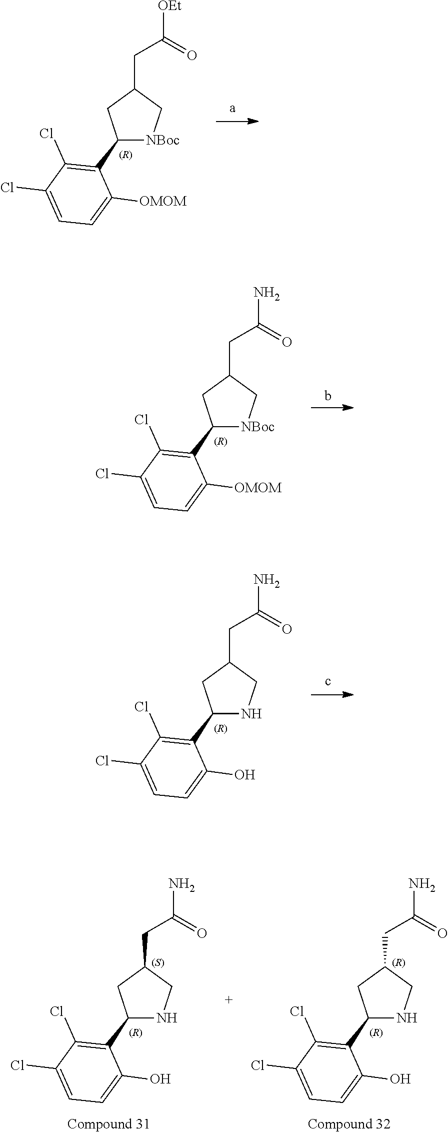

### Step a

To a stirred mixture of tert-butyl (2R)-2-[2,3-dichloro-6-(methoxymethoxy)phenyl]-4-(2-ethoxy-2-oxoethyl)pyrrolidine-1-carboxylate (0.600 g, 1.30 mmol) in MeOH (4 mL) and H₂O (2 mL) was added LiOH H₂O (0.110 g, 2.60 mmol) at room temperature. The reaction mixture was stirred at room temperature for 1 h and concentrated under reduced pressure. Then to the crude product in DMF (5 mL) were added HATU (0.740 g, 1.95 mmol), TEA (0.54 mL, 3.89 mmol) and NH₄Cl (0.140 g, 2.60 mmol) at room temperature. The reaction mixture was stirred at room temperature for 2 h, dissolved in MeOH (1 mL) and purified by reverse phase chromatography, eluting with 40% ACN in water (plus 0.05% TFA) to afford tert-butyl (2R)-4-(carbamoylmethyl)-2-[2,3-dichloro-6-(methoxymethoxy)phenyl]pyrrolidine-1-carboxylate as a light yellow solid (0.240 g, 38%): LCMS (ESI) calc'd for C₁₉H₂₆Cl₂N₂O₅ [M+H]⁺: 433, 435 (3:2) found 433, 435 (3:2); ¹H NMR (300 MHz, CDCl₃) δ 7.35-7.29 (m, 1H), 7.07-6.97 (m, 1H), 5.56-5.33 (m, 2H), 5.29-5.01 (m, 1H), 4.04-3.68 (m, 1H), 3.48 (s, 3H), 3.28-3.09 (m, 1H), 2.93-2.57 (m, 1H), 2.57-2.20 (m, 2H), 2.13-1.78 (m, 2H), 1.15 (s, 9H).

### Step b

To a stirred solution of tert-butyl (2R)-4-(carbamoylmethyl)-2-[2,3-dichloro-6-(methoxymethoxy)phenyl]pyrrolidine-1-carboxylate (0.240 g, 0.55 mmol) in DCM (2 mL) was added BBr₃ (0.5 mL) dropwise at room temperature. The reaction mixture was stirred at room temperature for 2 h, quenched with MeOH (3 mL) at 0° C. and concentrated under reduced pressure. The residue was purified by reverse phase chromatography, eluting with 30% ACN in water (plus 10 mM NH₄HCO₃) to afford 2-[(5R)-5-(2,3-dichloro-6-hydroxyphenyl)pyrrolidin-3-yl]acetamide as an off-white solid (96.0 mg, 59%): LCMS (ESI) calc'd for C₁₂H₁₄Cl₂N₂O₂ [M+H]⁺: 289, 291 (3:2) found 289, 291 (3:2); ¹H NMR (400 MHz, CD₃OD) δ 7.18 (d, J=8.8 Hz, 1H), 6.59 (dd, J=8.9, 1.1 Hz, 1H), 5.01-4.89 (m, 1H), 3.50-3.37 (m, 1H), 2.92-2.83 (m, 1H), 2.76-2.59 (m, 2H), 2.46-2.32 (m, 2H), 1.55-1.43 (m, 1H).

### Step c

2-[(5R)-5-(2,3-dichloro-6-hydroxyphenyl)pyrrolidin-3-yl]acetamide (96.0 mg, 0.330 mmol) was separated by Prep Chiral HPLC with the following conditions: Column: CHIRALPAK IH, 2×25 cm, 5 m; Mobile Phase A: Hex (plus 0.5% 2 MNH₃-MeOH)-HPLC, Mobile Phase B: EtOH-HPLC; Flow rate: 20 mL/min; Gradient: 30% B to 30% B in 25 min; Wavelength: 220/254 nm; Retention time 1: 16.45 min; Retention time 2: 22.00 min; Sample Solvent: EtOH-HPLC; Injection Volume: 1.2 mL; Number Of Runs: 8. The faster-eluting enantiomer at 16.45 min was obtained 2-[(3S,5R)-5-(2,3-dichloro-6-hydroxyphenyl)pyrrolidin-3-yl]acetamide. The product was purified by Prep-HPLC with the following conditions: Column: SunFire Prep C18 OBD Column, 19×150 mm, 5 m 10 nm; Mobile Phase A: water (plus 0.05% TFA), Mobile Phase B: ACN; Flow rate: 25 mL/min; Gradient: 10% B to 30% B in 6.8 min, 30% B; Wavelength: 210 nm; Retention time: 4.30 min. The fractions containing the desired product were collected and concentrated under reduced pressure to afford 2-[(3S,5R)-5-(2,3-dichloro-6-hydroxyphenyl)pyrrolidin-3-yl]acetamide as an off-white solid (Compound 31) (57.2 mg, 42%): LCMS (ESI) calc'd for C₁₂H₁₄Cl₂N₂O₂ [M+H]⁺: 289, 291 (3:2) found 289, 291 (3:2); ¹H NMR (400 MHz, CD₃OD) δ 7.47 (d, J=8.9 Hz, 1H), 6.93 (d, J=8.9 Hz, 1H), 5.30 (dd, J=11.6, 7.0 Hz, 1H), 3.67 (dd, J=11.3, 7.7 Hz, 1H), 3.38 (d, J=10.9 Hz, 1H), 2.90-2.78 (m, 1H), 2.58 (dd, J=15.2, 6.2 Hz, 1H), 2.52-2.42 (m, 2H), 2.21-2.10 (m, 1H). The slower-eluting enantiomer at 22.00 mi was obtained 2-[(3R,5R)-5-(2,3-dichloro-6-hydroxyphenyl)pyrrolidin-3-yl]acetamide. The product was purified by Prep-HPLC with the following conditions: Column: SunFire Prep C18 OBD Column, 19×150 mm, 5)ₘ 10 nm; Mobile Phase A: water (plus 0.050 TFA), Mobile Phase B: ACN; Flow rate: 25 mL/min; Gradient: 10% B to 300% B in 6.8 min, 30% B; Wavelength: 210 nm; Retention time: 4.35 min. The fractions containing the desired product were collected and concentrated under reduced pressure to afford 2-[(3R,5R)-5-(2,3-dichloro-6-hydroxyphenyl)pyrrolidin-3-yl]acetamide (Compound 32) as a purple solid (10.8 mg, 80%): LCMS (ESI) calc'd for C₁₂H₁₄C₁₂N₂O₂ [M+H]⁺: 289, 291 (3:2) found 289, 291 (3:2); ¹H NMVR (400 MHz, CD₃OD) δ 7.47 (d, J=8.9 Hz, 1H), 6.93 (d, J 8.9 Hz, 1H), 5.36 (t, J 9.1 Hz, 1H), 3.84 (dd, J 11.4, 7.0 Hz, 1H), 3.23 (dd, J=11.4, 8.3 Hz, 1H), 3.18-3.02 (m, 1H), 2.61-2.41 (in, 3H), 2.24-2.13 (m, 1H).

The compounds in Table 7A below were prepared in an analogous fashion to that described for Compound 31, starting from tert-butyl (2R)-2-[2,3-dichloro-6-(methoxymethoxy)phenyl]-4-(2-ethoxy-2-oxoethyl)pyrrolidine-1-carboxylate or tert-butyl (2R)-2-[3,4-dichloro-6-(methoxymethoxy)phenyl]-4-(2-ethoxy-2-oxoethyl)pyrrolidine-1-carboxylate and the corresponding amines, which were available from commercial sources.

TABLE 7A

| Compound | | | MS: (M + H)⁺ & ¹H |
|---|---|---|---|
| Number | Structure | Chemical Name | MNR |
| 33 | 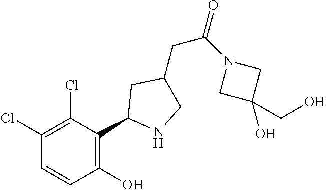 [SMILES: O=C(CC1CN[C@@H](c2c(O)ccc(Cl)c2Cl)C1)N1CC(O)(CO)C1] | 2-((5R)-5-(2,3- dichloro-6- hydroxyphenyl)pyrro- lidin-3-yl)-1-(3- hydroxy-3- (hydroxymethyl)azeti- din-1-yl)ethan-1- one | [M + H]⁺: 375, 377 (3:2); ¹H NMR (400 MHz, CD₃OD) δ 7.19 (d, J = 8.8 Hz, 1H), 6.61 (d, J = 8.8 Hz, 1H), 4.97-4.92 (m, 1H), 4.27-4.17 (m, 1H), 4.07-3.93 (m, 2H), 3.75 (d, J = 10.4 Hz, 1H), 3.58 (d, J = 8.4 Hz, 2H), 3.48- 3.39 (m, 1H), 2.91 (dd, J = 11.1, 8.2 Hz, 1H), 2.76- 2.59 (m, 2H), 2.48-2.31 |
| | | | (m, 2H), 1.57-1.43 (m, |
| | | | 1H). |
| 34 | 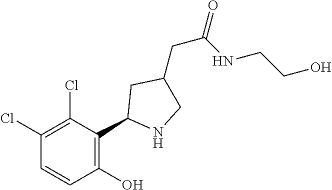 [SMILES: O=C(CC1CN[C@@H](c2c(O)ccc(Cl)c2Cl)C1)NCCO] | 2-((5R)-5-(2,3- dichloro-6- hydroxyphenyl)pyrro- lidin-3-yl)-N-(2- hydroxyethyl)acetamide | [M + H]⁺: 333, 335 (3:2); ¹H NMR (400 MHz, CD₃OD) δ 7.18 (d, J = 8.9 Hz, 1H), 6.59 (d, J = 8.9 Hz, 1H), 4.92 (dd, J = 11.2, 6.4 Hz, 1H), 3.60 (t, J = 5.8 Hz, 2H), 3.41 (dd, J = 11.1, 8.0 Hz, 1H), 3.32-3.29 (m, 2H), 2.89 (dd, J = 11.1, 8.7 Hz, 1H), 2.78-2.58 (m, 2H), 2.45- 2.33 (m, 2H), 1.54-1.42 |
| | | | (m, 1H). |
| 35 | 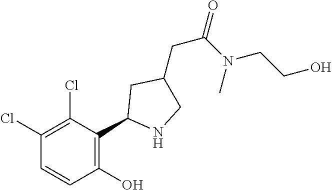 [SMILES: CN(CCO)C(=O)CC1CN[C@@H](c2c(O)ccc(Cl)c2Cl)C1] | 2-((5R)-5-(2,3- dichloro-6- hydroxyphenyl)pyrro- lidin-3-yl)-N-(2- hydroxyethyl)-N- methylacetamide | [M + H]⁺: 347, 349 (3:2); ¹H NMR (400 MHz, CD₃OD) δ 7.47 (d, J = 8.9 Hz, 1H), 6.94 (d, J = 8.9 Hz, 1H), 5.32 (dd, J = 11.6, 7.0 Hz, 1H), 4.44- 4.39 (m, 2H), 3.74 (dd, J = 11.4, 7.6 Hz, 1H), 3.39- 3.35 (m, 3H), 2.97-2.84 (m, 1H), 2.79-2.73 (m, 5H), 2.62-2.47 (m, 1H), 2.26-2.08 (m, 1H). |
| 36 | 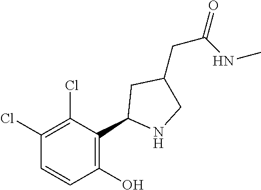 [SMILES: CNC(=O)CC1CN[C@@H](c2c(O)ccc(Cl)c2Cl)C1] | 2-((5R)-5-(2,3- dichloro-6- hydroxyphenyl)pyrro- lidin-3-yl)-N- methylacetamide | [M + H]⁺: 303, 305 (3:2); ¹H NMR (300 MHz, CD₃OD) δ 7.18 (d, J = 8.8 Hz, 1H), 6.59 (d, J = 8.9 Hz, 1H), 4.93 (d, J = 6.7 Hz, 1H), 3.45-3.35 (m, 1H), 2.87 (dd, J = 11.0, 8.7 Hz, 1H), 2.73 (s, 3H), 2.69-2.53 (m, 2H), 2.36 (d, J = 7.2 Hz, 2H), 1.49- 1.43 (m, 1H). |
| 37 | 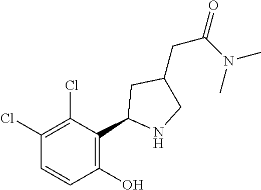 [SMILES: CN(C)C(=O)CC1CN[C@@H](c2c(O)ccc(Cl)c2Cl)C1] | 2-((5R)-5-(2,3- dichloro-6- hdyroxyphenyl)pyrro- lidin-3-yl)-N,N- dimethylacetamide | [M + H ]⁺: 317, 319 (3:2); ¹H NMR (300 MHz, CD₃OD) δ 7.25 (d, J = 8.9 Hz, 1H), 6.68 (d, J = 8.8 Hz, 1H), 5.03 (dd, J = 11.2, 6.7 Hz, 1H), 3.56 (dd, J = 11.0, 7.7 Hz, 1H), 3.07 (s, 3H), 3.05-2.97 (m, 1H), 2.96 (s, 3H), 2.84-2.53 (m, 4H), 1.69- 1.67 (m, 1H). |
| 38 | 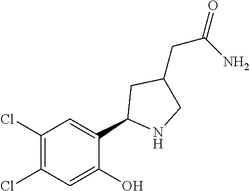 [SMILES: NC(=O)CC1CN[C@@H](c2cc(Cl)c(Cl)cc2O)C1] | 2-((5R)-5-(4,5- dichloro-2- hydroxyphenyl)pyrro- lidin-3- yl)acetamide | [M + H]⁺: 289, 291 (3:2); ¹H NMR (300 MHz, CD₃OD) δ 7.14 (s, 1H), 6.78 (s, 1H), 4.47 (dd, J = 11.1, 6.5 Hz, 1H), 3.40 (dd, J = 11.0, 8.2 Hz, 1H), 2.88 (dd, J = 11.2, 8.7 Hz, 1H), 2.75-2.60 (m, 1H), 2.50-2.30 (m, 3H), 1.66- 1.63 (m, 1H). |
| 39 | 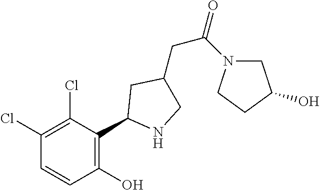 [SMILES: O=C(CC1CN[C@@H](c2c(O)ccc(Cl)c2Cl)C1)N1CC[C@@H](O)C1] | 2-((5R)-5-(2,3- dichloro-6- hydroxyphenyl)pyrro- lidin-3-yl)-1-((R)- 3-hydroxypyrrolidin- 1-yl)ethan-1-one | [M + H]⁺: 359, 361 (3:2); ¹H NMR (400 MHz, CD₃OD) δ 7.17 (d, J = 8.8 Hz, 1H), 6.59 (d, J = 8.9 Hz, 1H), 4.93 (dd, J = 11.0, 6.5 Hz, 1H), 4.49- 4.38 (m, 1H), 3.67-3.41 (m, 4H), 2.94-2.86 (m, 1H), 2.82-2.44 (m, 4H), 2.16-1.88 (m, 3H), 1.58- 1.45 (m, 1H). |
| 40 | 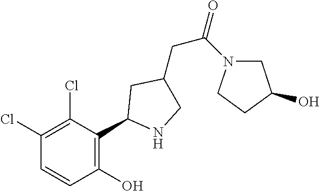 [SMILES: O=C(CC1CN[C@@H](c2c(O)ccc(Cl)c2Cl)C1)N1CC[C@H](O)C1] | 2-((5R)-5-(2,3- dichloro-6- hydroxyphenyl)pyrro- lidin-3-yl)-1-((S)-3- hydroxypyrrolidin-1- yl)ethan-1-one | [M + H]⁺: 359, 361 (3:2); ¹H NMR (400 MHz, CD₃OD) δ 7.17 (d, J = 8.9 Hz, 1H), 6.58 (d, J = 8.8 Hz, 1H), 4.93 (dd, J = 11.0, 6.6 Hz, 1H), 4.49- 4.39 (m, 1H), 3.65-3.43 (m, 5H), 2.96-2.87 (m, 1H), 2.82-2.49 (m, 4H), 2.15-1.89 (m, 2H), 1.57- 1.45 (m, 1H). |

The compounds in Table 7B below were prepared in an analogous fashion to that described for Compound 31, starting from tert-butyl (2R,4S′)-2-[2,3-dichloro-6-(methoxymethoxy)phenyl]-4-(2-ethoxy-2-oxoethyl)pyrrolidine-1l-carboxylate and the corresponding amines, which were available from commercial sources.

TABLE 7B

| Compound | | | |
|---|---|---|---|
| Number | Structure | Chemical Name | MS: (M + H)⁺ & ¹H NMR |
| 41 | 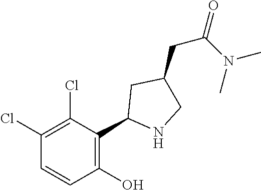 [SMILES: CN(C)C(=O)C[C@H]1CN[C@@H](c2c(O)ccc(Cl)c2Cl)C1] | 2-((3S,5R)-5-(2,3- dichloro-6- hydroxyphenyl)pyrro- lidin-3-yl)-N,N- dimethyalcetamide | [M + H]⁺: 317, 319 (3:2); ¹H NMR (400 MHz, CD₃OD) δ 7.15 (d, J = 8.9 Hz, 1H), 6.56 (d, J = 8.9 Hz, 1H), 4.90 (dd, J = 11.0, 6.4 Hz, 1H), 3.47 (dd, J = 11.1, 7.7 Hz, 1H), 3.04 (s, 3H), 2.93 (s, 3H), 2.86 (dd, J = 11.1, 8.1 Hz, 1H), 2.76- 2.49 (m, 4H), 1.54-1.43 (m, 1H). |
| 42 | 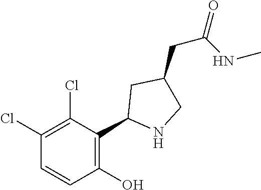 [SMILES: CNC(=O)C[C@H]1CN[C@@H](c2c(O)ccc(Cl)c2Cl)C1] | 2-((3S,5R)-5-(2,3- dichloro-6- hydroxyphenyl)pyrro- lidin-3-yl)-N- methylacetamide | [M + H]⁺: 303, 305 (3:2); ¹H NMR (400 MHz, CD₃OD) δ 7.18 (d, J = 8.8 Hz, 1H), 6.59 (d, J = 8.9 Hz, 1H), 4.95-4.90 (m, 1H), 3.40 (dd, J = 11.0, 8.0 Hz, 1H), 2.87 (dd, J = 11.1, 8.7 Hz, 1H), 2.77-2.65 (m, 4H), 2.65-2.56 (m, 1H), 2.41- 2.29 (m, 1H), 1.51-1.40 (m, 1H). |
| 43 | 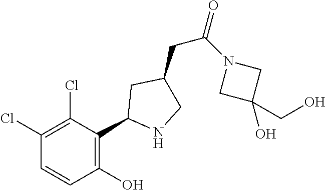 [SMILES: O=C(C[C@H]1CN[C@@H](c2c(O)ccc(Cl)c2Cl)C1)N1CC(O)(CO)C1] | 2-((3S,5R)-5-(2,3- dichloro-6- hydroxyphenyl)pyrro- lidin-3-yl)-1-(3- hydroxy-3- (hydroxymethyl)aze- tidin-1-yl)ethan-1- one | [M + H]⁺: 375, 3.77 (3:2); ¹H NMR (400 MHz, DMSO-d₆) δ 7.25 (d, J = 8.8 Hz, 1H), 6.58 (d, J = 8.9 Hz, 1H), 5.66 (s, 1H), 5.02- 4.90 (m, 1H), 4.74-4.62 (m, 1H), 4.04 (dd, J = 12.2, 8.6 Hz, 1H), 3.87-3.71 (m, 2H), 3.52 (d, J = 9.7 Hz, 1H), 3.38-3.34 (m, 2H), 3.27- 3.19 (m, 1H), 2.64-2.55 (m, 2H), 2.54-2.53 (m, 2H), |
| | | | 2.46-2.38 (m, 1H), 2.25- |
| | | | 2.12 (m, 2H), 1.22 (q, J = |
| | | | 10.8 Hz, 1H). |
| 56 | 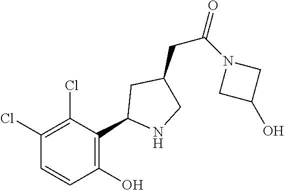 [SMILES: O=C(C[C@H]1CN[C@@H](c2c(O)ccc(Cl)c2Cl)C1)N1CC(O)C1] | 2-((3S,5R)-5-(2,3- dichloro-6- hydroxyphenyl)pyrro- lidin-3-yl)-1-(3- hydroxyazetidin-1- yl)ethan-1-one | [M + H]⁺: 345, 347 (3:2); ¹H NMR (400 MHz, DMSO-d₆ + D₂O) δ 7.30 (d, J = 8.80 Hz, 1H), 6.65 (d, J = 8.84 Hz, 1H), 5.81-5.64 (m, 1H), 4.76 (dd, J = 10.64, 6.07 Hz, 1H), 4.50- 4.36 (m, 1H), 4.31-4.16 (m, 1H), 4.01 (dd, J = 9.84, 7.22 Hz, 1H), 3.85-3.73 (m, 1H), 3.55 (dd, J = 10.06, 4.40 Hz, 1H), 3.31-3.22 (m, 1H), |
| | | | 2.72-2.58 (m, 1H), 2.48- |
| | | | 2.41 (m, 1H), 2.29-2.15 (m, |
| | | | 2H), 1.40-1.24 (m, 1H). |
| 57 | 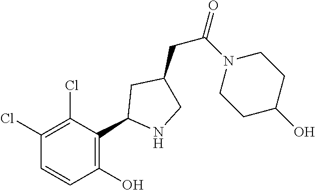 [SMILES: O=C(C[C@H]1CN[C@@H](c2c(O)ccc(Cl)c2Cl)C1)N1CCC(O)CC1] | 2-((3S,5R)-5-(2,3- dichloro-6- hydroxyphenyl)pyrro- lidin-3-yl)-1-(4- hydroxypiperidin- 1-yl)ethan-1-one | [M + H]⁺: 373, 375 (3:2); ¹H NMR (400 MHz, DMSO-d₆ + D₂O) δ 7.24 (d, J = 8.81 Hz, 1H), 6.57 (d, J = 8.82 Hz, 1H), 4.77-4.64 (m, 2H), 3.97-3.84 (m, 1H), 3.72-3.60 (m, 2H), 3.30- 3.22 (m, 1H), 3.17-3.06 (m, 1H), 3.03-2.90 (m, 1H), 2.63-2.52 (m, 2H), 2.48- 2.37 (m, 2H), 1.77-1.62 (m, 2H), 1.36-1.14 (m, 3H). |
| 58 | 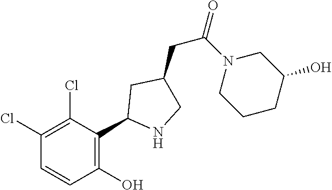 [SMILES: O=C(C[C@H]1CN[C@@H](c2c(O)ccc(Cl)c2Cl)C1)N1CCC[C@@H](O)C1] | 2-((3S,5R)-5-(2,3- dichloro-6- hydroxyphenyl)pyrro- lidin-3-yl)-1-((R)- 3- hydroxypipeiridn- 1-yl)ethan-1-one | [M + H]⁺: 373, 375 (3:2); ¹H NMR (300 MHz, DMSO-d₆ + D₂O) δ 7.25 (d, J = 8.84 Hz, 1H), 6.57 (d, J = 8.81 Hz, 1H), 4.94-4.76 (m, 1H), 4.70 (dd, J = 10.75, 6.18 Hz, 1H), 4.18- 3.47 (m, 3H), 3.32-3.22 (m, 1H), 3.22-2.89 (m, 2H), 2.64-2.53 (m, 2H), 2.48- 2.34 (m, 2H), 1.91-1.71 (m, 1H), 1.71-1.56 (m, 1H), |
| | | | 1.49-1.13 (m, 3H). |
| 59 | 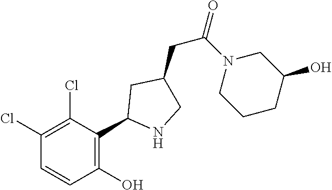 [SMILES: O=C(C[C@H]1CN[C@@H](c2c(O)ccc(Cl)c2Cl)C1)N1CCC[C@H](O)C1] | 2-((3S,5R)-5-(2,3- dichloro-6- hydroxyphenyl)pyrro- lidin-3-yl)-1-((S)- 3- hydroxypiperidin- 1-yl)ethan-1-one | [M + H]⁺: 373, 375 (3:2); 1H NMR (300 MHz, DMSO-d₆ + D₂O) δ 7.25 (d, J = 8.84 Hz, 1H), 6.57 (d, J = 8.81 Hz, 1H), 4.95-4.78 (m, 1H), 4.70 (dd, J = 10.77, 6.19 Hz, 1H), 4.16- 3.45 (m, 3H), 3.41-3.31 (m, 1H), 3.28-2.90 (m, 2H), 2.64-2.53 (m, 2H), 2.50- 2.39 (m, 2H), 1.90-1.72 (m, 1H), 1.72-1.57 (m, 1H), |
| | | | 1.48-1.14 (m, 3H). |
| 60 | 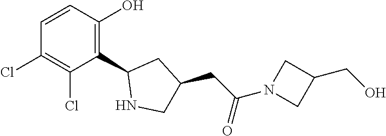 [SMILES: O=C(C[C@H]1CN[C@@H](c2c(O)ccc(Cl)c2Cl)C1)N1CC(CO)C1] | 2-((3S,5R)-5-(2,3- dichloro-6- hydroxyphenyl)pyrro- lidin-3-yl)-1-(3- (hydroxymethyl)aze- tidin-1-yl)ethan-1-one | [M + H]⁺: 359, 361 (3:2); ¹H NMR (400 MHz, CD₃OD) δ 7.18 (d, J = 8.81 Hz, 1H), 6.59 (d, J = 8.82 Hz, 1H), 4.95-4.91 (m, 1H), 4.28-4.19 (m, 1H), 4.06- 3.90 (m, 2H), 3.79-3.72 (m, 1H), 3.68 (dd, J = 8.55, 6.08 Hz, 2H), 3.47-3.39 (m, 1H), |
| | | | 2.92-2.85 (m, 1H), 2.82- |
| | | | 2.60 (m, 3H), 2.43-2.26 (m, |
| | | | 2H), 1.59-1.38 (m, 1H). |
| 61 | 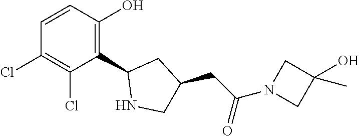 [SMILES: CC1(O)CN(C(=O)C[C@H]2CN[C@@H](c3c(O)ccc(Cl)c3Cl)C2)C1] | 2-((3S,5R)-5-(2,3- dichloro-6- hydroxyphenyl)pyrro- lidin-3-yl)-1-(3- hydroxy-3- methylazetidin-1- yl)ethan-1-one | [M + H]⁺: 359, 361 (3:2); ¹H NMR (400 MHz, CD₃OD) δ 7.19 (d, J = 8.90 Hz, 1H), 6.60 (d, J = 8.82 Hz, 1H), 4.96-4.91 (m, 1H), 4.12-4.00 (m, 2H), 3.90- 3.80 (m, 2H), 3.48-3.41 (m, 1H), 2.94-2.86 (m, 1H), 2.76-2.60 (m, 2H), 2.46- |
| | | | 2.29 (m, 2H), 1.54-1.44 (m, |
| | | | 4H). |
| 62 | 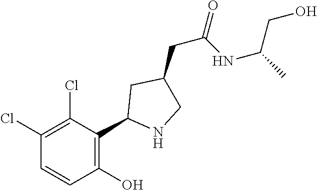 [SMILES: C[C@@H](CO)NC(=O)C[C@H]1CN[C@@H](c2c(O)ccc(Cl)c2Cl)C1] | 2-((3S,5R)-5-(2,3- dichloro-6- hydroxyphenyl)pyrro- lidin-3-yl)-N-((S)- 1-hydroxypropan- 2-yl)acetamide | [M + H]⁺; 347, 349 (3:2); ¹H NMR (300 MHz, CD₃OD) δ 7.18 (d, J = 8.88 Hz, 1H), 6.59 (d, J = 8.89 Hz, 1H), 4.97-4.90 (m, 1H), 4.01-3.89 (m, 1H), 3.48 (d, J = 5.67 Hz, 2H), 3.45-3.37 (m, 1H), 2.91 (dd, J = 11.09, 8.65 Hz, 1H), 2.77- 2.55 (m, 2H), 2.41-2.33 (m, 2H), 1.50 (q, J = 11.27 Hz, 1H), 1.13 (d, J = 6.77 Hz, |
| | | | 3H). |
| 63 | 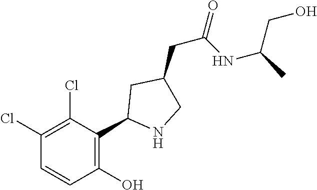 [SMILES: C[C@H](CO)NC(=O)C[C@H]1CN[C@@H](c2c(O)ccc(Cl)c2Cl)C1] | 2-((3S,5R)-5-(2,3- dichloro-6- hydroxyphenyl)pyrro- lidin-3-yl)-N-((R)- 1-hydroxypropan- 2-yl)acetamide | [M + H]⁺: 347, 349 (3:2); ¹H NMR (300 MHz, CD₃OD) δ 7.18 (d, J = 8.87 Hz, 1H), 6.59 (d, J = 8.85 Hz, 1H), 4.96-4.90 (m, 1H), 3.96 (q, J = 6.27 Hz, 1H), 3.52-3.37 (m, 3H), 2.89 (dd, J = 11.03, 8.63 Hz, 1H), 2.78-2.57 (m, 2H), 2.44- 2.30 (m, 2H), 1.49 (q, J = 11.81, 11.38 Hz, 1H), 1.14 (d, J = 6.79 Hz, 3H). |
| 64 | 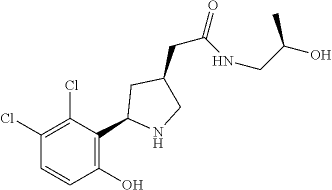 [SMILES: C[C@@H](O)CNC(=O)C[C@H]1CN[C@@H](c2c(O)ccc(Cl)c2Cl)C1] | 2-((3S,5R)-5-(2,3- dichloro-6- hydroxyphenyl)pyrro- lidiin-3-yl)-N-((R)- 2- hydroxypropyl)aceta- mide | [M + H]⁺: 347, 349 (3:2); ¹H NMR (400 MHz, CD₃OD) δ 7.47 (d, J = 8.85 Hz, 1H), 6.93 (d, J = 8.90 Hz, 1H), 5.30 (dd, J = 11.60, 6.98 Hz, 1H), 3.89- 3.77 (m, 1H), 3.66 (dd, J = 11.2,5 7.66 Hz, 1H), 3.38 (t, J = 11.02 Hz, 1H), 3.26 (dd, J = 13.58, 4.56 Hz, 1H), 3.14 (dd, J = 13.58, 7.03 Hz, 1H), 2.90-2.77 (m, 1H), |
| | | | 2.61-2.40 (m, 3H), 2.16 (q, |
| | | | J = 11.86 Hz, 1H), 1.17 (d, |
| | | | J = 6.28 Hz, 3H). |
| 65 | 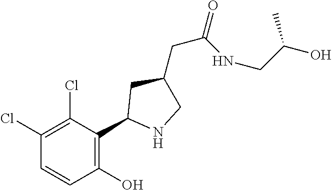 [SMILES: C[C@H](O)CNC(=O)C[C@H]1CN[C@@H](c2c(O)ccc(Cl)c2Cl)C1] | 2-((3S,5R)-5-(2,3- dichloro-6- hydroxyphenyl)pyrro- lidin-3-yl)-N-((S)- 2- hydroxypropyl)aceta- mide | [M + H]⁺: 347, 349 (3:2); ¹H NMR (400 MHz, CD₃OD) δ 7.47 (d, J = 8.85 Hz, 1H), 6.93 (d, J = 8.89 Hz, 1H), 5.30 (dd, J = 11.59, 6.96 Hz, 1H), 3.90- 3.78 (m, 1H), 3.66 (dd, J = 11.29, 7.65 Hz, 1H), 3.38 (t, J = 10.98 Hz, 1H), 3.26 (dd, J = 13.57, 4.53 Hz, 1H), 3.13 (dd, J = 13.57, 7.09 Hz, 1H), 2.91-2.78 (m, 1H), |
| | | | 2.61-2.40 (m, 3H), 2.16 (q, |
| | | | J = 11.88 Hz, 1H), 1.17 (d, |
| | | | J = 6.33 Hz, 3H). |
| 66 | 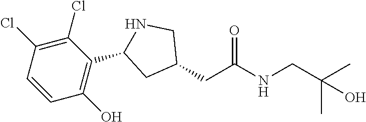 [SMILES: CC(C)(O)CNC(=O)C[C@H]1CN[C@@H](c2c(O)ccc(Cl)c2Cl)C1] | 2-((3S,5R)-5-(2,3- dichloro-6- hydroxyphenyl)pyrro- lidin-3-yl)-N-(2- hydroxy-2- methylpropyl)aceta- mide | [M + H]⁺: 361, 363 (3:2); ¹H NMR (300 MHz, CD₃OD) δ 7.18 (d, J = 8.85 Hz, 1H), 6.60 (d, J = 8.88 Hz, 1H), 4.97-4.91 (m, 1H), 3.42 (dd, J = 10.97, 7.95 Hz, 1H), 3.21 (s, 2H), 2.91 (dd, J = 11.04, 8.60 Hz, |
| | | | 1H), 2.81-2.56 (m, 2H), |
| | | | 2.50-2.36 (m, 2H), 1.58- |
| | | | 1.44 (m, 1H), 1.18 (s, 6H). |
| 67 | 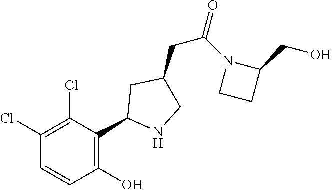 [SMILES: O=C(C[C@H]1CN[C@@H](c2c(O)ccc(Cl)c2Cl)C1)N1CC[C@@H]1CO] | 2-((3S,5R)-5-(2,3- dichloro-6- hydroxyphenyl)pyrro- lidin-3-yl)-1-((R)- 2- (hydroxymethyl)aze- tidin-1-yl)ethan-1- one | [M + H]⁺: 359, 361 (3:2); ¹H NMR (300 MHz, CD₃OD) δ 7.47 (d, J = 8.88 Hz, 1H), 6.93 (d, J = 8.90 Hz, 1H), 5.28 (dd, J = 11.61, 6.98 Hz, 1H), 4.62- 4.43 (m, 1H), 4.10 (t, J = 7.89 Hz, 1H), 3.98-3.81 (m, 2H), 3.79-3.59 (m, 2H), 3.43-3.34 (m, 1H), 2.92- 2.70 (m, 1H), 2.68-2.27 (m, 4H), 2.27-2.03 (m, 2H). |
| 68 | 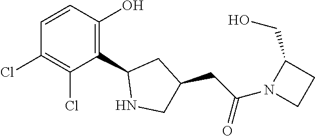 [SMILES: O=C(C[C@H]1CN[C@@H](c2c(O)ccc(Cl)c2Cl)C1)N1CC[C@H]1CO] | 2-((3S,5R)-5-(2,3- dichloro-6- hydroxyphenyl)pyrro- lidin-3-yl)-1-((S)- 2- (hydroxymethyl)aze- tidin-1-yl)ethan-1- one | [M + H]⁺: 359, 361 (3:2); ¹H NMR (400 MHz, CD₃OD) δ 7.19 (d, J = 8.89 Hz, 1H), 6.60 (d, J = 8.88 Hz, 1H), 4.99-4.91 (m, 1H), 4.61-4.41 (m, 1H), 4.08 (t, J = 7.74 Hz, 1H), 3.95-3.76 (m, 2H), 3.74-3.62 (m, 1H), 3.45 (dd, J = 11.08, 7.97 |
| | | | Hz, 1H), 2.98-2.88 (m, 1H), |
| | | | 2.80-2.60 (m, 2H), 2.58- |
| | | | 2.25 (m, 3H), 2.25-2.02 (m, |
| | | | 1H), 1.61-1.44 (m, 1H). |
| 69 | 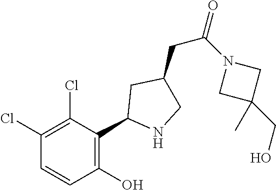 [SMILES: CC1(CO)CN(C(=O)C[C@H]2CN[C@@H](c3c(O)ccc(Cl)c3Cl)C2)C1] | 2-((3S,5R)-5-(2,3- dichloro-6- hydroxyphenyl)pyrro- lidin-3-yl)-1-(3- (hydroxymethyl)-3- methylazetidin-1- yl)ethan-1-one | [M + H]⁺: 373, 375 (3:2); ¹H NMR (300 MHz, CD₃OD) δ 7.47 (d, J = 8.88 Hz, 1H), 6.93 (d, J = 8.89 Hz, 1H), 5.28 (dd, J = 11.58, 6.98 Hz, 1H), 4.07 (d, J = 8.52 Hz, 1H), 3.84 (dd, J = 18.20, 9.15 Hz, 2H), 3.69 (dd, J = 11.35, 7.71 Hz, 1H), 3.58 (d, J = 9.74 Hz, 1H), 3.53 (d, J = 1.58 Hz, 2H), 3.41-3.33 (m, |
| | | | 1H), 2.94-2.74 (m, 1H), |
| | | | 2.60-2.32 (m, 3H), 2.13 (q, |
| | | | J = 11.78 Hz, 1H), 1.29 (s, |
| | | | 3H). |
| 70 | 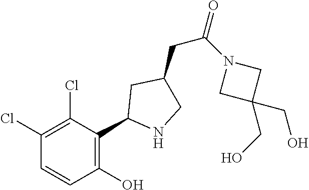 [SMILES: O=C(C[C@H]1CN[C@@H](c2c(O)ccc(Cl)c2Cl)C1)N1CC(CO)(CO)C1] | 1-(3,3- bis(hydroxymethyl) azetidin-1-yl)-2- ((3S,5R)-5-(2,3- dichloro-6- hydroxyphenyl)pyrro- lidin-3-yl)ethan-1- one | [M + H]⁺: 389, 391 (3:2); ¹H NMR (300 MHz, CD₃OD) δ 7.47 (d, J = 8.89 Hz, 1H), 6.93 (d, J = 8.88 Hz, 1H), 5.28 (dd, J = 11.54, 7.00 Hz, 1H), 3.98 (s, 2H), 3.77 (s, 2H), 3.75-3.62 (m, 5H), 3.37 (d, J = 10.68 Hz, 1H), 2.92-2.75 (m, 1H), 2.60-2.35 (m, 3H), 2.13 (q, J = 11.75 Hz, 1H). |
| 71 | 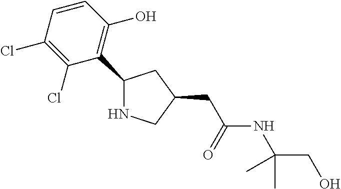 [SMILES: CC(C)(CO)NC(=O)C[C@H]1CN[C@@H](c2c(O)ccc(Cl)c2Cl)C1] | 2-((3S,5R)-5-(2,3- dichloro-6- hydroxyphenyl)pyrro- lidin-3-yl)-N-(1- hydroxy-2- methylpropan-2- yl)acetamide | [M + H ]⁺: 361, 363 (3:2); ¹H NMR (400 MHz, DMSO-d₆) δ 7.32 (s, 1H), 7.25 (d, J = 8.82 Hz, 1H), 6.58 (d, J = 8.84 Hz, 1H), 4.90-4.76 (m, 1H), 4.69 (dd, J = 10.89, 5.93 Hz, 1H), 3.37 (s, 2H), 3.25-3.13 (m, 1H), 2.60 (t, J = 9.68 Hz, 1H), 2.45 (dd, J = 15.43, 7.01 Hz, 1H), 2.22-2.12 (m, 2H), 1.29-1.18 (m, 1H), |
| | | | 1.16 (s, 6H). |

### Example 13. Compound 44 (2-[(3S,5R)-5-(2,3-dichloro-6-hydroxyphenyl)pyrrolidin-3-yl]propanamide isomer 1) and Compound 45 (2-[(3S,5R)-5-(2,3-dichloro-6-hydroxyphenyl)pyrrolidin-3-yl]propanamide isomer 2)

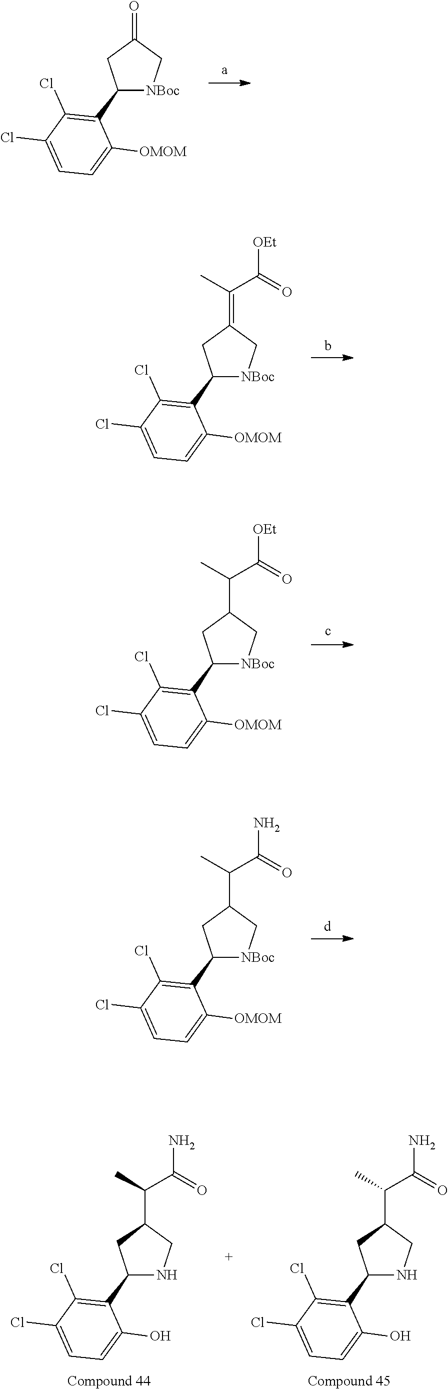

### Step a

A mixture of tert-butyl (2R)-2-[2,3-dichloro-6-(methoxymethoxy)phenyl]-4-oxopyrrolidine-1-carboxylate (0.300 g, 0.770 mmol) and ethyl 2-(triphenylphosphanylidene)propanoate (0.420 g, 1.15 mmol) in toluene (3 mL) was stirred at 110° C. for 24 h. After cooling to room temperature, the resulting mixture was concentrated under reduced pressure. The residue was purified by silica gel column chromatography, eluting with PE/EA (3/1) to afford tert-butyl (2R,4Z)-2-[2,3-dichloro-6-(methoxymethoxy)phenyl]-4-(1-ethoxy-1-oxopropan-2-ylidene)pyrrolidine-1-carboxylate as a light yellow oil (0.270 g, 67%): LCMS (ESI) calc'd for C₂₂H₂₉Cl₂NO₆ [M+H]⁺: 474, 476 (3:2) found 474, 476 (3:2); ¹H NMR (300 MHz, CDCl₃) δ 7.32 (dd, J=9.1, 3.3 Hz, 1H), 7.00 (dd, J=8.9, 4.9 Hz, 1H), 5.84-5.62 (m, 1H), 5.18-5.02 (m, 2H), 4.63 (s, 1H), 4.38-4.14 (m, 3H), 3.74-2.66 (m, 5H), 1.89 (d, J=21.7, 1.9 Hz, 3H), 1.40-1.26 (m, 3H), 1.21 (s, 9H).

### Step b

To a stirred mixture of tert-butyl (2R,4Z)-2-[2,3-dichloro-6-(methoxymethoxy)phenyl]-4-(1-ethoxy-1-oxopropan-2-ylidene)pyrrolidine-1-carboxylate (0.220 g, 0.460 mmol) in MeOH (3 mL) and aq. HCl (6M, 0.3 mL) was added PtO₂ (41.0 mg, 0.180 mmol) at room temperature. The reaction mixture was degassed under reduced pressure, purged with hydrogen three times and stirred under hydrogen atmosphere (1.5 atm) for 6 h. The resulting mixture was filtered and the filter cake was washed with MeOH (2×5 mL). The filtrate was concentrated under reduced pressure. The residue was purified by Prep-TLC (PE/EA 2/1) to afford tert-butyl (2R)-2-[2,3-dichloro-6-(methoxymethoxy)phenyl]-4-(1-ethoxy-1-oxopropan-2-yl)pyrrolidine-1-carboxylate as a light yellow oil (0.170 g, 69%): LCMS (ESI) calc'd for C₂₂H₃₁C₁₂NO₆ [M+H−56]⁺: 420, 422 (3:2) found 420, 422 (3:2); ¹H NMR (300 MHz, CDCl₃) δ 7.33-7.29 (m, 1H), 7.06-6.98 (m, 1H), 5.54-5.38 (m, 1H), 5.31-5.08 (m, 2H), 4.23-4.07 (m, 2H), 3.98-3.80 (m, 1H), 3.53-3.44 (m, 3H), 3.31-3.05 (m, 1H), 2.50-2.34 (m, 3H), 2.00-1.84 (m, 1H), 1.31-1.22 (m, 6H), 1.14 (d, J=1.8 Hz, 9H).

### Step c

To a stirred mixture of tert-butyl (2R)-2-[2,3-dichloro-6-(methoxymethoxy)phenyl]-4-(1-ethoxy-1-oxopropan-2-yl)pyrrolidine-1-carboxylate (0.170 g, 0.360 mmol) in MeOH (2 mL) and H₂O (1 mL) was added LiOH H₂O (30.0 mg, 0.710 mmol) at room temperature. The reaction mixture was stirred for 1 h and concentrated under reduced pressure. To the crude product in DMF (2 mL) were added HATU (0.200 g, 0.530 mmol), TEA (72.0 mg, 0.710 mmol) and NH₄Cl (38.0 mg, 0.710 mmol) at room temperature. The reaction mixture was stirred for 2 h and purified by reverse phase chromatography, eluting with 40% ACN in water (plus 0.05% TFA) to afford tert-butyl (2R)-4-(1-carbamoylethyl)-2-[2,3-dichloro-6-(methoxymethoxy)phenyl]pyrrolidine-1-carboxylate as a light yellow solid (0.130 g, 73%): LCMS (ESI) calc'd for C₂₀H₂₈Cl₂N₂O₅ [M+H]⁺: 447, 449 (3:2) found 447, 449 (3:2); ¹H NMR (300 MHz, CDCl₃) δ 7.35-7.29 (m, 1H), 7.06-6.98 (m, 1H), 5.49-5.40 (m, 1H), 5.27-5.01 (m, 2H), 3.98-3.87 (m, 1H), 3.49 (s, 3H), 3.33-3.09 (m, 1H), 2.49-2.36 (m, 2H), 2.31-2.19 (m, 2H), 1.32-1.25 (m, 3H), 1.13 (d, J=11.3 Hz, 9H).

### Step d

To a stirred mixture of tert-butyl (2R)-4-(1-carbamoylethyl)-2-[2,3-dichloro-6-(methoxymethoxy)phenyl]pyrrolidine-1-carboxylate (0.130 g, 0.290 mmol) in DCM (2 mL) was added BBr₃ (0.5 mL) at room temperature. The reaction mixture was stirred at room temperature for 2 h, quenched with MeOH (3 mL), basified to pH 8 with saturated aq. NaHCO₃ and extracted with EA (3×10 mL). The combined organic layers were washed with brine (2×10 mL) and dried over anhydrous Na₂SO₄. After filtration, the filtrate was concentrated under reduced pressure. The residue was purified by Prep-HPLC with the following conditions: Column: SunFire Prep C18 OBD Column, 19×150 mm, 5 m 10 nm; Mobile Phase A: water (plus 0.05% TFA), Mobile Phase B: ACN; Flow rate: 25 mL/min; Gradient: 10% B to 30% B in 6.8 min; Wavelength: 210 nm; Retention Time 1: 5.67 min, Retention Time 2: 6.80 min. The fractions containing the desired product at 5.67 min were collected and concentrated under reduced pressure to afford 2-[(3S,5R)-5-(2,3-dichloro-6-hydroxyphenyl)pyrrolidin-3-yl]propanamide Isomer 1 as an off-white solid (22.8 mg, 18%): LCMS (ESI) calc'd for C₁₃H₁₆Cl₂N₂O₂ [M+H]⁺: 303, 305 (3:2) found 303, 305 (3:2); ¹H NMR (400 MHz, CD₃OD) δ 7.47 (d, J=8.8 Hz, 1H), 6.93 (d, J=8.9 Hz, 1H), 5.30 (dd, J=11.6, 7.0 Hz, 1H), 3.62 (dd, J=11.3, 7.7 Hz, 1H), 3.40 (t, J=11.2 Hz, 1H), 2.74-2.61 (m, 1H), 2.58-2.48 (m, 1H), 2.39-2.30 (m, 1H), 2.22 (q, J=11.9 Hz, 1H), 1.26 (d, J=6.9 Hz, 3H). The fractions containing the desired product at 6.80 min were collected and concentrated under reduced pressure to afford 2-[(3S,5R)-5-(2,3-dichloro-6-hydroxyphenyl)pyrrolidin-3-yl]propanamide Isomer 2 as an off-white solid (5.60 mg, 4.6%): LCMS (ESI) calc'd for C₁₃H₁₆C₁₂N₂O₂ [M+H]⁺: 303, 305 (3:2) found 303, 305 (3:2); ¹H NMR (400 MHz, CD₃OD) δ 7.47 (d, J=8.9 Hz, 1H), 6.93 (d, J=8.8 Hz, 1H), 5.31 (dd, J=11.7, 6.9 Hz, 1H), 3.54 (dd, J=11.3, 7.8 Hz, 1H), 3.41 (t, J=10.9 Hz, 1H), 2.74-2.61 (m, 1H), 2.54-2.42 (m, 2H), 2.16 (q, J=11.9 Hz, 1H), 1.27 (d, J=7.0 Hz, 3H).

The compounds in Table 7C below were prepared in an analogous fashion to that described for Compound 45, starting from tert-butyl (2R)-2-[2,3-dichloro-6-(methoxymethoxy)phenyl]-4-oxopyrrolidine-1-carboxylate.

TABLE 7C

| Compound | | | |
|---|---|---|---|
| Number | Structure | Chemical Name | MS: (M + H)⁺ & ¹H NMR |
| 46 | 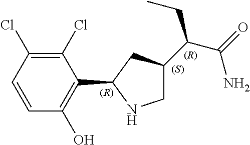 | 2-((3S,5R)-5-(2,3- dichloro-6- hydroxyphenyl)pyrro- lidin-3- yl)butanamide Isomer 1 | [M + H]⁺: 317, 319 (3:2); ¹H NMR (400 MHz, CD₃OD) δ 7.47 (d, J = 8.9 Hz, 1H), 6.93 (d, J = 8.9 Hz, 1H), 5.28 (dd, J = 11.4, 7.2 Hz, 1H), 3.63 (dd, J = 11.2, 7.6 Hz, 1H), 3.38 (t, J = 11.4 Hz, 1H), 2.74-2.58 (m, 1H), 2.37-2.20 (m, 3H), 1.75- 1.53 (m, 2H), 0.99 (t, J = 7.4 |
| | | | Hz, 3H). |
| 47 | 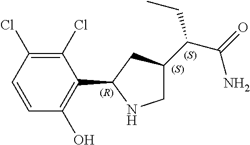 | 2-((3S,5R)-5-(2,3- dichloro-6- hydroxyphenyl)pyrro- lidiin-3- yl)butanamide Isomer 2 | [M + H]⁺: 317, 319 (3:2); ¹H NMR (400 MHz, CD₃OD) δ 7.47 (d, J = 8.9 Hz, 1H), 6.93 (d, J = 8.9 Hz, 1H), 5.31 (dd, J = 11.6, 6.9 Hz, 1H), 3.51-3.45 (m, 2H), 2.75-2.63 (m, 1H), 2.51-2.41 (m, 1H), 2.37-2.28 (m, 1H), 2.17 (q, J = 12.0 Hz, 1H), 1.76-1.62 (m, 2H), 0.98 |
| | | | (t, J = 7.4 Hz, 3H). |
| 72 | 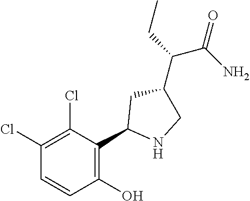 [SMILES: CC[C@H](C(N)=O)[C@@H]1CN[C@@H](c2c(O)ccc(Cl)c2Cl)C1] | 2-((3R,5R)-5-(2,3- dichloro-6- hydroxyphenyl)pyrro- lidin-3- yl)butanamide isomer 1 | [M + H]⁺: 317, 319 (3:2); ¹H NMR (400 MHz, CD₃OD) δ 7.19 (d, J = 8.86 Hz, 1H), 6.60 (dd, J = 8.89, 1.48 Hz, 1H), 5.00-4.90 (m, 1H), 3.38-3.34 (m, 1H), 2.97-2.88 (m, 1H), 2.66-2.46 (m, 1H), 2.28-2.10 (m, 2H), 1.99-1.89 (m, 1H), 1.68-1.44 (m, 2H), 0.99-0.93 (m, 3H). |
| 73 | 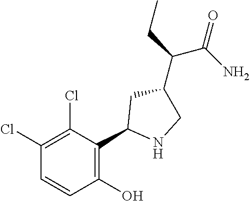 [SMILES: CC[C@@H](C(N)=O)[C@@H]1CN[C@@H](c2c(O)ccc(Cl)c2Cl)C1] | 2-((3R,5R)-5-(2,3- dichloro-6- hydroxyphenyl)pyrro- lidin-3- yl)butanamide isomer 2 | [M + H]⁺: 317, 319 (3:2); ¹H NMR (400 MHz, CD₃OD) δ 71.8 (d, J = 8.80 Hz, 1H), 6.60 (dd, J = 8.89, 2.93 Hz, 1H), 4.95 (dd, J = 9.04, 7.20 Hz, 1H), 3.43 (dd, J = 10.20, 6.65 Hz, 1H), 2.84 (dd, J = 10.21, 8.74 Hz, 1H), 2.58-2.42 (m, 1H), 2.33-2.11 (m, 2H), 1.90- 1.76 (m, 1H), 1.68-1.50 (m, 2H), 1.02-0.90 (m, 3H). |

### Example 14. Compound 48 (N-{[(3S,5R)-5-(2,3-dichloro-6-hydroxyphenyl) pyrrolidin-3-yl]methyl}-2-hydroxyacetamide)

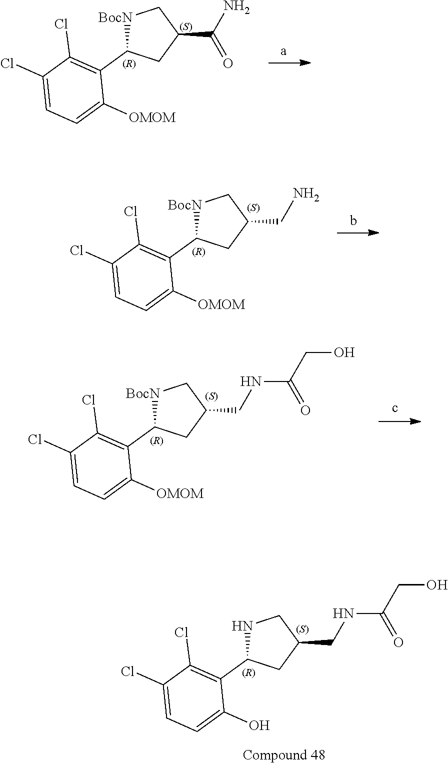

### Step a

To a stirred solution of tert-butyl (2R,4S)-4-carbamoyl-2-[2,3-dichloro-6-(methoxymethoxy)phenyl]pyrrolidine-1-carboxylate (0.150 g, 0.360 mmol) in THE (2 mL) was added BH₃-Me₂S (0.140 mL, 1.79 mmol, 10 M) at room temperature under nitrogen atmosphere. The reaction mixture was stirred at 70° C. for 4 h, quenched with MeOH (1 mL) at room temperature and concentrated under reduced pressure to afford tert-butyl (2R,4S)-4-(aminomethyl)-2-[2,3-dichloro-6-(methoxymethoxy)phenyl]pyrrolidine-1-carboxylate as a colorless oil (60.0 mg, 41%), which was used in the next step without purification: LCMS (ESI) calc'd C₁₈H₂₆Cl₂N₂O₄ for [M+H]⁺: 405, 407 (3:2) found 405, 407 (3:2).

### Step b

To a stirred solution of tert-butyl (2R,4S)-4-(aminomethyl)-2-[2,3-dichloro-6-(methoxymethoxy)phenyl]pyrrolidine-1-carboxylate (60.0 mg, 0.150 mmol) and glycolic acid (23.0 mg, 0.300 mmol) in DMF (2 mL) were added HATU (0.120 g, 0.300 mmol) and TEA (30.0 mg, 0.300 mmol) at room temperature. The reaction mixture was stirred for 2 h and dissolved in MeOH (1 mL). The resulting solution was purified by reverse phase chromatography, eluting with 48% ACN in water (plus 0.05% TFA) to afford tert-butyl (2R,4S)-2-[2,3-dichloro-6-(methoxymethoxy)phenyl]-4-[(2-hydroxyacetamido)methyl]pyrrolidine-1-carboxylate as a colorless oil (50.0 mg, 72.9%). LCMS (ESI) calc'd for C₂₀H₂₈Cl₂N₂O₆ [M+H]⁺: 463, 465 (3:2) found 463, 465 (3:2).

### Step c

To a stirred solution of tert-butyl (2R,4R)-2-[2,3-dichloro-6-(methoxymethoxy)phenyl]-4-[(2-hydroxyacetamido)methyl]pyrrolidine-1-carboxylate (50.0 mg, 0.110 mmol) in MeOH (0.4 mL) was added conc. HCl (0.8 mL) at room temperature. The reaction mixture was stirred for 2 h and concentrated under reduced pressure. The crude product was purified by Prep-HPLC with the following conditions: Column: XBridge Prep C18 OBD Column, 19×150 mm, 5 m; Mobile Phase A: water (plus 10 mM NH₄HCO₃), Mobile Phase B: ACN; Flow rate: 20 mL/min; Gradient: 30% B to 40% B in 4.5 min, 40% B; Wavelength: 210 nm; Retention Time: 4.35 min. The fractions containing the desired product were collected and concentrated under reduced pressure to afford N-{[(3S,5R)-5-(2,3-dichloro-6-hydroxyphenyl)pyrrolidin-3-yl]methyl}-2-hydroxyacetamide as an off-white solid (16.9 mg, 49%): LCMS (ESI) calc'd for C₁₃H₁₆C₁₂N₂O₃ [M+H]⁺: 319, 321 (3:2) found 319, 321 (3:2); ¹H NMR (400 MHz, CD3OD) δ 7.18 (d, J=8.8 Hz, 1H), 6.58 (d, J=8.8 Hz, 1H), 4.99 (t, J=8.4 Hz, 1H), 4.01 (s, 2H), 3.41-3.34 (m, 3H), 2.90 (dd, J=10.7, 6.8 Hz, 1H), 2.72-2.51 (m, 1H), 2.34-2.24 (m, 1H), 1.92-1.80 (m, 1H).

The compound in Table 7D below was prepared in an analogous fashion to that described for Compound 48, starting from tert-butyl (2R,4R)-4-carbamoyl-2-[2,3-dichloro-6-(methoxymethoxy) phenyl]pyrrolidine-1-carboxylate.

TABLE 7D

| Compound | | | MS: (M + H)⁺ & ¹H |
|---|---|---|---|
| No | Structure | Chemical Name | MNR |
| 49 | 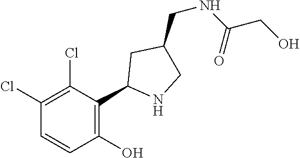 [SMILES: O=C(CO)NC[C@H]1CN[C@@H](c2c(O)ccc(Cl)c2Cl)C1] | N-(((3R,5R)-5-(2,3- dichloro-6- hydroxyphenyl)pyrrolidin- 3-yl)methyl)-2- hydroxyacetamide | [M + H]⁺: 319, 321 (3:2); ¹H NMR (300 MHz, CD₃OD) δ 7.18 (d, J = 8.9 Hz, 1H), 6.60 (d, J = 8.8 Hz, 1H), 4.95-4.92 (m, 1H), 3.99 (s, 2H), 3.46- 3.36 (m, 2H), 3.31- 3.25 (m, 1H), 2.99 (dd, J = 11.1, 7.6 Hz, 1H), |
| | | | 2.69-2.49 (m, 2H), |
| | | | 1.61-1.46 (m, 1H). |

### Example 15. Compound 50 (2-[5R-(2,3-dichloro-6-hydroxyphenyl)-3-methylpyrrolidin-3-yl]acetamide)

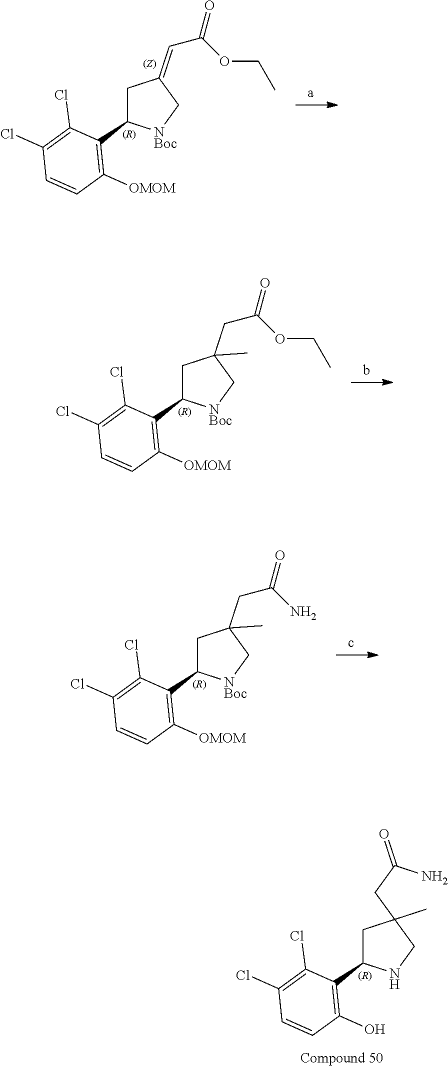

### Step a

To a stirred mixture of tert-butyl (2R,4Z)-2-[2,3-dichloro-6-(methoxymethoxy)phenyl]-4-(2-methoxy-2-oxoethylidene)pyrrolidine-1-carboxylate (0.300 g, 0.670 mmol) in THE (5 mL) were added CuI (0.260 g, 1.34 mmol) and SiMe₃Cl (0.290 g, 2.69 mmol) dropwise at 0° C. under nitrogen atmosphere. The reaction was stirred at room temperature for 1 h and then cooled to −60° C. CH₃MgBr (4 mL, 4.03 mmol, 1 Min THF) was added to the solution over 5 min. The reaction solution was stirred at −60° C. to room temperature for an additional 3 h, quenched with saturated aq NH₄Cl (20 mL) and extracted with EA (3×20 mL). The combined organic layers were washed with brine (2×20 mL) and dried over anhydrous Na₂SO₄. After filtration, the filtrate was concentrated under reduced pressure. The residue was purified by reverse phase chromatography, eluting with 70% ACN in water (plus 0.05% TFA) to afford tert-butyl (2R)-2-[2,3-dichloro-6-(methoxymethoxy)phenyl]-4-(2-methoxy-2-oxoethyl)-4-methylpyrrolidine-1-carboxylate as a yellow brown oil (0.180 g, 58%): LCMS (ESI) calc'd for C₂₂H₃₁Cl₂NO₆ [M+H]⁺: 476, 478 (3:2) found 476, 478 (3:2); ¹H NMR (400 MHz, CDCl₃) δ 7.32 (d, J=9.0 Hz, 1H), 7.04 (d, J=9.0 Hz, 1H), 5.63-5.46 (m, 1H), 5.30-5.07 (m, 2H), 4.23-4.11 (m, 2H), 3.76-3.57 (m, 1H), 3.55-3.26 (m, 4H), 2.52-2.39 (m, 2H), 2.16-2.06 (m, 2H), 1.44-1.21 (m, 6H), 1.15 (d, J=4.6 Hz, 9H).

### Step b

To a stirred solution of tert-butyl (2R)-2-[2,3-dichloro-6-(methoxymethoxy)phenyl]-4-(2-ethoxy-2-oxoethyl)-4-methylpyrrolidine-1-carboxylate (0.180 g, 0.380 mmol) in MeOH (3 mL) and H₂O (1 mL) was added LiOH (18.0 mg, 0.760 mmol) at room temperature. The reaction was stirred for 1 h and concentrated under reduced pressure. The residue was dissolved in DMF (2 mL) and HATU (0.220 g, 0.570 mmol), NH₄Cl (0.100 g, 1.89 mmol) and TEA (76.0 mg, 0.760 mmol) were added. The reaction mixture was stirred at room temperature for an additional 1 h, diluted with EA (20 mL) and water (20 mL), and extracted with EA (3×20 mL). The combined organic layers were washed with brine (2×20 mL) and dried over anhydrous Na₂SO₄. After filtration, the filtrate was concentrated under reduced pressure. The residue was purified by reverse phase chromatography, eluting with 50% ACN in water (plus 0.05% TFA) to afford tert-butyl (2R)-4-(carbamoylmethyl)-2-[2,3-dichloro-6-(methoxymethoxy)phenyl]-4-methylpyrrolidine-1-carboxylate as a yellow oil (0.110 g, 65%): LCMS (ESI) calc'd for C₂₀H₂₈C₁₂N₂O₅ [M+H]⁺: 447, 449 (3:2) found 447, 449 (3:2); ¹H NMR (400 MHz, CDCl₃) δ 7.33 (dd, J=9.0, 3.0 Hz, 1H), 7.07-6.99 (m, 1H), 5.80-5.46 (m, 1H), 5.29-5.07 (m, 2H), 3.63 (d, J=10.3 Hz, 1H), 3.54-3.43 (m, 4H), 2.53-1.87 (m, 4H), 1.43-1.31 (m, 3H), 1.15 (s, 9H).

### Step c

To a stirred solution of tert-butyl (2R)-4-(carbamoylmethyl)-2-[2,3-dichloro-6-(methoxymethoxy)phenyl]-4-methylpyrrolidine-1-carboxylate (0.100 g, 0.220 mmol) in MeOH (2.00 mL) was added conc. HCl (2.00 mL) at room temperature. The reaction was stirred for 1 h, concentrated under reduced pressure and purified by Prep-HPLC with the following conditions: Column: XBridge Prep C18 OBD Column, 19×150 mm, 5 m; Mobile Phase A: water (plus 10 mM NH₄HCO₃), Mobile Phase B: ACN; Flow rate: 20 mL/min; Gradient: 30% B to 50% B in 5.5 min, 50% B; Wavelength: 210 nm; Retention Time: 5.2 min. The fractions containing the desired product were collected and concentrated under reduced pressure to afford 2-[5R-(2,3-dichloro-6-hydroxyphenyl)-3-methylpyrrolidin-3-yl]acetamide as an off-white solid (35.0 mg, 52%): LCMS (ESI) calc'd for C₁₃H₁₆Cl₂N₂O₂ [M+H]⁺: 303, 305 (3:2) found 303, 305 (3:2); ¹H NMR (400 MHz, CD₃OD) δ 7.17 (dd, J=8.9, 1.1 Hz, 1H), 6.59 (dd, J=8.9, 1.2 Hz, 1H), 5.08-4.99 (m, 1H), 3.21 (d, J=11.3 Hz, 1H), 3.09-2.95 (m, 1H), 2.66-2.29 (m, 3H), 1.78-1.54 (m, 1H), 1.28 (d, J=5.8 Hz, 3H).

### Example 16. Compound 51 (2-[(3R,5R)-5-(2,3-dichloro-6-hydroxyphenyl)pyrrolidin-3-yl]-2-methylpropanamide) and Compound 52 (2-[(3S,5R)-5-(2,3-dichloro-6-hydroxyphenyl)pyrrolidin-3-yl]-2-methylpropanamide)

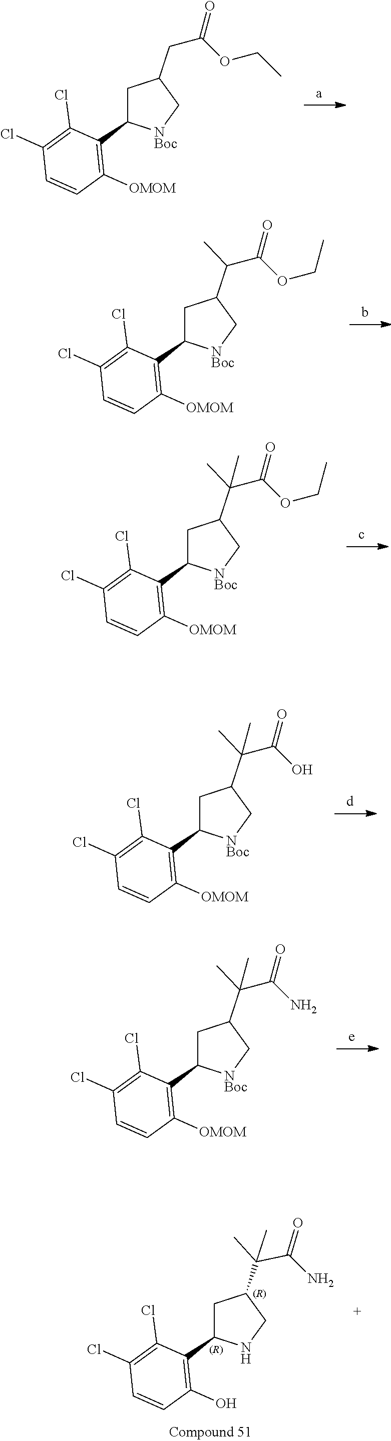

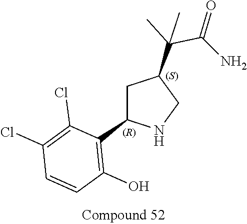

### Step a

To a stirred solution of i-Pr₂NH (0.180 g, 1.95 mmol) in THE (1 mL) was added n-BuLi (0.9 mL, 2.27 mmol, 2.5 Min Hexane) dropwise at −60° C. under nitrogen atmosphere. After 30 min, tert-butyl (2R)-2-[2,3-dichloro-6-(methoxymethoxy)phenyl]-4-(2-ethoxy-2-oxoethyl)pyrrolidine-1-carboxylate (0.300 g, 0.650 mmol) in THE (2 mL) was added at −78° C. The reaction solution was stirred at −78° C. for 30 min and CH₃I (0.920 g, 6.49 mmol) was added. The reaction mixture was stirred at −78° C. for an additional 2 h. The resulting mixture was quenched with saturated aq. NH₄Cl (20 mL) at room temperature and extracted with EA (3×30 mL). The combined organic layers were washed with brine (3×30 mL) and dried over anhydrous Na₂SO₄. After filtration, the filtrate was concentrated under reduced pressure to afford tert-butyl (2R)-2-[2,3-dichloro-6-(methoxymethoxy)phenyl]-4-(1-ethoxy-1-oxopropan-2-yl)pyrrolidine-1-carboxylate as a yellow oil (0.330 g, crude), which was used directly in the next step without purification: LCMS (ESI) calc'd for C₂₂H₃₁Cl₂NO₆ [M+H]⁺: 476, 478 (3:2), found 476, 478 (3:2).

### Step b

To a stirred solution of i-Pr₂NH (0.210 g, 2.08 mmol) in THE (1 mL) was added n-BuLi (1 mL, 2.43 mmol, 2.5 Min Hexane) dropwise at −60° C. under nitrogen atmosphere. After 30 min, tert-butyl(2R)-2-[2,3-dichloro-6-(methoxymethoxy)phenyl]-4-(1-ethoxy-1-oxopropan-2-yl)pyrrolidine-1-carboxylate (0.330 g, 0.690 mmol) in THE (2 mL) was added at −78° C. The reaction solution was stirred at −78° C. for 30 min under nitrogen atmosphere and CH₃I (0.980 g, 6.93 mmol) was added. The reaction mixture was stirred at −78° C. for an additional 2 h under nitrogen atmosphere. The resulting mixture was quenched by the addition of saturated aq. NH₄Cl (20 mL) at room temperature and extracted with EA (3×30 mL). The combined organic layers were washed with brine (3×30 mL) and dried over anhydrous Na₂SO₄. After filtration, the filtrate was concentrated under reduced pressure. The residue was purified by reverse phase chromatography, eluting with 50% ACN in water (plus 20 mM NH₄HCO₃) to afford tert-butyl (2R)-2-[2,3-dichloro-6-(methoxymethoxy)phenyl]-4-(1-ethoxy-2-methyl-1-oxopropan-2-yl)pyrrolidine-1-carboxylate as a yellow oil (0.150 g, 47% overall two steps): LCMS (ESI) calc'd for C₂₃H₃₃Cl₂NO₆ [M+H]⁺: 490, 492 (3:2), found 490, 492 (3:2); ¹H NMR (300 MHz, CDCl₃) δ 7.35-7.30 (m, 1H), 7.09-6.98 (m, 1H), 5.56-5.34 (m, 1H), 5.29-5.05 (m, 2H), 4.16 (q, J =7.1 Hz, 2H), 3.80-3.63 (m, 1H), 3.55-3.27 (m, 4H), 2.85-2.47 (m, 1H), 2.40-1.92 (m, 1H), 1.73-1.53 (m, 1H), 1.32-1.20 (m, 9H), 1.19-1.08 (m, 9H).

### Step c

To a stirred solution of tert-butyl (2R)-2-[2,3-dichloro-6-(methoxymethoxy)phenyl]-4-(1-ethoxy-2-methyl-1-oxopropan-2-yl)pyrrolidine-1-carboxylate (0.300 g, 0.610 mmol) in MeOH (3 mL) and H₂O (0.6 mL) was added NaOH (98.0 mg, 2.45 mmol) at room temperature. The reaction mixture was stirred at 50° C. for 16 h. The resulting mixture was acidified to pH 2 with saturated aq. citric acid, diluted with water (20 mL) and extracted with EA (3×20 mL). The combined organic layers were washed with brine (3×20 mL) and dried over anhydrous Na₂SO₄. After filtration, the filtrate was concentrated under reduced pressure to afford 2-[(5R)-1-(tert-butoxycarbonyl)-5-[2,3-dichloro-6-(methoxymethoxy)phenyl]pyrrolidin-3-yl]-2-methylpropanoic acid as a yellow solid (0.300 g, crude), which was used directly in the next step without purification: LCMS (ESI) calc'd for C₂₁H₂₉Cl₂NO₆ [M+H−56]⁺: 406, 408 (3:2), found 406, 408 (3:2).

### Step d

To a stirred solution of 2-[(5R)-1-(tert-butoxycarbonyl)-5-[2,3-dichloro-6-(methoxymethoxy)phenyl]pyrrolidin-3-yl]-2-methylpropanoic acid (0.300 g, 0.650 mmol) and HATU (0.370 g, 0.970 mmol) in DMF (3 mL) were added TEA (0.200 g, 1.95 mmol) and NH₄Cl (69.0 mg, 1.30 mmol) at room temperature. The reaction mixture was stirred for 3 h, diluted with water (20 mL) and extracted with EA (3×20 mL). The combined organic layers were washed with brine (3×20 mL) and dried over anhydrous Na₂SO₄. After filtration, the filtrate was concentrated under reduced pressure to afford tert-butyl (2R)-4-(1-carbamoyl-1-methylethyl)-2-[2,3-dichloro-6-(methoxymethoxy)phenyl]pyrrolidine-1-carboxylate as a yellow oil (0.300 g, crude), which was used directly in the next step without purification: LCMS (ESI) calc'd for C₂₁H₃₀Cl₂N₂O₅ [M+H]⁺: 461, 463 (3:2), found 461, 463 (3:2).

### Step e

To a stirred solution of tert-butyl (2R)-4-(1-carbamoyl-1-methylethyl)-2-[2,3-dichloro-6-(methoxymethoxy)phenyl]pyrrolidine-1-carboxylate (0.300 g, crude) in MeOH (3 mL) was added conc. HCl (3 mL) at room temperature. The reaction mixture was stirred at room temperature for 3 h and concentrated under reduced pressure. The residue was purified by Prep-HPLC with the following conditions: Column: SunFire Prep C18 OBD Column, 19×150 mm, 5 m 10 nm; Mobile Phase A: water (plus 0.05% TFA), Mobile Phase B: ACN; Flow rate: 25 mL/min; Gradient: 20% B to 20% B in 6.8 min, 20% B; Detector UV: 210 nm; Retention Time 1: 6.27 min, Retention Time 2: 7.58 min. The faster-eluting enantiomer at 6.27 min was obtained 2-[(3R,5R)-5-(2,3-dichloro-6-hydroxyphenyl)pyrrolidin-3-yl]-2-methylpropanamide (Compound 51) as a purple solid (6.00 mg, 2.3% over three steps): LCMS (ESI) calc'd for C₁₄H₁₈C₁₂N₂O₂ [M+H]⁺: 317, 319 (3:2), found 317, 319 (3:2); ¹H NMR (400 MHz, CD₃OD) δ 7.46 (d, J=8.9 Hz, 1H), 6.93 (d, J=8.9 Hz, 1H), 5.26 (t, J=9.4 Hz, 1H), 3.70 (dd, J=11.4, 7.8 Hz, 1H), 3.49-3.41 (m, 1H), 3.02-2.87 (m, 1H), 2.42-2.24 (m, 2H), 1.30 (d, J=3.1 Hz, 6H). The slower-eluting enantiomer at 7.58 min was obtained 2-[(3S,5R)-5-(2,3-dichloro-6-hydroxyphenyl)pyrrolidin-3-yl]-2-methylpropanamide as an off-white solid (Compound 52) (10.6 mg, 4.0% over three steps): LCMS (ESI) calc'd for C₁₄H₁₈Cl₂N₂O₂ [M+H]⁺: 317, 319 (3:2), found 317, 319 (3:2); ¹H NMR (400 MHz, CD₃OD) δ 7.45 (d, J=9.0 Hz, 1H), 6.92 (d, J=8.9 Hz, 1H), 5.27 (dd, J=10.3, 8.2 Hz, 1H), 3.69-3.56 (m, 1H), 3.49 (dd, J=11.4, 8.4 Hz, 1H), 2.88-2.74 (m, 1H), 2.39-2.25 (m, 2H), 1.30 (d, J=4.8 Hz, 6H).

### Example 17. Compound 53 (2-((2S,5R)-5-(2,3-dichloro-6-hydroxyphenyl)pyrrolidin-2-yl)acetamide)

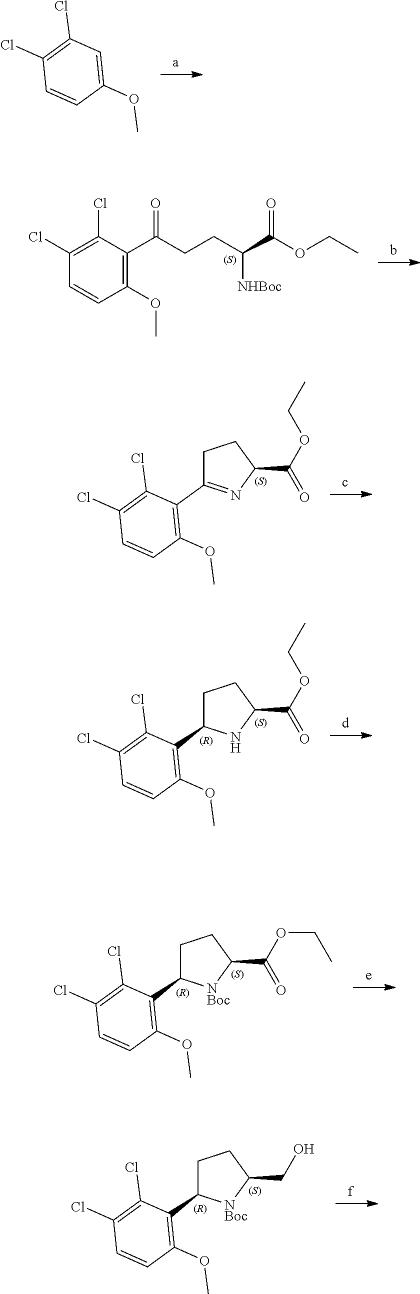

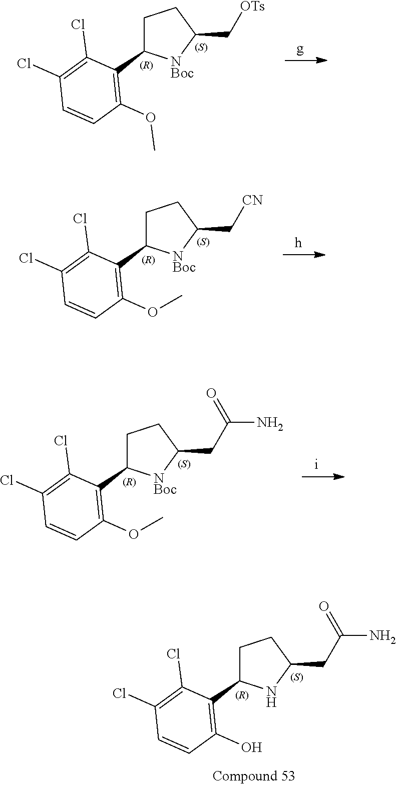

### Step a

To a stirred solution of 1,2-dichloro-4-methoxybenzene (2.00 g, 11.3 mmol) in THE (50 mL) was added n-BuLi (6.78 mL, 16.9 mmol, 2.5 M in hexane) dropwise at −78° C. under nitrogen atmosphere. After stirring for 30 min 1-tert-butyl 2-ethyl (2S′)-5-oxopyrrolidine-1,2-dicarboxylate (4.36 g, 17.0 mmol) in THE (50 mL) was added. The resulting solution was stirred for 2 h, quenched with saturated aq. NH₄Cl (10 mL) at 0° C., diluted with water (50 mL) and extracted with EA (3×40 mL). The combined organic layers were washed with brine (3×30 mL) and dried over anhydrous Na₂SO₄. After filtration, the filtrate was concentrated under reduced pressure. The residue was purified by silica gel column chromatography, eluting with PE/EA (3/1) to afford ethyl (2S)-2-[(tert-butoxycarbonyl)amino]-5-(2,3-dichloro-6-methoxyphenyl)-5-oxopentanoate as an off-white solid (1.26 g, 25%): LCMS (ESI) calc'd for C₁₉H₂₅Cl₂NO₆ [M+Na]⁺: 456, 458 (3:2) found 456, 458 (3:2); ¹H NMR (400 MHz, CDCl₃) δ 7.43 (d, J=8.9 Hz, 1H), 6.81 (d, J=8.9 Hz, 1H), 5.13 (d, J=8.4 Hz, 1H), 4.38-4.29 (m, 1H), 4.24 (qd, J=7.1, 2.3 Hz, 2H), 3.83 (s, 3H), 3.00-2.78 (m, 2H), 2.37-2.24 (m, 1H), 2.15-2.00 (m, 1H), 1.46 (s, 9H), 1.31 (t, J=7.1 Hz, 3H).

### Step b

To a stirred solution of ethyl (2S)-2-[(tert-butoxycarbonyl)amino]-5-(2,3-dichloro-6-methoxyphenyl)-5-oxopentanoate (1.20 g, 2.76 mmol) in DCM (12 mL) was added TFA (3 mL) at room temperature. The reaction mixture was stirred at 40° C. for 3 h, neutralized with saturated aq. NaHCO₃ (20 mL) to pH 7 at 0° C. and extracted with EA (3×30 mL). The combined organic layers were washed with brine (3×30 mL) and dried over anhydrous Na₂SO₄. After filtration, the filtrate was concentrated under reduced pressure to afford ethyl (2S)-5-(2,3-dichloro-6-methoxyphenyl)-3,4-dihydro-2H-pyrrole-2-carboxylate as a yellow oil (1.20 g, crude), which was used directly in the next step without purification: LCMS (ESI) calc'd for C₁₄H₁₅Cl₂NO₃ [M+H]⁺: 316, 318 (3:2) found 316, 318 (3:2): ¹H NMR (300 MHz, CDCl₃) δ 7.42 (d, J=8.9 Hz, 1H), 6.80 (d, J=8.9 Hz, 1H), 5.02-4.88 (m, 1H), 4.27 (q, J=7.1 Hz, 2H), 3.81 (s, 3H), 3.06-2.76 (m, 2H), 2.46-2.25 (m, 2H), 1.33 (t, J=7.1 Hz, 3H).

### Step c

To a stirred solution of ethyl (2S)-5-(2,3-dichloro-6-methoxyphenyl)-3,4-dihydro-2H-pyrrole-2-carboxylate (1.20 g, 3.76 mmol) in EA (12 mL) was added PtO₂ (0.172 g, 0.759 mmol) at room temperature. The reaction mixture was stirred for 2 h under hydrogen atmosphere (1.5 atm). The resulting mixture was filtered and the filter cake was washed with EA (3×20 mL). The filtrate was concentrated under reduced pressure and the residue was purified by reverse phase chromatography, eluting with 37% ACN in water (plus 0.05% TFA) to afford ethyl (2S,5R)-5-(2,3-dichloro-6-methoxyphenyl)pyrrolidine-2-carboxylate trifluoroacetic acid as a yellow oil (0.800 g, 67% overall two steps): LCMS (ESI) calc'd for C₁₄H₁₇C₁₂NO₃ [M+H]⁺: 318, 320 (3:2) found 318, 320 (3:2); ¹H NMR (400 MHz, CDCl₃) δ 7.52 (d, J=9.0 Hz, 1H), 6.90 (d, J=9.0 Hz, 1H), 5.47-5.34 (m, 1H), 4.79 (d, J=10.7 Hz, 1H), 4.46-4.30 (m, 2H), 4.03 (s, 3H), 2.84-2.59 (m, 1H), 2.47-2.27 (m, 2H), 2.27-2.13 (m, 1H), 1.40 (t, J=7.2 Hz, 3H).

### Step d

To a stirred solution of ethyl (2S,5R)-5-(2,3-dichloro-6-methoxyphenyl)pyrrolidine-2-carboxylate (0.800 g, 2.51 mmol) and NaHCO₃ (0.422 g, 5.03 mmol) in THE (8 mL) and H₂O (2 mL) was added Boc₂O (0.690 g, 3.02 mmol) at room temperature. The reaction mixture was stirred for 2 h, diluted with water (20 mL) and extracted with EA (3×20 mL). The combined organic layers were washed with brine (3×20 mL) and dried over anhydrous Na₂SO₄. After filtration, the filtrate was concentrated under reduced pressure. The residue was purified by reverse phase chromatography, eluting with 67% ACN in water (plus 10 mmol/L NH₄HCO₃) to afford 1-tert-butyl 2-ethyl (2S, 5R)-5-(2,3-dichloro-6-methoxyphenyl)pyrrolidine-1,2-dicarboxylate as a yellow oil (0.400 g, 38%): LCMS (ESI) calc'd for C₁₉H₂₅Cl₂NO₅ [M+H]⁺: 418, 420 (3:2) found 418, 420 (3:2); ¹H NMR (300 MHz, CDCl₃) δ 7.38-7.30 (m, 1H), 6.76 (d, J=8.9 Hz, 1H), 5.49-5.34 (m, 1H), 4.60-4.44 (m, 1H), 4.34-4.16 (m, 2H), 3.79 (s, 3H), 2.49-2.10 (m, 4H), 1.49-1.09 (m, 12H).

### Step e

To a stirred solution of 1-tert-butyl 2-ethyl (2S,5R)-5-(2,3-dichloro-6-methoxyphenyl)pyrrolidine-1,2-dicarboxylate (0.400 g, 0.960 mmol) in MeOH (8 mL) was added NaBH4 (0.720 g, 19.1 mmol) in portions at room temperature. The reaction solution was stirred for 4 h, quenched with water (10 mL) and extracted with EA (3×30 mL). The combined organic layers were washed with brine (3×30 mL) and dried over anhydrous Na₂SO₄. After filtration, the filtrate was concentrated under reduced pressure. The residue was purified by reverse phase chromatography, eluting with 62% ACN in water (plus 10 mmol/L NH₄HCO₃) to afford tert-butyl (2R, 5S)-2-(2,3-dichloro-6-methoxyphenyl)-5-(hydroxymethyl)pyrrolidine-1-carboxylate as a colorless oil (0.200 g, 56%): LCMS (ESI) calc'd for C₁₇H₂₃Cl₂NO₄ [M+H]⁺: 376, 378 (3:2) found 376, 378 (3:2); ¹H NMR (300 MHz, CDCl₃) δ 7.36 (d, J=8.9 Hz, 1H), 6.80 (d, J=8.9 Hz, 1H), 5.38 (t, J=8.4 Hz, 1H), 4.32-4.16 (m, 1H), 3.99-3.89 (m, 1H), 3.86 (s, 3H), 3.81-3.67 (m, 1H), 2.27-2.04 (m, 3H), 1.89-1.74 (m, 1H), 1.14 (s, 9H).

### Step f

To a stirred solution of tert-butyl (2R, 5S)-2-(2,3-dichloro-6-methoxyphenyl)-5-(hydroxymethyl)pyrrolidine-1-carboxylate (0.200 g, 0.530 mmol) and TEA (0.110 g, 1.06 mmol) in DCM (3 mL) was added TsCl (0.200 g, 1.06 mmol) at room temperature. The reaction mixture was stirred for 16 h and concentrated under reduced pressure. The residue was purified by silica gel column chromatography, eluting with PE/EA (3/1) to afford tert-butyl (2R,5S)-2-(2,3-dichloro-6-methoxyphenyl)-5-{[(4-methylbenzenesulfonyl)oxy]methyl}pyrrolidine-1-carboxylate as a colorless oil (0.160 g, 57%): LCMS (ESI) calc'd for C₂₄H₂₉C₁₂NO₆S [M+H]⁺: 530, 532 (3:2) found 530, 532 (3:2); ¹H NMR (300 MHz, CDCl₃) δ 7.89-7.81 (m, 2H), 7.38 (d, J=8.1 Hz, 2H), 7.33 (d, J=9.1 Hz, 1H), 6.74 (d, J=8.8 Hz, 1H), 5.30 (t, J=8.8 Hz, 1H), 4.35-4.06 (m, 3H), 3.74 (s, 3H), 2.47 (s, 3H), 2.20-2.07 (m, 2H), 2.07-1.93 (m, 2H), 1.20 (d, J=74.1 Hz, 9H).

### Step g

To a stirred solution of tert-butyl (2R,5S)-2-(2,3-dichloro-6-methoxyphenyl)-5-{[(4-methylbenzenesulfonyl)oxy]methyl}pyrrolidine-1-carboxylate (0.140 g, 0.26 mmol) in DMF (2 mL) was added Bu₄NCN (0.280 g, 1.06 mmol) at room temperature. The reaction mixture was stirred at 80° C. for 16 h, diluted with water (30 mL) and extracted with EA (3×30 mL). The combined organic layers were washed with brine (3×20 mL) and dried over anhydrous Na₂SO₄. After filtration, the filtrate was concentrated under reduced pressure. The residue was purified by reverse phase chromatography, eluting with 70% ACN in water (plus 10 mmol/L NH₄HCO₃) to afford tert-butyl (2S,5R)-2-(cyanomethyl)-5-(2,3-dichloro-6-methoxyphenyl)pyrrolidine-1-carboxylate as a light yellow oil (50.0 mg, 49%): LCMS (ESI) calc'd for C₁₈H₂₂Cl₂N₂O₃ [M−56]⁺: 329, 331 (3:2) found 329, 331 (3:2); ¹H NMR (300 MHz, CDCl₃) δ 7.36 (d, J=8.9 Hz, 1H), 6.78 (d, J=8.9 Hz, 1H), 5.41-5.30 (m, 1H), 4.38-4.27 (m, 1H), 3.80 (s, 3H), 3.17-3.03 (m, 1H), 2.73 (dd, J=16.5, 10.0 Hz, 1H), 2.34-2.02 (m, 4H), 1.11 (s, 9H).

### Step h

To a stirred solution of tert-butyl (2S,5R)-2-(cyanomethyl)-5-(2,3-dichloro-6-methoxyphenyl)pyrrolidine-1-carboxylate (50.0 mg, 0.130 mmol) and NaOH (16.0 mg, 0.390 mmol) in MeOH (1 mL) and H₂O (0.2 mL) was added H₂O₂ (13.0 mg, 0.390 mmol) at room temperature. The reaction mixture was stirred for 4 h, quenched with saturated aq. Na₂SO₃ (2 mL) at 0° C., diluted with water (20 mL) and extracted with EA (3×20 mL) respectively. The combined organic layers were washed with brine (3×20 mL) and dried over anhydrous Na₂SO₄. After filtration, the filtrate was concentrated under reduced pressure. The residue was purified by reverse phase chromatography, eluting with 40% ACN in water (plus 10 mmol/L NH₄HCO₃) to afford tert-butyl (2S,5R)-2-(carbamoylmethyl)-5-(2,3-dichloro-6-methoxyphenyl)pyrrolidine-1-carboxylate as a yellow oil (30.0 mg, 57%): LCMS (ESI) calc'd for C₁₈H₂₄C₁₂N₂O₄ [M+H]⁺: 403, 405 (3:2) found 403, 405 (3:2).

### Step i

To a stirred solution of tert-butyl (2S,5R)-2-(carbamoylmethyl)-5-(2,3-dichloro-6-methoxyphenyl)pyrrolidine-1-carboxylate (50.0 mg, 0.120 mmol) in DCM (1 mL) was added BBr₃ (0.190 g, 0.740 mmol) at room temperature. The resulting mixture was stirred at 40° C. for 4 h, quenched with water (2 mL) and concentrated under reduced pressure. The residue was purified by Prep-HPLC with the following conditions: Column: XBridge Prep OBD C18 Column, 19×250 mm, 5 m; Mobile Phase A: Water (plus 10 mmol/L NH₄HCO₃), Mobile Phase B: ACN; Flow rate: 30 mL/min; Gradient: 20% B to 50% B in 5.2 min, 50% B; Detector: UV 254/220 nm; Retention time: 5.08 min. The fractions containing the desired product were collected and concentrated under reduced pressure to afford 2-[(2S,5R)-5-(2,3-dichloro-6-hydroxyphenyl)pyrrolidin-2-yl]acetamide as a light yellow solid (4.00 mg, 18%): LCMS (ESI) calc'd for C₁₂H₁₄Cl₂N₂O₂ [M+H]⁺: 289, 291 (3:2) found 289, 291 (3:2); ¹H NMR (400 MHz, CD₃OD) δ 7.20 (d, J=8.8 Hz, 1H), 6.60 (d, J=8.8 Hz, 1H), 4.93-4.91 (m, 1H), 3.78-3.70 (m, 1H), 2.59-2.55 (m, 2H), 2.48-2.37 (m, 1H), 2.22-2.11 (m, 1H), 1.86-1.66 (m, 2H).

### Example 18. Compound 54 (3,4-dichloro-2-[(2R,4R)-4-(1H-pyrazol-4-ylmethyl)pyrrolidin-2-yl] phenol)

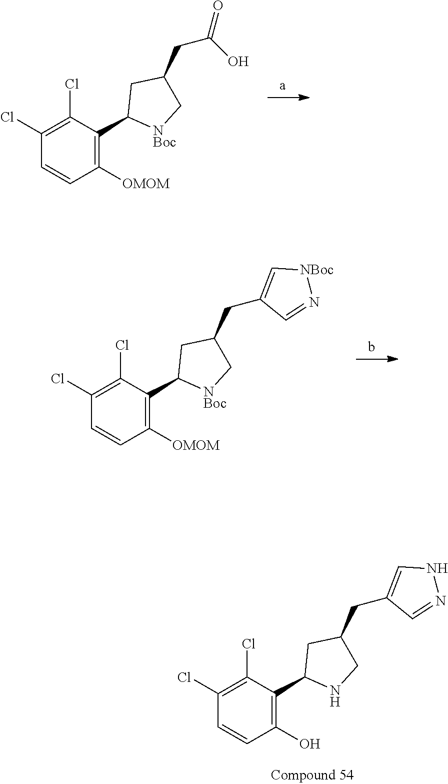

### Step a

A solution of nickel II chloride DME complex (5.00 mg, 0.02 mmol) and dtbpy (6.00 mg, 0.02 mmol) in DMSO (1 mL) was stirred at 60° C. for 30 min under nitrogen atmosphere to afford the solution A. Meanwhile, a solution of [(3S,5R)-1-(tert-butoxycarbonyl)-5-[2,3-dichloro-6-(methoxymethoxy)phenyl] pyrrolidin-3-yl] acetic acid (Intermediate 5a) (0.100 g, 0.23 mmol), tert-butyl 4-iodopyrazole-1-carboxylate (0.100 g, 0.35 mmol), 2-tert-butyl-1,1,3,3-tetramethylguanidine (59.0 mg, 0.35 mmol), 2,3-dihydro-1H-isoindole-1,3-dione (51.0 mg, 0.35 mmol) and Ir[dF(CF₃)ppy]2(dtbpy)PF₆ (3.00 mg, 0.002 mmol) in DMSO (5 mL) was stirred at room temperature for 5 min under nitrogen atmosphere to afford the solution B. The solution A was then added into the solution B at room temperature under nitrogen atmosphere. The final reaction mixture was irradiated with blue LEDs at room temperature for 5 h, diluted with water (20 mL) and extracted with EA (3×20 mL). The combined organic layers were washed with brine (3×20 mL) and dried over anhydrous Na₂SO₄. After filtration, the filtrate was concentrated under reduced pressure. The residue was purified by reverse phase chromatography, eluting with 30% ACN in water (plus 0.05% TFA) to afford tert-butyl 4-{[(3R,5R)-1-(tert-butoxycarbonyl)-5-[2,3-dichloro-6-(methoxymethoxy)phenyl]pyrrolidin-3-yl]methyl}pyrazole-1-carboxylate as a yellow oil (40.0 mg, 31%): LCMS (ESI) calc'd for C₂₆H₃₅Cl₂N₃O₆ [M+H]⁺: 556, 558 (3:2) found 556, 558 (3:2).

### Step b

A solution of tert-butyl 4-{[(3R,5R)-1-(tert-butoxycarbonyl)-5-[2,3-dichloro-6-(methoxymethoxy)phenyl] pyrrolidin-3-yl]methyl}pyrazole-1-carboxylate (40.0 mg, 0.07 mmol) in conc. HCl (0.5 mL) and MeOH (0.5 mL) was stirred at room temperature for 2 h and concentrated under reduced pressure. The residue was purified by reverse phase chromatography, eluting with 40% ACN in water (plus 10 mmol/L NH₄HCO₃) to afford 3,4-dichloro-2-[(2R,4R)-4-(1H-pyrazol-4-ylmethyl)pyrrolidin-2-yl] phenol as an off-white solid (12.3 mg, 55%): LCMS (ESI) calc'd for C₁₄H₁₅Cl₂N₃O [M+H]⁺: 312, 314 (3:2) found 312, 314 (3:2): ¹H NMR (400 MHz, CD₃OD) δ 7.45 (s, 1H), 7.37 (s, 1H), 7.18 (d, J=8.8 Hz, 1H), 6.59 (d, J=8.9 Hz, 1H), 4.95-4.92 (m, 1H), 3.38-3.34 (m, 1H), 2.93 (dd, J=11.1, 8.2 Hz, 1H), 2.74-2.68 (m, 2H)), 2.65-2.51 (m, 2H), 1.64-1.48 (m, 1H).

The compound in Table 7E below was prepared in an analogous fashion to that described for Compound 54, starting from Intermediate 5a and the corresponding heteroaryl halide.

TABLE 7E

| Compound | | | MS: (M + H)⁺ & ¹H |
|---|---|---|---|
| No | Structure | Chemical Name | MNR |
| 55 | 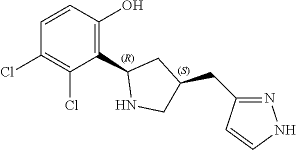 | 2-((2R,4S)-4-((1H- pyrazol-3- yl)methyl)pyrrolidin- 2-yl)-3,4- dichlorophenol | [M + H]⁺: 312, 314 (3:2); ¹H NMR (400 MHz, CD₃OD) δ 7.54 (s, 1H), 7.20 (d, J = 8.9 Hz, 1H), 6.62 (d, J = 8.9 Hz, 1H), 6.18 (d, J = 2.2 Hz, 1H), 4.95 (dd, J = 11.1, 6.6 Hz, 1H), 3.40-3.34 (m, 1H), 2.97 (t, J = |
| | | | 9.9 Hz, 1H), 2.85 (d, J = |
| | | | 7.3 Hz, 2H), 2.75- |
| | | | 2.63 (m, 1H), 2.60- |
| | | | 2.50 (m, 1H), 1.68- |
| | | | 1.54 (m, 1H). |

### Example 19. Compound 74 (2-[(3S,5R)-5-(2,3-dichloro-6-hydroxyphenyl)pyrrolidin-3-yl]-3-methoxypropanamide isomer 1) and Compound 75 (2-[(3S,5R)-5-(2,3-dichloro-6-hydroxyphenyl)pyrrolidin-3-yl]-3-methoxypropanamide isomer 2)

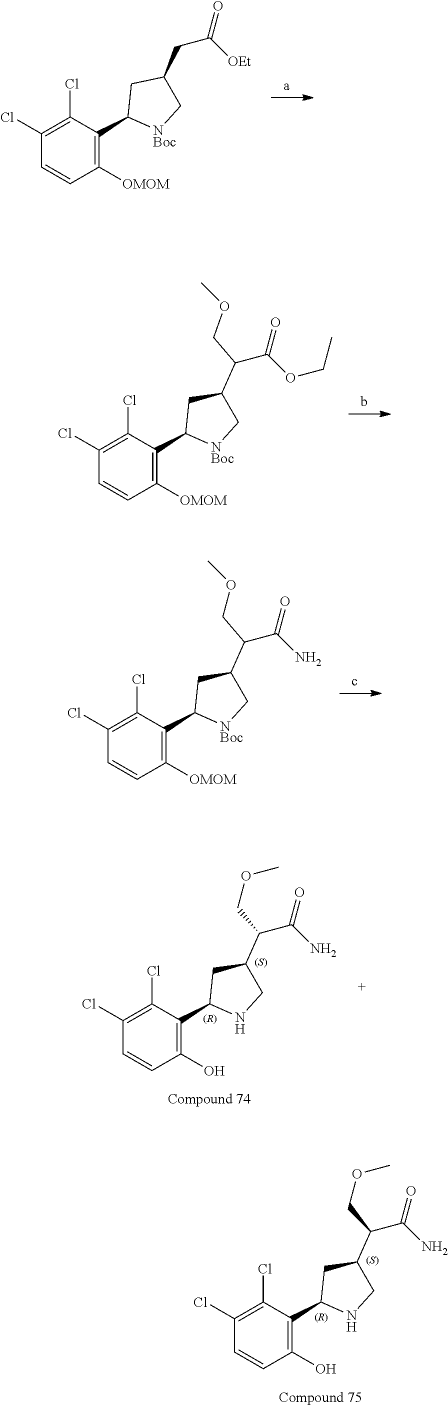

### Step a

To a stirred solution of bis(propan-2-yl)amine (0.853 g, 8.44 mmol) in THE (10 mL) was added n-BuLi (3.94 mL, 9.84 mmol, 2.5M in hexane) dropwise at −78° C. under nitrogen atmosphere. After stirring for 30 min, tert-butyl (2R,4S)-2-[2,3-dichloro-6-(methoxymethoxy)phenyl]-4-(2-ethoxy-2-oxoethyl)pyrrolidine-1-carboxylate (1.30 g, 2.81 mmol) in THE (10 mL) was added dropwise over 20 min. After stirring for 30 min, bromo(methoxy)methane (1.76 g, 14.1 mmol) in THE (10 mL) was added. The resulting reaction mixture was stirred for 2 h, quenched with saturated aq. NH₄Cl (30 mL), and extracted with EA (3×50 mL). The combined organic layers were washed with brine (3×30 mL) and dried over anhydrous Na₂SO₄. After filtration, the filtrate was concentrated under reduced pressure. The residue was purified by reverse phase chromatography, eluting with 78% ACN in water (plus 10 mmol/L NH₄HCO₃) to afford tert-butyl (2R,4S)-2-(2,3-dichloro-6-(methoxymethoxy)phenyl)-4-(1-ethoxy-3-methoxy-1-oxopropan-2-yl)pyrrolidine-1-carboxylate as a yellow oil (0.550 g, 38%): LCMS (ESI) calc'd for C₂₃H₃₃Cl₂NO₇ [M+H]⁺: 506, 508 (3:2) found 506, 508 (3:2); ¹H NMR (300 MHz, CDCl₃) δ 7.29 (d, J=9.28 Hz, 1H), 7.00 (d, J=8.95 Hz, 1H), 5.53-5.33 (m, 1H), 5.29-5.06 (m, 2H), 4.26-4.08 (m, 2H), 4.00-3.70 (m, 1H), 3.69-3.51 (m, 1H), 3.51-3.28 (m, 7H), 3.28-3.06 (m, 1H), 2.73-2.22 (m, 3H), 2.04-1.76 (m, 1H), 1.43-1.02 (m, 12H).

### Step b

To a stirred solution of tert-butyl (2R,4S)-2-[2,3-dichloro-6-(methoxymethoxy)phenyl]-4-(1-ethoxy-3-methoxy-1-oxopropan-2-yl)pyrrolidine-1-carboxylate (0.550 g, 1.09 mmol) in MeOH (6 mL) were added LiOH H₂O (91.2 mg, 2.17 mmol) and H₂O (2 mL) at room temperature. The reaction was stirred for 3 h and concentrated under reduced pressure. The residue was dissolved in DMF (6 mL) and HATU (0.619 g, 1.63 mmol), NH₄Cl (87.1 mg, 1.63 mmol) and TEA (0.329 g, 3.26 mmol) were added. The resulting reaction mixture was stirred for 1 h, diluted with water (20 mL), and extracted with EA (3×20 mL). The combined organic layers were washed with brine (3×20 mL) and dried over anhydrous Na₂SO₄. After filtration, the filtrate was concentrated under reduced pressure. The residue was purified by reverse phase chromatography, eluting with 45% ACN in water (plus 0.05% TFA) to afford tert-butyl (2R,4S)-4-(1-carbamoyl-2-methoxyethyl)-2-[2,3-dichloro-6-(methoxymethoxy)phenyl]pyrrolidine-1-carboxylate as a yellow oil (0.420 g, 81%) LCMS (ESI) calc'd for C₂₁H₃₀C₁₂N₂O₆ [M+H]⁺: 477, 479 (3:2) found 477,479 (3:2); ¹H NMR (300 MHz, CDCl₃) δ 7.34-7.27 (m, 1H), 7.00 (dd, J=9.09, 4.29 Hz, 1H), 6.52-5.83 (m, 2H), 5.65-5.34 (m, 1H), 5.34-5.01 (m, 2H), 3.99-3.84 (m, 1H), 3.79-3.51 (m, 1H), 3.46-3.26 (m, 4H), 3.27-3.08 (m, 4H), 2.67-2.26 (m, 3H), 2.07-1.79 (m, 1H), 1.48-0.94 (m, 9H).

### Step c

To a stirred solution of tert-butyl (2R,4S)-4-(1-carbamoyl-2-methoxyethyl)-2-[2,3-dichloro-6-(methoxymethoxy)phenyl]pyrrolidine-1-carboxylate (0.150 g, 0.314 mmol) in MeOH (1 mL) was added aq. HCl (1 mL, 4 M) at room temperature. The reaction mixture was stirred for 1 h and concentrated under reduced pressure. The residue was purified by Prep-HPLC with the following conditions: Column: Sun Fire Prep C18 OBD Column, 19×150 mm, 5 m 10 nm; Mobile Phase A: Water (plus 0.05% TFA), Mobile Phase B: ACN; Flow rate: 20 mL/min; Gradient: 18% B to 23% B in 5 min, Detector: UV 254/220 nm; Retention time 1: 3.85 min, Retention time 2: 5.65 min. The faster-eluting isomer at 3.85 min was obtained 2-[(3S,5R)-5-(2,3-dichloro-6-hydroxyphenyl)pyrrolidin-3-yl]-3-methoxypropanamide isomer 1 as an off-white solid (8.50 mg, 6%): LCMS (ESI) calc'd for C₁₄H₁₈C₁₂N₂O₃ [M+H]⁺: 333, 335 (3:2) found 333, 335 (3:2); ¹H NMR (400 MHz, CD₃OD) δ 7.44 (d, J=8.90 Hz, 1H), 6.91 (d, J=8.91 Hz, 1H), 5.24 (dd, J=11.49, 7.08 Hz, 1H), 3.66-3.59 (m, 2H), 3.54 (dd, J=9.53, 5.76 Hz, 1H), 3.41 (t, J=11.04 Hz, 1H), 3.36 (s, 3H), 2.74-2.64 (m, 2H), 2.39-2.19 (m, 2H). The slower-eluting isomer at 5.65 min was obtained 2-[(3S,5R)-5-(2,3-dichloro-6-hydroxyphenyl)pyrrolidin-3-yl]-3-methoxypropanamide isomer 2 as an off-white solid (12.1 mg, 9%): LCMS (ESI) calc'd for C₁₄H₁₈Cl₂N₂O₃ [M+H]⁺: 333, 335 (3:2) found 333, 335 (3:2); ¹H NMR (400 MHz, CD₃OD) δ 7.45 (dd, J=8.92, 0.85 Hz, 1H), 6.91 (d, J=8.92 Hz, 1H), 5.27 (dd, J=11.64, 6.85 Hz, 1H), 3.67-3.54 (m, 2H), 3.54-3.45 (m, 2H), 3.34 (s, 3H), 2.79-2.62 (m, 2H), 2.50-2.36 (m, 1H), 2.21 (q, J=11.86 Hz, 1H).

### Example 20. Compound 76 (2-[(3S,5R)-5-(2,3-dichloro-6-hydroxyphenyl)pyrrolidin-3-yl]-3-hydroxypropanamide isomer 1) and Compound 77 (2-[(3S,5R)-5-(2,3-dichloro-6-hydroxyphenyl)pyrrolidin-3-yl]-3-hydroxypropanamide isomer 2)

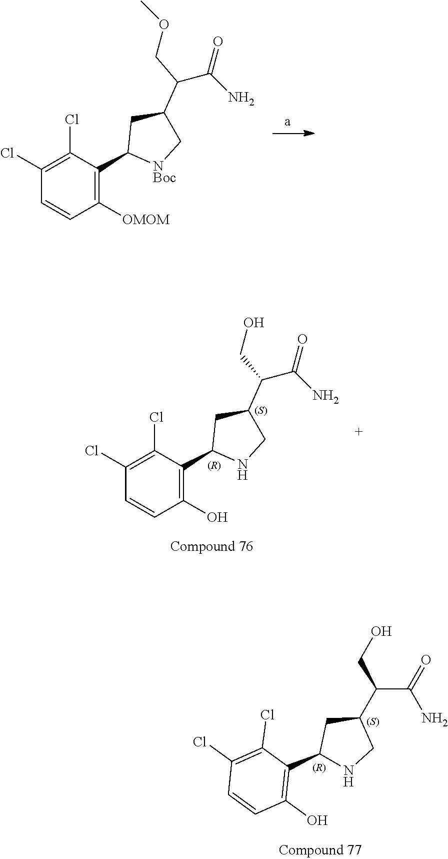

### Step a

To a stirred solution of tert-butyl (2R,4S)-4-(1-carbamoyl-2-methoxyethyl)-2-[2,3-dichloro-6-(methoxymethoxy)phenyl]pyrrolidine-1-carboxylate (0.270 g, 0.566 mmol) in DCM (3 mL) was added BBr₃ (1.42 g, 5.66 mmol) at room temperature. The reaction mixture was stirred for 1 h, quenched with water (5 mL) and concentrated under reduced pressure. The residue was purified by Prep-HPLC with the following conditions: Column: Sun Fire Prep C18 OBD Column, 19×150 mm, 5 m 10 nm; Mobile Phase A: Water (plus 0.05% TFA), Mobile Phase B: ACN; Flow rate: 20 mL/min; Gradient: 13% B to 24% B in 5 min, 24% B; Detector: UV 254/220 nm; Retention time 1: 4.51 min, Retention time 2: 4.91 min. The faster-eluting isomer at 4.51 min was obtained 2-[(3S,5R)-5-(2,3-dichloro-6-hydroxyphenyl)pyrrolidin-3-yl]-3-hydroxypropanamide isomer 1 as an off-white solid (18.6 mg, 8%): LCMS (ESI) calc'd for C₁₃H₁₆Cl₂N₂O₃ [M+H]⁺: 319, 321 (3:2) found 319, 321 (3:2); ¹H NMR (400 MHz, CD₃OD) δ 7.47 (d, J=8.89 Hz, 1H), 6.92 (d, J=8.93 Hz, 1H), 5.26 (dd, J=11.52, 7.00 Hz, 1H), 3.84 (dd, J=10.85, 6.51 Hz, 1H), 3.77-3.64 (m, 2H), 3.45 (t, J=11.37 Hz, 1H), 2.81-2.66 (m, 1H), 2.61-2.53 (m, 1H), 2.42-2.32 (m, 1H), 2.27 (q, J=11.89 Hz, 1H). The slower-eluting isomer at 4.91 min was obtained 2-[(3S,5R)-5-(2,3-dichloro-6-hydroxyphenyl)pyrrolidin-3-yl]-3-hydroxypropanamide isomer 2 as an off-white solid (16.6 mg, 7%): LCMS (ESI) calc'd for C₁₃H₁₆Cl₂N₂O₃ [M+H]⁺: 319, 321 (3:2) found 319, 321 (3:2); ¹H NMR (400 MHz, CD₃OD) δ 7.44 (d, J=8.90 Hz, 1H), 6.91 (d, J=8.89 Hz, 1H), 5.28 (dd, J=11.63, 6.84 Hz, 1H), 3.85-3.71 (m, 2H), 3.58-3.42 (m, 2H), 2.86-2.71 (m, 1H), 2.60-2.39 (m, 2H), 2.20 (q, J=11.98 Hz, 1H).

The compounds in Table 7F below were prepared in an analogous fashion to that described for Compounds 74 and 75, starting from tert-butyl (2R,4S)-2-[2,3-dichloro-6-(methoxymethoxy)phenyl]-4-(2-ethoxy-2-oxoethyl)pyrrolidine-1-carboxylate.

TABLE 7F

| Compound | | | |
|---|---|---|---|
| No. | Structure | Chemical Name | MS: (M + H)⁺ & ¹H MNR |
| 78 | 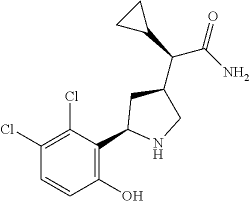 [SMILES: NC(=O)[C@H](C1CC1)[C@H]1CN[C@@H](c2c(O)ccc(Cl)c2Cl)C1] | 2-cyclopropyl-2- ((3S,5R)-5-(2,3- dichloro-6- hydroxyphenyl) pyrrolidin-3- yl)acetamide isomer 1 | [M + H]⁺: 329, 331 (3:2); ¹H NMR (300 MHz, CD₃OD) δ 7.47 (dd, J = 8.89, 0.90 Hz, 1H), 6.92 (d, J = 8.90 Hz, 1H), 5.30 (dd, J = 11.32, 7.18 Hz, 1H), 3.75 (dd, J = 11.19, 7.45 Hz, 1H), 3.50 (t, J = 11.43 Hz, 1H), 2.96-2.77 (m, 1H), 2.39-2.15 (m, 2H), 1.64 (t, J = 9.50 Hz, 1H), 1.12-0.96 (m, 1H), 0.77-0.65 (m, 1H), 0.65-0.53 (m, 1H), 0.44-0.25 (m, 2H). |
| 79 | 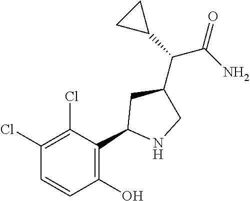 [SMILES: NC(=O)[C@@H](C1CC1)[C@H]1CN[C@@H](c2c(O)ccc(Cl)c2Cl)C1] | 2-cyclopropyl-2- ((3S,5R)-5-(2,3- dichloro-6- hydroxyphenyl) pyrrolidin-3- yl)acetamide isomer 2 | [M + H]⁺: 329, 331 (3:2); ¹H NMR (300 MHz, CD₃OD) δ 7.47 (d, J = 8.92 Hz, 1H), 6.93 (d, J = 8.88 Hz, 1H), 5.33 (dd, J = 11.64, 6.79 Hz, 1H), 3.57-3.41 (m, 2H), 2.98-2.76 (m, 1H), 2.68-2.45 (m, 1H), 2.25 (q, J = 12.03 Hz, 1H), 1.64 (t, J = 9.41 Hz, 1H), 1.14- 0.96 (m, 1H), 0.75-0.61 (m, 1H), 0.61-0.46 (m, 1H), 0.39-0.23 (m, 2H). |

### Example 21. Evaluation of Kv1.3 Potassium Channel Blocker Activities

This assay is used to evaluate the disclosed compounds' activities as Kv1.3 potassium channel blockers.

### Cell Culture

CHO-K1 cells stably expressing Kv1.3 were grown in DMEM containing 10% heat-inactivated FBS, 1 mM sodium pyruvate, 2 mM L-glutamine, and G418 (500 μg/ml). Cells were grown in culture flasks at 37° C. in a 5% C₀₂-humidified incubator.

### Solutions

The cells were bathed in an extracellular solution containing 140 mM NaCl, 4 mM KCl, 2 mM CaCl₂), 1 mM MgCl₂, 5 mM glucose, 10 mM HEPES; pH adjusted to 7.4 with NaOH; 295-305 mOsm. The internal solution contained 50 mM KCl, 10 mM NaCl, 60 mM KF, 20 mM EGTA, 10 mM HEPES; pH adjusted to 7.2 with KOH; 285 mOsm. All compounds were dissolved in DMSO at 30 mM. Compound stock solutions were freshly diluted with external solution to concentrations of 30 nM, 100 nM, 300 nM, 1 μM, 3 μM, 10 μM, 30 μM and 100 μM. The highest content of DMSO (0.3%) was present in 100 μM.

### Voltage Protocol

The currents were evoked by applying 100 ms depolarizing pulses from −90 mV (holding potential) to +40 mV were applied with 0.1 Hz frequency. Control (compound-free) and compound pulse trains for each compound concentration applied contained 20 pulses. 10-second breaks were used between pulse trains (see Table A below).

TABLE A
Voltage Protocol

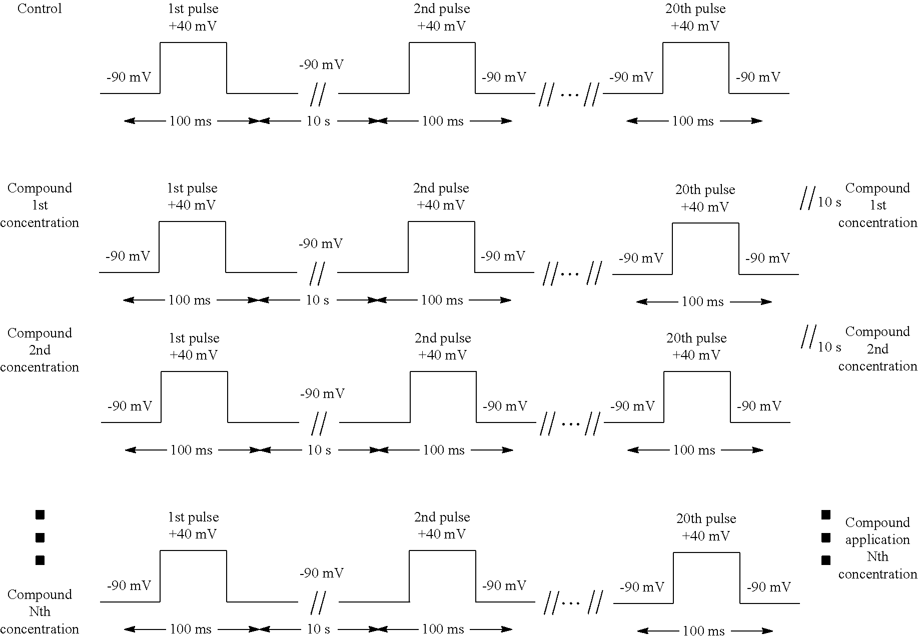

### Patch Clamp Recordings and Compound Application

Whole-cell current recordings and compound application were enabled by means of an automated patch clamp platform Patchliner (Nanion Technologies GmbH). EPC 10 patch clamp amplifier (HEKA Elektronik Dr. Schulze GmbH) along with Patchmaster software (HEKA Elektronik Dr. Schulze GmbH) was used for data acquisition. Data were sampled at 10 kHz without filtering. Passive leak currents were subtracted online using a P/4 procedure (HEKA Elektronik Dr. Schulze GmbH). Increasing compound concentrations were applied consecutively to the same cell without washouts in between. Total compound incubation time before the next pulse train was not longer than 10 seconds. Peak current inhibition was observed during compound equilibration.

### Data Analysis

AUC and peak values were obtained with Patchmaster (HEKA Elektronik Dr. Schulze GmbH). To determine IC₅₀, the last single pulse in the pulse train corresponding to a given compound concentration was used. Obtained AUC and peak values in the presence of compound were normalized to control values in the absence of compound. Using Origin (OridinLab), IC₅₀ was derived from data fit to Hill equation: I_{compound}/I_{control}=(100-A)/(1+([compound]/IC₅₀)nH)+A, where IC₅₀ value is the concentration at which current inhibition is half-maximal, [compound] is the applied compound concentration, A is the fraction of current that is not blocked and nH is the Hill coefficient.

### Example 22. Evaluation of hERG Activities

This assay is used to evaluate the disclosed compounds's inhibition activities against the hERG channel.

hERG Electrophysiology

This assay is used to evaluate the disclosed compounds' inhibition activities against the hERG channel.

### Cell Culture

CHO-K1 cells stably expressing hERG were grown in Ham's F-12 Medium with glutamine containing 10% heat-inactivated FBS, 1% penicillin/streptomycin, hygromycin (100 μg/ml) and G418 (100 μg/ml). Cells were grown in culture flasks at 37° C. in a 5% CO₂— humidified incubator.

### Solutions

The cells were bathed in an extracellular solution containing 140 mM NaCl, 4 mM KCl, 2 mM CaCl₂), 1 mM MgCl₂, 5 mM Glucose, 10 mM HEPES; pH adjusted to 7.4 with NaOH; 295-305 mOsm. The internal solution contained 50 mM KCl, 10 mM NaCl, 60 mM KF, 20 mM EGTA, 10 mM HEPES; pH adjusted to 7.2 with KOH; 285 mOsm. All compounds were dissolved in DMSO at 30 mM. Compound stock solutions were freshly diluted with external solution to concentrations of 30 nM, 100 nM, 300 nM, 1 μM, 3 μM, 10 μM, 30 μM and 100 μM. The highest content of DMSO (0.3%) was present in 100 μM.

### Voltage Protocol

The voltage protocol (see Table B) was designed to simulate voltage changes during a cardiac action potential with a 300 ms depolarization to +20 mV (analogous to the plateau phase of the cardiac action potential), a repolarization for 300 ms to −50 mV (inducing a tail current) and a final step to the holding potential of −80 mV. The pulse frequency was 0.3 Hz. Control (compound-free) and compound pulse trains for each compound concentration applied contained 70 pulses.

TABLE B
hERG voltage protocol

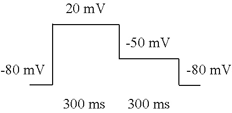

### Patch Clamp Recordings and Compound Application

Whole-cell current recordings and compound application were enabled by means of an automated patch clamp platform Patchliner (Nanion). EPC 10 patch clamp amplifier (HEKA) along with Patchmaster software (HEKA Elektronik Dr. Schulze GmbH) was used for data acquisition. Data were sampled at 10 kHz without filtering. Increasing compound concentrations were applied consecutively to the same cell without washouts in between.

### Data Analysis

AUC and PEAK values were obtained with Patchmaster (HEKA Elektronik Dr. Schulze GmbH). To determine IC₅₀ the last single pulse in the pulse train corresponding to a given compound concentration was used. Obtained AUC and PEAK values in the presence of compound were normalized to control values in the absence of compound. Using Origin (OridinLab), IC₅₀ was derived from data fit to Hill equation: I_{compound}/I_{control}=(100−A)/(1+([compound]/IC₅₀)nH)+A, where IC₅₀ is the concentration at which current inhibition is half-maximal, [compound] is the applied compound concentration, A is the fraction of current that is not blocked and nH is the Hill coefficient.

Table 7 provides a summary of the inhibition activities of certain selected compounds of the instant invention against Kv1.3 potassium channel and hERG channel.

TABLE 7
IC₅₀ (μM) values of certain exemplified compounds against Kv1.3 potassium channel
and hERG channel

| Compound | | Kv1.3 IC₅₀ | hERG |
|---|---|---|---|
| Number | Structure | (μM) | IC₅₀ (μM) |
| 31 | 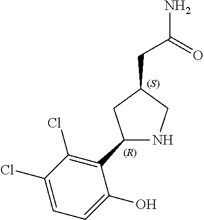 | <1 | >100 |
| 32 | 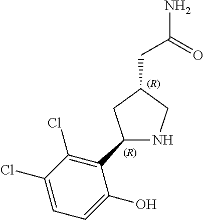 | <1 | * |
| 33 | 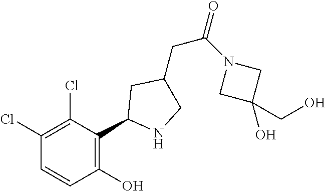 [SMILES: O=C(CC1CN[C@@H](c2c(O)ccc(Cl)c2Cl)C1)N1CC(O)(CO)C1] | <0.1 | >100 |
| 34 | 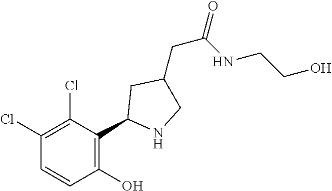 [SMILES: O=C(CC1CN[C@@H](c2c(O)ccc(Cl)c2Cl)C1)NCCO] | <0.1 | >100 |
| 35 | 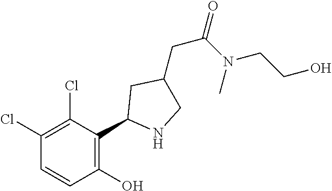 [SMILES: CN(CCO)C(=O)CC1CN[C@@H](c2c(O)ccc(Cl)c2Cl)C1] | <0.1 | >30 |
| 36 | 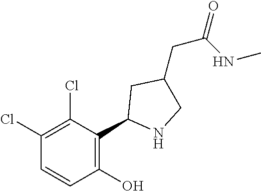 [SMILES: CNC(=O)CC1CN[C@@H](c2c(O)ccc(Cl)c2Cl)C1] | <0.1 | >30 |
| 37 | 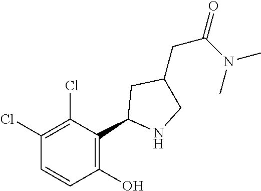 [SMILES: CN(C)C(=O)CC1CN[C@@H](c2c(O)ccc(Cl)c2Cl)C1] | <1 | >30 |
| 38 | 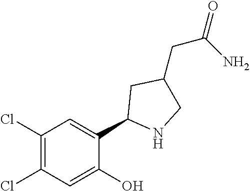 [SMILES: NC(=O)CC1CN[C@@H](c2cc(Cl)c(Cl)cc2O)C1] | <1 | >10 |
| 39 | 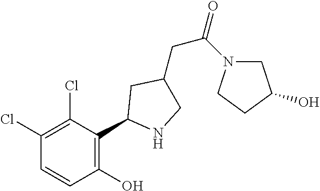 [SMILES: O=C(CC1CN[C@@H](c2c(O)ccc(Cl)c2Cl)C1)N1CC[C@@H](O)C1] | <0.1 | >100 |
| 40 | 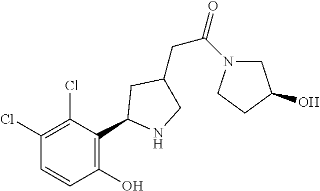 [SMILES: O=C(CC1CN[C@@H](c2c(O)ccc(Cl)c2Cl)C1)N1CC[C@H](O)C1] | <1 | >100 |
| 41 | 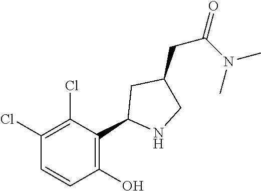 [SMILES: CN(C)C(=O)C[C@H]1CN[C@@H](c2c(O)ccc(Cl)c2Cl)C1] | <1 | >30 |
| 42 | 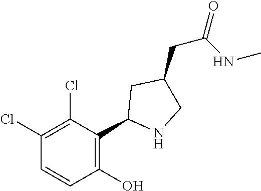 [SMILES: CNC(=O)C[C@H]1CN[C@@H](c2c(O)ccc(Cl)c2Cl)C1] | <0.1 | >30 |
| 43 | 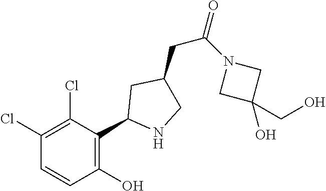 [SMILES: O=C(C[C@H]1CN[C@@H](c2c(O)ccc(Cl)c2Cl)C1)N1CC(O)(CO)C1] | <0.1 | >100 |
| 44 | 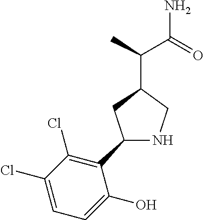 [SMILES: C[C@@H](C(N)=O)[C@H]1CN[C@@H](c2c(O)ccc(Cl)c2Cl)C1] | <1 | >100 |
| 45 | 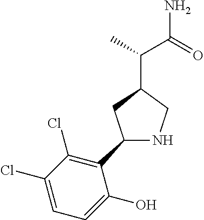 [SMILES: C[C@H](C(N)=O)[C@H]1CN[C@@H](c2c(O)ccc(Cl)c2Cl)C1] | <0.1 | >100 |
| 46 | 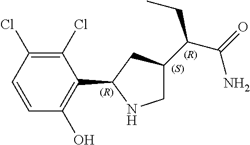 | <1 | >100 |
| 47 | 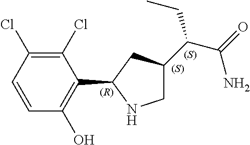 | <0.1 | 100 |
| 48 | 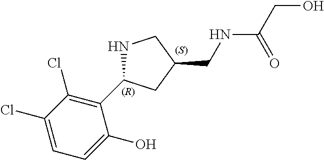 | <1 | >30 |
| 49 | 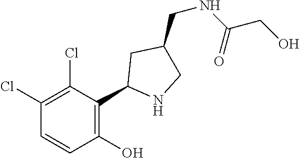 [SMILES: O=C(CO)NC[C@H]1CN[C@@H](c2c(O)ccc(Cl)c2Cl)C1] | <1 | >100 |
| 50 | 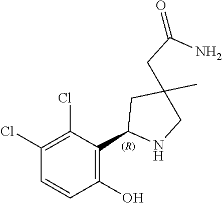 | <1 | * |
| 51 | 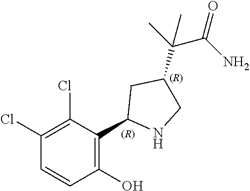 | <1 | * |
| 52 | 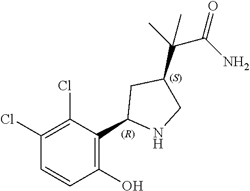 | <1 | >30 |
| 53 | 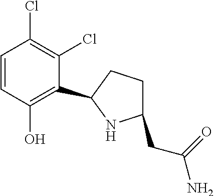 [SMILES: NC(=O)C[C@@H]1CC[C@H](c2c(O)ccc(Cl)c2Cl)N1] | <1 | >100 |
| 54 | 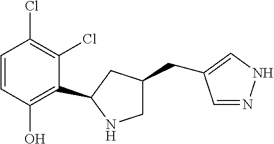 [SMILES: Oc1ccc(Cl)c(Cl)c1[C@H]1C[C@@H](Cc2cn[nH]c2)CN1] | <0.1 | >10 |
| 55 | 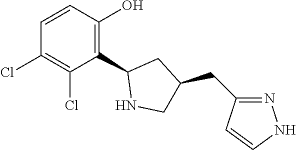 [SMILES: Oc1ccc(Cl)c(Cl)c1[C@H]1C[C@@H](Cc2cc[nH]n2)CN1] | <1 | >10 |
| 56 | 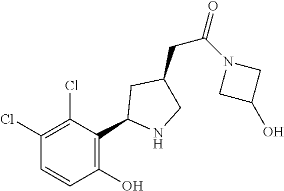 [SMILES: O=C(C[C@H]1CN[C@@H](c2c(O)ccc(Cl)c2Cl)C1)N1CC(O)C1] | <0.1 | >100 |
| 57 | 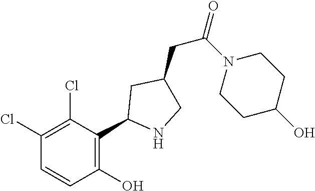 [SMILES: O=C(C[C@H]1CN[C@@H](c2c(O)ccc(Cl)c2Cl)C1)N1CCC(O)CC1] | <1 | * |
| 58 | 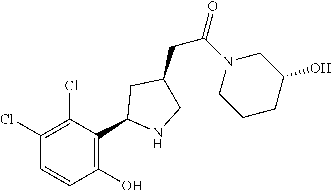 [SMILES: O=C(C[C@H]1CN[C@@H](c2c(O)ccc(Cl)c2Cl)C1)N1CCC[C@@H](O)C1] | <1 | >30 |
| 59 | 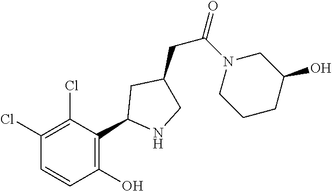 [SMILES: O=C(C[C@H]1CN[C@@H](c2c(O)ccc(Cl)c2Cl)C1)N1CCC[C@H](O)C1] | <1 | * |
| 60 | 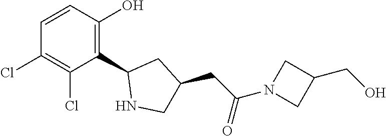 [SMILES: O=C(C[C@H]1CN[C@@H](c2c(O)ccc(Cl)c2Cl)C1)N1CC(CO)C1] | <1 | * |
| 61 | 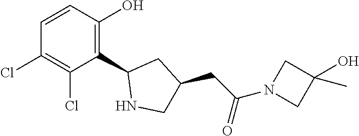 [SMILES: CC1(O)CN(C(=O)C[C@H]2CN[C@@H](c3c(O)ccc(Cl)c3Cl)C2)C1] | <0.1 | >100 |
| 62 | 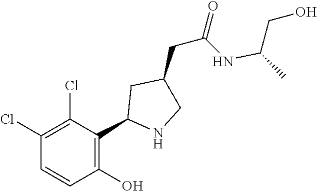 [SMILES: C[C@@H](CO)NC(=O)C[C@H]1CN[C@@H](c2c(O)ccc(Cl)c2Cl)C1] | <1 | * |
| 63 | 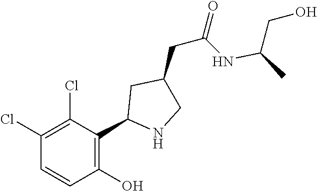 [SMILES: C[C@H](CO)NC(=O)C[C@H]1CN[C@@H](c2c(O)ccc(Cl)c2Cl)C1] | <0.1 | >100 |
| 64 | 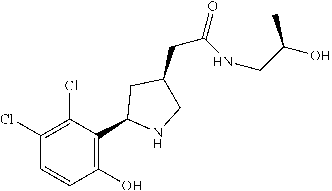 [SMILES: C[C@@H](O)CNC(=O)C[C@H]1CN[C@@H](c2c(O)ccc(Cl)c2Cl)C1] | <0.1 | >100 |
| 65 | 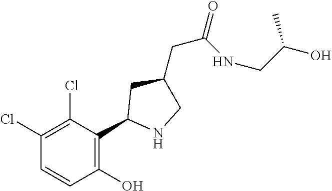 [SMILES: C[C@H](O)CNC(=O)C[C@H]1CN[C@@H](c2c(O)ccc(Cl)c2Cl)C1] | <0.1 | >100 |
| 66 | 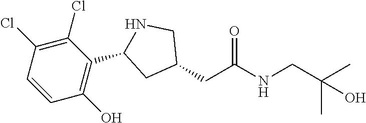 [SMILES: CC(C)(O)CNC(=O)C[C@H]1CN[C@@H](c2c(O)ccc(Cl)c2Cl)C1] | <1 | >100 |
| 67 | 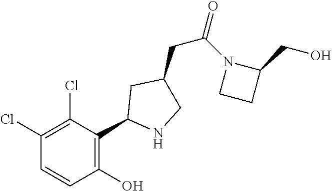 [SMILES: O=C(C[C@H]1CN[C@@H](c2c(O)ccc(Cl)c2Cl)C1)N1CC[C@@H]1CO] | <1 | >100 |
| 68 | 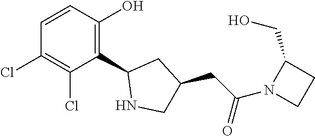 [SMILES: O=C(C[C@H]1CN[C@@H](c2c(O)ccc(Cl)c2Cl)C1)N1CC[C@H]1CO] | <1 | >100 |
| 69 | 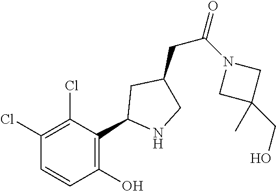 [SMILES: CC1(CO)CN(C(=O)C[C@H]2CN[C@@H](c3c(O)ccc(Cl)c3Cl)C2)C1] | <1 | * |
| 70 | 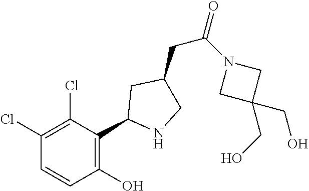 [SMILES: O=C(C[C@H]1CN[C@@H](c2c(O)ccc(Cl)c2Cl)C1)N1CC(CO)(CO)C1] | <1 | * |
| 71 | 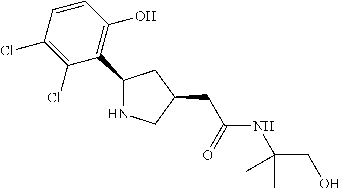 [SMILES: CC(C)(CO)NC(=O)C[C@H]1CN[C@@H](c2c(O)ccc(Cl)c2Cl)C1] | <1 | >100 |
| 72 | 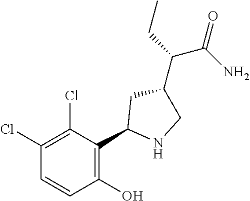 [SMILES: CC[C@H](C(N)=O)[C@@H]1CN[C@@H](c2c(O)ccc(Cl)c2Cl)C1] | <1 | * |
| 73 | 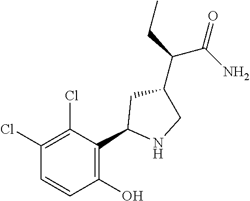 [SMILES: CC[C@@H](C(N)=O)[C@@H]1CN[C@@H](c2c(O)ccc(Cl)c2Cl)C1] | <1 | * |
| 74 | 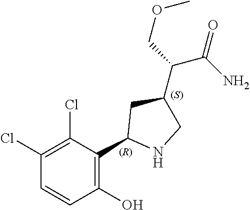 | <1 | >100 |
| 75 | 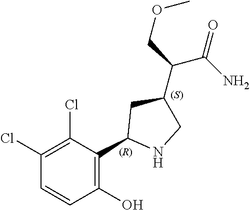 | <1 | >100 |
| 76 | 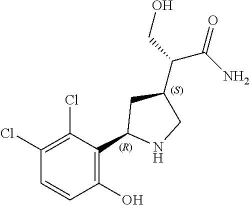 | <1 | >100 |
| 77 | 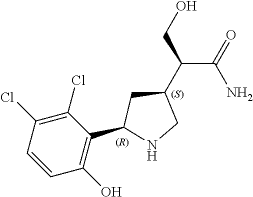 | <0.1 | >100 |
| 78 | 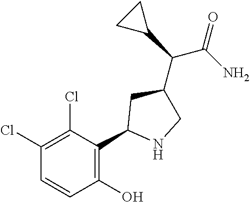 [SMILES: NC(=O)[C@H](C1CC1)[C@H]1CN[C@@H](c2c(O)ccc(Cl)c2Cl)C1] | <0.1 | * |
| 79 | 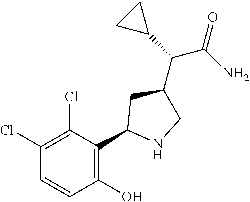 [SMILES: NC(=O)[C@@H](C1CC1)[C@H]1CN[C@@H](c2c(O)ccc(Cl)c2Cl)C1] | <1 | * |

*Not Tested.

We claim:

1. A compound of Formula I, I′, II, II′, III, or IV, or a pharmaceutically-acceptable salt thereof, 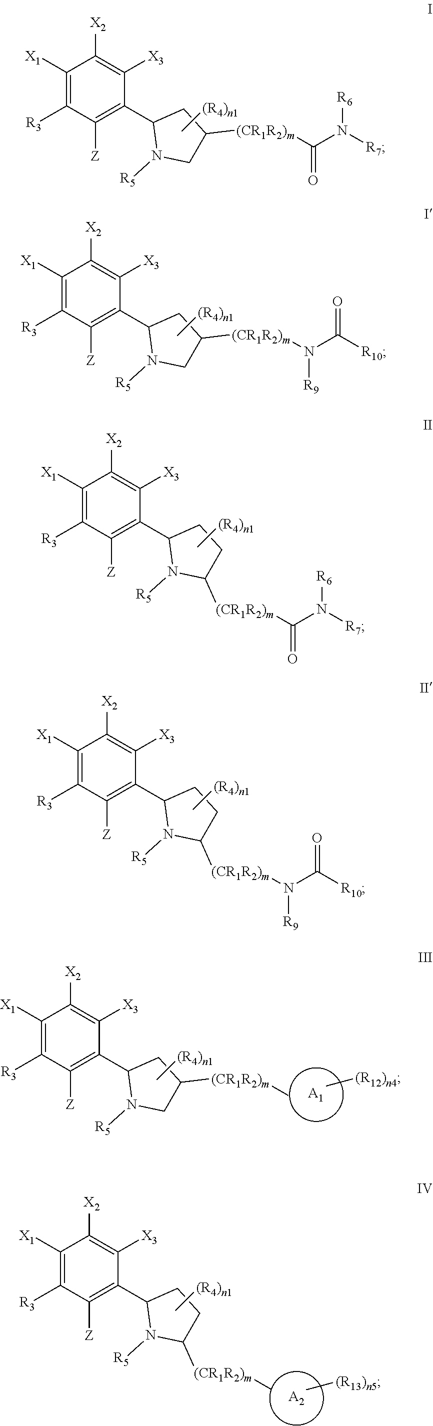
wherein
each occurrence of Z is independently ORₐ;
each occurrence of X₁ is independently H, halogen, CN, alkyl, cycloalkyl, halogenated cycloalkyl, or halogenated alkyl;
each occurrence of X₂ is independently H, halogen, CN, alkyl, cycloalkyl, halogenated cycloalkyl, or halogenated alkyl;
each occurrence of X₃ is independently H, halogen, CN, alkyl, cycloalkyl, halogenated cycloalkyl, or halogenated alkyl;
or alternatively X₁ and X₂ and the carbon atoms they are connected to taken together form an optionally substituted 5- or 6-membered aryl;
or alternatively X₂ and X₃ and the carbon atoms they are connected to taken together form an optionally substituted 5- or 6-membered aryl;
each occurrence of R₁ is independently H, alkyl, cycloalkyl, saturated heterocycle, aryl, heteroaryl, alkylaryl, alkylheteroaryl, CN, CF₃, OCF₃, ORₐ, SRₐ, halogen, NRₐR_{b}, or NR_{b}(C═O)Rₐ;
each occurrence of R₂ is independently H, alkyl, cycloalkyl, saturated heterocycle, aryl, heteroaryl, alkylaryl, alkylheteroaryl, CN, CF₃, OCF₃, ORₐ, SRₐ, halogen, NRₐR_{b}, or NR_{b}(C═O)Rₐ;
or alternatively R₁ and R₂ taken together with the carbon atom they are connected to form a cycloalkyl or saturated heterocycle;
each occurrence of R₃ is independently H, alkyl, cycloalkyl, saturated heterocycle, aryl, heteroaryl, CN, CF₃, OCF₃, ORₐ, SRₐ, halogen, NRₐR_{b}, or NR_{b}(C═O)Rₐ;
each occurrence of R₄ is independently H, alkyl, cycloalkyl, saturated heterocycle, (CRₐR_{b})ₙ₂ORₐ, or (CRₐR_{b})ₙ₂NRₐR_{b};
or alternatively two R₄ groups taken together with the carbon atom(s) that they are connected to form a 3-7 membered optionally substituted cycloalkyl or heterocycle;
each occurrence of R₅ is independently H, alkyl, cycloalkyl, saturated heterocycle, aryl, heteroaryl, alkylaryl, alkylheteroaryl, (C═O)Rₐ, (C═O)(CRₐR_{b})ₙ₂ORₐ, (C═O)(CRₐR_{b})ₙ₂NRₐR_{b}, or SO₂Rₐ;
each occurrence of R₆ is independently H, alkyl, cycloalkyl, heterocycle, aryl, heteroaryl, alkylaryl, or alkylheteroaryl;
each occurrence of R₇ is independently H, alkyl, cycloalkyl, heterocycle, aryl, heteroaryl, alkylaryl, or alkylheteroaryl;
or alternatively R₆ and R₇ taken together with the nitrogen atom they are connected to form a heterocycle comprising the nitrogen atom and 0-3 additional heteroatoms each selected from the group consisting of N, O, and S; wherein the heterocycle is optionally substituted by 1-4 substituents each independently selected from the group consisting of alkyl, cycloalkyl, halogenated cycloalkyl, halogenated alkyl, halogen, CN, OR₈, —(CH₂)₀₋₂OR₈, N(R₈)₂, (C═O)R₈, (C═O)N(R₈)₂, NR₈(C═O)R₈, and oxo where valence permits;
each occurrence of R₉ is independently H, alkyl, cycloalkyl, saturated heterocycle, aryl, heteroaryl, alkylaryl, alkylheteroaryl, (C═O)Rₐ, (C═O)(CRₐR_{b})ₙ₂ORₐ, (C═O)(CRₐR_{b})ₙ₂NRₐR_{b}, or SO₂Rₐ;
each occurrence of R₁₀ is independently H, alkyl, cycloalkyl, heterocycle, aryl, heteroaryl, alkylaryl, or alkylheteroaryl;
A₁ is aryl or heteroaryl;
A₂ is aryl or heteroaryl;
each occurrence of R₁₂ is independently H, alkyl, CN, CF₃, OCF₃, ORₐ, SRₐ, halogen, NRₐR_{b}, (CRₐR_{b})ₙ₂ORₐ, (C═O)NRₐR_{b}, (CRₐR_{b})ₙ₂NRₐR_{b}, or (CRₐR_{b})ₙ₂NR_{b}(C═O)Rₐ;
each occurrence of R₁₃ is independently H, alkyl, CN, CF₃, OCF₃, ORₐ, SRₐ, halogen, NRₐR_{b}, (CRₐR_{b})ₙ₂ORₐ, (C═O)NRₐR_{b}, (CRₐR_{b})ₙ₂NRₐR_{b}, or (CRₐR_{b})ₙ₂NR_{b}(C═O)Rₐ;
each occurrence of Rₐ and R_{b} are independently H, alkyl, alkenyl, cycloalkyl, saturated heterocycle comprising 1-3 heteroatoms each selected from the group consisting of N, O, and S, aryl, or heteroaryl; or alternatively Rₐ and R_{b} together with the carbon or nitrogen atom that they are connected to form a cycloalkyl or heterocycle comprising the nitrogen atom and 0-3 additional heteroatoms each selected from the group consisting of N, O, and S;
the alkyl, cycloalkyl, heterocycle, aryl, and heteroaryl in X₁, X₂, X₃, A₁, A₂, R₁, R₂, R₃, R₄, R₅, R₆, R₇, R₉, R₁₀, R₁₂, R₁₃, Rₐ, or R_{b}, where applicable, are optionally substituted by 1-4 substituents each independently selected from the group consisting of alkyl, cycloalkyl, halogenated cycloalkyl, halogenated alkyl, halogen, CN, OR₈, —(CH₂)₀₋₂OR₈, N(R₈)₂, (C═O)R₈, (C═O)N(R₈)₂, NR₈(C═O)R₈, and oxo where valence permits;
each occurrence of R₈ is independently H, alkyl, or optionally substituted heterocycle wherein the heterocycle is optionally substituted by alkyl, halogen, or OH; or alternatively the two R₈ groups together with the nitrogen atom that they are connected to form an optionally substituted heterocycle, wherein the heterocycle is optionally substituted by alkyl, halogen, or OH, comprising the nitrogen atom and 0-3 additional heteroatoms each selected from the group consisting of N, O, and S;
each occurrence of m is independently 1, 2, or 3;
each occurrence of n₁ is independently an integer from 0-3 wherein valence permits;
each occurrence of n₂ is independently an integer from 0-3;
n₄ is an integer from 0-3; and
n₅ is an integer from 0-3.

2. (canceled)

3. (canceled)

4. (canceled)

5. The compound of claim 1, or a pharmaceutically-acceptable salt thereof, wherein the compound has the structure of Formula Ia, Ia′, IIa, IIa′, IIIa, or IVa:

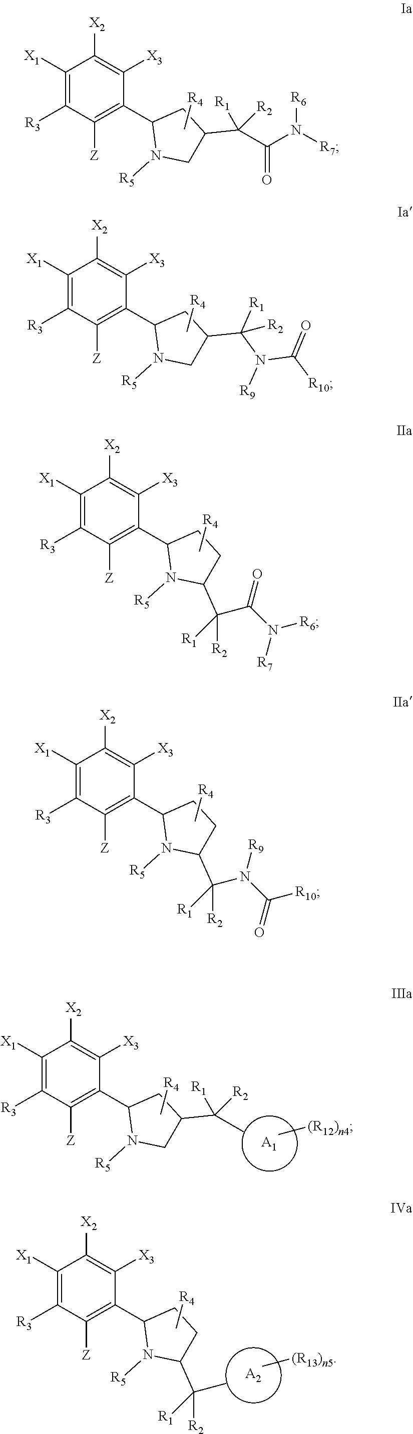

6. The compound of claim 1, or a pharmaceutically-acceptable salt thereof, wherein the compound has the structure of Formula Ib, Ib′, IIb, IIb′, IIIb, or IVb:

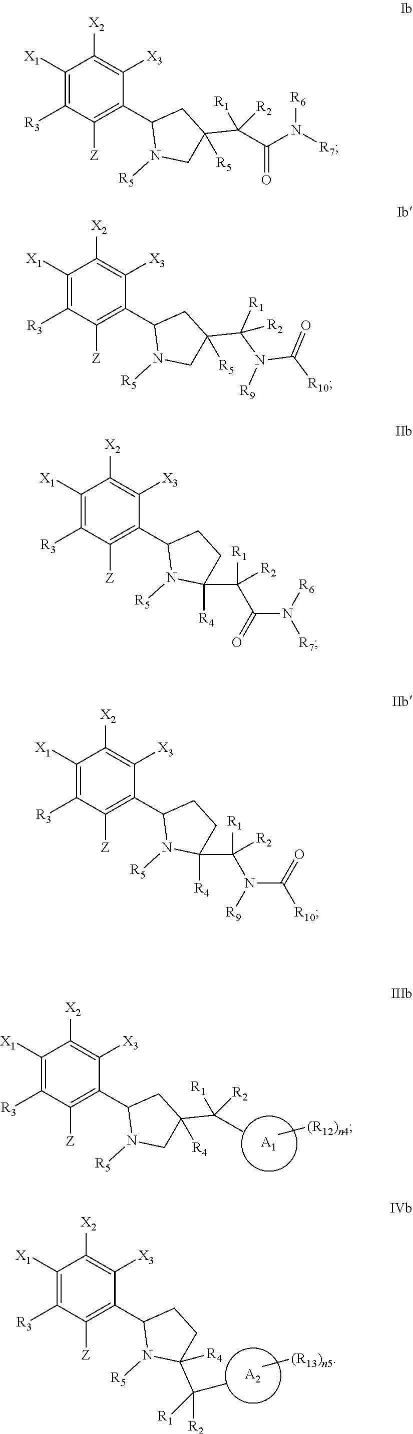

7. (canceled)

8. (canceled)

9. The compound of claim 1, or a pharmaceutically-acceptable salt thereof, wherein one or more occurrences of R₄ are H or CH₃.

10. (canceled)

11. (canceled)

12. (canceled)

13. (canceled)

14. (canceled)

15. (canceled)

16. The compound of claim 1, or a pharmaceutically acceptable salt thereof, wherein each occurrence of R₅ is independently H or CH₃.

17. (canceled)

18. (canceled)

19. (canceled)

20. The compound of claim 1, or a pharmaceutically acceptable salt thereof, wherein R₁ and R₂ are H and H, H and Me, Me and Me, H and Et, Me and Et, Et and Et, H and CH₂OH, H and CH₂CH₂OH, H and CH₂OCH₃, H and CH₂CH₂OCH₃, or H and

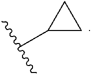 [SMILES: *C1CC1]

21. (canceled)

22. (canceled)

23. (canceled)

24. The compound of claim 1, or a pharmaceutically acceptable salt thereof, wherein each occurrence of R₆ and R₇ is independently H, —CH₃, —CH₂OH, —CH₂CH₂OH or —CH₂CH₂CH₂OH.

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. The compound of claim 1, or a pharmaceutically acceptable salt thereof, wherein each occurrence of the structural moiety 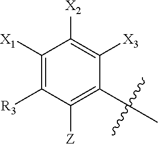
independently has the structure of

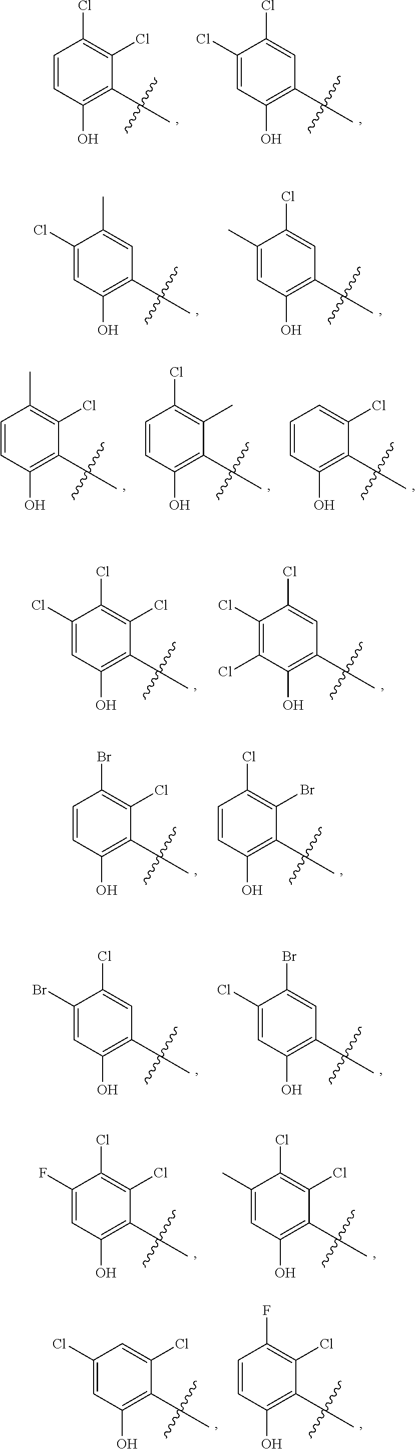

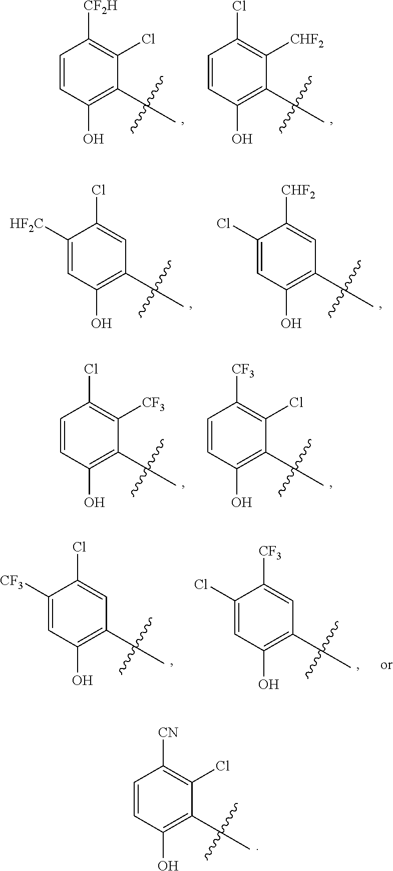

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. The compound of claim 1, wherein the compound is selected from the group consisting of: 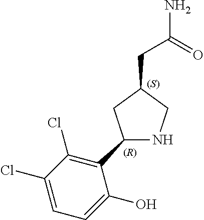 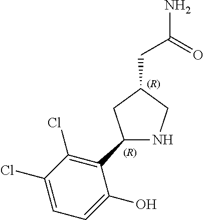 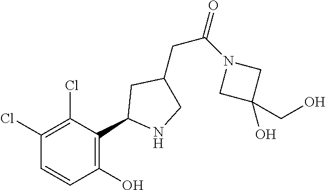 [SMILES: O=C(CC1CN[C@@H](c2c(O)ccc(Cl)c2Cl)C1)N1CC(O)(CO)C1] 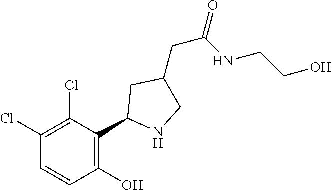 [SMILES: O=C(CC1CN[C@@H](c2c(O)ccc(Cl)c2Cl)C1)NCCO] 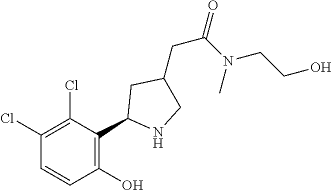 [SMILES: CN(CCO)C(=O)CC1CN[C@@H](c2c(O)ccc(Cl)c2Cl)C1] 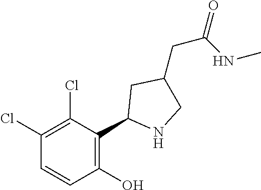 [SMILES: CNC(=O)CC1CN[C@@H](c2c(O)ccc(Cl)c2Cl)C1] 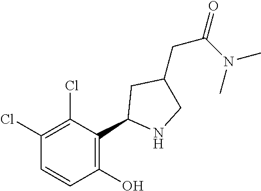 [SMILES: CN(C)C(=O)CC1CN[C@@H](c2c(O)ccc(Cl)c2Cl)C1] 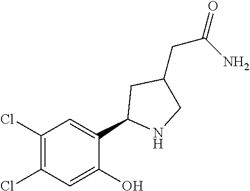 [SMILES: NC(=O)CC1CN[C@@H](c2cc(Cl)c(Cl)cc2O)C1] 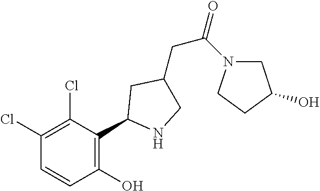 [SMILES: O=C(CC1CN[C@@H](c2c(O)ccc(Cl)c2Cl)C1)N1CC[C@@H](O)C1] 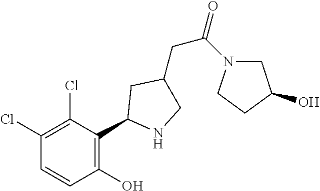 [SMILES: O=C(CC1CN[C@@H](c2c(O)ccc(Cl)c2Cl)C1)N1CC[C@H](O)C1] 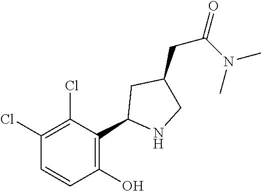 [SMILES: CN(C)C(=O)C[C@H]1CN[C@@H](c2c(O)ccc(Cl)c2Cl)C1] 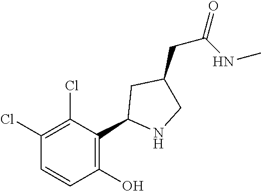 [SMILES: CNC(=O)C[C@H]1CN[C@@H](c2c(O)ccc(Cl)c2Cl)C1] 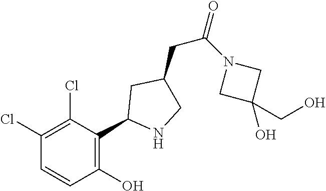 [SMILES: O=C(C[C@H]1CN[C@@H](c2c(O)ccc(Cl)c2Cl)C1)N1CC(O)(CO)C1] 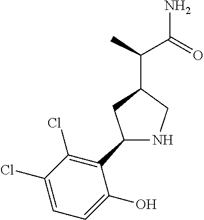 [SMILES: C[C@@H](C(N)=O)[C@H]1CN[C@@H](c2c(O)ccc(Cl)c2Cl)C1] 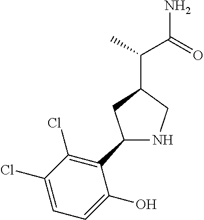 [SMILES: C[C@H](C(N)=O)[C@H]1CN[C@@H](c2c(O)ccc(Cl)c2Cl)C1] 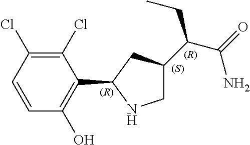 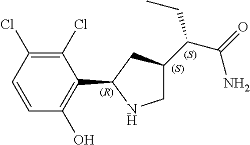 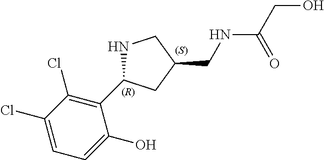 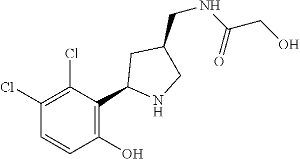 [SMILES: O=C(CO)NC[C@H]1CN[C@@H](c2c(O)ccc(Cl)c2Cl)C1] 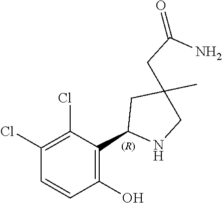 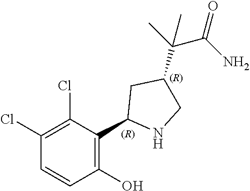 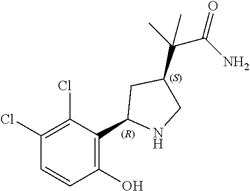 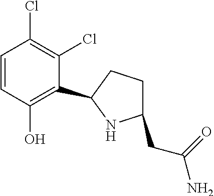 [SMILES: NC(=O)C[C@@H]1CC[C@H](c2c(O)ccc(Cl)c2Cl)N1] 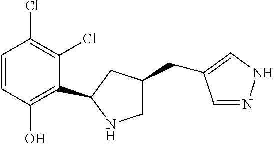 [SMILES: Oc1ccc(Cl)c(Cl)c1[C@H]1C[C@@H](Cc2cn[nH]c2)CN1] 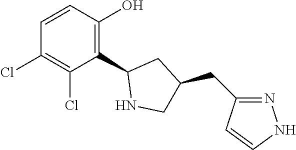 [SMILES: Oc1ccc(Cl)c(Cl)c1[C@H]1C[C@@H](Cc2cc[nH]n2)CN1] 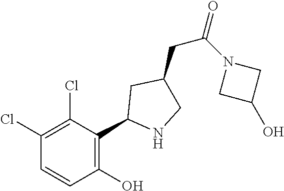 [SMILES: O=C(C[C@H]1CN[C@@H](c2c(O)ccc(Cl)c2Cl)C1)N1CC(O)C1] 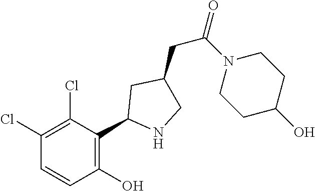 [SMILES: O=C(C[C@H]1CN[C@@H](c2c(O)ccc(Cl)c2Cl)C1)N1CCC(O)CC1] 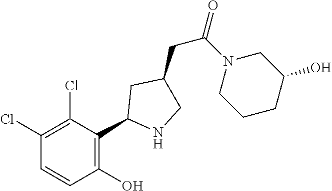 [SMILES: O=C(C[C@H]1CN[C@@H](c2c(O)ccc(Cl)c2Cl)C1)N1CCC[C@@H](O)C1] 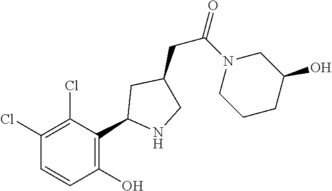 [SMILES: O=C(C[C@H]1CN[C@@H](c2c(O)ccc(Cl)c2Cl)C1)N1CCC[C@H](O)C1] 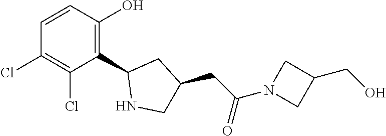 [SMILES: O=C(C[C@H]1CN[C@@H](c2c(O)ccc(Cl)c2Cl)C1)N1CC(CO)C1] 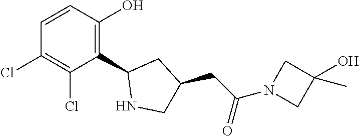 [SMILES: CC1(O)CN(C(=O)C[C@H]2CN[C@@H](c3c(O)ccc(Cl)c3Cl)C2)C1] 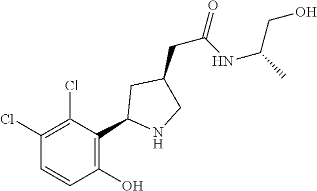 [SMILES: C[C@@H](CO)NC(=O)C[C@H]1CN[C@@H](c2c(O)ccc(Cl)c2Cl)C1] 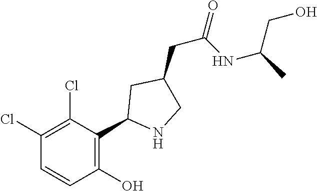 [SMILES: C[C@H](CO)NC(=O)C[C@H]1CN[C@@H](c2c(O)ccc(Cl)c2Cl)C1] 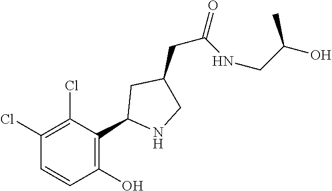 [SMILES: C[C@@H](O)CNC(=O)C[C@H]1CN[C@@H](c2c(O)ccc(Cl)c2Cl)C1] 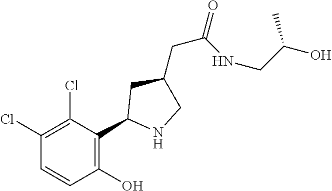 [SMILES: C[C@H](O)CNC(=O)C[C@H]1CN[C@@H](c2c(O)ccc(Cl)c2Cl)C1] 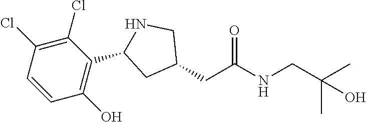 [SMILES: CC(C)(O)CNC(=O)C[C@H]1CN[C@@H](c2c(O)ccc(Cl)c2Cl)C1] 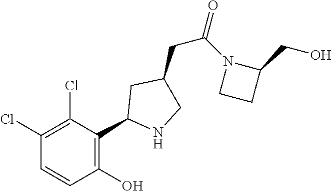 [SMILES: O=C(C[C@H]1CN[C@@H](c2c(O)ccc(Cl)c2Cl)C1)N1CC[C@@H]1CO] 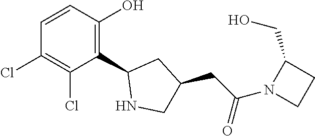 [SMILES: O=C(C[C@H]1CN[C@@H](c2c(O)ccc(Cl)c2Cl)C1)N1CC[C@H]1CO] 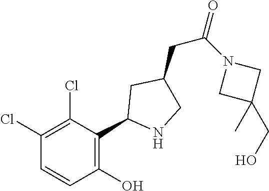 [SMILES: CC1(CO)CN(C(=O)C[C@H]2CN[C@@H](c3c(O)ccc(Cl)c3Cl)C2)C1] 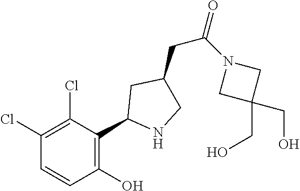 [SMILES: O=C(C[C@H]1CN[C@@H](c2c(O)ccc(Cl)c2Cl)C1)N1CC(CO)(CO)C1] 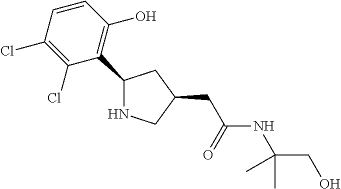 [SMILES: CC(C)(CO)NC(=O)C[C@H]1CN[C@@H](c2c(O)ccc(Cl)c2Cl)C1] 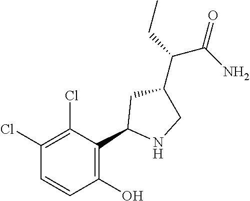 [SMILES: CC[C@H](C(N)=O)[C@@H]1CN[C@@H](c2c(O)ccc(Cl)c2Cl)C1] 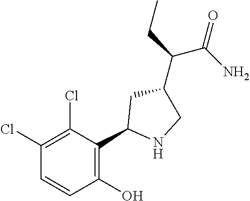 [SMILES: CC[C@@H](C(N)=O)[C@@H]1CN[C@@H](c2c(O)ccc(Cl)c2Cl)C1] 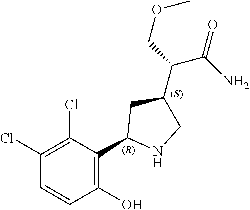 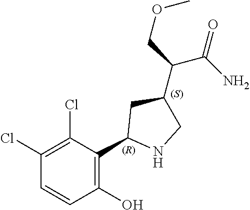 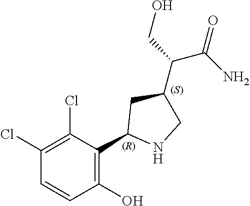 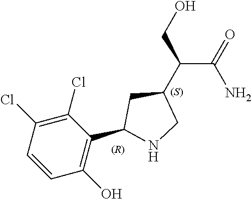 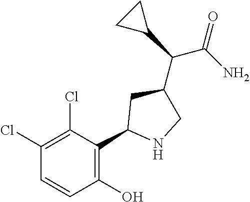 [SMILES: NC(=O)[C@H](C1CC1)[C@H]1CN[C@@H](c2c(O)ccc(Cl)c2Cl)C1] 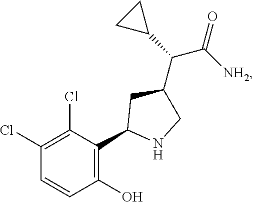 [SMILES: NC(=O)[C@@H](C1CC1)[C@H]1CN[C@@H](c2c(O)ccc(Cl)c2Cl)C1]
or a pharmaceutically-acceptable salt thereof.

84. A compound selected from the group consisting of compounds 1-15 as shown in Table 1, compounds 16-30 as shown in Table 2, compounds 1a-15a as shown in Table 3, compounds 16a-30a as shown in Table 4, compounds 1b-15b as shown in Table 5, and compounds 16b-30b as shown in Table 6:
TABLE 1

| No. | 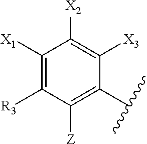 | 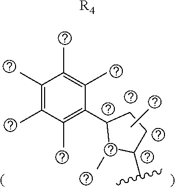 | R₅ | R [symbol] | R₂ | m | 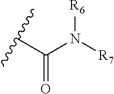 |
|---|---|---|---|---|---|---|---|
| 1 | 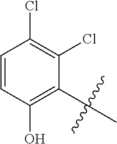 [SMILES: *c1c(O)ccc(Cl)c1Cl] | Me (4-position) | Me | OCH₂CH₃ | Et | 1 | 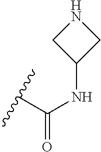 [SMILES: *C(=O)NC1CNC1] |
| 2 | 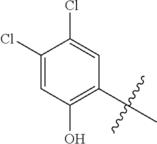 [SMILES: *c1cc(Cl)c(Cl)cc1O] | 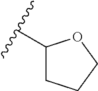 [SMILES: *C1CCCO1] (4-position) | 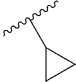 [SMILES: *C1CC1] | Et | Me | 2 | 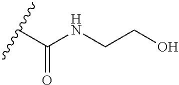 [SMILES: *C(=O)NCCO] |
| 3 | 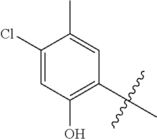 [SMILES: *c1cc(C)c(Cl)cc1O] | 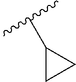 [SMILES: *C1CC1] (4-position) | 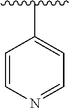 [SMILES: *c1ccncc1] | OCF₃ | Me | 3 | 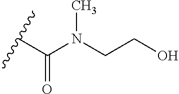 [SMILES: *C(=O)N(C)CCO] |
| 4 | 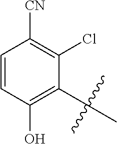 [SMILES: *c1c(O)ccc(C#N)c1Cl] | OH (4-position) | 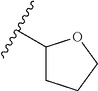 [SMILES: *C1CCCO1] | CN | H | 1 | 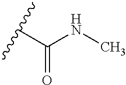 [SMILES: *C(=O)NC] |
| 5 | 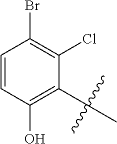 [SMILES: *c1c(O)ccc(Br)c1Cl] | CH₂CH₂OH (4-position) | 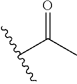 [SMILES: *C(C)=O] | SCH₂CH₃ | CF₃ | 2 | 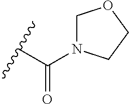 [SMILES: *C(=O)N1CCOC1] |
| 6 | 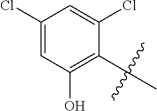 [SMILES: *c1c(O)cc(Cl)cc1Cl] | CH₂CH₂OCH₃ (4-position) | (C═O)CH₂OCH₃ | Cl | H | 3 | 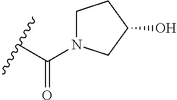 [SMILES: *C(=O)N1CC[C@H](O)C1] |
| 7 | 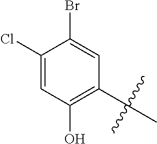 [SMILES: *c1cc(Br)c(Cl)cc1O] | 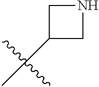 [SMILES: *C1CNC1] (4-position) | (C═O)CH [symbol] NHCH [symbol] | NH [symbol] | Me | 1 | 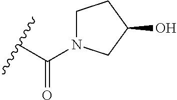 [SMILES: *C(=O)N1CC[C@@H](O)C1] |
| 8 | 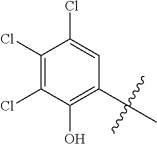 [SMILES: *c1cc(Cl)c(Cl)c(Cl)c1O] | 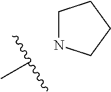 (4-position) | SO [symbol] CH₃ | NH(C═O)CH₃ | H | 2 | 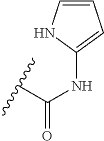 [SMILES: *C(=O)Nc1ccc[nH]1] |
| 9 | 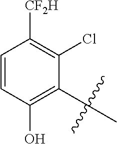 [SMILES: *c1c(O)ccc(C(F)F)c1Cl] | 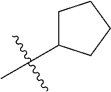 [SMILES: *C1CCCC1] (4-position) | 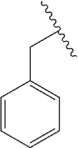 [SMILES: *Cc1ccccc1] | 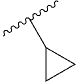 [SMILES: *C1CC1] | H | 3 | 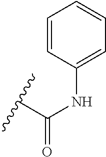 [SMILES: *C(=O)Nc1ccccc1] |
| 10 | 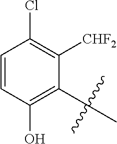 [SMILES: *c1c(O)ccc(Cl)c1C(F)F] | CH₂OCH₃ (4-position) | 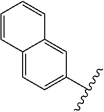 [SMILES: *c1ccc2ccccc2c1] | 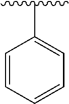 [SMILES: *c1ccccc1] | H | 1 | 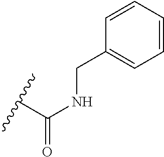 [SMILES: *C(=O)NCc1ccccc1] |
| 11 | 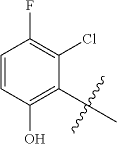 [SMILES: *c1c(O)ccc(F)c1Cl] | CH₂NHCH₃ (4-position) | 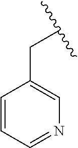 [SMILES: *Cc1cccnc1] | 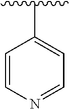 [SMILES: *c1ccncc1] | H | 2 | 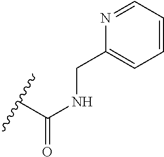 [SMILES: *C(=O)NCc1ccccn1] |
| 12 | 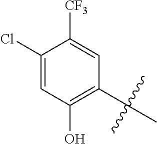 [SMILES: *c1cc(C(F)(F)F)c(Cl)cc1O] | Et (2-position) | CF₃ | 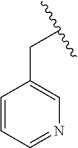 [SMILES: *Cc1cccnc1] | H | 3 | 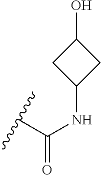 [SMILES: *C(=O)NC1CC(O)C1] |
| 13 | 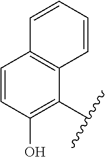 [SMILES: *c1c(O)ccc2ccccc12] | i-Pr (3-position) | CH₂CH₂OH | F | F | 1 | 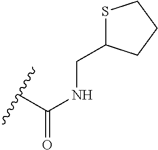 [SMILES: *C(=O)NCC1CCCS1] |
| 14 | 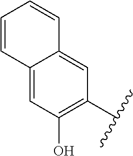 [SMILES: *c1cc2ccccc2cc1O] | Me (5-position) | (C═O)Et | 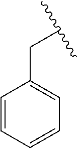 [SMILES: *Cc1ccccc1] | H | 2 | 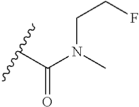 [SMILES: *C(=O)N(C)CCF] |
| 15 | 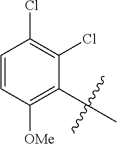 [SMILES: *c1c(OC)ccc(Cl)c1Cl] | Et (5-position) | Et | CH₂OH | H | 3 | 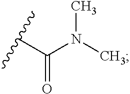 [SMILES: *C(=O)N(C)C] |

[symbol]

indicates data missing or illegible when filed
TABLE 2

| No. | 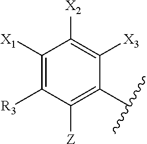 | 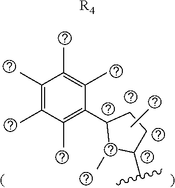 | R₅ | R [symbol] | R₂ | m | 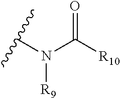 |
|---|---|---|---|---|---|---|---|
| 16 | 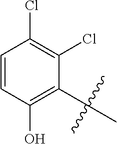 [SMILES: *c1c(O)ccc(Cl)c1Cl] | Me (4-position) | Me | OCH₂CH₃ | Et | 1 | 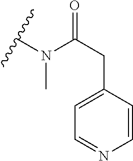 [SMILES: *N(C)C(=O)Cc1ccncc1] |
| 17 | 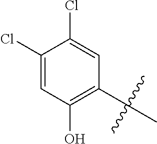 [SMILES: *c1cc(Cl)c(Cl)cc1O] | 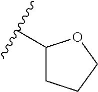 [SMILES: *C1CCCO1] (4-position) | 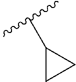 [SMILES: *C1CC1] | Et | Me | 2 | 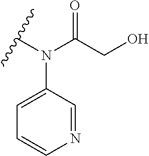 [SMILES: *N(C(=O)CO)c1cccnc1] |
| 18 | 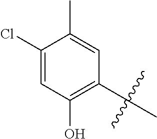 [SMILES: *c1cc(C)c(Cl)cc1O] | 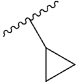 [SMILES: *C1CC1] (4-position) | 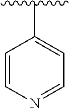 [SMILES: *c1ccncc1] | OCF₃ | Me | 3 | 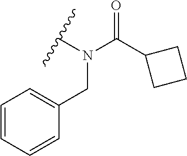 [SMILES: *N(Cc1ccccc1)C(=O)C1CCC1] |
| 19 | 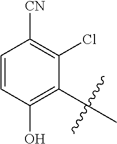 [SMILES: *c1c(O)ccc(C#N)c1Cl] | OH (4-position) | 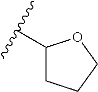 [SMILES: *C1CCCO1] | CN | H | 1 | 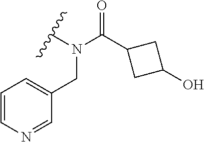 [SMILES: *N(Cc1cccnc1)C(=O)C1CC(O)C1] |
| 20 | 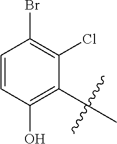 [SMILES: *c1c(O)ccc(Br)c1Cl] | CH₂CH₂OH (4-position) | 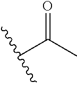 [SMILES: *C(C)=O] | SCH₂CH₃ | CF₃ | 2 | 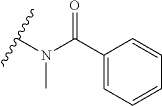 [SMILES: *N(C)C(=O)c1ccccc1] |
| 21 | 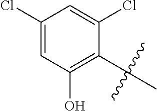 [SMILES: *c1c(O)cc(Cl)cc1Cl] | CH₂CH₂OCH [symbol] (4-position) | (C═O)CH₂OCH₃ | Cl | H | 3 | 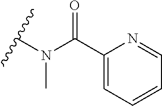 [SMILES: *N(C)C(=O)c1ccccn1] |
| 22 | 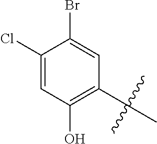 [SMILES: *c1cc(Br)c(Cl)cc1O] | 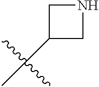 [SMILES: *C1CNC1] (4-position) | (C═O)CH₂NHCH [symbol] | NH [symbol] | Me | 1 | 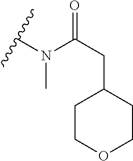 [SMILES: *N(C)C(=O)CC1CCOCC1] |
| 23 | 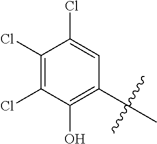 [SMILES: *c1cc(Cl)c(Cl)c(Cl)c1O] | 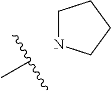 (4-position) | SO₂CH₃ | NH(C═O)CH₃ | H | 2 | 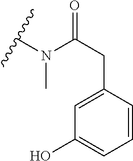 [SMILES: *N(C)C(=O)Cc1cccc(O)c1] |

[symbol]

indicates data missing or illegible when filed
TABLE 3

| No. | 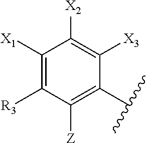 | 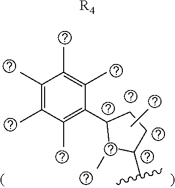 | R₅ | R [symbol] | R₂ | m | 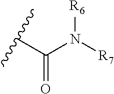 |
|---|---|---|---|---|---|---|---|
| 1a | 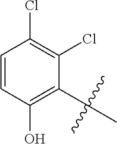 [SMILES: *c1c(O)ccc(Cl)c1Cl] | Me (5-position) | Me | OCH₂CH₃ | Et | 1 | 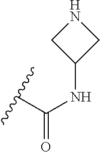 [SMILES: *C(=O)NC1CNC1] |
| 2a | 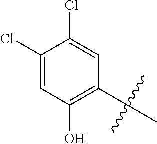 [SMILES: *c1cc(Cl)c(Cl)cc1O] | 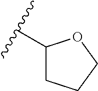 [SMILES: *C1CCCO1] (5-position) | 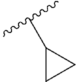 [SMILES: *C1CC1] | Et | Me | 2 | 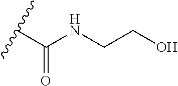 [SMILES: *C(=O)NCCO] |
| 3a | 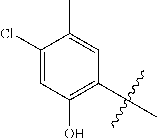 [SMILES: *c1cc(C)c(Cl)cc1O] | 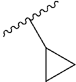 [SMILES: *C1CC1] (5-position) | 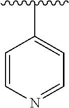 [SMILES: *c1ccncc1] | OCF₃ | Me | 3 | 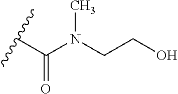 [SMILES: *C(=O)N(C)CCO] |
| 4a | 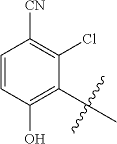 [SMILES: *c1c(O)ccc(C#N)c1Cl] | OH (5-position) | 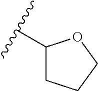 [SMILES: *C1CCCO1] | CN | H | 1 | 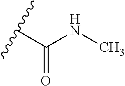 [SMILES: *C(=O)NC] |
| 5a | 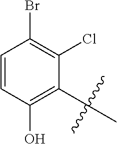 [SMILES: *c1c(O)ccc(Br)c1Cl] | CH₂CH₂OH (5-position) | 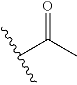 [SMILES: *C(C)=O] | SCH₂CH₃ | CF₃ | 2 | 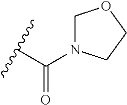 [SMILES: *C(=O)N1CCOC1] |
| 6a | 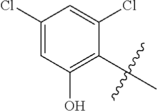 [SMILES: *c1c(O)cc(Cl)cc1Cl] | CH₂CH₂OCH₃ (5-position) | (C═O)CH₂OCH₃ | Cl | H | 3 | 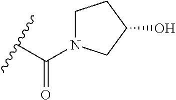 [SMILES: *C(=O)N1CC[C@H](O)C1] |
| 7a | 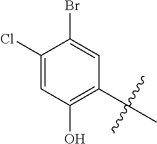 [SMILES: *c1cc(Br)c(Cl)cc1O] | 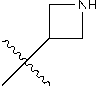 [SMILES: *C1CNC1] (5-position) | (C═O)CH₂NHCH₃ | NH [symbol] | Me | 1 | 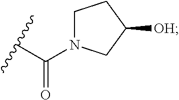 [SMILES: *C(=O)N1CC[C@@H](O)C1] |
| 8a | 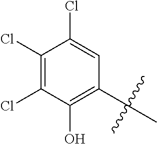 [SMILES: *c1cc(Cl)c(Cl)c(Cl)c1O] | 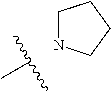 (5-position) | SO₂CH₃ | NH(C═O)CH₃ | H | 2 | 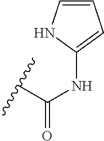 [SMILES: *C(=O)Nc1ccc[nH]1] |
| 9a | 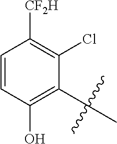 [SMILES: *c1c(O)ccc(C(F)F)c1Cl] | 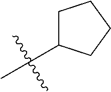 [SMILES: *C1CCCC1] (5-position) | 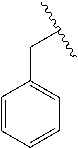 [SMILES: *Cc1ccccc1] | 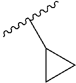 [SMILES: *C1CC1] | H | 3 | 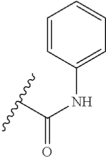 [SMILES: *C(=O)Nc1ccccc1] |
| 10a | 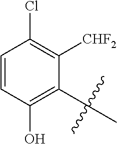 [SMILES: *c1c(O)ccc(Cl)c1C(F)F] | CH₂OCH₃ (5-position) | 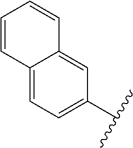 [SMILES: *c1ccc2ccccc2c1] | 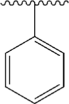 [SMILES: *c1ccccc1] | H | 1 | 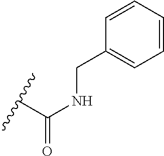 [SMILES: *C(=O)NCc1ccccc1] |
| 11a | 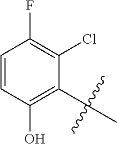 [SMILES: *c1c(O)ccc(F)c1Cl] | CH₂NHCH₃ (5-position) | 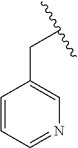 [SMILES: *Cc1cccnc1] | 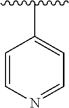 [SMILES: *c1ccncc1] | H | 2 | 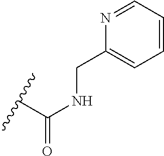 [SMILES: *C(=O)NCc1ccccn1] |
| 12a | 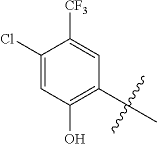 [SMILES: *c1cc(C(F)(F)F)c(Cl)cc1O] | Et (2-position) | CF [symbol] | 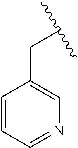 [SMILES: *Cc1cccnc1] | H | 3 | 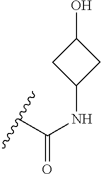 [SMILES: *C(=O)NC1CC(O)C1] |
| 13a | 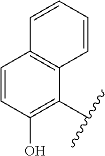 [SMILES: *c1c(O)ccc2ccccc12] | i-Pr (3-position) | CH₂CH₂OH | F | F | 1 | 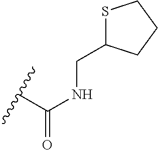 [SMILES: *C(=O)NCC1CCCS1] |
| 14a | 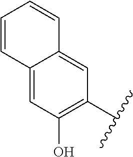 [SMILES: *c1cc2ccccc2cc1O] | Me (4-position) | (C═O)Et | 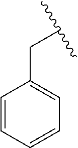 [SMILES: *Cc1ccccc1] | H | 2 | 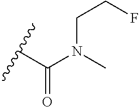 [SMILES: *C(=O)N(C)CCF] |
| 15a | 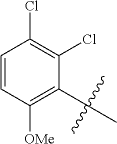 [SMILES: *c1c(OC)ccc(Cl)c1Cl] | Et (4-position) | Et | CH₂OH | H | 3 | 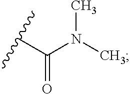 [SMILES: *C(=O)N(C)C] |

[symbol]

indicates data missing or illegible when filed
TABLE 4

| No. | 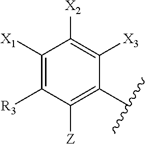 | 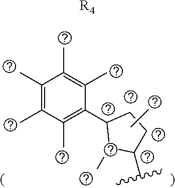 | R₅ | R [symbol] | R₂ | m | 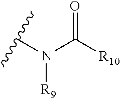 |
|---|---|---|---|---|---|---|---|
| 16a | 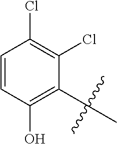 [SMILES: *c1c(O)ccc(Cl)c1Cl] | Me (5-position) | Me | OCH₂CH₃ | Et | 1 | 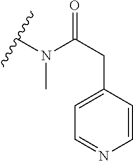 [SMILES: *N(C)C(=O)Cc1ccncc1] |
| 17a | 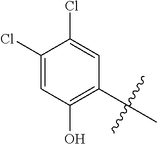 [SMILES: *c1cc(Cl)c(Cl)cc1O] | 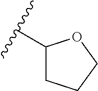 [SMILES: *C1CCCO1] (5-position) | 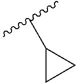 [SMILES: *C1CC1] | Et | Me | 2 | 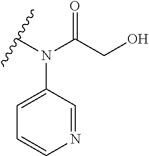 [SMILES: *N(C(=O)CO)c1cccnc1] |
| 18a | 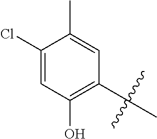 [SMILES: *c1cc(C)c(Cl)cc1O] | 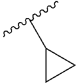 [SMILES: *C1CC1] (5-position) | 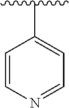 [SMILES: *c1ccncc1] | OCF₃ | Me | 3 | 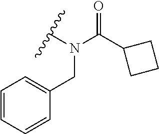 [SMILES: *N(Cc1ccccc1)C(=O)C1CCC1] |
| 19a | 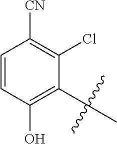 [SMILES: *c1c(O)ccc(C#N)c1Cl] | OH (5-position) | 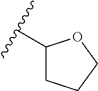 [SMILES: *C1CCCO1] | CN | H | 1 | 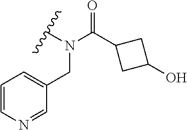 [SMILES: *N(Cc1cccnc1)C(=O)C1CC(O)C1] |
| 20a | 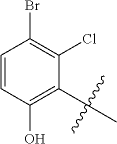 [SMILES: *c1c(O)ccc(Br)c1Cl] | CH₂CH₂OH (5-position) | 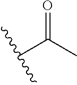 [SMILES: *C(C)=O] | SCH [symbol] CH [symbol] | CF₃ | 2 | 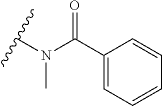 [SMILES: *N(C)C(=O)c1ccccc1] |
| 21a | 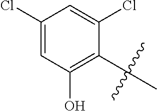 [SMILES: *c1c(O)cc(Cl)cc1Cl] | CH₂CH₂OCH₃ (5-position) | (C═O)CH₂OCH₃ | Cl | H | 3 | 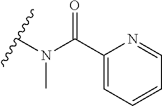 [SMILES: *N(C)C(=O)c1ccccn1] |
| 22a | 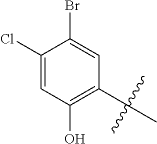 [SMILES: *c1cc(Br)c(Cl)cc1O] | 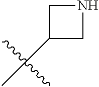 [SMILES: *C1CNC1] (5-position) | (C═O)CH₂NHCH₃ | NH₂ | Me | 1 | 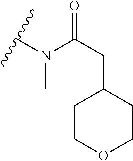 [SMILES: *N(C)C(=O)CC1CCOCC1] |
| 23a | 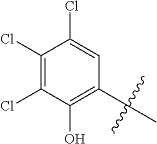 [SMILES: *c1cc(Cl)c(Cl)c(Cl)c1O] | 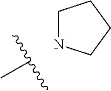 (5-position) | SO₂CH₃ | NH(C═O)CH₃ | H | 2 | 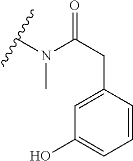 [SMILES: *N(C)C(=O)Cc1cccc(O)c1] |
| 24a | 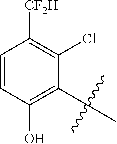 [SMILES: *c1c(O)ccc(C(F)F)c1Cl] | 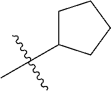 [SMILES: *C1CCCC1] (5-position) | 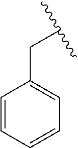 [SMILES: *Cc1ccccc1] | 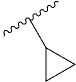 [SMILES: *C1CC1] | H | 3 | 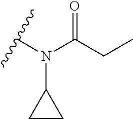 [SMILES: *N(C(=O)CC)C1CC1] |
| 25a | 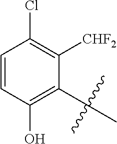 [SMILES: *c1c(O)ccc(Cl)c1C(F)F] | CH₂OCH₃ (5-position) | 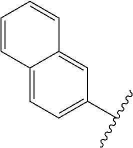 [SMILES: *c1ccc2ccccc2c1] | 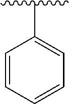 [SMILES: *c1ccccc1] | H | 1 | 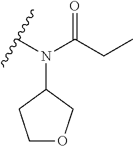 [SMILES: *N(C(=O)CC)C1CCOC1] |
| 26a | 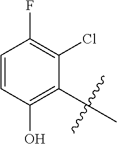 [SMILES: *c1c(O)ccc(F)c1Cl] | CH₂NHCH₃ (5-position) | 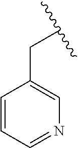 [SMILES: *Cc1cccnc1] | 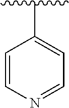 [SMILES: *c1ccncc1] | H | 2 | 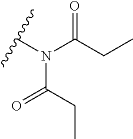 [SMILES: *N(C(=O)CC)C(=O)CC] |
| 27a | 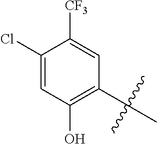 [SMILES: *c1cc(C(F)(F)F)c(Cl)cc1O] | Et (2-position) | CF₃ | 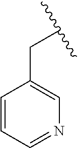 [SMILES: *Cc1cccnc1] | H | 3 | 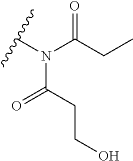 [SMILES: *N(C(=O)CC)C(=O)CCO] |
| 28a | 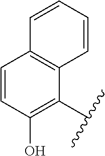 [SMILES: *c1c(O)ccc2ccccc12] | i-Pr (3-position) | CH₂CH₂OH | F | F | 1 | 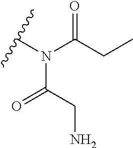 [SMILES: *N(C(=O)CC)C(=O)CN] |
| 29a | 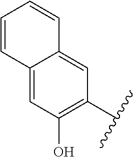 [SMILES: *c1cc2ccccc2cc1O] | Me (4-position) | (C═O)Et | 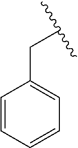 [SMILES: *Cc1ccccc1] | H | 2 | 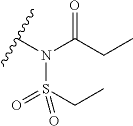 [SMILES: *N(C(=O)CC)S(=O)(=O)CC] |
| 30a | 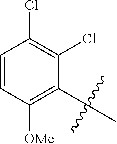 [SMILES: *c1c(OC)ccc(Cl)c1Cl] | Et (4-position) | Et | CH₂OH | H | 3 | 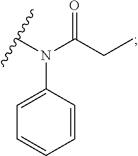 [SMILES: *N(C(=O)CC)c1ccccc1] |

[symbol]

indicates data missing or illegible when filed
TABLE 5

| No. | 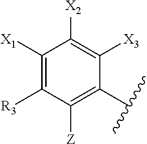 | 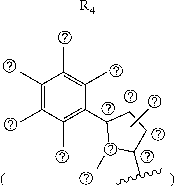 | R₅ | R [symbol] | R₂ | m | 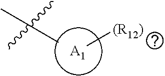 |
|---|---|---|---|---|---|---|---|
| 1b | 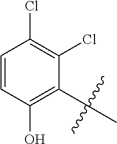 [SMILES: *c1c(O)ccc(Cl)c1Cl] | Me (4-position) | Me | OCH₂CH₃ | Et | 1 | 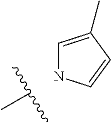 |
| 2b | 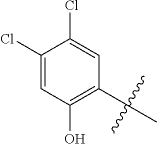 [SMILES: *c1cc(Cl)c(Cl)cc1O] | 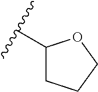 [SMILES: *C1CCCO1] (4-position) | 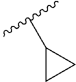 [SMILES: *C1CC1] | Et | Me | 2 | 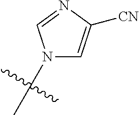 [SMILES: *n1cnc(C#N)c1] |
| 3b | 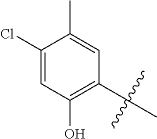 [SMILES: *c1cc(C)c(Cl)cc1O] | 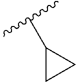 [SMILES: *C1CC1] (4-position) | 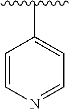 [SMILES: *c1ccncc1] | OCF₃ | Me | 3 | 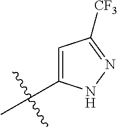 [SMILES: *c1cc(C(F)(F)F)n[nH]1] |
| 4b | 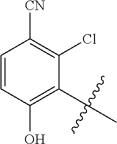 [SMILES: *c1c(O)ccc(C#N)c1Cl] | OH (4-position) | 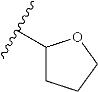 [SMILES: *C1CCCO1] | CN | H | 1 | 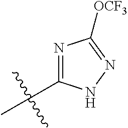 [SMILES: *c1nc(OC(F)(F)F)n[nH]1] |
| 5b | 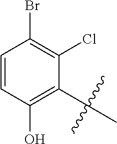 [SMILES: *c1c(O)ccc(Br)c1Cl] | CH₂CH₂OH (4-position) | 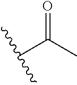 [SMILES: *C(C)=O] | SCH₂CH₃ | CF₃ | 2 | 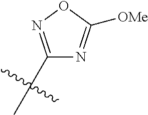 [SMILES: *c1noc(OC)n1] |
| 6b | 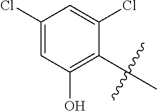 [SMILES: *c1c(O)cc(Cl)cc1Cl] | CH₂CH₂OCH₃ (4-position) | (C═O)CH₂OCH₃ | Cl | H | 3 | 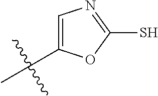 [SMILES: *c1cnc(S)o1] |
| 7b | 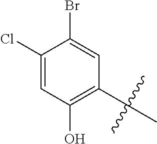 [SMILES: *c1cc(Br)c(Cl)cc1O] | 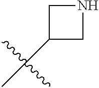 [SMILES: *C1CNC1] (4-position) | (C═O)CH [symbol] NHCH₃ | NH [symbol] | Me | 1 | 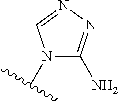 [SMILES: *n1cnnc1N] |
| 8b | 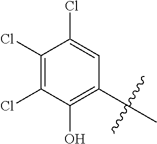 [SMILES: *c1cc(Cl)c(Cl)c(Cl)c1O] | 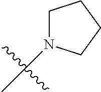 [SMILES: *N1CCCC1] (4-position) | SO₂CH₃ | NH(C═O)CH₃ | H | 2 | 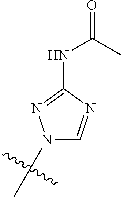 [SMILES: *n1cnc(NC(C)=O)n1] |
| 9b | 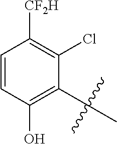 [SMILES: *c1c(O)ccc(C(F)F)c1Cl] | 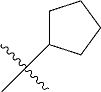 [SMILES: *C1CCCC1] (4-position) | 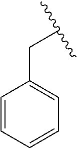 [SMILES: *Cc1ccccc1] | 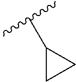 [SMILES: *C1CC1] | H | 3 | 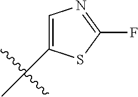 [SMILES: *c1cnc(F)s1] |
| 10b | 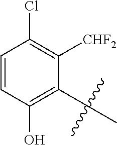 [SMILES: *c1c(O)ccc(Cl)c1C(F)F] | CH [symbol] OCH₃ (4-position) | 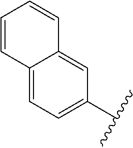 [SMILES: *c1ccc2ccccc2c1] | 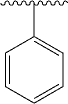 [SMILES: *c1ccccc1] | H | 1 | 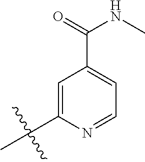 [SMILES: *c1cc(C(=O)NC)ccn1] |
| 11b | 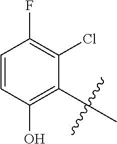 [SMILES: *c1c(O)ccc(F)c1Cl] | CH [symbol] NHCH [symbol] (4-position) | 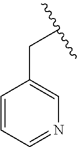 [SMILES: *Cc1cccnc1] | 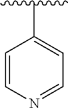 [SMILES: *c1ccncc1] | H | 2 | 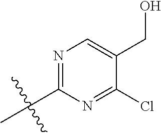 [SMILES: *c1ncc(CO)c(Cl)n1] |
| 12b | 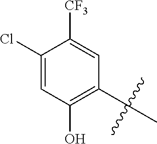 [SMILES: *c1cc(C(F)(F)F)c(Cl)cc1O] | Et (2-position) | CF₃ | 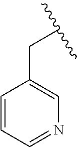 [SMILES: *Cc1cccnc1] | H | 3 | 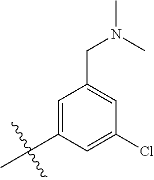 [SMILES: *c1cc(Cl)cc(CN(C)C)c1] |
| 13b | 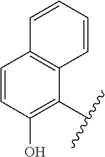 [SMILES: *c1c(O)ccc2ccccc12] | i-Pr (3-position) | CH [symbol] CH [symbol] OH | F | F | 1 | 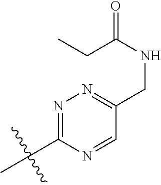 [SMILES: *c1ncc(CNC(=O)CC)nn1] |
| 14b | 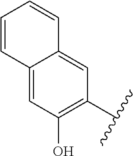 [SMILES: *c1cc2ccccc2cc1O] | Me (5-position) | (C═O)Et | 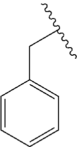 [SMILES: *Cc1ccccc1] | H | 2 | 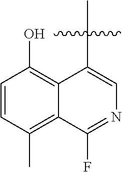 [SMILES: *c1cnc(F)c2c(C)ccc(O)c12] |
| 15b | 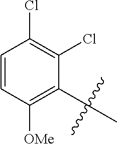 [SMILES: *c1c(OC)ccc(Cl)c1Cl] | Et (5-position) | Et | CH₂OH | H | 3 | 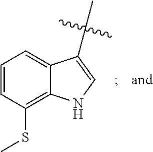 [SMILES: *c1c[nH]c2c(SC)cccc12] |

[symbol]

indicates data missing or illegible when filed
TABLE 6

| No. | 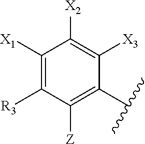 | 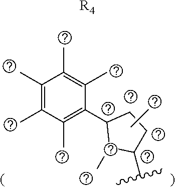 | R₅ | R [symbol] | R₂ | m | 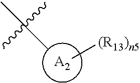 |
|---|---|---|---|---|---|---|---|
| 16b | 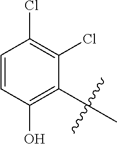 [SMILES: *c1c(O)ccc(Cl)c1Cl] | Me (5-position) | Me | CH [symbol] CH [symbol] | Et | 1 | 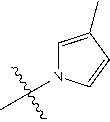 [SMILES: *n1ccc(C)c1] |
| 17b | 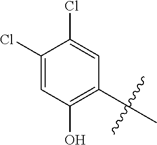 [SMILES: *c1cc(Cl)c(Cl)cc1O] | 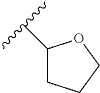 [SMILES: *C1CCCO1] (5-position) | 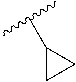 [SMILES: *C1CC1] | Et | Me | 2 | 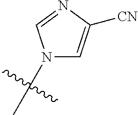 [SMILES: *n1cnc(C#N)c1] |
| 18b | 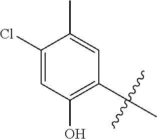 [SMILES: *c1cc(C)c(Cl)cc1O] | 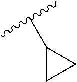 [SMILES: *C1CC1] (5-position) | 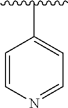 [SMILES: *c1ccncc1] | OCF₃ | Me | 3 | 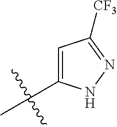 [SMILES: *c1cc(C(F)(F)F)n[nH]1] |
| 19b | 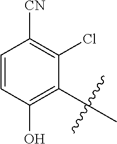 [SMILES: *c1c(O)ccc(C#N)c1Cl] | OH (5-position) | 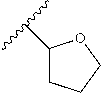 [SMILES: *C1CCCO1] | CN | H | 1 | 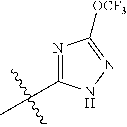 [SMILES: *c1nc(OC(F)(F)F)n[nH]1] |
| 20b | 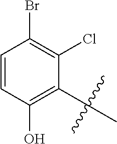 [SMILES: *c1c(O)ccc(Br)c1Cl] | CH₂CH₂OH (5-position) | 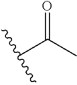 [SMILES: *C(C)=O] | SCH [symbol] CH [symbol] | CF₃ | 2 | 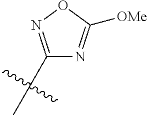 [SMILES: *c1noc(OC)n1] |
| 21b | 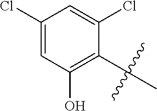 [SMILES: *c1c(O)cc(Cl)cc1Cl] | CH₂CH₂OCH₃ (5-position) | (C═O)CH₂OCH₃ | Cl | H | 3 | 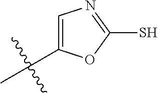 [SMILES: *c1cnc(S)o1] |
| 22b | 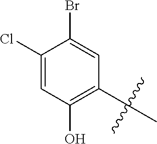 [SMILES: *c1cc(Br)c(Cl)cc1O] | 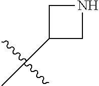 [SMILES: *C1CNC1] (5-position) | (C═O)CH₂NHCH₃ | NH₂ | Me | 1 | 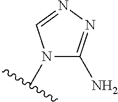 [SMILES: *n1cnnc1N] |
| 23b | 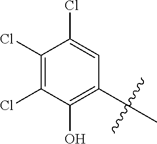 [SMILES: *c1cc(Cl)c(Cl)c(Cl)c1O] | 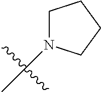 [SMILES: *N1CCCC1] (5-position) | SO₂CH₃ | NH(C═O)CH₃ | H | 2 | 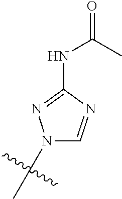 [SMILES: *n1cnc(NC(C)=O)n1] |
| 24b | 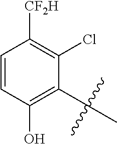 [SMILES: *c1c(O)ccc(C(F)F)c1Cl] | 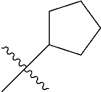 [SMILES: *C1CCCC1] (5-position) | 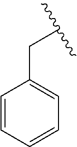 [SMILES: *Cc1ccccc1] | 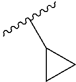 [SMILES: *C1CC1] | H | 3 | 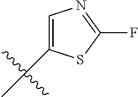 [SMILES: *c1cnc(F)s1] |
| 25b | 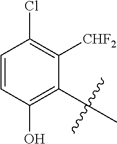 [SMILES: *c1c(O)ccc(Cl)c1C(F)F] | CH₂OCH₃ (5-position) | 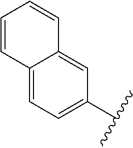 [SMILES: *c1ccc2ccccc2c1] | 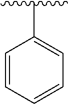 [SMILES: *c1ccccc1] | H | 1 | 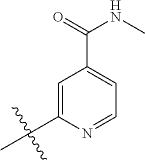 [SMILES: *c1cc(C(=O)NC)ccn1] |
| 26b | 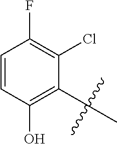 [SMILES: *c1c(O)ccc(F)c1Cl] | CH₂NHCH₃ (5-position) | 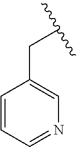 [SMILES: *Cc1cccnc1] | 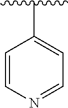 [SMILES: *c1ccncc1] | H | 2 | 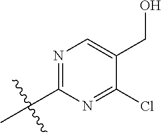 [SMILES: *c1ncc(CO)c(Cl)n1] |
| 27b | 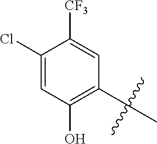 [SMILES: *c1cc(C(F)(F)F)c(Cl)cc1O] | Et (2-position) | CF₃ | 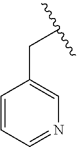 [SMILES: *Cc1cccnc1] | H | 3 | 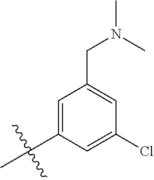 [SMILES: *c1cc(Cl)cc(CN(C)C)c1] |
| 28b | 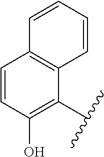 [SMILES: *c1c(O)ccc2ccccc12] | i-Pr (3-position) | CH₂CH₂OH | F | F | 1 | 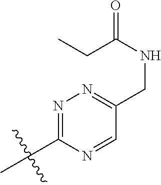 [SMILES: *c1ncc(CNC(=O)CC)nn1] |
| 29b | 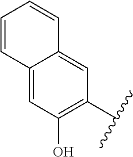 [SMILES: *c1cc2ccccc2cc1O] | Me (4-position) | (C═O)Et | 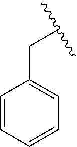 [SMILES: *Cc1ccccc1] | H | 2 | 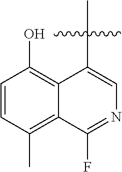 [SMILES: *c1cnc(F)c2c(C)ccc(O)c12] |
| 30b | 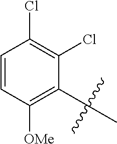 [SMILES: *c1c(OC)ccc(Cl)c1Cl] | Et (4-position) | Et | CH₂OH | H | 3 | 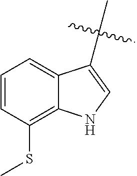 [SMILES: *c1c[nH]c2c(SC)cccc12] |

[symbol]

indicates data missing or illegible when filed
or a pharmaceutically-acceptable salt thereof.

85. A pharmaceutical composition comprising at least one compound of Formula I, I′, II, II′, III, or IV, or a pharmaceutically-acceptable salt thereof, 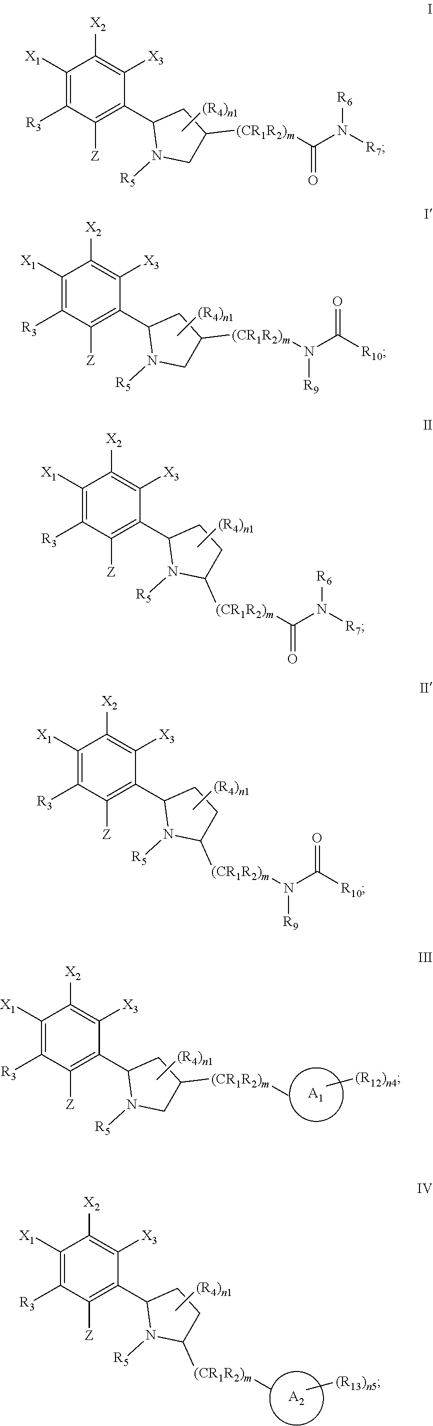
wherein
each occurrence of Z is independently ORₐ;
each occurrence of X₁ is independently H, halogen, CN, alkyl, cycloalkyl, halogenated cycloalkyl, or halogenated alkyl;
each occurrence of X₂ is independently H, halogen, CN, alkyl, cycloalkyl, halogenated cycloalkyl, or halogenated alkyl;
each occurrence of X₃ is independently H, halogen, CN, alkyl, cycloalkyl, halogenated cycloalkyl, or halogenated alkyl;
or alternatively X₁ and X₂ and the carbon atoms they are connected to taken together form an optionally substituted 5- or 6-membered aryl;
or alternatively X₂ and X₃ and the carbon atoms they are connected to taken together form an optionally substituted 5- or 6-membered aryl;
each occurrence of R₁ is independently H, alkyl, cycloalkyl, saturated heterocycle, aryl, heteroaryl, alkylaryl, alkylheteroaryl, CN, CF₃, OCF₃, ORₐ, SRₐ, halogen, NRₐR_{b}, or NR_{b}(C═O)Rₐ;
each occurrence of R₂ is independently H, alkyl, cycloalkyl, saturated heterocycle, aryl, heteroaryl, alkylaryl, alkylheteroaryl, CN, CF₃, OCF₃, ORₐ, SRₐ, halogen, NRₐR_{b}, or NR_{b}(C═O)Rₐ;
or alternatively R₁ and R₂ taken together with the carbon atom they are connected to form a cycloalkyl or saturated heterocycle:
each occurrence of R₃ is independently H, alkyl, cycloalkyl, saturated heterocycle, aryl, heteroaryl, CN, CF₃, OCF₃, ORₐ, SRₐ, halogen, NRₐR_{b}, or NR_{b}(C═O)Rₐ;
each occurrence of R₄ is independently H, alkyl, cycloalkyl, saturated heterocycle, (CRₐR_{b})ₙ₂ORₐ, or (CRₐR_{b})ₙ₂NRₐR_{b}:
or alternatively two R₄ groups taken together with the carbon atom(s) that they are connected to form a 3-7 membered optionally substituted cycloalkyl or heterocycle:
each occurrence of R₅ is independently H, alkyl, cycloalkyl, saturated heterocycle, aryl, heteroaryl, alkylaryl, alkylheteroaryl, (C═O)Rₐ, (C═O)(CRₐR_{b})ₙ₂ORₐ, (C═O)(CRₐR_{b})ₙ₂NRₐR_{b}, or SO₂Rₐ,
each occurrence of R₆ is independently H, alkyl, cycloalkyl, heterocycle, aryl, heteroaryl, alkylaryl, or alkylheteroaryl;
each occurrence of R₇ is independently H, alkyl, cycloalkyl, heterocycle, aryl, heteroaryl, alkylaryl, or alkylheteroaryl;
or alternatively R₆ and R₇ taken together with the nitrogen atom they are connected to form a heterocycle comprising the nitrogen atom and 0-3 additional heteroatoms each selected from the group consisting of N, O, and S; wherein the heterocycle is optionally substituted by 1-4 substituents each independently selected from the group consisting of alkyl, cycloalkyl, halogenated cycloalkyl, halogenated alkyl, halogen, CN, OR₈, —(CH₂)₀₋₂OR₈, N(R₈)₂, (C═O)R₈, (C═O)N(R₈)₂, NR₈(C═O)R₈, and oxo where valence permits;
each occurrence of R₉ is independently H, alkyl, cycloalkyl, saturated heterocycle, aryl, heteroaryl, alkylaryl, alkylheteroaryl, (C═O)Rₐ, (C═O)(CRₐR_{b})ₙ₂ORₐ, (C═O)(CRₐR_{b})ₙ₂NRₐR_{b}, or SO₂Rₐ;
each occurrence of R₁₀ is independently H, alkyl, cycloalkyl, heterocycle, aryl, heteroaryl, alkylaryl, or alkylheteroaryl;
A₁ is aryl or heteroaryl;
A₂ is aryl or heteroaryl;
each occurrence of R₁₂ is independently H, alkyl, CN, CF₃, OCF₃, ORₐ, SRₐ, halogen, NRₐR_{b}, (CRₐR_{b})ₙ₂ORₐ, (C═O)NRₐR_{b}, (CRₐR_{b})ₙ₂NRₐR_{b}, or (CRₐR_{b})ₙ₂NR_{b}(C═O)Rₐ:
each occurrence of R₁₃ is independently H, alkyl, CN, CF₃, OCF₃, ORₐ, SRₐ, halogen, NRₐR_{b}, (CRₐR_{b})ₙ₂ORₐ, (C═O)NRₐR_{b}, (CRₐR_{b})ₙ₂NRₐR_{b}, or (CRₐR_{b})ₙ₂NR_{b}(C═O)Rₐ:
each occurrence of Rₐ and R_{b} are independently H, alkyl, alkenyl, cycloalkyl, saturated heterocycle comprising 1-3 heteroatoms each selected from the group consisting of N, O, and S, aryl, or heteroaryl; or alternatively Rₐ and R_{b} together with the carbon or nitrogen atom that they are connected to form a cycloalkyl or heterocycle comprising the nitrogen atom and 0-3 additional heteroatoms each selected from the group consisting of N, O, and S;
the alkyl, cycloalkyl, heterocycle, aryl, and heteroaryl in X₁, X₂, X₃, A₁, A₂, R₁, R₂, R₃, R₄, R₅, R₆, R₇, R₉, R₁₀, R₁₂, R₁₃, Rₐ, or R_{b}, where applicable, are optionally substituted by 1-4 substituents each independently selected from the group consisting of alkyl, cycloalkyl, halogenated cycloalkyl, halogenated alkyl, halogen, CN, OR₈, —(CH₂)₀₋₂OR₈, N(R₈)₂, (C═O)R₈, (C═O)N(R₈)₂, NR₈(C═O)R₈, and oxo where valence permits;
each occurrence of R₈ is independently H, alkyl, or optionally substituted heterocycle wherein the heterocycle is optionally substituted by alkyl, halogen, or OH: or alternatively the two R₈ groups together with the nitrogen atom that they are connected to form an optionally substituted heterocycle, wherein the heterocycle is optionally substituted by alkyl, halogen, or OH, comprising the nitrogen atom and 0-3 additional heteroatoms each selected from the group consisting of N, O, and S;
each occurrence of m is independently 1, 2, or 3:
each occurrence of n₁ is independently an integer from 0-3 wherein valence permits:
each occurrence of n₂ is independently an integer from 0-3:
n₄ is an integer from 0-3; and
n₅ is an integer from 0-3:
and a pharmaceutically-acceptable carrier or diluent.

86. A method of treating a condition in a mammalian species in need thereof, comprising administering to the mammalian species a therapeutically effective amount of at least one compound of Formula I, I′, II, II′, III, or IV, or a pharmaceutically-acceptable salt thereof, 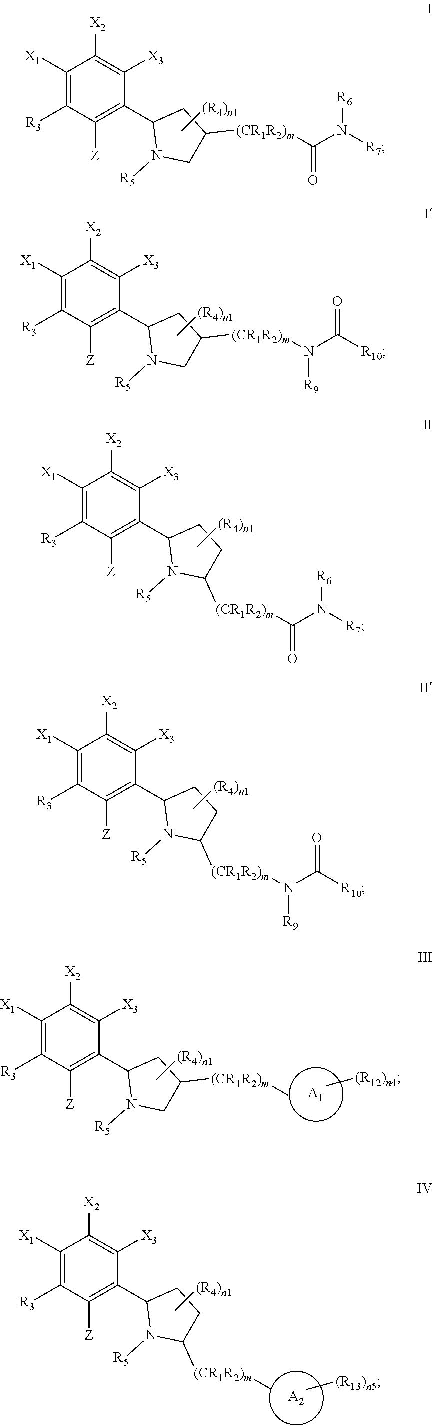
wherein
each occurrence of Z is independently ORₐ;
each occurrence of X₁ is independently H, halogen, CN, alkyl, cycloalkyl, halogenated cycloalkyl, or halogenated alkyl;
each occurrence of X₂ is independently H, halogen, CN, alkyl, cycloalkyl, halogenated cycloalkyl, or halogenated alkyl;
each occurrence of X₃ is independently H, halogen, CN, alkyl, cycloalkyl, halogenated cycloalkyl, or halogenated alkyl;
or alternatively X₁ and X₂ and the carbon atoms they are connected to taken together form an optionally substituted 5- or 6-membered aryl;
or alternatively X₂ and X₃ and the carbon atoms they are connected to taken together form an optionally substituted 5- or 6-membered aryl;
each occurrence of R₁ is independently H, alkyl, cycloalkyl, saturated heterocycle, aryl, heteroaryl, alkylaryl, alkylheteroaryl, CN, CF₃, OCF₃, ORₐ, SRₐ, halogen, NRₐR_{b}, or NR_{b}(C═O)Rₐ;
each occurrence of R₂ is independently H, alkyl, cycloalkyl, saturated heterocycle, aryl, heteroaryl, alkylaryl, alkylheteroaryl, CN, CF₃, OCF₃, ORₐ, SRₐ, halogen, NRₐR_{b}, or NR_{b}(C═O)Rₐ;
or alternatively R₁ and R₂ taken together with the carbon atom they are connected to form a cycloalkyl or saturated heterocycle:
each occurrence of R₃ is independently H, alkyl, cycloalkyl, saturated heterocycle, aryl, heteroaryl, CN, CF₃, OCF₃, ORₐ, SRₐ, halogen, NRₐR_{b}, or NR_{b}(C═O)Rₐ;
each occurrence of R₄ is independently H, alkyl, cycloalkyl, saturated heterocycle, (CRₐR_{b})ₙ₂ORₐ, or (CRₐR_{b})ₙ₂NRₐR_{b}:
or alternatively two R₄ groups taken together with the carbon atom(s) that they are connected to form a 3-7 membered optionally substituted cycloalkyl or heterocycle:
each occurrence of R₅ is independently H, alkyl, cycloalkyl, saturated heterocycle, aryl, heteroaryl, alkylaryl, alkylheteroaryl, (C═O)Rₐ, (C═O)(CRₐR_{b})ₙ₂ORₐ, (C═O)(CRₐR_{b})ₙ₂NRₐR_{b}, or SO₂Rₐ;
each occurrence of R₆ is independently H, alkyl, cycloalkyl, heterocycle, aryl, heteroaryl, alkylaryl, or alkylheteroaryl;
each occurrence of R₇ is independently H, alkyl, cycloalkyl, heterocycle, aryl, heteroaryl, alkylaryl, or alkylheteroaryl;
or alternatively R₆ and R₇ taken together with the nitrogen atom they are connected to form a heterocycle comprising the nitrogen atom and 0-3 additional heteroatoms each selected from the group consisting of N, O, and S; wherein the heterocycle is optionally substituted by 1-4 substituents each independently selected from the group consisting of alkyl, cycloalkyl, halogenated cycloalkyl, halogenated alkyl, halogen, CN, OR₈, —(CH₂)₀₋₂OR₈, N(R₈)₂, (C═O)R₈, (C═O)N(R₈)₂, NR₈(C═O)R₈, and oxo where valence permits;
each occurrence of R₉ is independently H, alkyl, cycloalkyl, saturated heterocycle, aryl, heteroaryl, alkylaryl, alkylheteroaryl, (C═O)Rₐ, (C═O)(CRₐR_{b})ₙ₂ORₐ, (C═O)(CRₐR_{b})ₙ₂NRₐR_{b}, or SO₂Rₐ;
each occurrence of R₁₀ is independently H, alkyl, cycloalkyl, heterocycle, aryl, heteroaryl, alkylaryl, or alkylheteroaryl;
A₁ is aryl or heteroaryl;
A₂ is aryl or heteroaryl;
each occurrence of R₁₂ is independently H, alkyl, CN, CF₃, OCF₃, ORₐ, SRₐ, halogen, NRₐR_{b}, (CRₐR_{b})ₙ₂ORₐ, (C═O)NRₐR_{b}, (CRₐR_{b})ₙ₂NRₐR_{b}, or (CRₐR_{b})ₙ₂NR_{b}(C═O)Rₐ:
each occurrence of R₁₃ is independently H, alkyl, CN, CF₃, OCF₃, ORₐ, SRₐ, halogen, NRₐR_{b}, (CRₐR_{b})ₙ₂ORₐ, (C═O)NRₐR_{b}, (CRₐR_{b})ₙ₂NRₐR_{b}, or (CRₐR_{b})ₙ₂NR_{b}(C═O)Rₐ:
each occurrence of Rₐ and R_{b} are independently H, alkyl, alkenyl, cycloalkyl, saturated heterocycle comprising 1-3 heteroatoms each selected from the group consisting of N, O, and S, aryl, or heteroaryl; or alternatively Rₐ and R_{b} together with the carbon or nitrogen atom that they are connected to form a cycloalkyl or heterocycle comprising the nitrogen atom and 0-3 additional heteroatoms each selected from the group consisting of N, O, and S;
the alkyl, cycloalkyl, heterocycle, aryl, and heteroaryl in X₁, X₂, X₃, A₁, A₂, R₁, R₂, R₃, R₄, R₅, R₆, R₇, R₉, R₁₀, R₁₂, R₁₃, Rₐ, or R_{b}, where applicable, are optionally substituted by 1-4 substituents each independently selected from the group consisting of alkyl, cycloalkyl, halogenated cycloalkyl, halogenated alkyl, halogen, CN, OR₈, —(CH₂)₀₋₂OR₈, N(R₈)₂, (C═O)R₈, (C═O)N(R₈)₂, NR₈(C═O)R₈, and oxo where valence permits;
each occurrence of R₈ is independently H, alkyl, or optionally substituted heterocycle wherein the heterocycle is optionally substituted by alkyl, halogen, or OH: or alternatively the two R₈ groups together with the nitrogen atom that they are connected to form an optionally substituted heterocycle, wherein the heterocycle is optionally substituted by alkyl, halogen, or OH, comprising the nitrogen atom and 0-3 additional heteroatoms each selected from the group consisting of N, O, and S;
each occurrence of m is independently 1, 2, or 3:
each occurrence of n₁ is independently an integer from 0-3 wherein valence permits:
each occurrence of n₂ is independently an integer from 0-3:
n₄ is an integer from 0-3; and
n₅ is an integer from 0-3,
wherein the condition is selected from the group consisting of cancer, an immunological disorder, a central nervous system disorder, an inflammatory disorder, a gastroenterological disorder, a metabolic disorder, a cardiovascular disorder, and a kidney disease and wherein the mammalian species is human.

87. (canceled)

88. The method of claim 86, wherein the autoimmune disease is rheumatoid arthritis, multiple sclerosis, systemic lupus erythematosus, or type I diabetes mellitus.

89. (canceled)

90. The method of claim 86, wherein the inflammatory disorder is an inflammatory skin condition, arthritis, psoriasis, spondylitis, parodontitits, or an inflammatory neuropathy.

91. (canceled)

92. (canceled)

93. (canceled)

94. (canceled)

95. The method of claim 86, wherein the condition is selected from the group consisting of cancer, transplant rejection, rheumatoid arthritis, multiple sclerosis, systemic lupus erythematosus, type I diabetes mellitus, Alzheimer's disease, inflammatory skin condition, inflammatory neuropathy, psoriasis, spondylitis, parodontitis, Crohn's disease, ulcerative colitis, obesity, type II diabetes mellitus, ischemic stroke, chronic kidney disease, nephritis, chronic renal failure, and a combination thereof.

96. (canceled)

97. (canceled)

98. (canceled)

99. The compound of claim 1, wherein the compound is 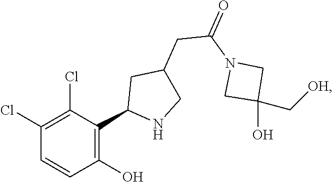 [SMILES: O=C(CC1CN[C@@H](c2c(O)ccc(Cl)c2Cl)C1)N1CC(O)(CO)C1]
or a pharmaceutically-acceptable salt thereof.

100. The compound of claim 1, wherein the compound is 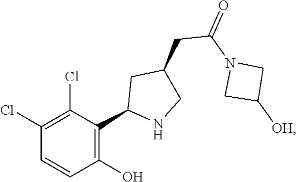 [SMILES: O=C(C[C@H]1CN[C@@H](c2c(O)ccc(Cl)c2Cl)C1)N1CC(O)C1]
or a pharmaceutically-acceptable salt thereof.

101. The compound of claim 1, wherein the compound is 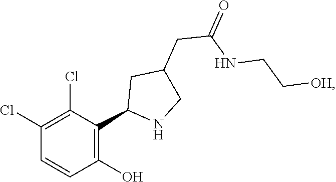 [SMILES: O=C(CC1CN[C@@H](c2c(O)ccc(Cl)c2Cl)C1)NCCO]
or a pharmaceutically-acceptable salt thereof.

102. The compound of claim 1, wherein the compound is 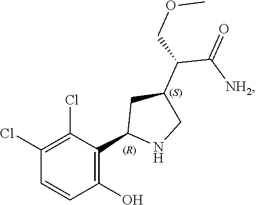
or a pharmaceutically-acceptable salt thereof.

103. The compound of claim 1, wherein the compound is 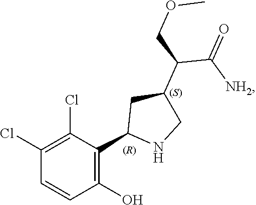
or a pharmaceutically-acceptable salt thereof.

104. The compound of claim 1, wherein the compound is 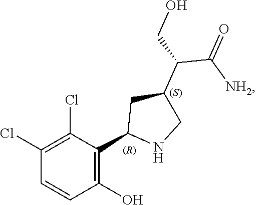
or a pharmaceutically-acceptable salt thereof.

105. The compound of claim 1, wherein the compound is 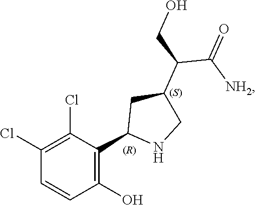
or a pharmaceutically-acceptable salt thereof.
